(19) United States
(12) Patent Application Publication
(10) Pub. No.: US 2013/0091598 A1
(43) Pub. Date: Apr. 11, 2013

(54) Nematode-Resistant Transgenic Plants

(71) Applicants: Aaron Wiig, Chapel Hill, NC (US); Bonnie C. McCaig, Durham, NC (US)

(72) Inventors: Aaron Wiig, Chapel Hill, NC (US); Bonnie C. McCaig, Durham, NC (US)

(73) Assignee: BASF Plant Science Company GmbH, Ludwigshafen (DE)

(21) Appl. No.: 13/580,458

(22) PCT Filed: Feb. 16, 2011
(86) PCT No.: PCT/EP2011/052285

§ 371 Date: Aug. 22, 2012

Related U.S. Application Data
(60) Provisional application No. 61/307,013, filed on Feb. 23, 2010.

(51) Int. Cl. C12N 15/82; A01H 5/00

(52) U.S. Cl. 800/279; 435/320.1; 800/301

(57) ABSTRACT

The present invention concerns double stranded RNA compositions and transgenic plants capable of inhibiting expression of plants genes, and methods associated therewith. Specifically, the invention relates to the use of RNA interference to inhibit expression of a target plant gene which is a plant a CLASP1 gene, an Aspartic Proteinase Delta Subunit gene, a Secreted Protein 1 gene, a Lectin Receptor Kinase-like gene, a Pectin Methylesterase-like gene, and an N PY1 gene, and relates to the generation of plants that have increased resistance to parasitic nematodes.

The field of this invention is the control of nematodes, in particular the control of soybean cyst nematodes. The invention also relates to the introduction of genetic material into plants that are susceptible to nematodes in order to increase resistance to nematodes.

### BACKGROUND OF THE INVENTION

Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore pathogenic on a large number of crop species. The cyst- and lesion-forming nematode species have a more limited host range, but still cause considerable losses in susceptible crops.

Pathogenic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. However, nematode infestation can cause significant yield losses without any obvious above-ground disease symptoms. The primary causes of yield reduction are due to root damage underground. Roots infected by SCN are dwarfed or stunted. Nematode infestation also can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

The life cycle of SCN has been the subject of many studies, and as such are a useful example for understanding the nematode life cycle. After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN nematodes, which are not swollen as adults, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. The promoters of these plant target genes can then be used to direct the specific expression of detrimental proteins or enzymes, or the expression of antisense RNA to the target gene or to general cellular genes. The plant promoters may also be used to confer nematode resistance specifically at the feeding site by transforming the plant with a construct comprising the promoter of the plant target gene linked to a gene whose product induces lethality in the nematode after ingestion.

Recently, RNA interference (RNAi), also referred to as gene silencing, has been proposed as a method for controlling nematodes. When double-stranded RNA (dsRNA) corresponding essentially to the sequence of a target gene or mRNA is introduced into a cell, expression from the target gene is inhibited (See e.g., U.S. Pat. No. 6,506,559). U.S. Pat. No. 6,506,559 demonstrates the effectiveness of RNAi against known genes in Caenorhabditis elegans, but does not demonstrate the usefulness of RNAi for controlling plant parasitic nematodes.

Use of RNAi to target essential nematode genes has been proposed, for example, in PCT Publication WO 01/96584, WO 01/17654, US 2004/0098761, US 2005/0091713, US 2005/0188438, US 2006/0037101, US 2006/0080749, US 2007/0199100, and US 2007/0250947.

A number of models have been proposed for the action of RNAi. In mammalian systems, dsRNAs larger than 30 nucleotides trigger induction of interferon synthesis and a global shut-down of protein syntheses, in a non-sequence-specific manner. However, U.S. Pat. No. 6,506,559 discloses that in nematodes, the length of the dsRNA corresponding to the target gene sequence may be at least 25, 50, 100, 200, 300, or 400 bases, and that even larger dsRNAs were also effective at inducing RNAi in C. elegans. It is known that when hairpin RNA constructs comprising double stranded regions ranging from 98 to 854 nucleotides were transformed into a number of plant species, the target plant genes were efficiently silenced. There is general agreement that in many organisms, including nematodes and plants, large pieces of dsRNA are cleaved into about 19-24 nucleotide fragments (siRNA) within cells, and that these siRNAs are the actual mediators of the RNAi phenomenon.

Although there have been numerous efforts to use RNAi to control plant parasitic nematodes, to date no transgenic nematode-resistant plant has been deregulated in any country. Accordingly, there continues to be a need to identify safe and effective compositions and methods for the controlling plant parasitic nematodes using RNAi, and for the production of plants having increased resistance to plant parasitic nematodes.

### SUMMARY OF THE INVENTION

The present invention provides nucleic acids, transgenic plants, and methods to overcome or alleviate nematode infestation of valuable agricultural crops such as soybeans and potatoes. The nucleic acids of the invention are capable of decreasing expression of plant target genes by RNA interference (RNAi). In accordance with the invention, the plant target gene is selected from a group consisting of a CLASP1 gene, an Aspartic Proteinase Delta Subunit gene, a Secreted Protein1 gene, a Lectin Receptor Kinase-like gene (LRK-like), a Pectin Methylesterase-like gene (PME-like), and an NPY gene.

In one embodiment, the invention provides an isolated expression vector encoding a double stranded RNA comprising a first strand and a second strand complementary to the first strand, wherein the first strand is substantially identical to at least 19, 20, or 21 consecutive nucleotides of a plant polynucleotide selected from the group consisting of a CLASP1 gene, an Aspartic Proteinase Delta Subunit gene, a Secreted Protein1 gene, a Lectin Receptor Kinase-like gene, a Pectin Methylesterase-like gene, and an NPY gene, wherein the double stranded RNA inhibits expression of the target gene.

The invention is further embodied as an isolated expression vector comprising a nucleic acid encoding a multiplicity of double stranded RNA molecules each comprising a double stranded region having a length of at least 19, 20, or 21 nucleotides, wherein one strand of said double stranded region is derived from a plant target polynucleotide selected from the group consisting of a plant CLASP1 gene, an Aspartic Proteinase Delta Subunit gene, a Secreted Protein1 gene, a Lectin Receptor Kinase-like gene, a Pectin Methylesterase-like gene, and an NPY gene, wherein the double stranded RNA inhibits expression of the target gene.

In another embodiment, the invention provides a transgenic plant capable of expressing at least one a dsRNA that is substantially identical to at least 19, 20, or 21 consecutive nucleotides of a plant target gene selected from the group consisting of a plant CLASP1 gene, an Aspartic Proteinase Delta Subunit gene, a Secreted Protein1 gene, a Lectin Receptor Kinase-like gene, a Pectin Methylesterase-like gene, and an NPY gene, wherein the dsRNA inhibits expression of the target gene in the plant root.

The invention further encompasses a method of making a transgenic plant capable of expressing a dsRNA comprising a first strand that is substantially identical to portion of a plant target gene and a second strand complementary to the first strand, wherein the target gene is selected from the group consisting of a plant a CLASP1 gene, an Aspartic Proteinase Delta Subunit gene, a Secreted Protein1 gene, a Lectin Receptor Kinase-like gene, a Pectin Methylesterase-like gene, and an NPY gene, said method comprising the steps of: (a) preparing an expression vector comprising a nucleic acid encoding the dsRNA, wherein the nucleic acid is able to form a double-stranded transcript once expressed in the plant; (b) transforming a recipient plant with said expression vector; (c) producing one or more transgenic offspring of said recipient plant; and (d) selecting the offspring for resistance to nematode infection.

The invention further provides a method of conferring nematode resistance to a plant, said method comprising the steps of: (a) selecting a plant target gene from the group consisting of a plant a CLASP1 gene, an Aspartic Proteinase Delta Subunit gene, a Secreted Protein1 gene, a Lectin Receptor Kinase-like gene, a Pectin Methylesterase-like gene, and an NPY gene; (b) preparing an expression vector comprising a nucleic acid encoding a dsRNA comprising a first strand that is substantially identical to a portion of the target gene and a second strand complementary to the first strand, wherein the nucleic acid is able to form a double-stranded transcript once expressed in the plant; (c) transforming a recipient plant with said nucleic acid; (d) producing one or more transgenic offspring of said recipient plant; and (e) selecting the offspring for nematode resistance.

### BRIEF DESCRIPTION OF THE DRAWINGS

FIGS. 1a-1b show the table of SEQ ID NOs assigned to corresponding nucleotide and amino acid sequences from Glycine max and other plant species.

FIGS. 2a-2c show the amino acid alignment of the open reading frame encoded by GmCLASP1 (SEQ ID NO:2) with related soybean amino acid sequences described by soybean gene model identifiers Glyma03g32710.1 (SEQ ID NO:5), Glyma13g19230.1 (SEQ ID NO:7) and Glyma10g04850.1 (SEQ ID NO:9), using the Vector NTI software suite v10.3.0 (gap opening penalty=10, gap extension penalty=0.05, gap separation penalty=8).

FIG. 3 shows the amino acid alignment of the open reading frame encoded by GmAspartic Proteinase Delta Subunit (SEQ ID NO:11) with related soybean amino acid sequences described by soybean gene model identifiers Glyma15g11670.1 (SEQ ID NO:14) and Glyma07g39240.1 (SEQ ID NO:16), using the Vector NTI software suite v10.3.0 (gap opening penalty=10, gap extension penalty=0.05, gap separation penalty=8).

FIG. 4 shows the amino acid alignment of the open reading frame encoded by GmSecreted Protein1 (SEQ ID NO:18) with a related soybean amino acid sequences described by GmSecreted Protein2 (SEQ ID NO:21) and soybean gene model identifier Glyma20g26600.1 (SEQ ID NO:23), using the Vector NTI software suite v10.3.0 (gap opening penalty=10, gap extension penalty=0.05, gap separation penalty=8).

FIG. 5 shows the amino acid alignment of the open reading frame encoded by GmLectin Receptor Kinase-like (SEQ ID NO:25) with a related soybean amino acid sequence described by soybean gene model identifier Glyma18g40290.1 (SEQ ID NO:28), using the Vector NTI software suite v10.3.0 (gap opening penalty=10, gap extension penalty=0.05, gap separation penalty=8).

FIG. 6 shows the amino acid alignment of GmPectin Methylesterase-like (SEQ ID NO:30) with a related soybean amino acid sequence described by soybean gene model identifier Glyma16g01650.1 (SEQ ID NO:33), using the Vector NTI software suite v10.3.0 (gap opening penalty=10, gap extension penalty=0.05, gap separation penalty=8).

FIG. 7 shows the amino acid alignment of GmNPY1 from soybean gene model Glyma05g22370.1 (SEQ ID NO:35) with related soybean amino acid sequences GmNPY-like2 (SEQ ID NO:38), GmNPY-like3 (SEQ ID NO:40) and GmNPY-like4 from soybean gene model Glyma17g17470.1 (SEQ ID NO:42), using the Vector NTI software suite v10.3.0 (gap opening penalty=10, gap extension penalty=0.05, gap separation penalty=8).

FIG. 8 shows the amino acid alignment of GmNPY-like5 (SEQ ID NO:44) with a related soybean amino acid sequence GmNPY-like6 (SEQ ID NO:48) using the Vector NTI software suite v10.3.0 (gap opening penalty=10, gap extension penalty=0.05, gap separation penalty=8).

FIGS. 9a-9j show the DNA alignment of the open reading frame sequence of GmCLASP1 (SEQ ID NO:1) with open reading frame sequences of related soybean gene models Glyma03g32710.1 (SEQ ID NO:4), Glyma13g19230.1 (SEQ ID NO:6), and Glyma10g04850.1 (SEQ ID NO:8) using the Vector NTI software suite v10.3.0 (gap opening penalty=15, gap extension penalty=6.66, gap separation penalty=8). The hairpin stem generated by binary vector RTP2593-3 with the sense strand described by SEQ ID NO:3 is capable of targeting the corresponding DNA sequences described by SEQ ID NO:1, SEQ ID NO:4, SEQ ID NO:6 and SEQ ID NO:8 as shown in the alignment.

FIGS. 10a-10c show the DNA alignment of the open reading frame sequence of GmAspartic Proteinase (SEQ ID NO:10) with open reading frame sequences of related soybean gene models Glyma15g11670.1 (SEQ ID NO:13) and Glyma07g39240.1 (SEQ ID NO:15) using the Vector NTI software suite v10.3.0 (gap opening penalty=15, gap extension penalty=6.66, gap separation penalty=8). The hairpin stem generated by binary vector RTP3113-1 with the sense strand described by SEQ ID NO:12 is capable of targeting the corresponding DNA sequences described by SEQ ID NO:10, SEQ ID NO:13 and SEQ ID NO:15 as shown in the alignment.

FIGS. 11a-11b show the DNA alignment of the open reading frame sequence of GmSecreted Protein1 (SEQ ID NO:17) with open reading frame sequences of related soybean gene GmSecreted Protein2 (SEQ ID NO:20) and gene model Glyma20g26600.1 (SEQ ID NO:22) using the Vector NTI software suite v10.3.0 (gap opening penalty=15, gap extension penalty=6.66, gap separation penalty=8). The hairpin stem generated by binary vectors RTP3923-4 and RTP3924-1 with the sense strand described by SEQ ID NO:19 are capable of targeting the corresponding DNA sequences described by SEQ ID NO:17, SEQ ID NO:20 and SEQ ID NO:22 as shown in the alignment.

FIGS. 12a-12d show the DNA alignment of the open reading frame sequence of GmLectin Receptor Kinase-like (SEQ ID NO:24) with the open reading frame sequence of related soybean gene model Glyma18g40290.1 (SEQ ID NO:27) using the Vector NTI software suite v10.3.0 (gap opening penalty=15, gap extension penalty=6.66, gap separation penalty=8). The hairpin stem generated by binary vectors RTP4279-1 and RTP4280-2 with the sense strand described by SEQ ID NO:26 are capable of targeting the corresponding DNA sequences described by SEQ ID NO:24 and SEQ ID NO:27 as shown in the alignment.

FIGS. 13a-13c show the DNA alignment of the open reading frame sequence of GmPectin Methylesterase-like (SEQ ID NO: 29) with the open reading frame sequence of related soybean gene model Glyma16g01650.1 (SEQ ID NO: 32) using the Vector NTI software suite v10.3.0 (gap opening penalty=15, gap extension penalty=6.66, gap separation penalty=8). The hairpin stem generated by binary vector RTP3856-4 with the sense strand described by SEQ ID NO: 31 is capable of targeting the corresponding DNA sequences described by SEQ ID NO:29 and SEQ ID NO:32 as shown in the alignment.

FIGS. 14a-14d show the DNA alignment of the sequence of GmNPY1 gene (SEQ ID NO: 34) with sequences of related soybean genes GmNPY-like2 (SEQ ID NO: 37), GmNPY-like3 (SEQ ID NO:39) and GmNPY-like4, from soybean gene model Glyma17g17470.1, (SEQ ID NO:41) using the Vector NTI software suite v10.3.0 (gap opening penalty=15, gap extension penalty=6.66, gap separation penalty=8). The hairpin stem generated by binary vectors RTP2361-4 and RTP2362-1 with the sense strand described by SEQ ID NO:36 are capable of targeting the corresponding DNA sequences described by SEQ ID NO:34, SEQ ID NO:37, SEQ ID NO:39 and SEQ ID NO:41 as shown in the alignment.

FIGS. 15a-15d show the DNA alignment of GmNPY-like5 (SEQ ID NO: 43) with related soybean gene GmNPY-like6 (SEQ ID NO: 47) using the Vector NTI software suite v10.3.0 (gap opening penalty=15, gap extension penalty=6.66, gap separation penalty=8). The hairpin stem generated by binary vector RTP4082-1 with the sense strand described by SEQ ID NO:45 and binary vector RTP4083 with the sense strand described by SEQ ID NO:46 are capable of targeting the corresponding DNA sequences described by SEQ ID NO:43 and SEQ ID NO:47 as shown in the alignment.

FIGS. 16a-16n show global percent identity of exemplary GmCLASP1 sequences (FIG. 16a, amino acid; FIG. 16b, nucleotide), GmAspartic Proteinase Delta Subunit sequences (FIG. 16c, amino acid; FIG. 16d, nucleotide), GmSecreted Protein1 sequences (FIG. 16e, amino acid; FIG. 16f, nucleotide), GmLectin Receptor Kinase-like sequences (FIG. 16g, amino acid; FIG. 16h, nucleotide), GmPectin Methylesterase-like sequences (FIG. 16i, amino acid; FIG. 16j, nucleotide), GmNPY1 sequences (FIG. 16k, amino acid; FIG. 16l, nucleotide) and GmNPY-like5 sequences (FIG. 16m, amino acid; FIG. 16n, nucleotide). Percent identity was calculated from multiple alignments using the Vector NTI software suite v10.3.0. Nucleotide percent identity was calculated from multiple alignments of predicted coding regions.

FIGS. 17a-17c shows the amino acid alignment of the GmCLASP1 gene (SEQ ID NO:2) with related homologs from soybean Glyma03g32710.1, Glyma13g19230.1 and Glyma10g04850.1 (SEQ ID NO:5, SEQ ID NO:7 and SEQ ID NO:9, respectively) and the partial potato StCLASP sequence from Genbank EST BQ506533 (SEQ ID NO:65) using the Vector NTI software suite v10.3.0 (gap opening penalty=10, gap extension penalty=0.05, gap separation penalty=8).

FIGS. 18a-18d shows the amino acid alignment of the GmNPY1 gene (SEQ ID NO:35) with related homologs from soybean GmNPY-like2, GmNPY-like3, GmNPY-like4, GmNPY-like5, GmNPY-like6 and GmNPY-like7 (SEQ ID NO:38, SEQ ID NO:40, SEQ ID NO:42, SEQ ID NO:44, SEQ ID NO:48 and SEQ ID NO:50 respectively), from corn ZmLOC100280048 and ZM07MC01162_BFb0263J23 (SEQ ID NO:52 and SEQ ID NO:54, respectively), from rice OsAK103674.1, Os12g0583500 and Os09g0420900 (SEQ ID NO:56, SEQ ID NO:58 and SEQ ID NO:60, respectively) and from cotton TA26692_{—}3635_Gh (SEQ ID NO:62) using the Vector NTI software suite v10.3.0 (gap opening penalty=10, gap extension penalty=0.05, gap separation penalty=8).

FIGS. 19a-19n shows the nucleotide alignment of the open reading frame sequence of the GmCLASP1 gene (SEQ ID NO:1) with open reading frame sequences of related homologs from soybean Glyma03g32710.1, Glyma13g19230.1, Glyma10g04850.1 (SEQ ID NO:4, SEQ ID NO:6, SEQ ID NO:8, respectively) and the partial homolog from potato StCLASP BQ506533 EST (SEQ ID NO:63) using the Vector NTI software suite v10.3.0 (gap opening penalty=15, gap extension penalty=6.66, gap separation penalty=8).

FIGS. 20a-20l shows the nucleotide alignment the open reading frame sequence of the GmNPY1 gene (SEQ ID NO:34) with open reading frame sequences of related homologs from soybean gene GmNPY-like2, GmNPY-like3, GmNPY-like4, GmNPY-like5, GmNPY-like6 and GmNPY-like7 (SEQ ID NO:37, SEQ ID NO:39, SEQ ID NO:41, SEQ ID NO:43, SEQ ID NO:47 and SEQ ID NO:49 respectively), from corn ZmLOC100280048 and ZM07MC01162_BFb0263J23 (SEC) ID NO:51 and SEQ ID NO:53, respectively), from rice OsAK103674.1, Os12g0583500 and Os09g0420900 (SEQ ID NO:55, SEQ ID NO:57 and SEQ ID NO:59, respectively) and from cotton TA26692_{—}3635_Gh (SEQ ID NO:61) using the Vector NTI software suite v10.3.0 (gap opening penalty=15, gap extension penalty=6.66, gap separation penalty=8).

FIGS. 21a-21d show global percent identity of exemplary GmCLASP1 sequences (FIG. 21a, amino acid; FIG. 21b, nucleotide) and GmNPY1 sequences (FIG. 21c, amino acid; FIG. 21d, nucleotide). Percent identity was calculated from multiple alignments using the Vector NTI software suite v10.3.0. Nucleotide percent identity was calculated from multiple alignments of predicted coding regions.

FIGS. 22a-22aa show various 21mers possible in SEQ ID NO:1, 3, 4, 6, 8, 10, 12, 13, 15, 17, 19, 20, 22, 24, 26, 27, 29, 31, 32, 34, 36, 37, 39, 41, 43, 45, 46, 47, 49, 51, 53, 55, 57, 59, 61, 63 or 65 by nucleotide position.

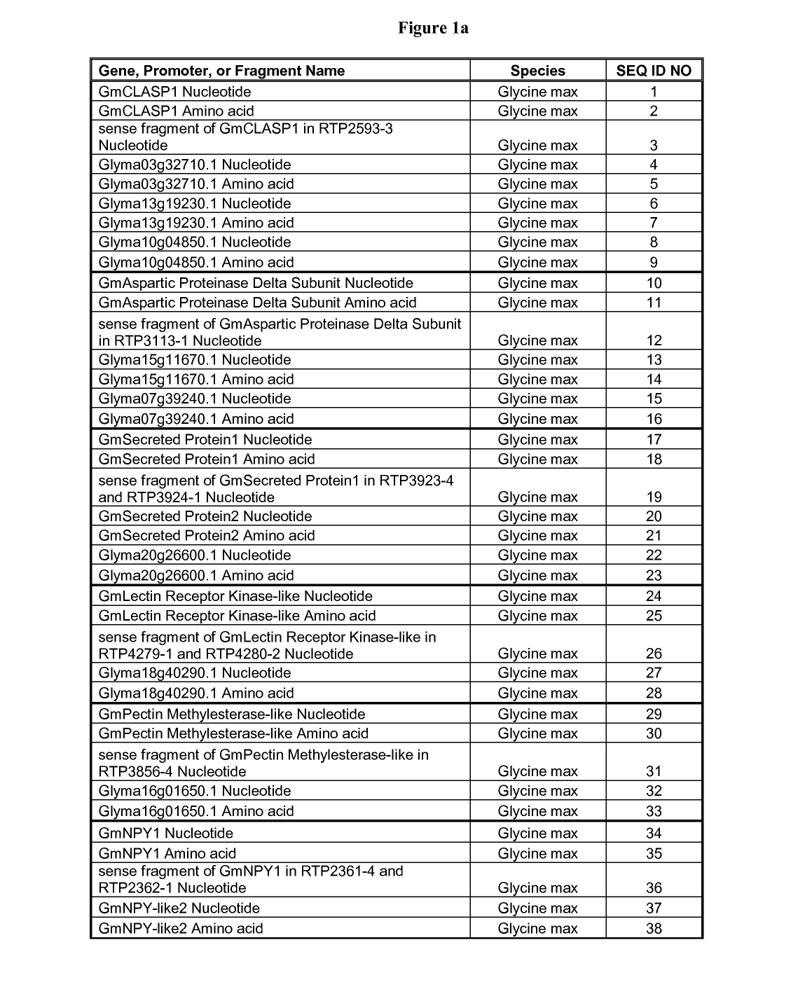

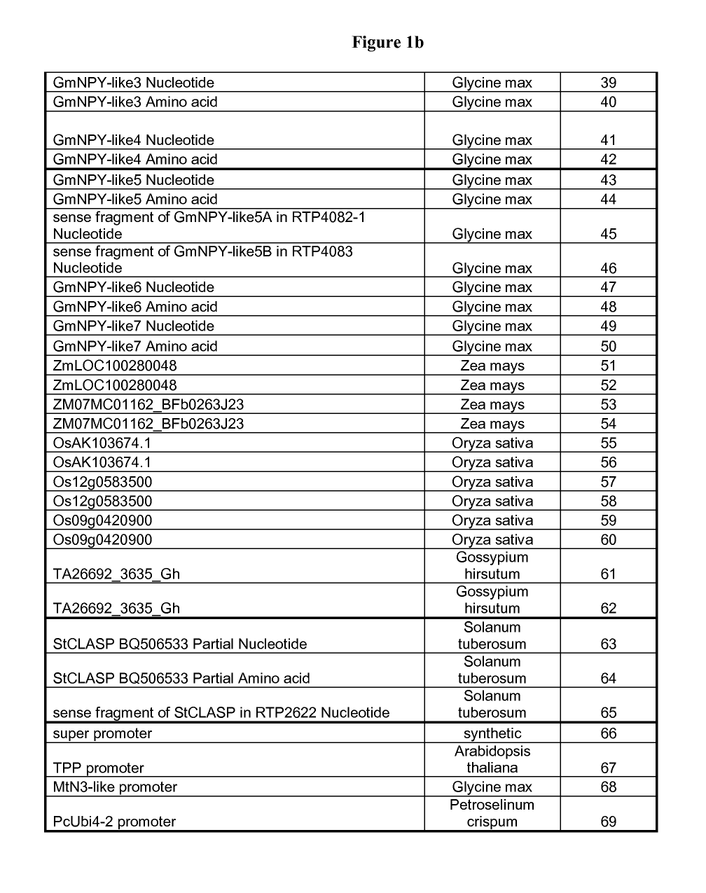

### DETAILED DESCRIPTION OF THE PREFERRED EMBODIMENTS

The present invention may be understood more readily by reference to the following detailed description of the preferred embodiments of the invention and the examples included herein. Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al. Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized It is to be understood that the terminology used herein is for the purpose of describing specific embodiments only and is not intended to be limiting.

Throughout this application, various publications are referenced. The disclosures of all of these publications and those references cited within those publications in their entireties are hereby incorporated by reference into this application in order to more fully describe the state of the art to which this invention pertains. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

As used herein, “RNAi” or “RNA interference” refers to the process of sequence-specific post-transcriptional gene silencing in plants, mediated by double-stranded RNA (dsRNA). As used herein, “dsRNA” refers to RNA that is partially or completely double stranded. Double stranded RNA is also referred to as short interfering RNA (siRNA), short interfering nucleic acid (siNA), micro-RNA (miRNA), and the like. In the RNAi process, dsRNA comprising a first strand that is substantially identical to a portion of a target gene and a second strand that is complementary to the first strand is introduced into a plant. After introduction into the plant, the target gene-specific dsRNA is processed into relatively small fragments (siRNAs) by a plant cell containing the RNAi processing machinery resulting in target gene silencing.

As used herein, taking into consideration the substitution of uracil for thymine when comparing RNA and DNA sequences, the term “substantially identical” as applied to dsRNA means that the nucleotide sequence of one strand of the dsRNA is at least about 80%-90% identical to 20 or more contiguous nucleotides of the target gene, more preferably, at least about 90-95% identical to 20 or more contiguous nucleotides of the target gene, and most preferably at least about 95%, 96%, 97%, 98% or 99% identical or absolutely identical to 20 or more contiguous nucleotides of the target gene. 20 or more nucleotides means a portion, being at least about 20, 21, 22, 23, 24, 25, 50, 100, 200, 300, 400, 500, 1000, 1500, consecutive bases or up to the full length of the target gene.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. As used herein, the term “substantially complementary” means that two nucleic acid sequences are complementary over at least at 80% of their nucleotides. Preferably, the two nucleic acid sequences are complementary over at least at 85%, 90%, 95%, 96%, 97%, 98%, 99% or more or all of their nucleotides. Alternatively, “substantially complementary” means that two nucleic acid sequences can hybridize under high stringency conditions. As used herein, the term “substantially identical” or “corresponding to” means that two nucleic acid sequences have at least 80% sequence identity. Preferably, the two nucleic acid sequences have at least 85%, 90%, 95%, 96%, 97%, 98%, 99% or 100% of sequence identity.

Also as used herein, the terms “nucleic acid” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the terms “contacting” and “administering” are used interchangeably, and refer to a process by which dsRNA of the present invention is transcribed in a plant in order to inhibit expression of an essential target gene in the plant. The dsRNA may be administered in a number of ways, including, but not limited to, direct introduction into a cell (i.e., intracellularly); or extracellular introduction, or into the vascular system of the plant, or the dsRNA may be transcribed by the plant. For example, the dsRNA may be sprayed onto a plant, or the dsRNA may be applied to soil in the vicinity of roots, taken up by the plant, or a plant may be genetically engineered to express the dsRNA targeting a plant target gene in an amount sufficient to kill or adversely affect some or all of the parasitic nematode to which the plant is exposed by dsRNA silencing (RNAi) of the plant target gene.

As used herein, the term “control,” when used in the context of an infection, refers to the reduction or prevention of an infection. Reducing or preventing an infection by a nematode will cause a plant to have increased resistance to the nematode; however, such increased resistance does not imply that the plant necessarily has 100% resistance to infection. In preferred embodiments, the resistance to infection by a nematode in a resistant plant is greater than 10%, 20%, 30%, 40%, 50%, 60%, 70%, 80%, 90%, or 95% in comparison to a wild type plant that is not resistant to nematodes. Preferably the wild type plant is a plant of a similar, more preferably identical genotype as the plant having increased resistance to the nematode, but does not comprise a dsRNA directed to the target gene. The plant's resistance to infection by the nematode may be due to the death, sterility, arrest in development, or impaired mobility of the nematode upon exposure to the dsRNA specific to a plant gene having some effect on feeding site development, maintenance, or overall ability of the feeding site to provide nutrition to the nematode. The term “resistant to nematode infection” or “a plant having nematode resistance” as used herein refers to the ability of a plant, as compared to a wild type plant, to avoid infection by nematodes, to kill nematodes or to hamper, reduce or stop the development, growth or multiplication of nematodes. This might be achieved by an active process, e.g. by producing a substance detrimental to the nematode, or by a passive process, like having a reduced nutritional value for the nematode or not developing structures induced by the nematode feeding site like syncytia or giant cells. The level of nematode resistance of a plant can be determined in various ways, e.g. by counting the nematodes being able to establish parasitism on that plant, or measuring development times of nematodes, proportion of male and female nematodes or, for cyst nematodes, counting the number of cysts or nematode eggs produced on roots of an infected plant or plant assay system.

The term “plant” is intended to encompass plants at any stage of maturity or development, as well as any tissues or organs (plant parts) taken or derived from any such plant unless otherwise clearly indicated by context. Plant parts include, but are not limited to, stems, roots, flowers, ovules, stamens, seeds, leaves, embryos, meristematic regions, callus tissue, anther cultures, gametophytes, sporophytes, pollen, microspores, protoplasts, hairy root cultures, and the like. The present invention also includes seeds produced by the plants of the present invention. In one embodiment, the seeds are true breeding for an increased resistance to nematode infection as compared to a wild-type variety of the plant seed. As used herein, a “plant cell” includes, but is not limited to, a protoplast, gamete producing cell, and a cell that regenerates into a whole plant. Tissue culture of various tissues of plants and regeneration of plants therefrom is well known in the art and is widely published.

As used herein, the term “transgenic” refers to any plant, plant cell, callus, plant tissue, or plant part that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

As used herein, the term “amount sufficient to inhibit expression” refers to a concentration or amount of the dsRNA that is sufficient to reduce levels or stability of mRNA or protein produced from a target gene in a plant. As used herein, “inhibiting expression” refers to the absence or observable decrease in the level of protein and/or mRNA product from a target gene. Inhibition of the plant target gene expression may result in lethality to the parasitic nematode, or such inhibition may delay or prevent entry into a particular developmental step (e.g., metamorphosis), if plant disease is associated with a particular stage of the parasitic nematode's life cycle. The consequences of inhibition can be confirmed by examination of the outward properties of the nematode (as presented below in the examples).

In accordance with the invention, a plant transcribes a dsRNA, which specifically inhibits expression of a plant target gene that effects nematode feeding site development, feeding site maintenance, nematode survival, nematode metamorphosis, or nematode reproduction. In a preferred embodiment, the dsRNA is encoded by an expression vector that has been transformed into an ancestor of the infected plant. More preferably, the expression vector comprises a nucleic acid encoding the dsRNA under the transcriptional control of a root specific promoter or a parasitic nematode induced feeding cell-specific promoter. Most preferably, the expression vector comprises a nucleic acid encoding the dsRNA under the transcriptional control of a parasitic nematode induced feeding cell-specific promoter.

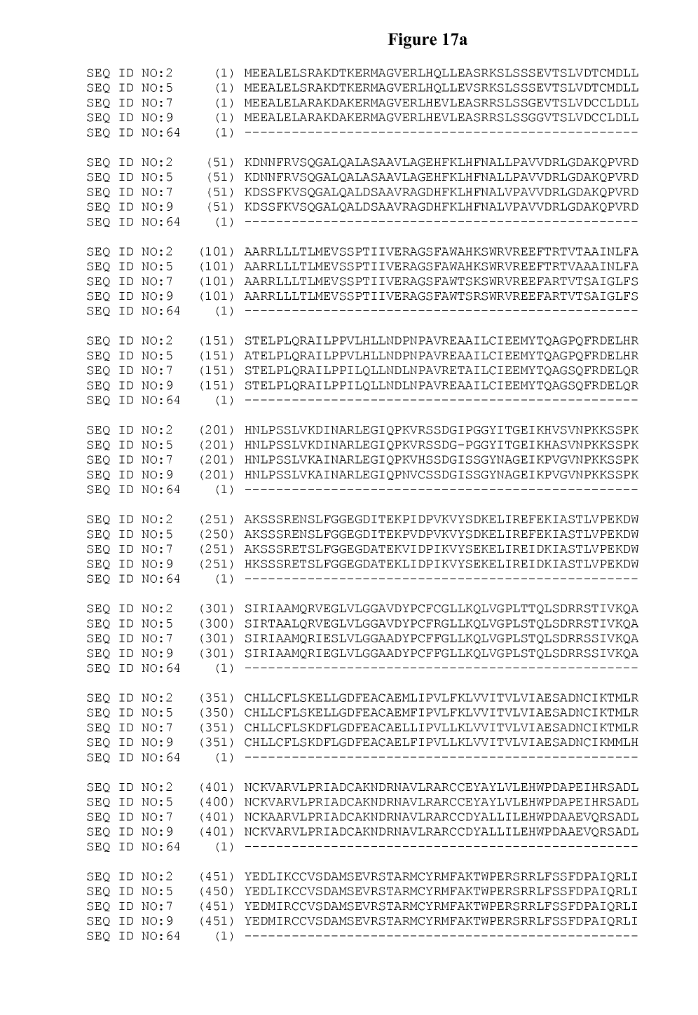

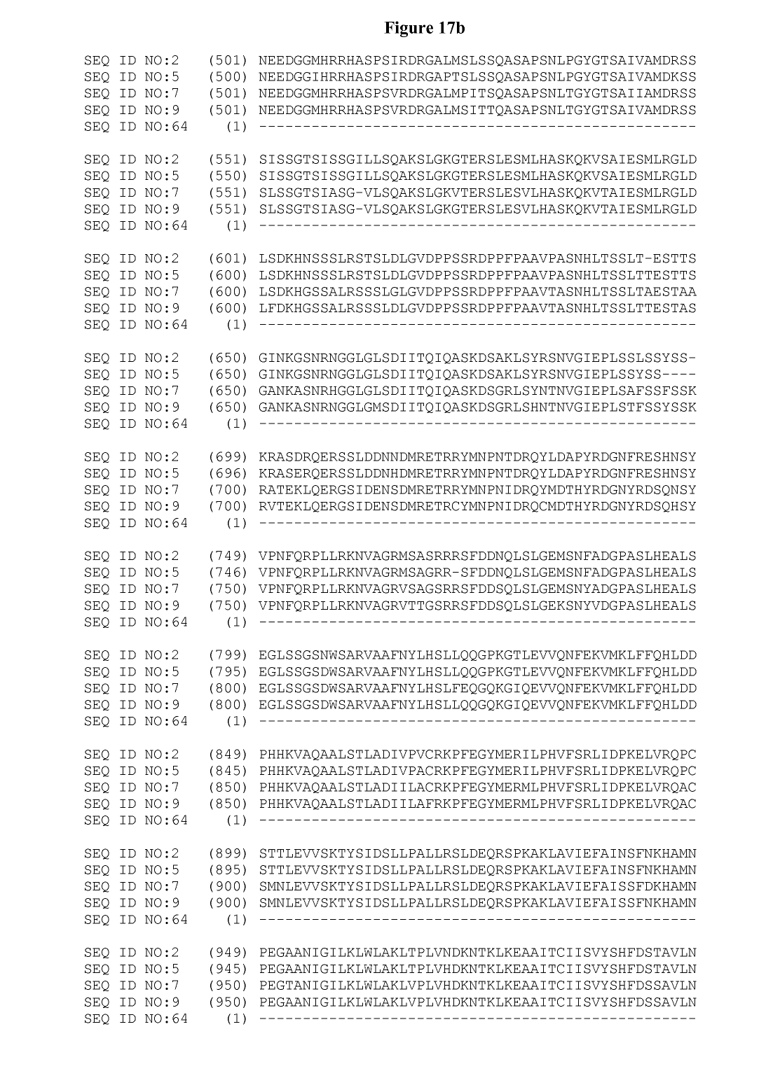

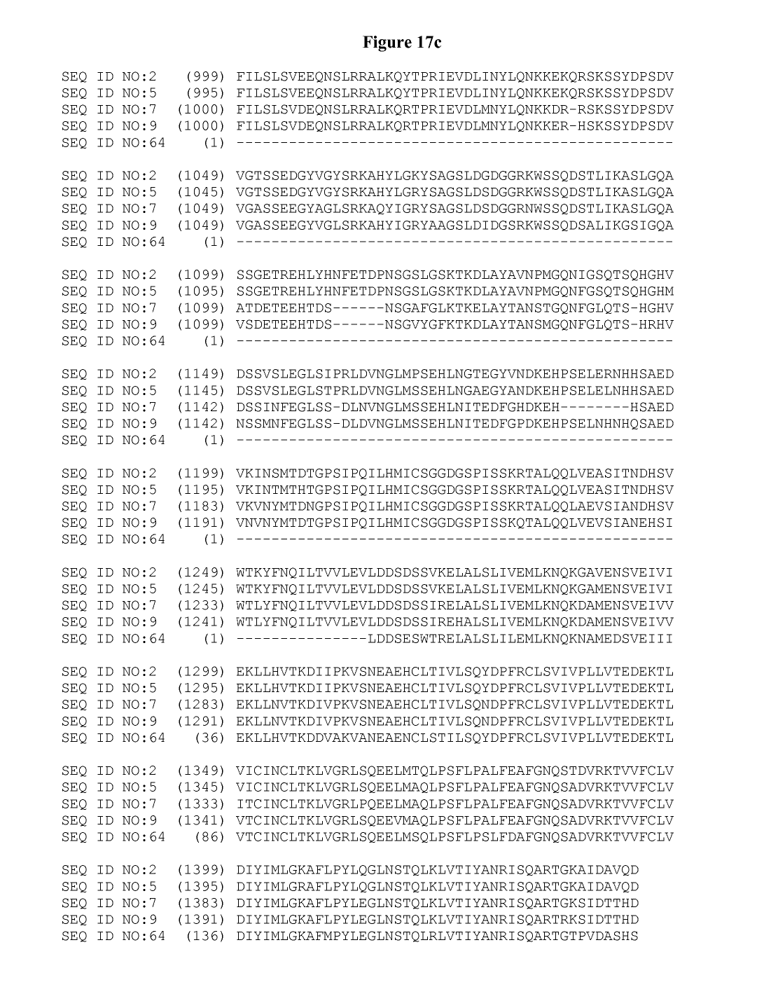

In one embodiment, the dsRNA of the invention targets a plant CLASP1 gene. CLASP, or CLIP-ASSOCIATED PROTEIN, genes in plants have been shown to be involved with microtubule stability and therefore may be involved in a range of cellular functions such as cell division and expansion, organellar movement and intracellular trafficking. As shown in Example 1, the full length G. max GmCLASP1 gene was isolated and is represented in SEQ ID NO:1. The G. max GmCLASP1 gene sequence described by SEQ ID NO:1 contains an open reading frame with the amino acid sequence disclosed as SEQ ID NO:2. As disclosed in Example 6, the amino acid sequence described by SEQ ID NO:2 was used to identify a homologous CLASP amino acid sequence from potato, StCLASP BQ506533. The corresponding homologous amino acid sequence is set forth in SEQ ID NO:64. The amino acid alignment of representative CLASP protein sequences or sequence fragments are set forth in SEQ ID NO:2, 5, 7, 9 and 64 is shown in FIG. 17a-c. Exemplary plant CLASP1 genes targeted by the dsRNA of this embodiment include genes having sequences as set forth in SEQ ID NO:1, 3, 4, 6, 8, 63, or 65; plant CLASP1 genes having at least 80% sequence identity to SEQ ID NO:1, 3, 4, 6, 8, 63, or 65; and plant CLASP1 genes that hybridize under stringent conditions to the sequence set forth in SEQ ID NO:1, 3, 4, 6, 8, 63, or 65.

In accordance with this embodiment, the dsRNA encoded by the expression vector of the invention comprises a first strand that is substantially identical to at least 19, 20, or 21 consecutive nucleotides of a CLASP1 target gene of a plant genome and a second strand that is substantially complementary to the first strand. Preferably, the CLASP1 dsRNA first strand comprises at least 19, 20, or 21 consecutive nucleotides of a plant CLASP1 polynucleotide selected from the group consisting of: (a) polynucleotide having the sequence set forth in SEQ ID NO:1, 3, 4, 6, 8, 63, or 65; (b) a plant CLASP1 polynucleotide having at least 80% sequence identity to SEQ ID NO:1, 3, 4, 6, 8, 63, or 65; and (c) a plant CLASP1 polynucleotide that hybridizes under stringent conditions to the polynucleotide having the sequence set forth in SEQ ID NO:1, 3, 4, 6, 8, 63, or 65.

In another embodiment, the dsRNA of the invention targets a plant Aspartic Proteinase Delta Subunit gene. Aspartic Proteinase Delta Subunit genes are localized to plant cell vacuoles and are involved in protein degradation. As shown in Example 1, the full length G. max GmAspartic Proteinase Delta Subunit gene was isolated and is represented in SEQ ID NO:10. Exemplary plant Aspartic Proteinase Delta Subunit genes targeted by the dsRNA of this embodiment include genes having sequences as set forth in SEQ ID NO:10, 12, 13 or 15; plant Aspartic Proteinase Delta Subunit genes having at least 80% sequence identity to SEQ ID NO:10, 12, 13 or 15; and plant Aspartic Proteinase Delta Subunit genes that hybridize under stringent conditions to the sequence set forth in SEQ ID NO:10, 12, 13 or 15.

In accordance with this embodiment, the dsRNA encoded by the expression vector of the invention comprises a first strand that is substantially identical to at least 19, 20, or 21 consecutive nucleotides of an Aspartic Proteinase Delta Subunit target gene of a plant genome and a second strand that is substantially complementary to the first strand. Preferably, the Aspartic Proteinase Delta Subunit dsRNA first strand comprises at least 19, 20, or 21 consecutive nucleotides of a plant Aspartic Proteinase Delta Subunit polynucleotide selected from the group consisting of: (a) a polynucleotide having the sequence set forth in SEQ ID NO:10, 12, 13 or 15; (b) a plant Aspartic Proteinase Delta Subunit polynucleotide having at least 80% sequence identity to SEQ ID NO:10, 12, 13 or 15; and (c) a plant Aspartic Proteinase Delta Subunit polynucleotide that hybridizes under stringent conditions to the polynucleotide having the sequence set forth in SEQ ID NO:10, 12, 13 or 15.

In another embodiment, the dsRNA of the invention targets a plant Secreted Protein1 gene. Secreted Proteins genes contain a basic secretory protein motif, and their function in plants is generally unknown although some secretory proteins may be involved with the plant defense response. As shown in Example 1, the full length G. max GmSecreted Protein1 gene was isolated and is represented in SEQ ID NO:17. Exemplary plant Secreted Protein1 genes targeted by the deRNA of this embodiment include genes having sequences as set forth in SEQ ID NO:17, 19, 20 or 22; plant Secreted Protein1 genes having at least 80% sequence identity to SEQ ID NO:17, 19, 20 or 22; and plant Secreted Protein1 genes that hybridize under stringent conditions to the sequence set forth in SEQ ID NO:17, 19, 20 or 22.

In accordance with this embodiment, the dsRNA encoded by the expression vector of the invention comprises a first strand that is substantially identical to at least 19, 20, or 21 consecutive nucleotides of a Secreted Protein1 target gene of a plant genome and a second strand that is substantially complementary to the first strand. Preferably, the Secreted Protein 1 dsRNA first strand comprises at least 19, 20, or 21 consecutive nucleotides of a plant Secreted Protein1 polynucleotide selected from the group consisting of: (a) a polynucleotide having the sequence set forth in SEQ ID NO:17, 19, 20 or 22; (b) a plant Secreted Protein1 polynucleotide having at least 80% sequence identity to SEQ ID NO:17, 19, 20 or 22; and (c) a plant Secreted Protein1 polynucleotide that hybridizes under stringent conditions to the polynucleotide having the sequence set forth in SEQ ID NO:17, 19, 20 or 22.

In another embodiment, the dsRNA of the invention targets a plant Lectin Receptor Kinase-like gene. Lectin Receptor Kinase-like genes contain extracellular lectin motifs and a kinase domain and can be involved with a variety of plant processes including growth, development, and response to stimuli. As shown in Example 1, the full length G. max GmLectin Receptor Kinase-like gene was isolated and is represented in SEQ ID NO:24. Exemplary Lectin Receptor Kinase-like genes targeted by the dsRNA of this embodiment include the sequences as set forth in SEQ ID NO:24, 26 or 27; plant Lectin Receptor Kinase-like genes having at least 80% sequence identity to SEQ ID NO:24, 26 or 27; and plant Lectin Receptor Kinase-like genes that hybridize under stringent conditions to the sequence set forth in SEQ ID NO:24, 26 or 27.

In accordance with this embodiment, the dsRNA encoded by the expression vector of the invention comprises a first strand that is substantially identical to at least 19, 20, or 21 consecutive nucleotides of a Lectin Receptor Kinase-like target gene of a plant genome. Preferably, the Lectin Receptor Kinase dsRNA first strand comprises at least 19, 20, or 21 consecutive nucleotides of a plant Lectin Receptor Kinase-like polynucleotide selected from the group consisting of: (a) a polynucleotide having the sequence set forth in SEQ ID NO:24, 26 or 27; (b) a plant Lectin Receptor Kinase-like polynucleotide having at least 80% sequence identity to SEQ ID NO:24, 26 or 27; and (c) a plant Lectin Receptor Kinase-like polynucleotide that hybridizes under stringent conditions to the polynucleotide having the sequence set forth in SEQ ID NO:24, 26 or 27.

In another embodiment, the dsRNA of the invention targets a plant Pectin Methylesterase-like gene. As shown in Example 1, the full length G. max Pectin Methylesterase-like gene was isolated and is represented in SEQ ID NO:29. Exemplary plant Lectin Receptor Kinase-like genes targeted by the dsRNA of this embodiment include the sequences set forth in SEQ ID NO:29, 31, or 32; plant Lectin Receptor Kinase-like genes having at least 80% sequence identity to SEQ ID NO: 29, 31, or 32; and plant Lectin Receptor Kinase-like genes that hybridize under stringent conditions to the sequence set forth in SEQ ID NO: 29, 31, or 32.

In accordance with this embodiment, the dsRNA encoded by the expression vector of the invention comprises a first strand that is substantially identical to at least 19, 20, or 21 consecutive nucleotides of a Pectin Methylesterase-like target gene of a plant genome and a second strand that is substantially complementary to the first strand. Preferably, the Pectin Methylesterase dsRNA first strand comprises at least 19, 20, or 21 consecutive nucleotides of a polynucleotide selected from the group consisting of: (a) a polynucleotide having the sequence set forth in SEQ ID NO: 29, 31 or 32; (b) a plant Pectin Methylesterase-like polynucleotide having at least 80% sequence identity to SEQ ID NO: 29, 31 or 32; and (c) a plant Pectin Methylesterase-like polynucleotide that hybridizes under stringent conditions to the polynucleotide having the sequence set forth in SEQ ID NO: 29, 31 or 32.

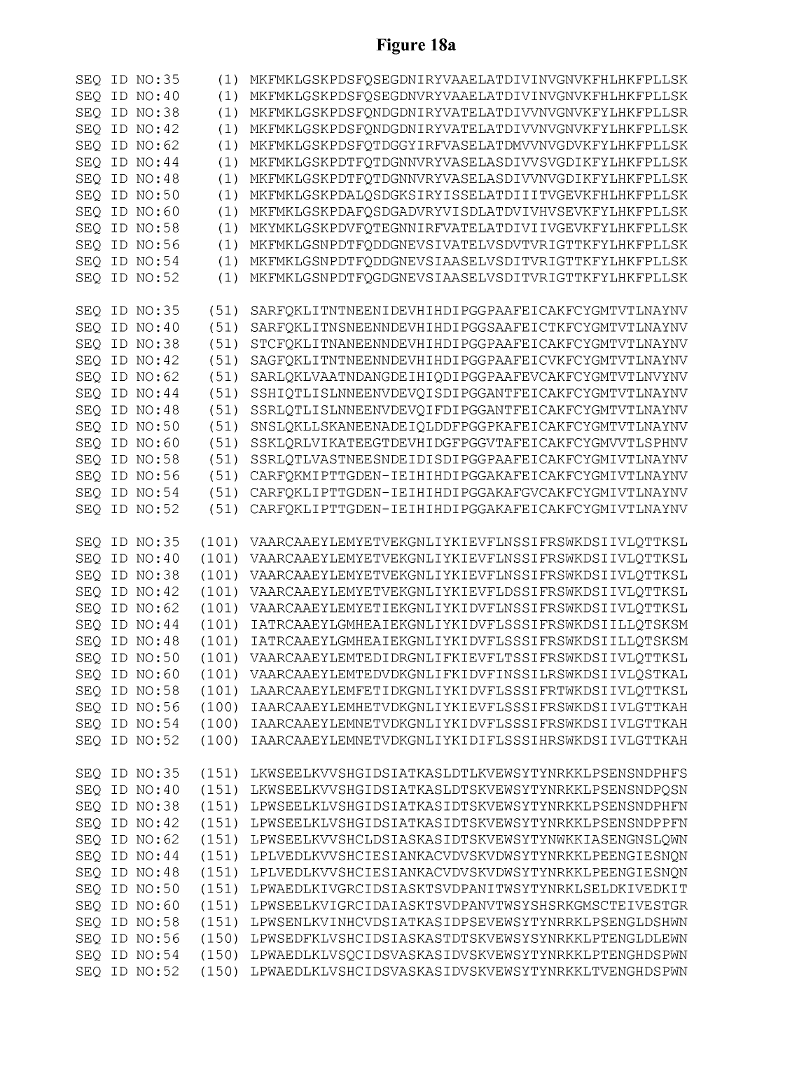

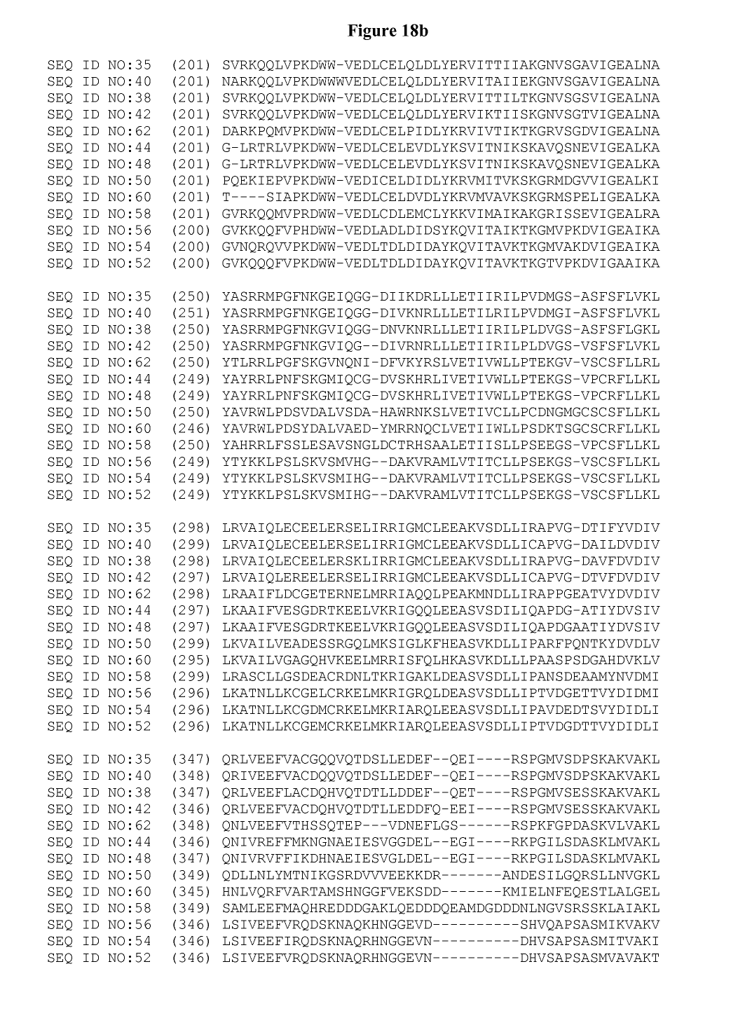

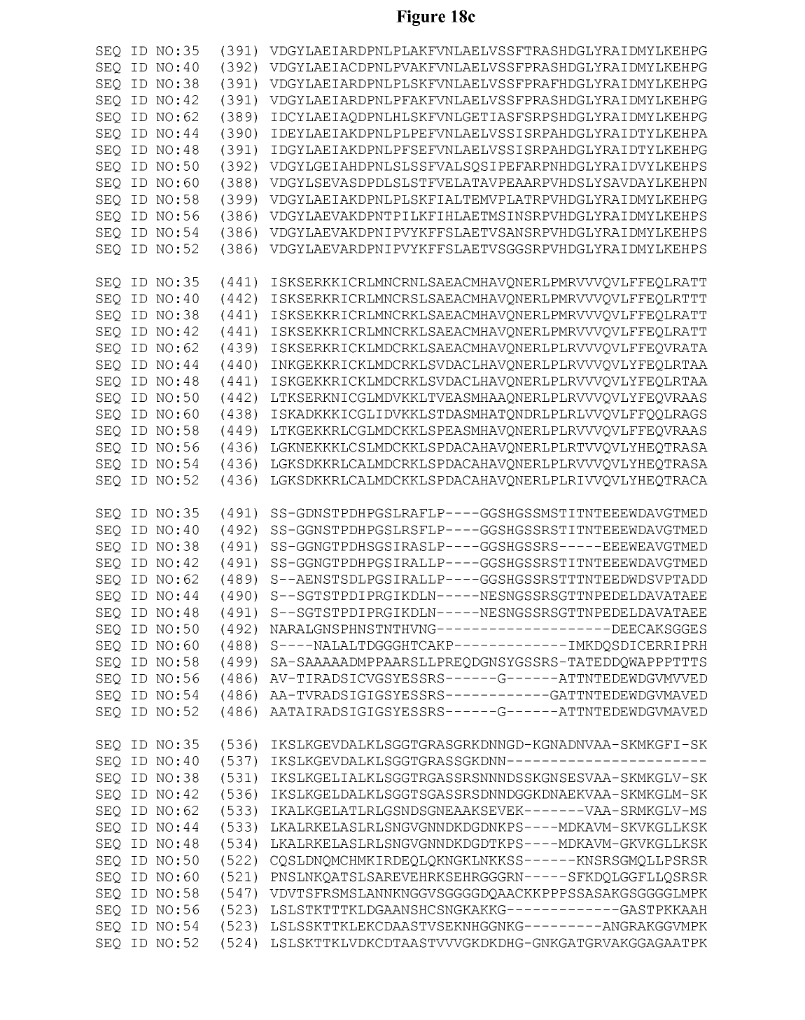

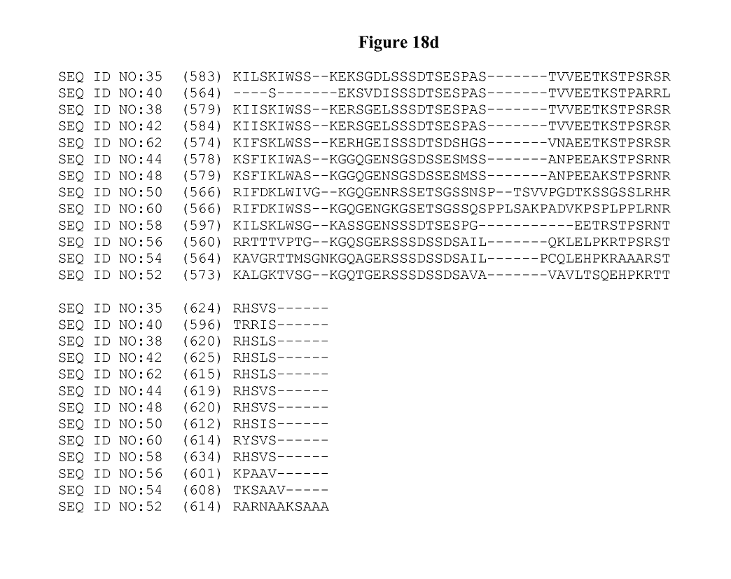

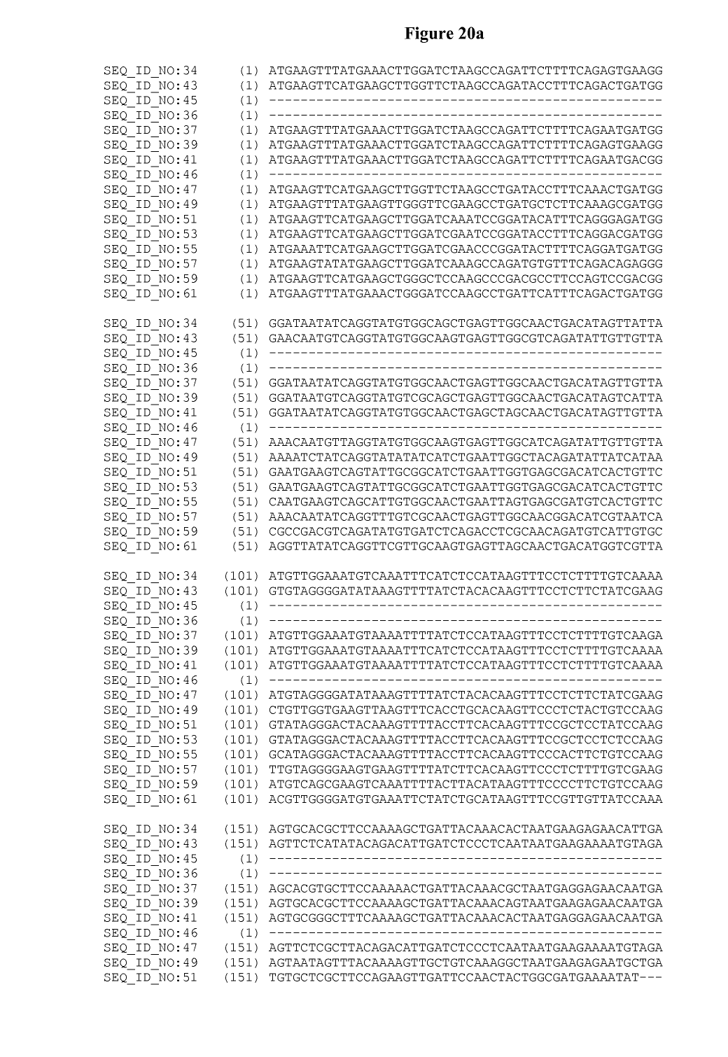

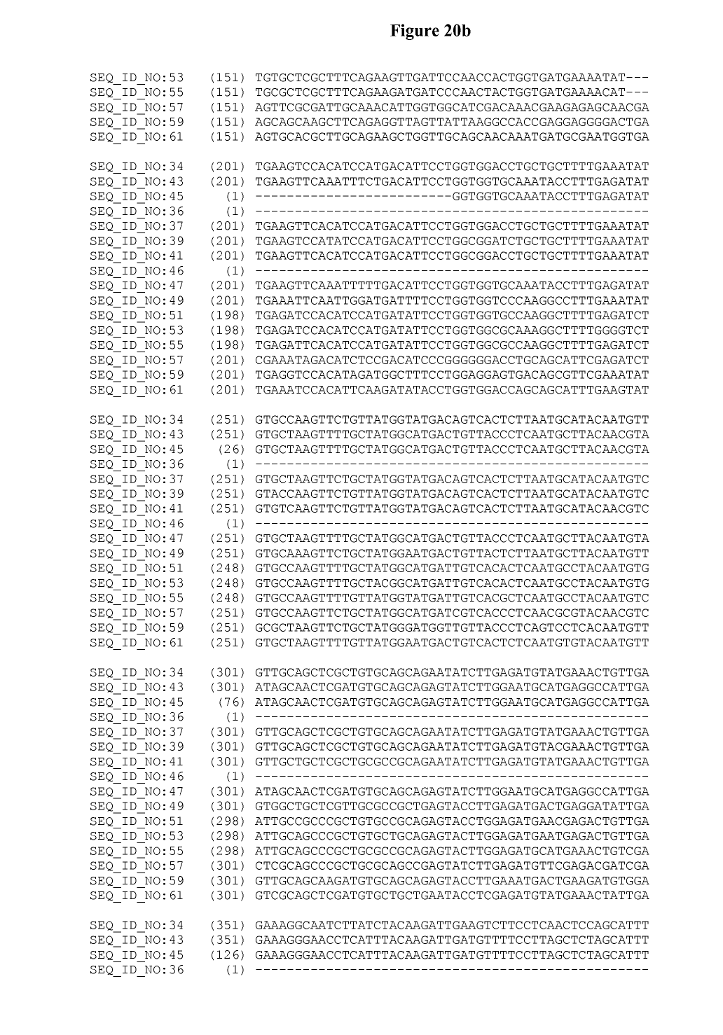

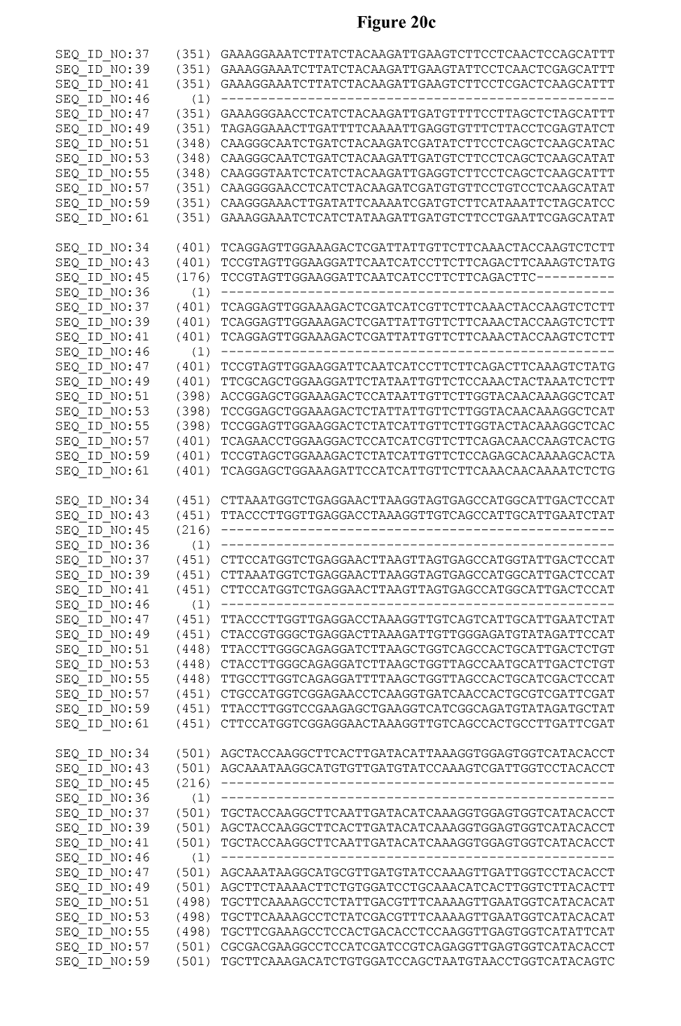

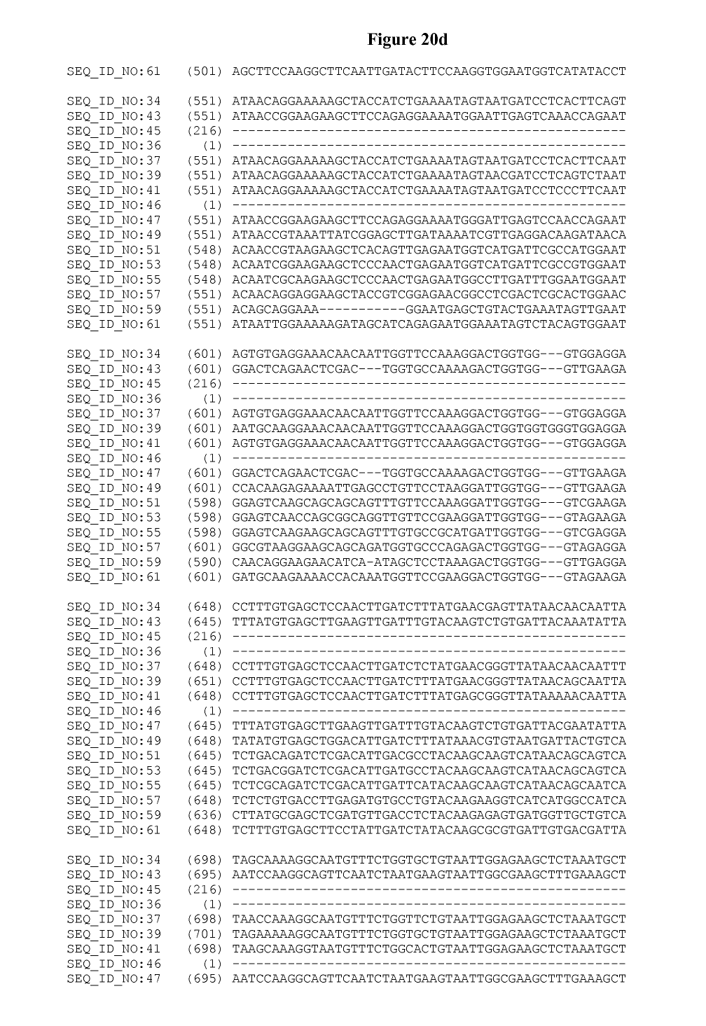

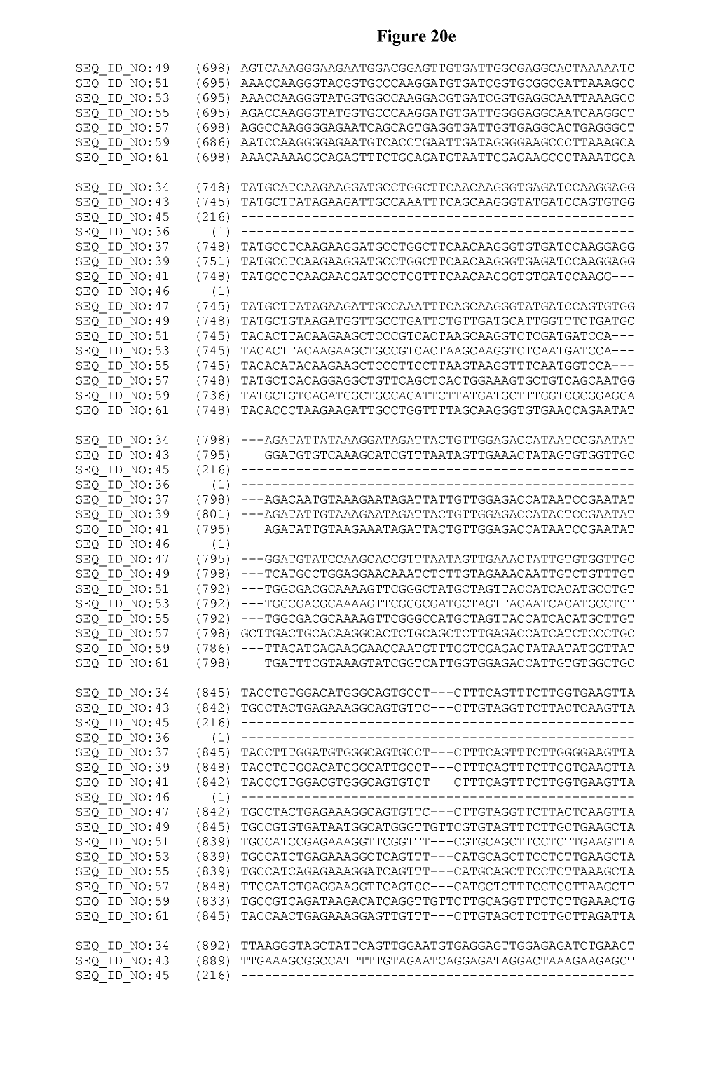

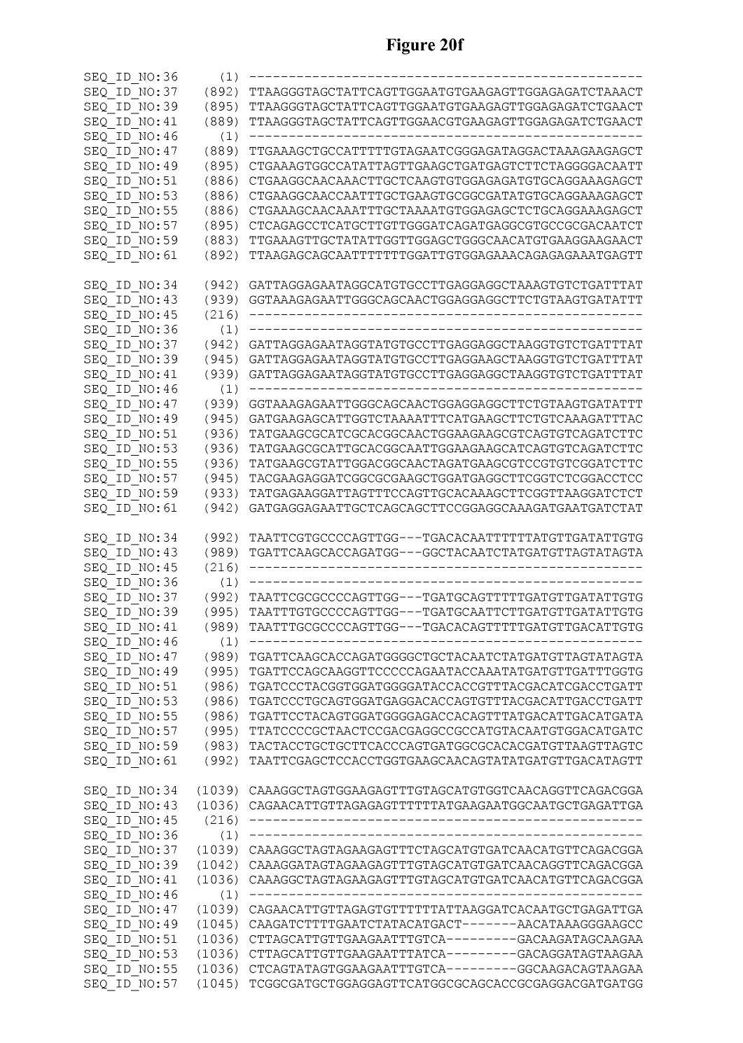

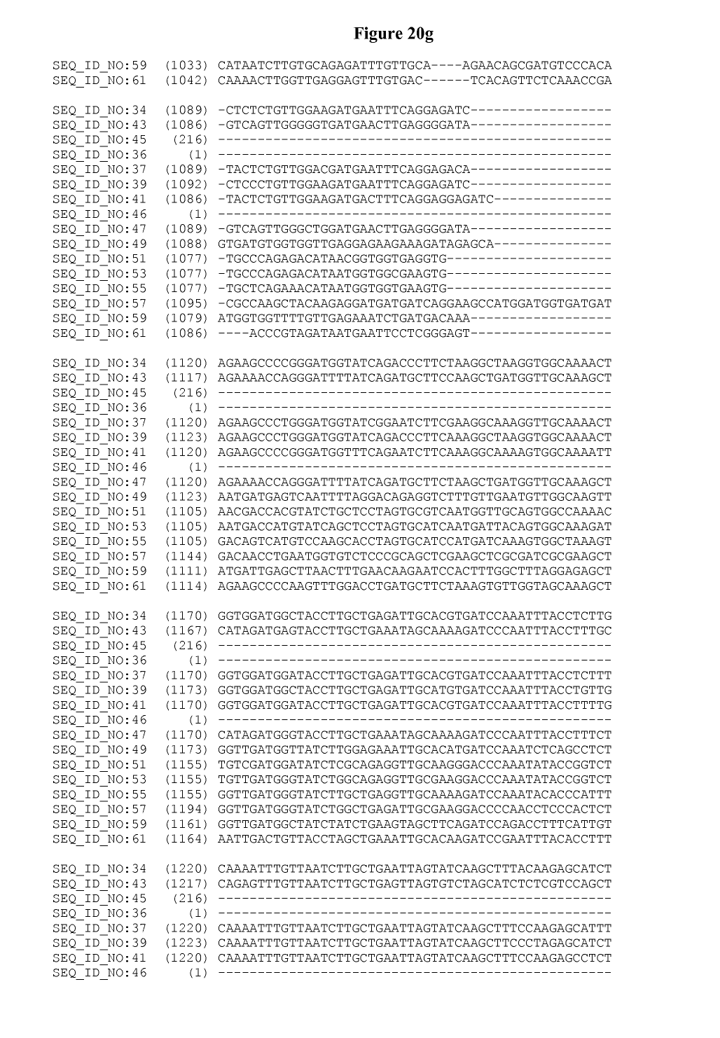

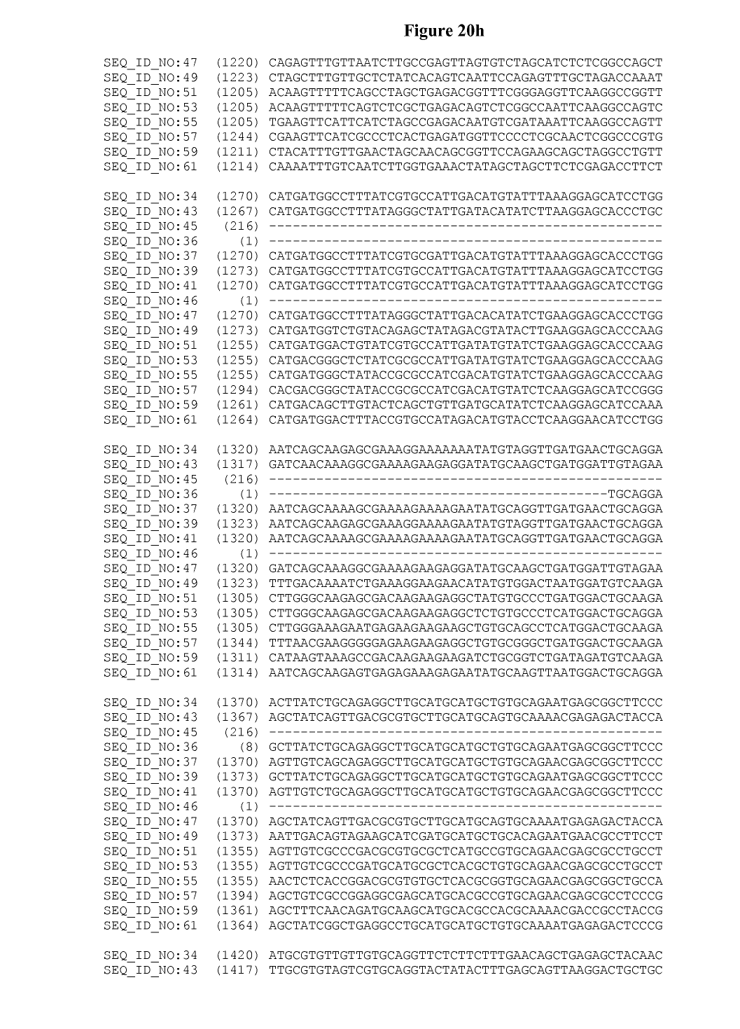

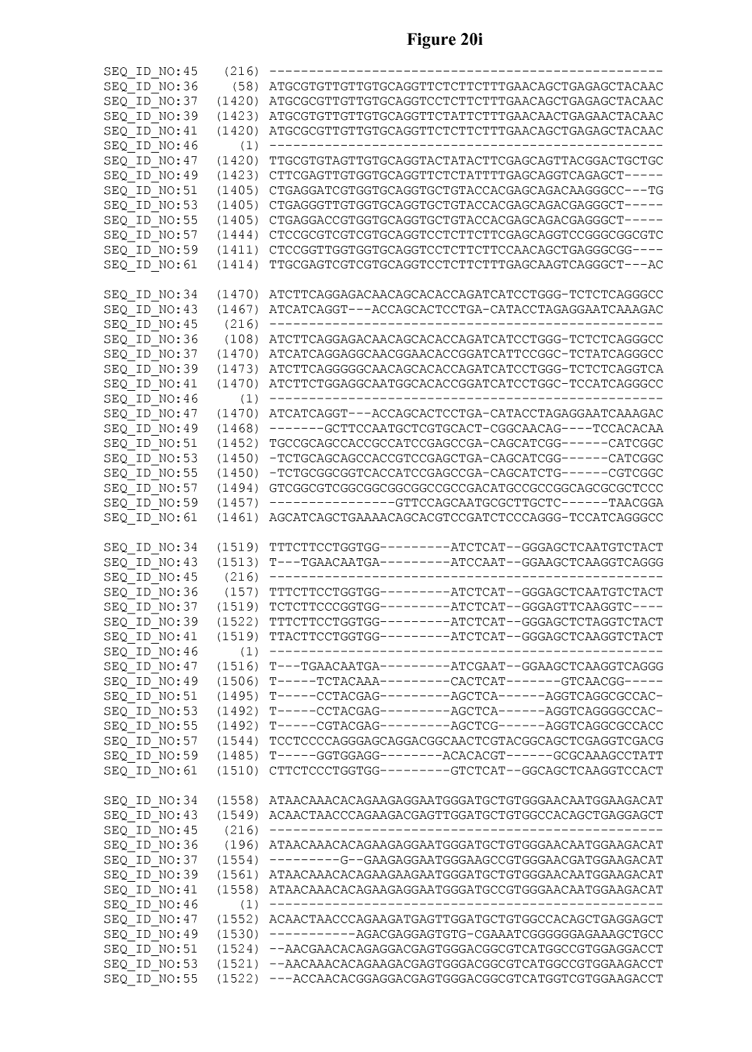

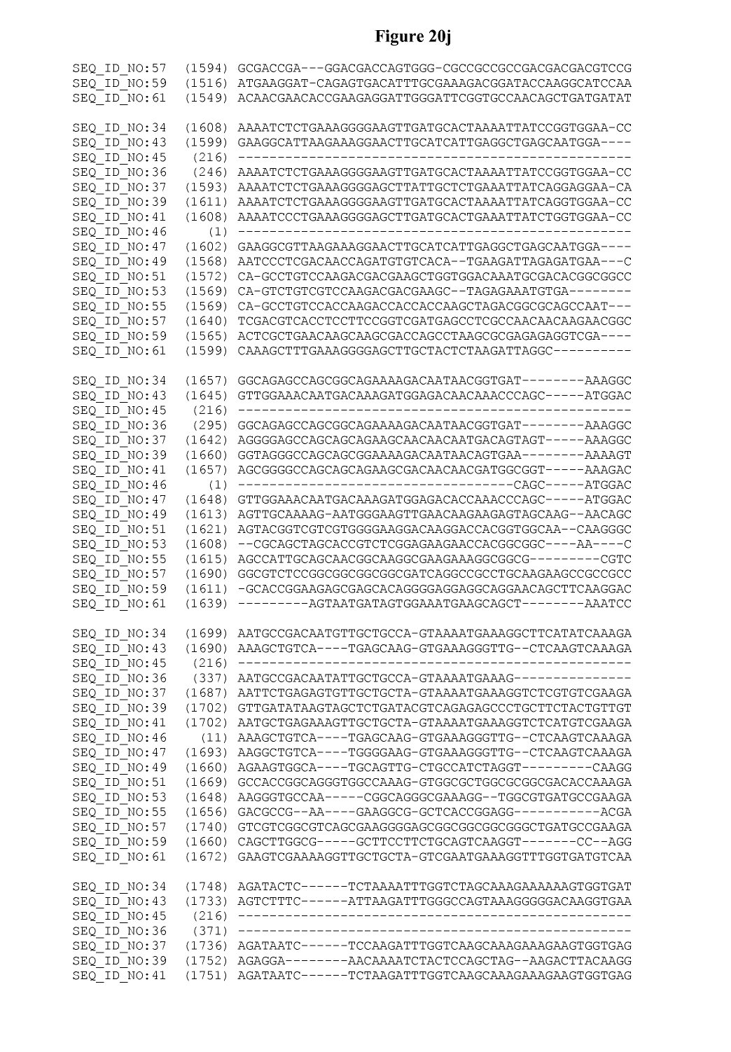

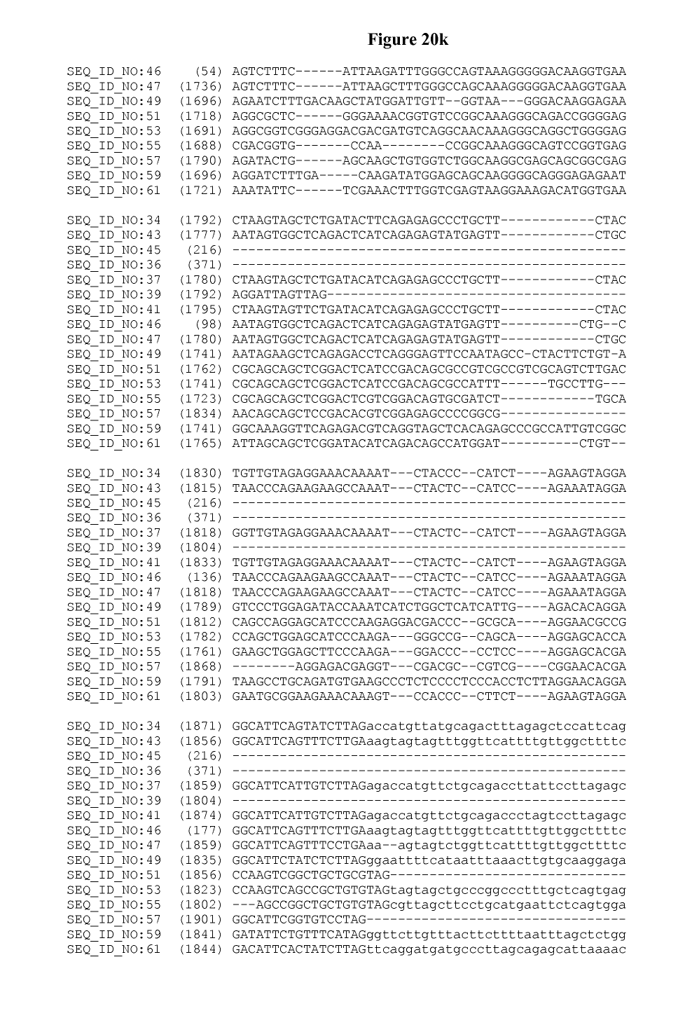

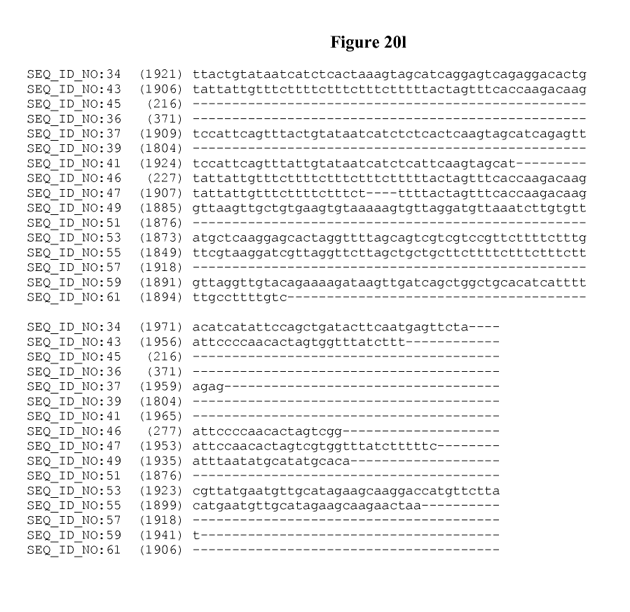

In another embodiment, the dsRNA targets a plant NPY gene. NPY genes belong to a gene family involved in PIN localization in the plant cell effecting auxin response and localization. GmNPY1 (SEQ ID NO:34), GmNPY-like2 (SEQ ID NO:37), GmNPY-like3 (SEQ ID NO:39), GmNPY-like4 (SEQ ID NO:41), GmNPY-like5 (SEQ ID NO:43), GmNPY-like6 (SEQ ID NO:47) and GmNPY-like7 (SEQ ID NO:49) belong to the NPY (Naked Pins in Yuc Mutants) gene family, which includes NPY1 (At4g31820) from Arabidopsis thaliana. The genes in this family contain a BTB/POZ (pfam00651) protein-protein interaction domain and a NPH3 (pfam03000) domain. As shown in Example 1, the full length G. max GmNPY1 gene was isolated and is represented in SEQ ID NO:34. The G. max GmNPY1 gene sequence described by SEQ ID NO:34 contains an open reading frame with the amino acid sequence disclosed as SEQ ID NO:35. The G. max GmNPY-like5 gene sequence described by SEQ ID NO:43 contains an open reading frame with the amino acid sequence disclosed as SEQ ID NO:44. As disclosed in Example 6, the amino acid sequences described by SEQ ID NO:35 and SEQ ID NO:44 were used to identify homologous NPY amino acid sequences from soybean, GmNPY-like7, corn, ZmLOC100280048 and ZM07MC01162_BFb0263J23, rice, OsAK103674.1, Os12g0583500 and Os09g0420900, and cotton, TA26692_{—}3635_Gh. The corresponding homologous amino acid sequences are set forth in SEQ ID NO:50, 52, 54, 56, 58, 60 and 62. The amino acid alignment of representative NPY protein sequences or sequence fragments as set forth in SEQ ID NO:35, 38, 40, 42, 44, 48, 50, 52, 54, 56, 58, 60 and 62 is shown in FIG. 18a-d. The corresponding homologous NPY DNA sequences or sequence fragments are described by SEQ ID NO:49, 51, 53, 55, 57, 59 and 61. The DNA sequence alignment of the representative NPY genes described by SEQ ID NO:34 to SEQ ID NO: 36, 37, 39, 41, 43, 45, 46, 47, 49, 51, 53, 55, 57, 59 and 61 is shown in FIG. 20a-l. Exemplary NPY1 genes targeted by the dsRNA of this embodiment include the sequences set forth in SEQ ID NO:34, 36, 37, 39, 41, 43, 45, 46, 47, 52, 54, 56, 58, 60, or 62; plant NPY genes having at least 80% sequence identity to SEQ ID NO:34, 36, 37, 39, 41, 43, 45, 46, 47, 52, 54, 56, 58, 60, or 62; and plant NPY genes that hybridize under stringent conditions to the sequence set forth in SEQ ID NO:34, 36, 37, 39, 41, 43, 45, 46, 47, 52, 54, 56, 58, 60, or 62.

In accordance with this embodiment, the dsRNA encoded by the expression vector of the invention comprises a first strand that is substantially identical to at least 19, 20, or 21 consecutive nucleotides of an NPY1 target gene of a plant genome and a second strand that is substantially complementary to the first strand. Preferably, the NPY dsRNA first strand comprises at least 19, 20, or 21 consecutive nucleotides of a plant NPY polynucleotide selected from the group consisting of: (a) a polynucleotide having the sequence set forth in SEQ ID NO:34, 36, 37, 39, 41, 43, 45, 46, 47, 52, 54, 56, 58, 60, or 62; (b) a plant NPY polynucleotide having at least 80% sequence identity to SEQ ID NO:34, 36, 37, 39, 41, 43, 45, 46, 47, 52, 54, 56, 58, 60, or 62; and (c) a plant NPY polynucleotide that hybridizes under stringent conditions to the polynucleotide having the sequence set forth in SEQ ID NO:34, 36, 37, 39, 41, 43, 45, 46, 47, 52, 54, 56, 58, 60, or 62.

Additional cDNAs corresponding to the plant target genes of the invention may be isolated from plants other than G. max using the information provided herein and techniques known to those of skill in the art of biotechnology. For example, a nucleic acid molecule from a plant that hybridizes under stringent conditions to a nucleotide sequence of SEQ ID NO:1, 3, 4, 6, 8, 10, 12, 13, 15, 17, 19, 20, 22, 24, 26, 27, 29, 31, 32, 34, 36, 37, 39, 41, 43, 45, 46 or 47 can be isolated from plant cDNA libraries. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; well known in the art. Alternatively, mRNA can be isolated from plant cells, and cDNA can be prepared using reverse transcriptase. Synthetic oligonucleotide primers for polymerase chain reaction amplification can be designed based upon the nucleotide sequence shown in SEQ ID NO:1, 3, 4, 6, 8, 10, 12, 13, 15, 17, 19, 20, 22, 24, 26, 27, 29, 31, 32, 34, 36, 37, 39, 41, 43, 45, 46 or 47. Nucleic acid molecules corresponding to the plant target genes of the invention can be amplified using cDNA or, alternatively, genomic DNA, as a template and appropriate oligonucleotide primers according to standard PCR amplification techniques. The nucleic acid molecules so amplified can be cloned into appropriate vectors and characterized by DNA sequence analysis.

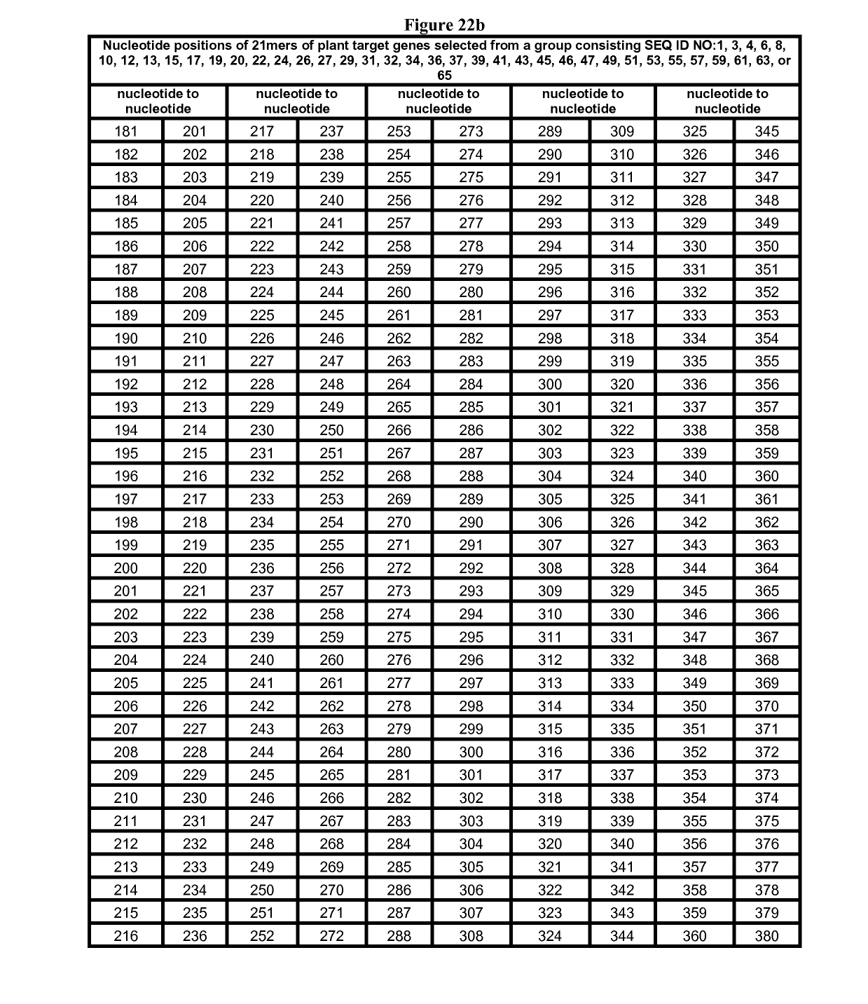

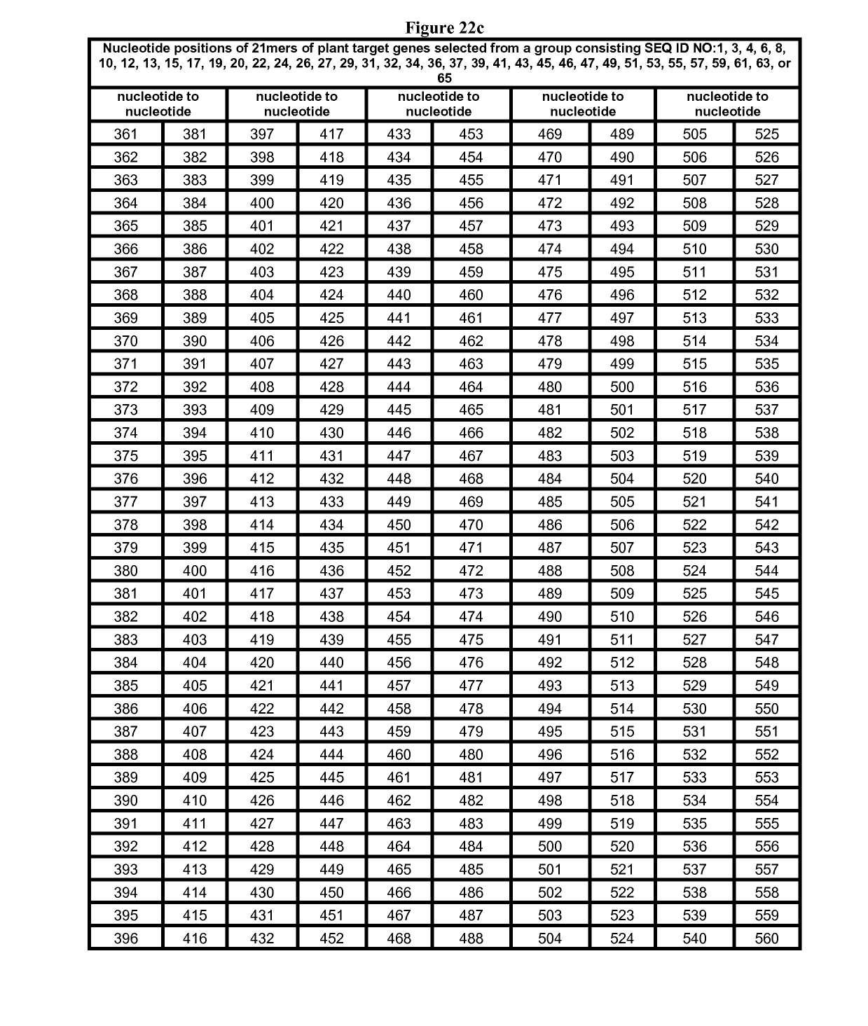

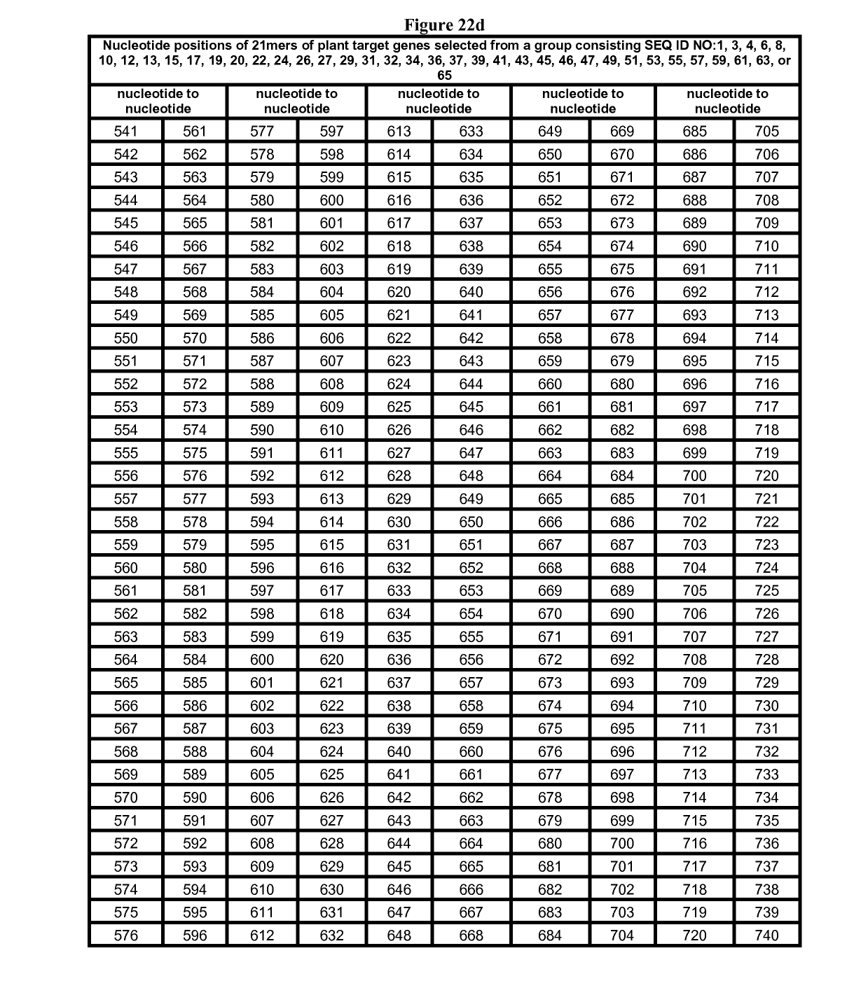

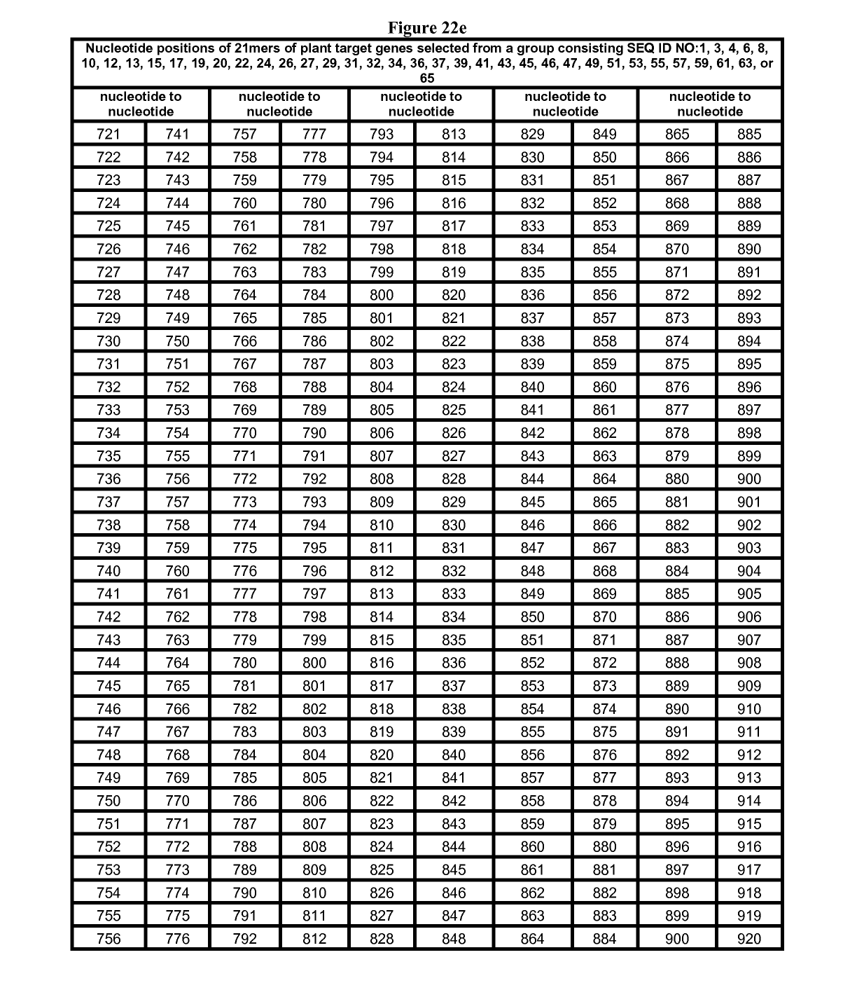

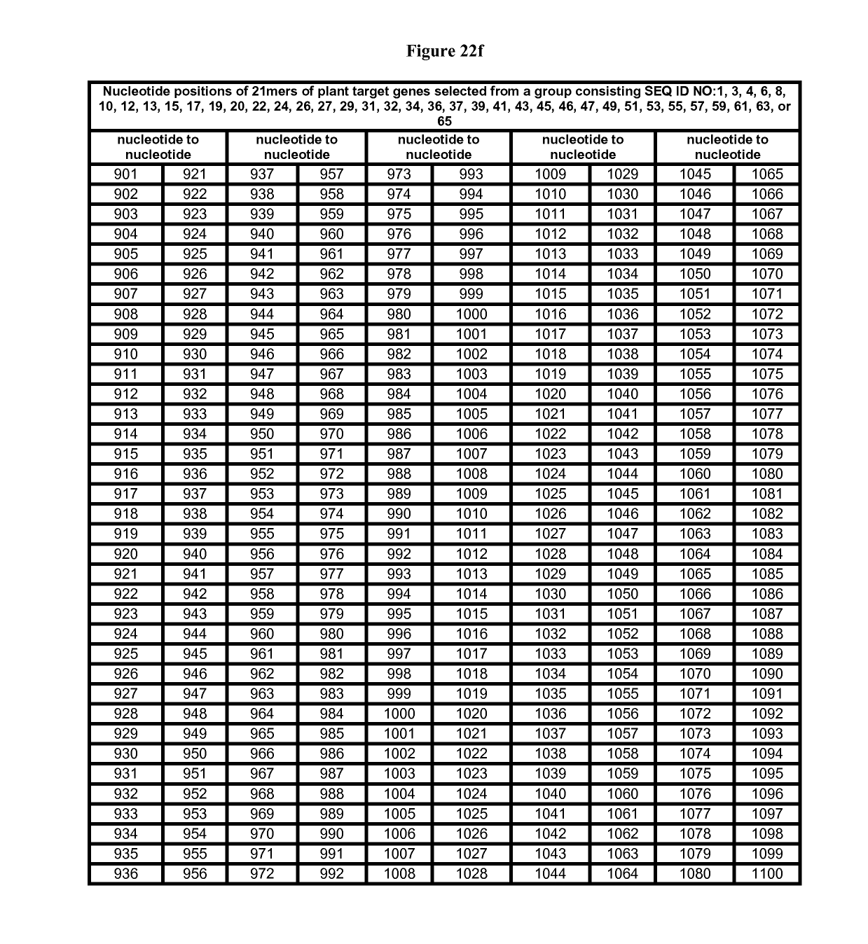

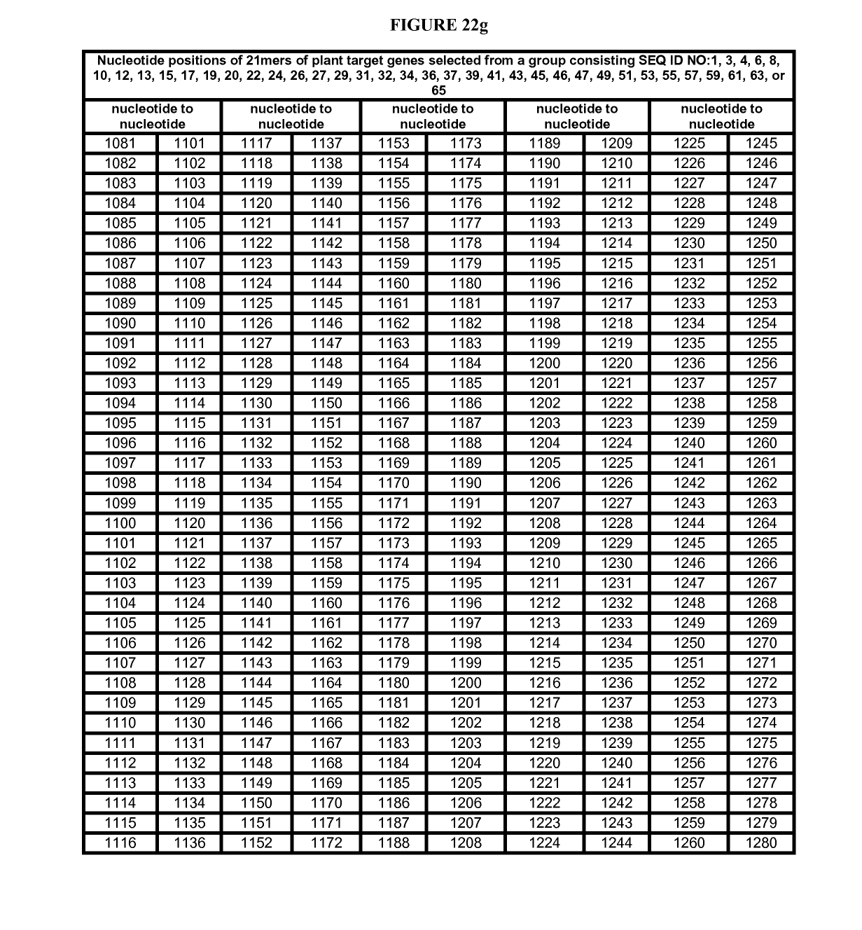

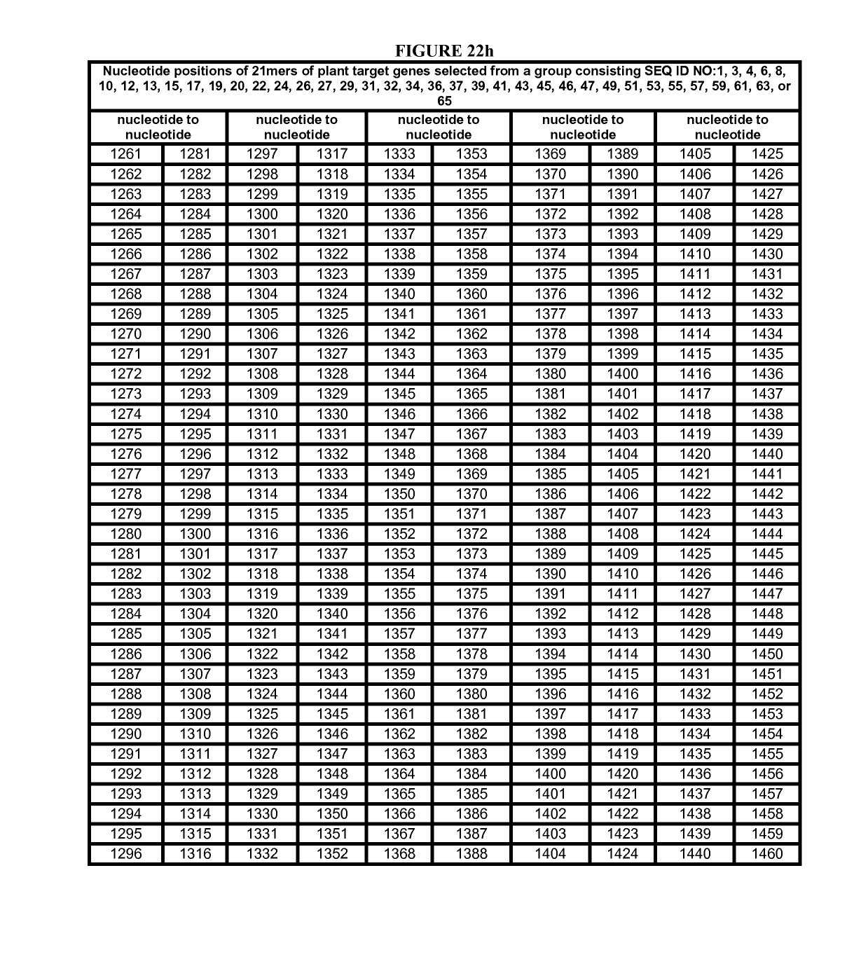

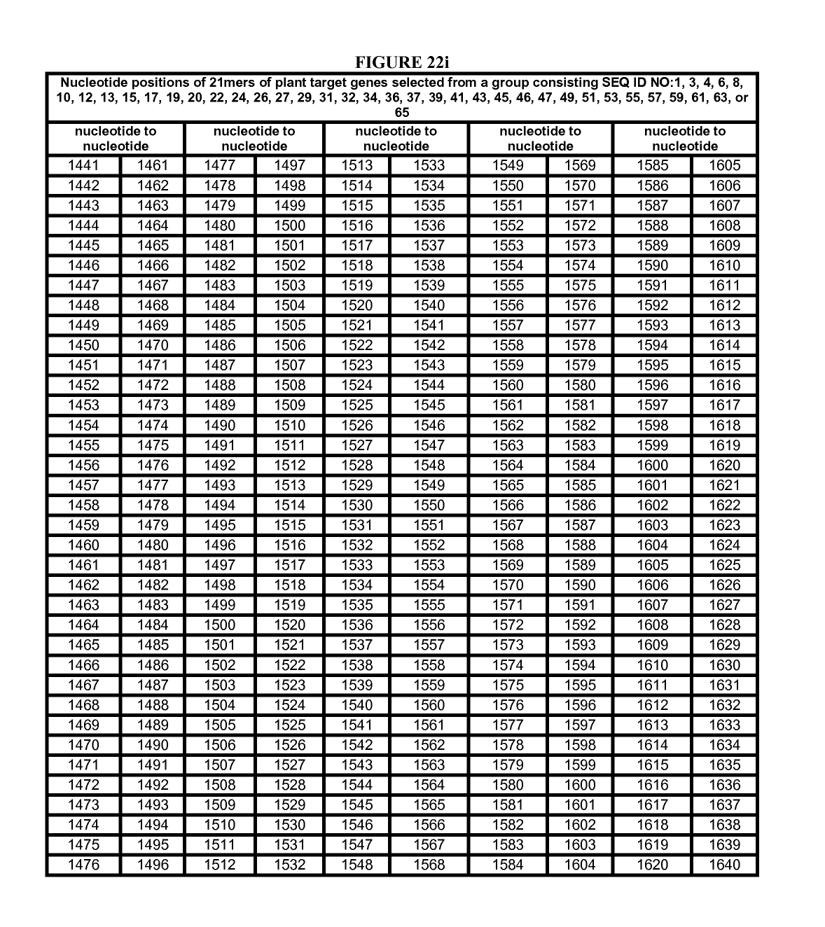

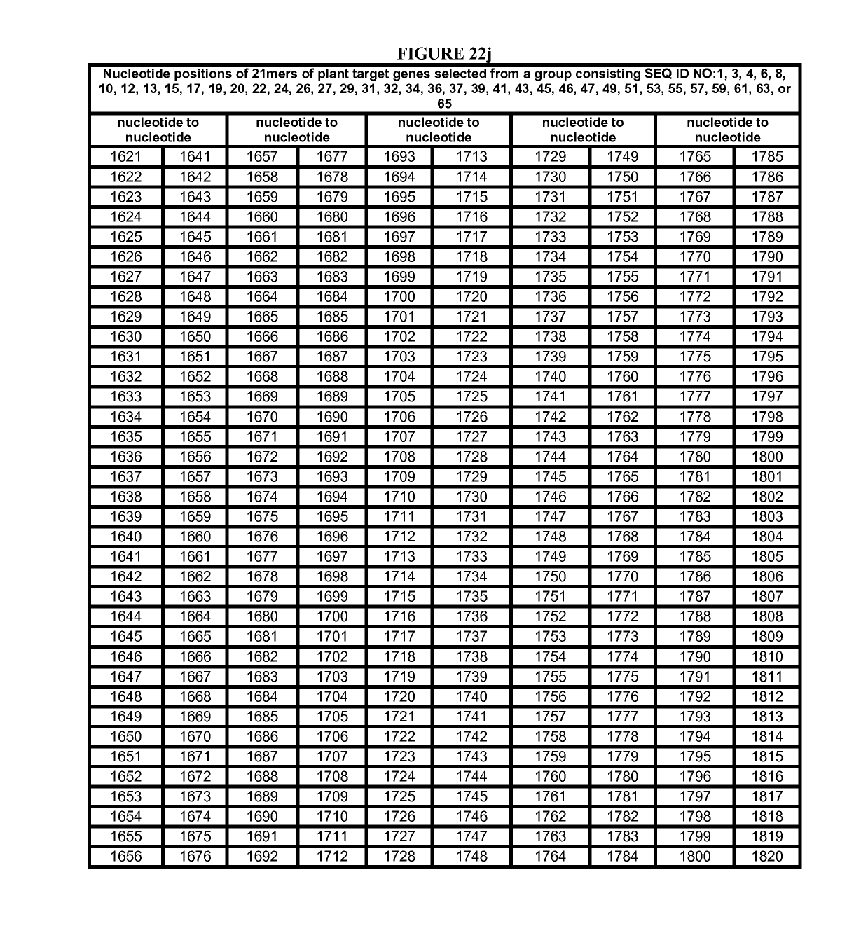

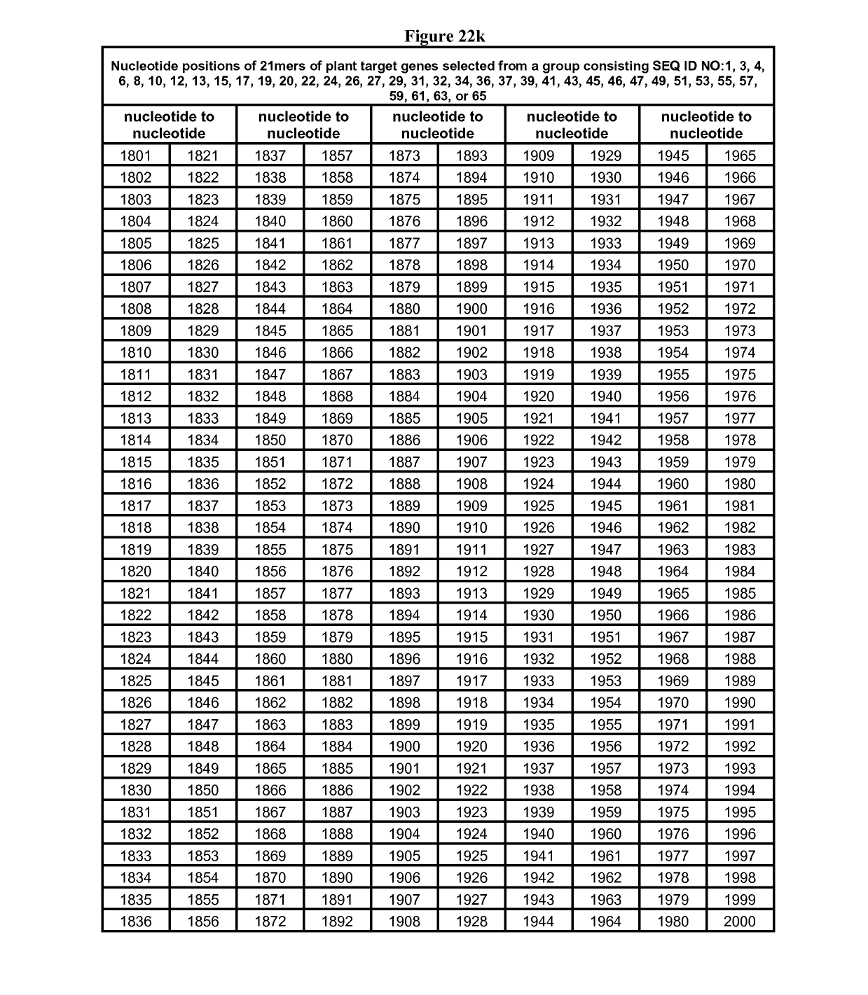

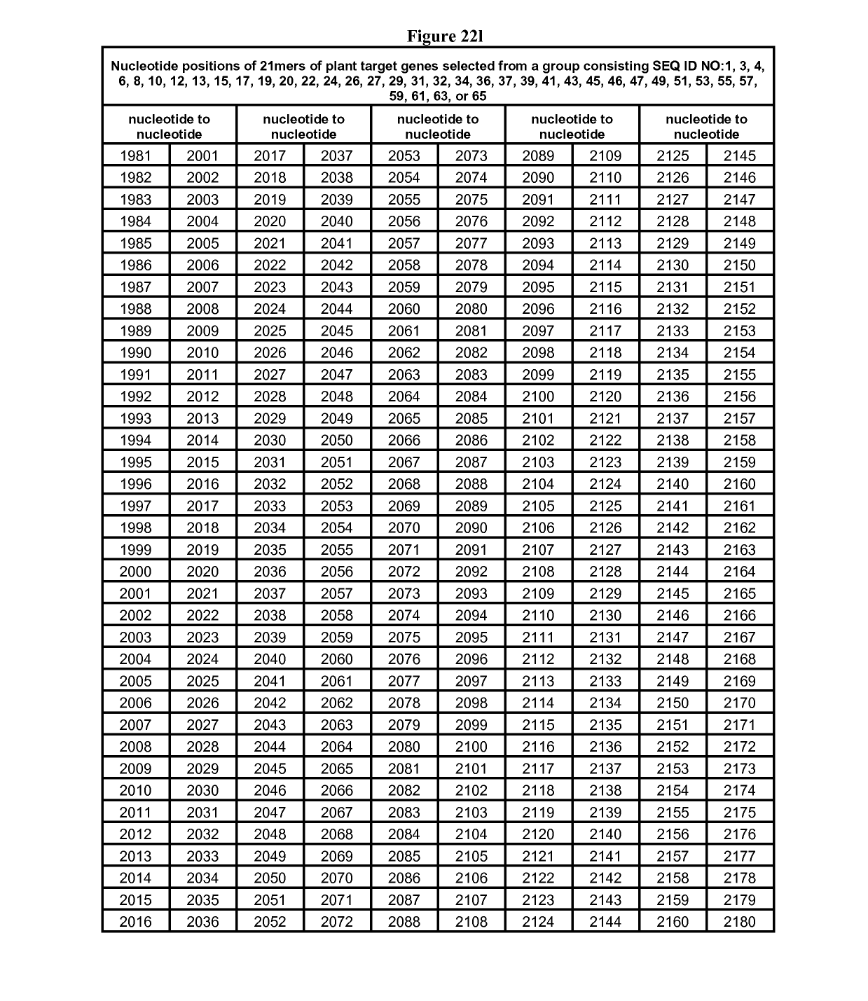

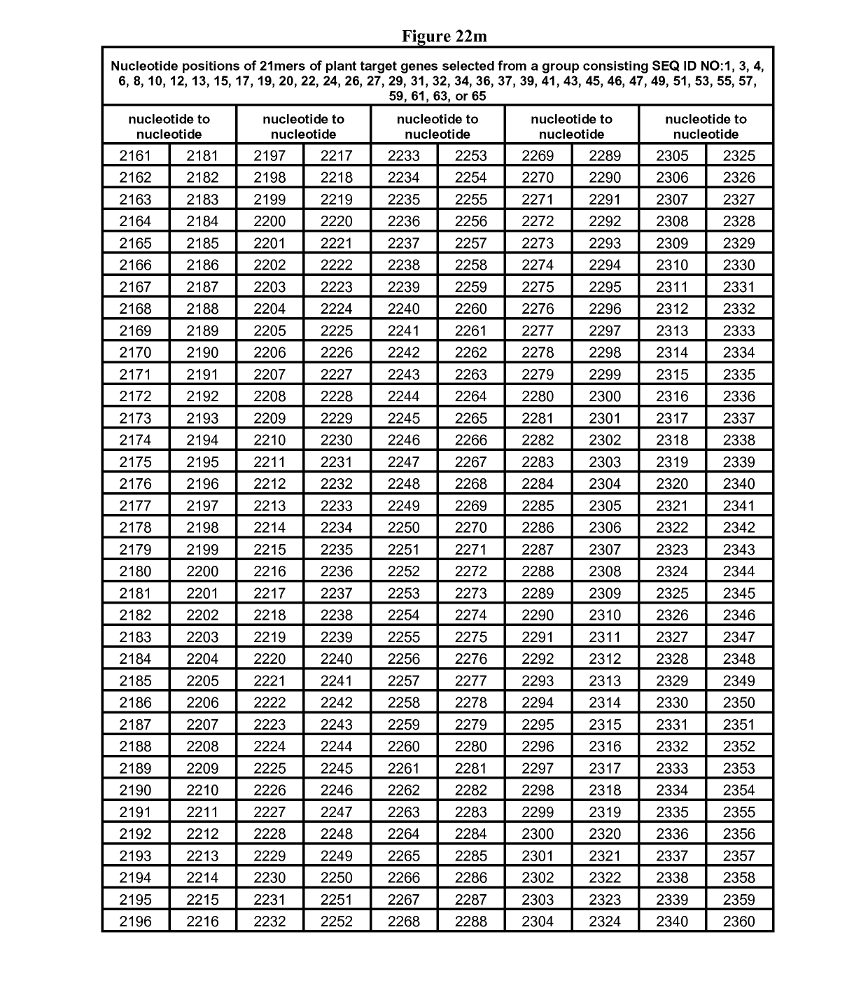

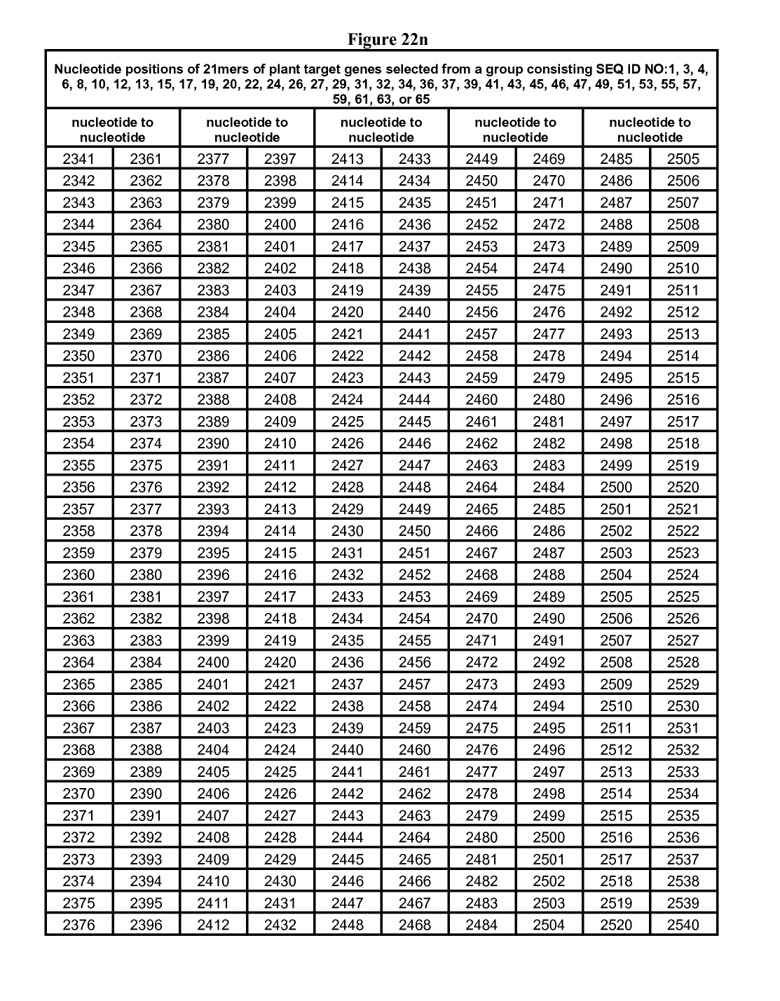

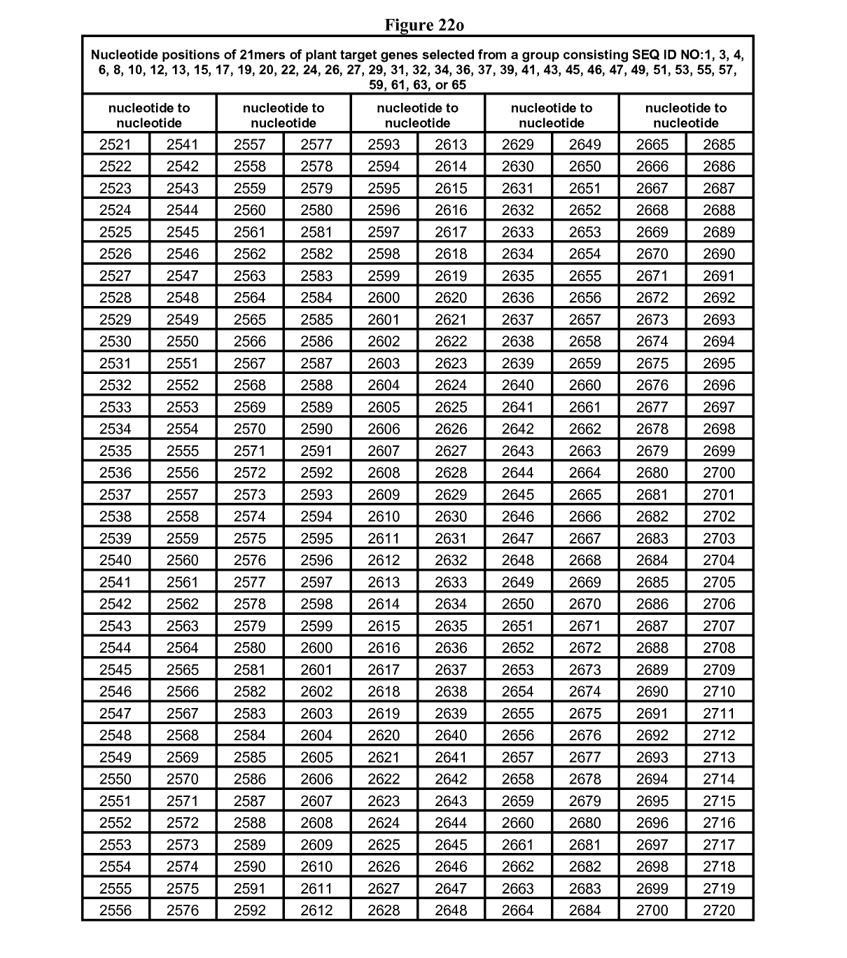

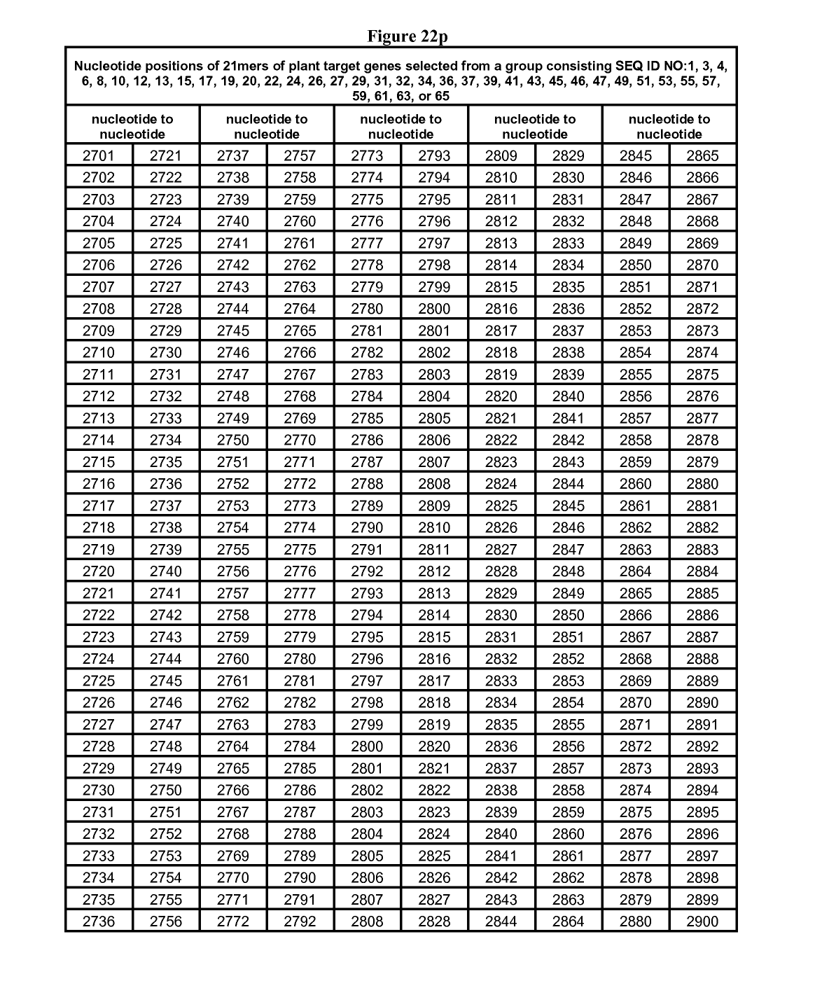

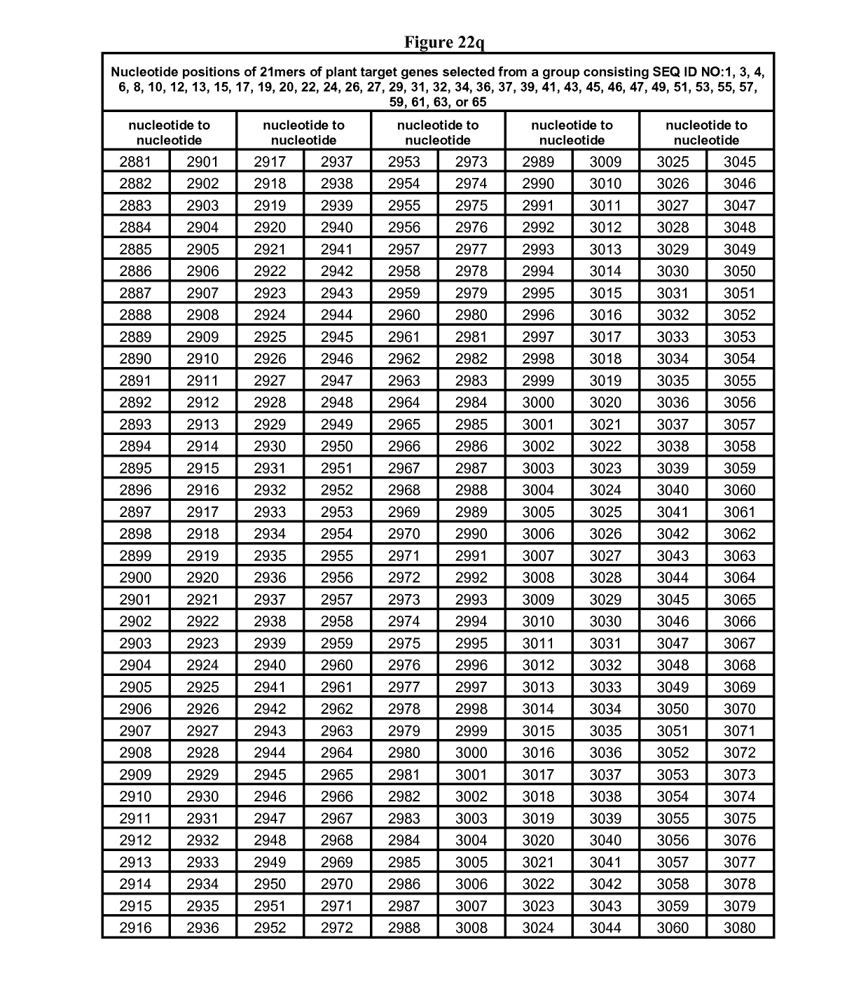

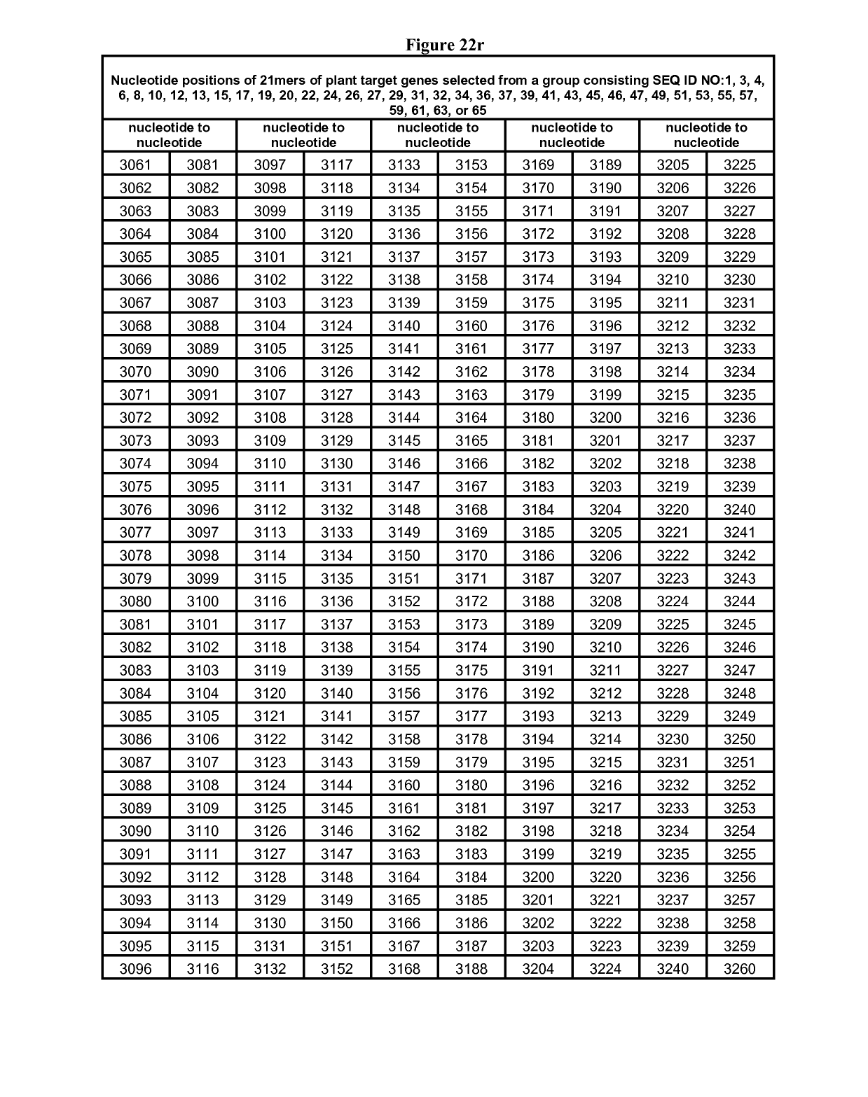

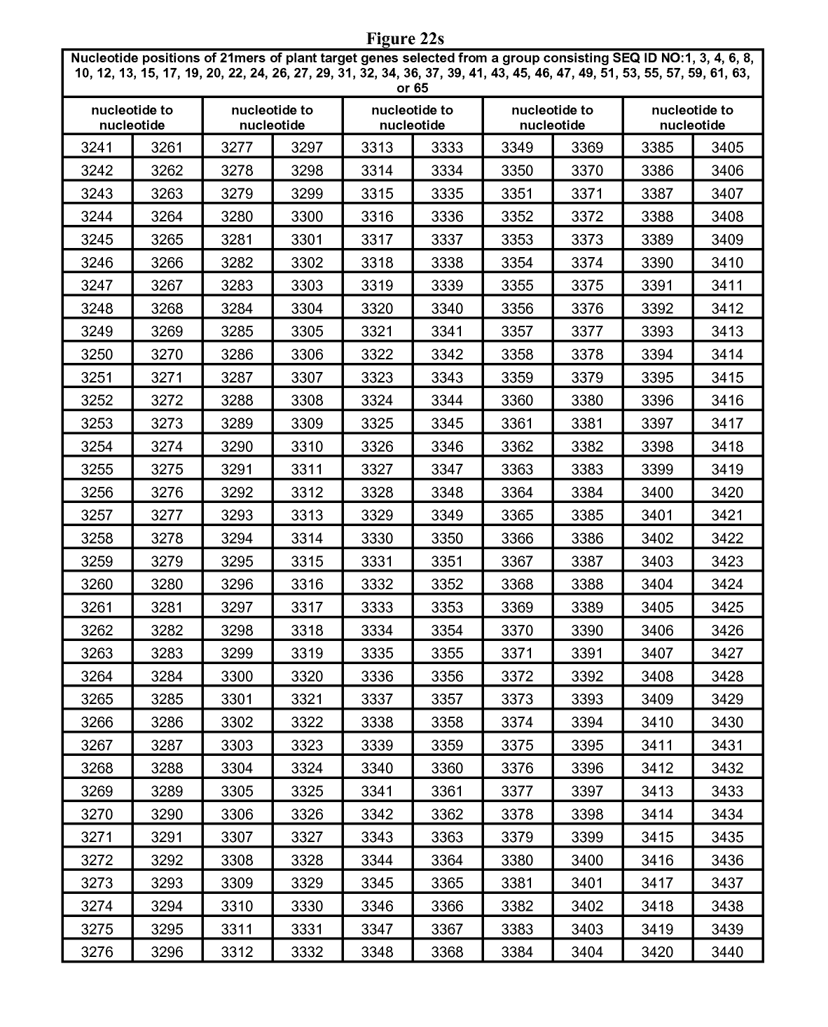

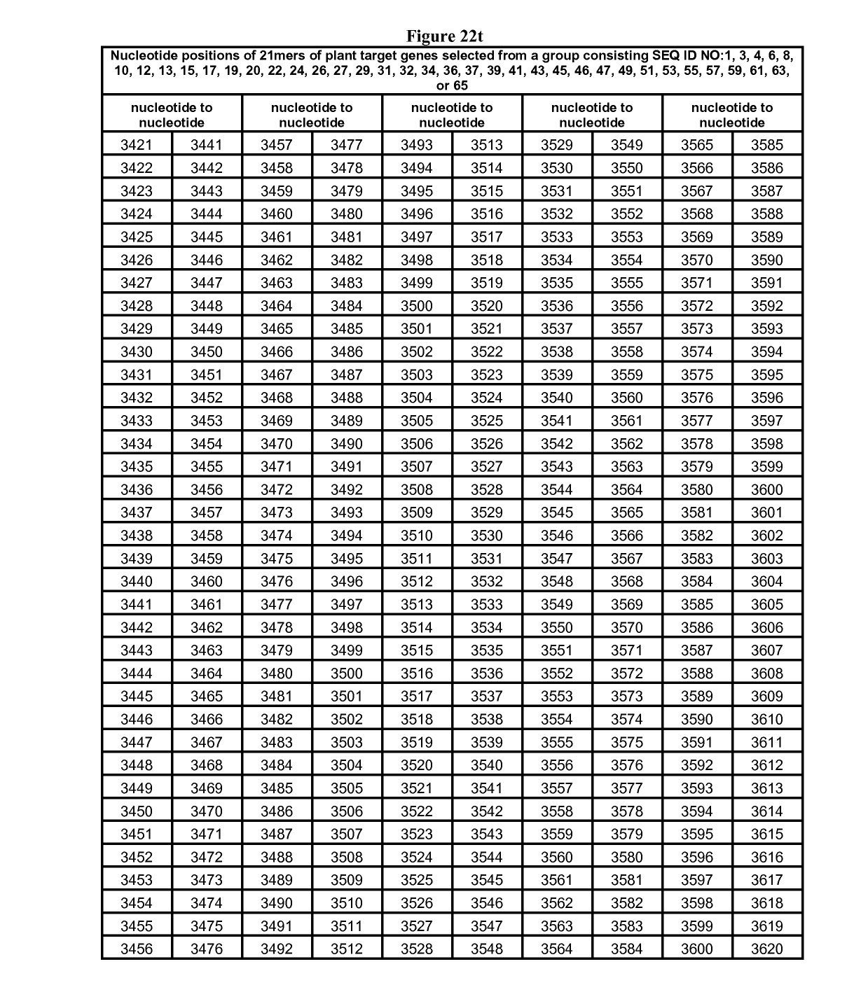

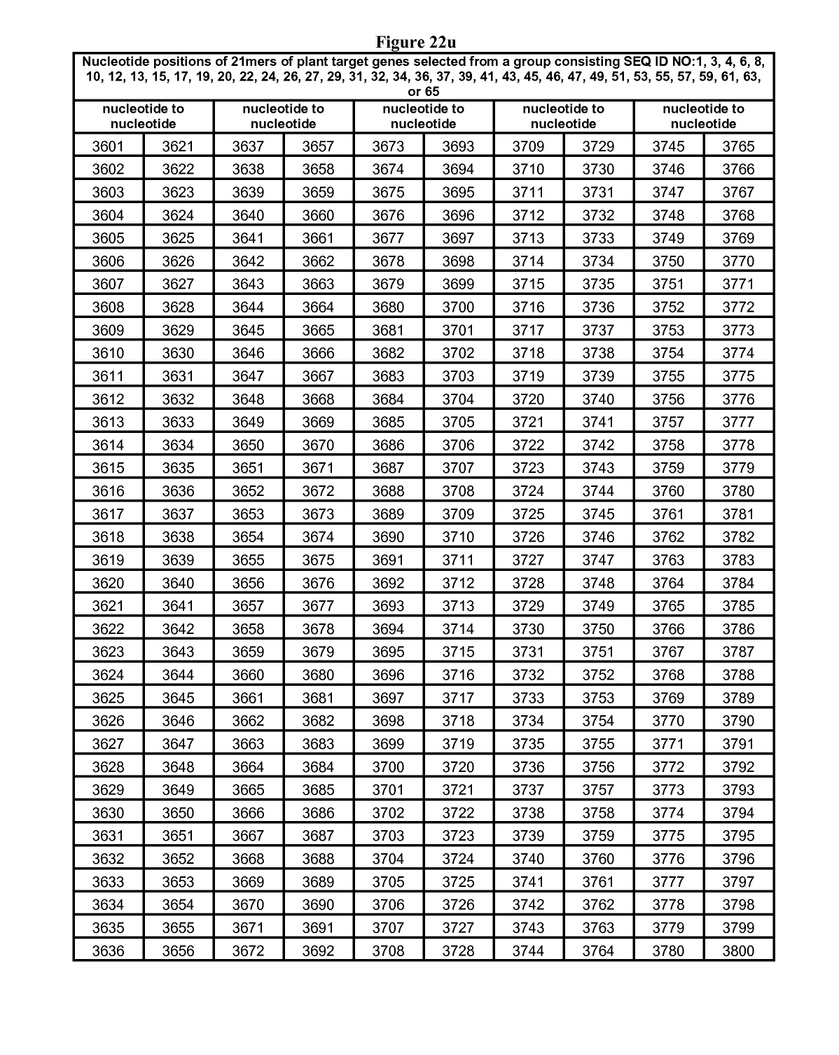

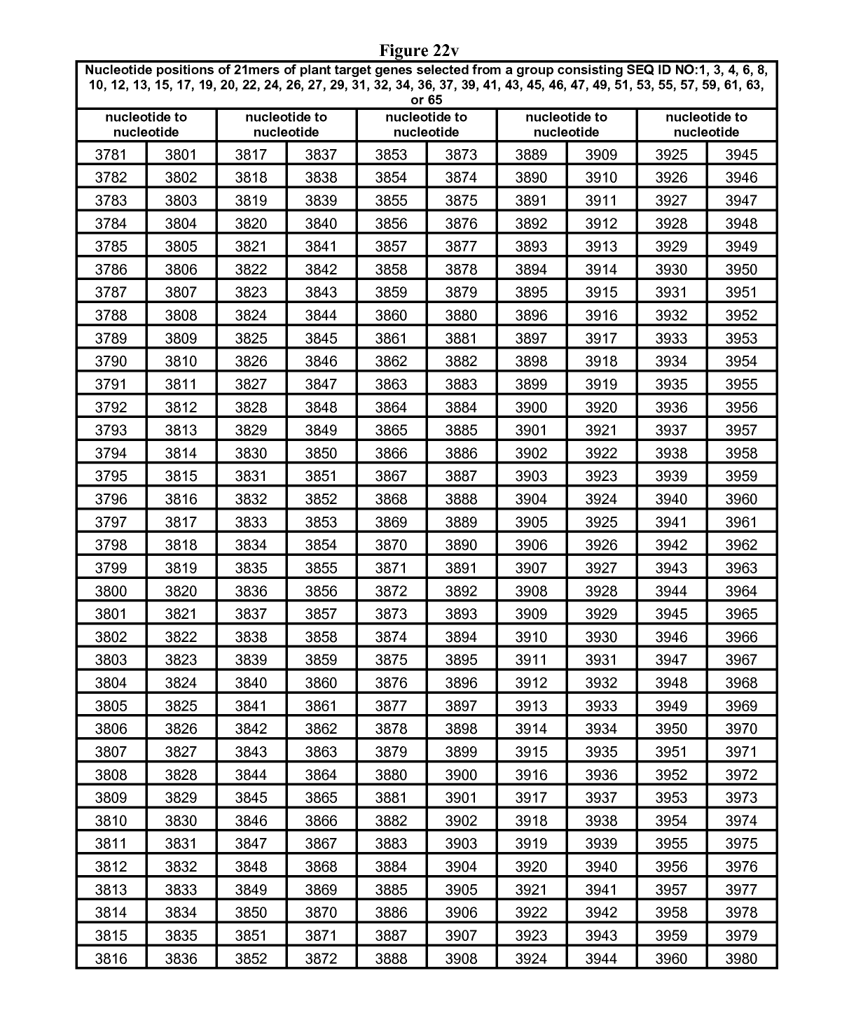

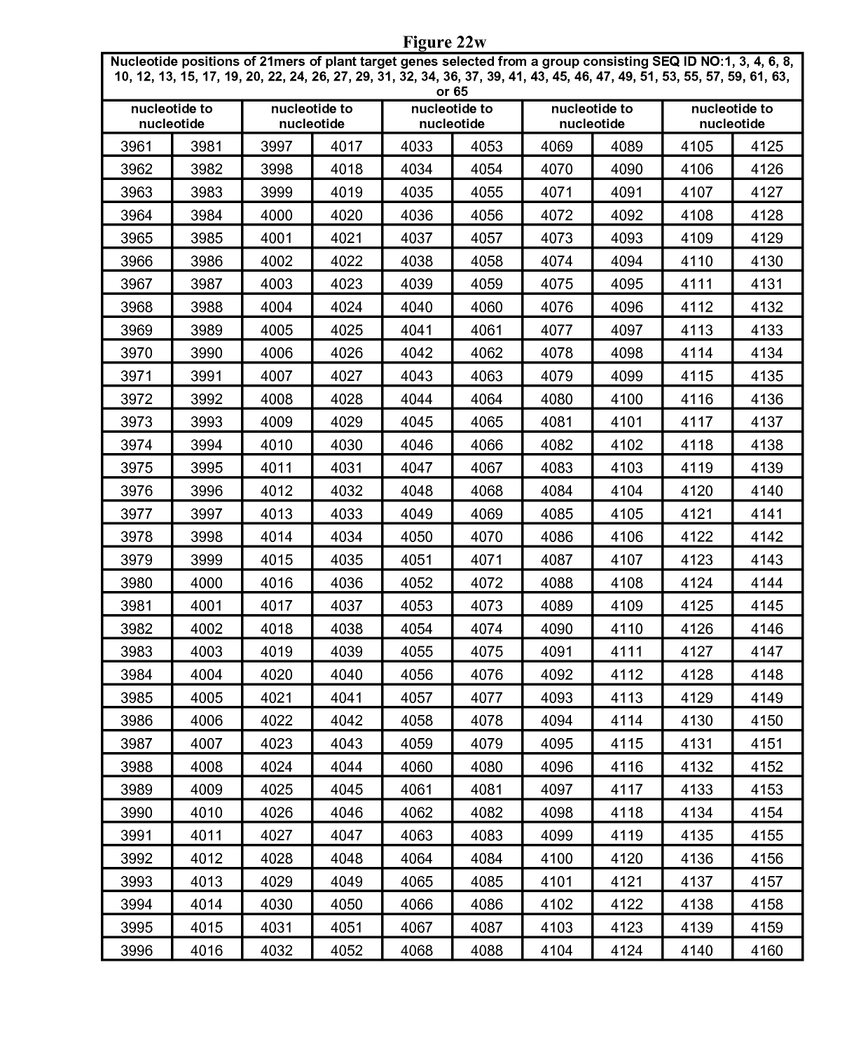

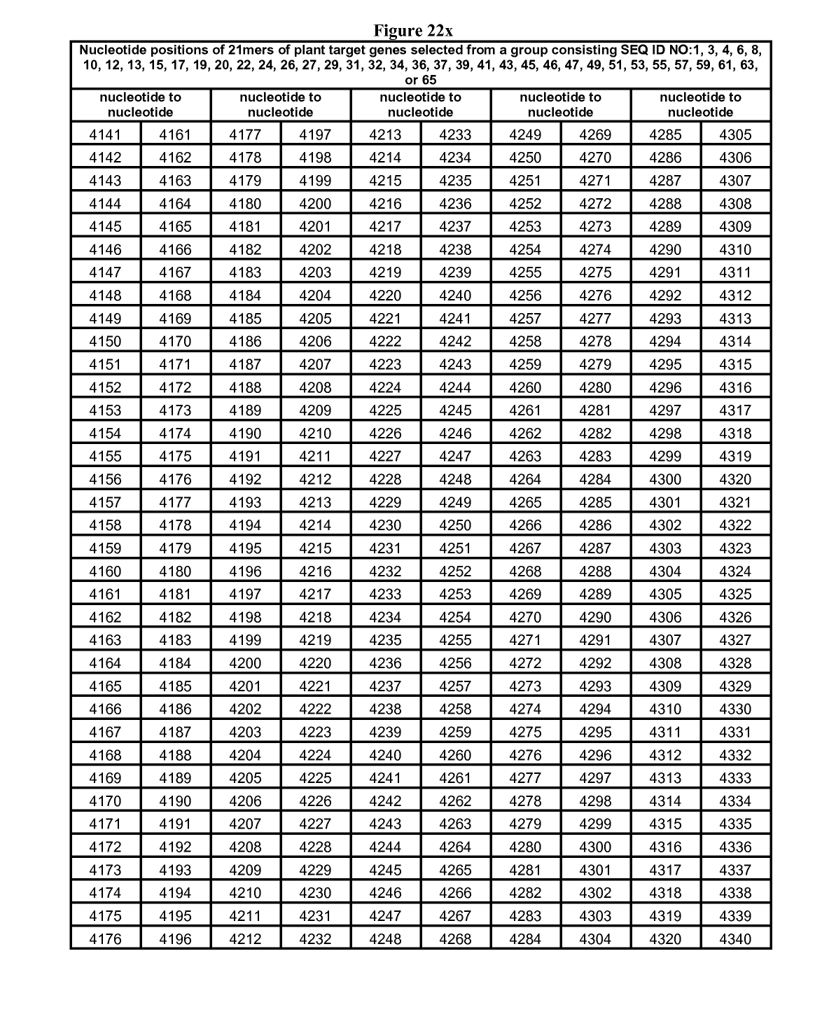

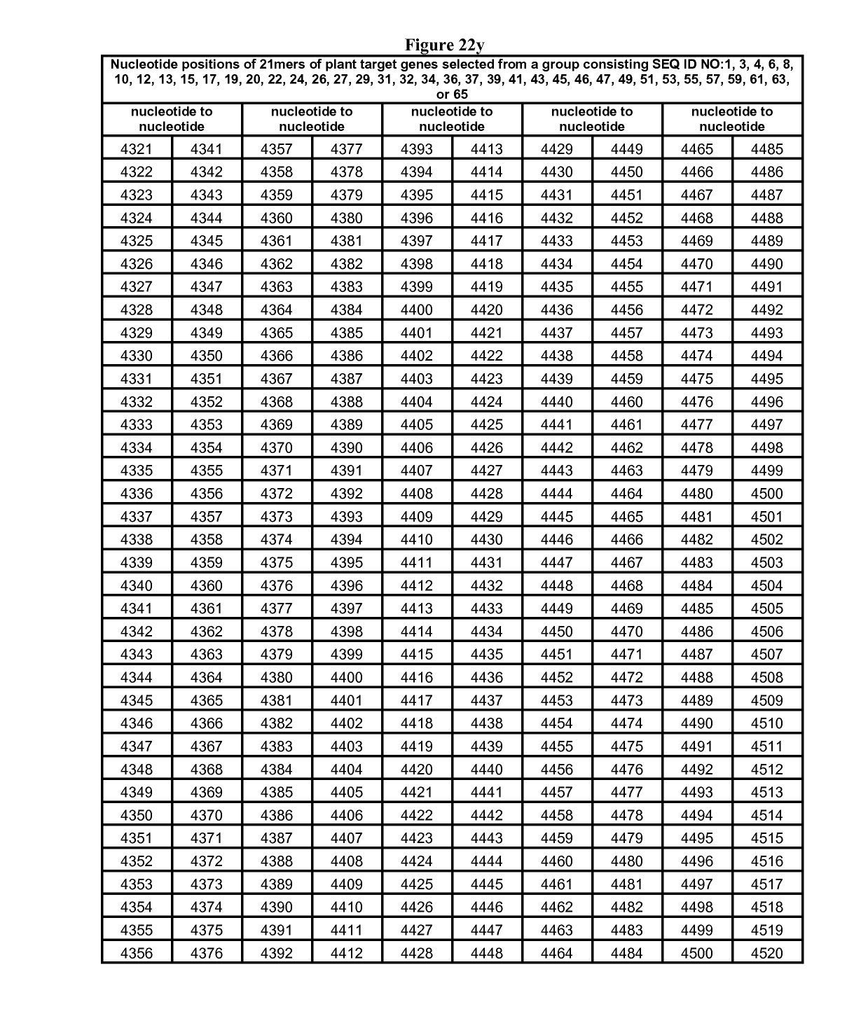

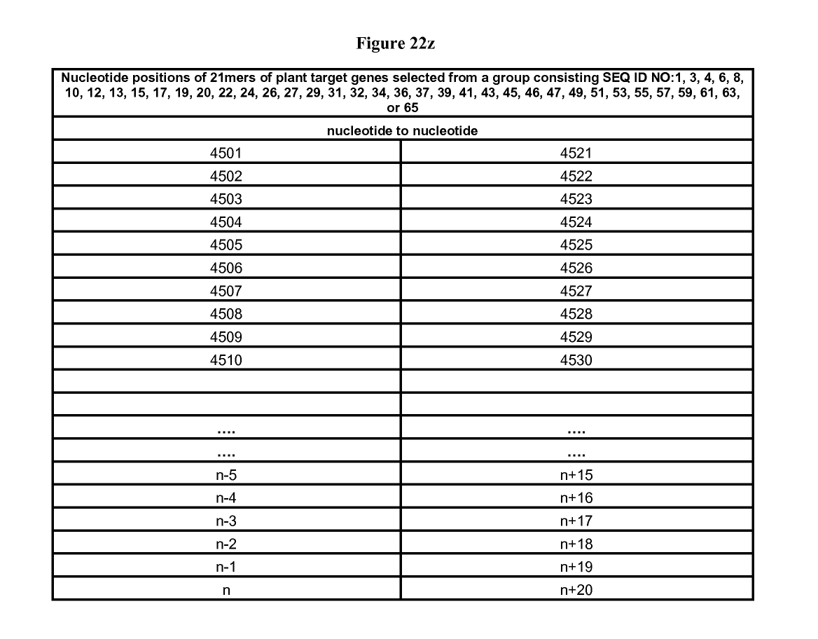

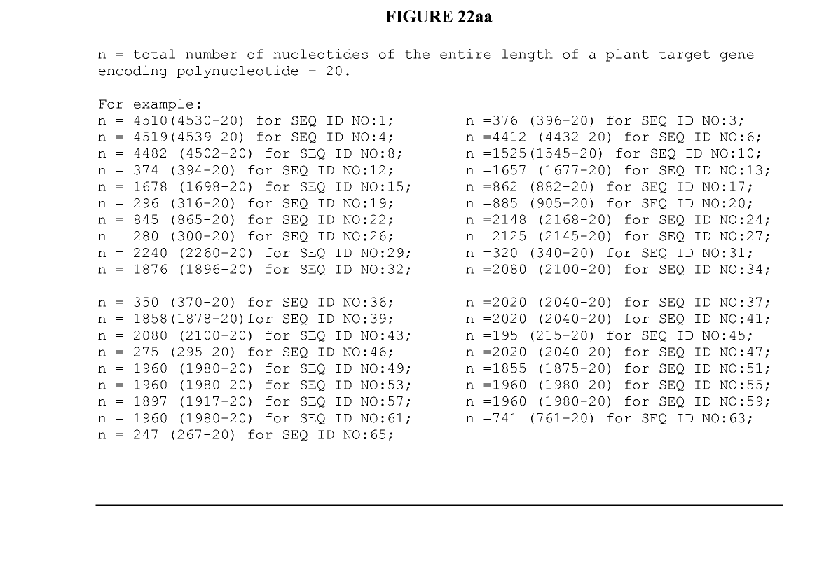

As discussed above, fragments of dsRNA larger than about 19-24 nucleotides in length are cleaved intracellularly by nematodes and plants to siRNAs of about 19-24 nucleotides in length, and these siRNAs are the actual mediators of the RNAi phenomenon. The table in FIGS. 22a-aa sets forth exemplary 21-mers of the soybean CLASP1 gene, SEQ ID NO:1, Aspartic Proteinase Delta Subunit gene, SEQ ID NO:10, Secreted Protein1 gene, SEQ ID NO:17, Lectin Receptor Kinase-like gene, SEQ ID NO:24, Pectin Methylesterase-like gene, SEQ ID NO:29, NPY1 gene, SEQ ID NO:34, and NPY-like5 gene, SEQ ID NO:43, and the respective fragments and homologs thereof, as indicated by SEQ ID NOs set forth in the table. This table can also be used to calculate the 19, 20, 22, 23, or 24-mers by adding or subtracting the appropriate number of nucleotides from each 21mer.

The expression vector of the invention encodes at least one dsRNA which may range in length from about 19 nucleotides to about 200 consecutive nucleotides or up to the whole length of the target gene. The dsRNA encoded by the expression vector of the invention may be embodied as a miRNA which targets a single site corresponding to a portion of the target gene comprising 19, 20, or 21 consecutive nucleotides thereof. Alternatively, the dsRNA encoded by the expression vector of the invention has a length from about 19, 20, or 21 consecutive nucleotides to about 200 consecutive nucleotides of the target gene. In another embodiment, the dsRNA encoded by the expression vector of the invention has a length from about 19, 20, or 21 consecutive nucleotides to about 400 consecutive nucleotides, or from about 19, 20, or 21 consecutive nucleotides to about 600 consecutive nucleotides of the target gene.

As disclosed herein, 100% sequence identity between the dsRNA and the target gene is not required to practice the present invention. Preferably, the dsRNA of the invention comprises a 19-nucleotide portion which is substantially identical to a 19 contiguous nucleotide portion of the target gene. While a dsRNA comprising a nucleotide sequence that is identical to a portion of the plant target gene is preferred for inhibition, the invention can tolerate sequence variations within the dsRNA that might be expected due to gene manipulation or synthesis, genetic mutation, strain polymorphism, or evolutionary divergence. Thus the dsRNAs of the invention also encompass dsRNAs comprising a mismatch with the target gene of at least 1, 2, or more nucleotides. For example, it is contemplated in the present invention that the 21mer dsRNA sequences exemplified in FIGS. 22a-22aa may contain an addition, deletion or substitution of 1, 2, or more nucleotides, so long as the resulting sequence still interferes with the plant target gene function.

Sequence identity between the dsRNAs of the invention and the plant target genes may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 80% sequence identity, 90% sequence identity, or even 100% sequence identity, between the inhibitory RNA and at least 19 contiguous nucleotides of the target gene is preferred.

Because multiple specialized Dicer enzymes in plants generate siRNAs typically ranging in size from 19 nt to 24 nt (See Henderson et al., 2006. Nature Genetics 38:721-725.), the siRNAs encoded by the expression vector of the present invention can may range from about 19 contiguous nucleotide sequences to about 24 contiguous nucleotide sequences across the length of a target gene. Thus when dsRNA encoded by the expression vector of the invention has a length longer than about 21 nucleotides, for example from about 50 nucleotides to about 1000 nucleotides, it will be cleaved randomly to siRNAs of 19-24 nucleotides within the plant cell. The cleavage of a longer dsRNA of the invention will yield a pool comprising a multiplicity of siRNAs derived from the longer dsRNA. For example, a pool of siRNA produced by the expression vector of the invention derived from the G. max target genes disclosed herein may comprise a multiplicity of siRNA molecules which are selected from the group consisting of oligonucleotides substantially identical to any 19mer, 20mer, 21mer, 22mer, 23mer, or 24mer derived from SEQ ID NO:1, SEQ ID NO:4, SEQ ID NO:6, SEQ ID NO:8, SEQ ID NO:10, SEQ ID NO:13, SEQ ID NO:15, SEQ ID NO:17, SEQ ID NO:20, SEQ ID NO:22, SEQ ID NO:24, SEQ ID NO:27, SEQ ID NO:29, SEQ ID NO:32, SEQ ID NO:34, SEQ ID NO:37, SEQ ID NO:39, SEQ ID NO:41, SEQ ID NO:43, SEQ ID NO: 45, SEQ ID NO: 46, SEQ ID NO:47; SEQ ID NO:52, SEQ ID NO:54, SEQ ID NO:56, SEQ ID NO:58, SEQ ID NO:60, or SEQ ID NO:62, SEQ ID NO: 63, or SEQ ID NO: 65, as described in FIGS. 22a-22aa. Alternatively, the pool of siRNA encoded by the expression vector of the invention may comprise a multiplicity of RNA molecules having a combination of any 19, 20, 21, 22, 23, and/or 24 contiguous nucleotide sequences derived from SEQ ID NO:1, SEQ ID NO:4, SEQ ID NO:6, SEQ ID NO:8, SEQ ID NO:10, SEQ ID NO:13, SEQ ID NO:15, SEQ ID NO:17, SEQ ID NO:20, SEQ ID NO:22, SEQ ID NO:24, SEQ ID NO:27, SEQ ID NO:29, SEQ ID NO:32, SEQ ID NO:34, SEQ ID NO:37, SEQ ID NO:39, SEQ ID NO:41, SEQ ID NO:43, SEQ ID NO: 45, SEQ ID NO: 46, SEQ ID NO:47; SEQ ID NO:52, SEQ ID NO:54, SEQ ID NO:56, SEQ ID NO:58, SEQ ID NO:60, or SEQ ID NO:62.

Thus the invention is also embodied as an isolated expression vector comprising a nucleic acid encoding a multiplicity of double stranded RNA molecules each comprising a double stranded region having a length of at least 19, 20, or 21 nucleotides, wherein one strand of said double stranded region is derived from a polynucleotide selected from the group consisting of (a) a polynucleotide having a sequence as set forth in SEQ ID NO:1, SEQ ID NO:4, SEQ ID NO:6, SEQ ID NO:8, SEQ ID NO: 63, or SEQ ID NO: 65; (b) a polynucleotide having a sequence as set forth in SEQ ID NO:10, SEQ ID NO:13, or SEQ ID NO:15; (c) a polynucleotide having a sequence as set forth in SEQ ID NO:17, SEQ ID NO:20, or SEQ ID NO:22; (d) a polynucleotide having a sequence as set forth in SEQ ID NO:24 or SEQ ID NO:27; (e) a polynucleotide comprising a sequence as set forth in SEQ ID NO:29 or SEQ ID NO:32; (f) a polynucleotide having a sequence as set forth in SEQ ID NO:34, SEQ ID NO:37, SEQ ID NO:39, SEQ ID NO:41, SEQ ID NO:43, SEQ ID NO: 45, SEQ ID NO: 46, SEQ ID NO:47; SEQ ID NO:52, SEQ ID NO:54, SEQ ID NO:56, SEQ ID NO:58, SEQ ID NO:60, or SEQ ID NO:62.

The dsRNA of the invention may optionally comprise a single stranded overhang at either or both ends. Preferably, the single stranded overhang comprises at least two nucleotides at the 3′ end of each strand of the dsRNA molecule. The double-stranded structure may be formed by a single self-complementary RNA strand (i.e. forming a hairpin loop) or two complementary RNA strands. RNA duplex formation may be initiated either inside or outside the cell. When the dsRNA of the invention forms a hairpin loop, it may optionally comprise an intron, as set forth in US 2003/0180945A1 or a nucleotide spacer, which is a stretch of sequence between the complementary RNA strands to stabilize the hairpin transgene in cells. Methods for making various dsRNA molecules are set forth, for example, in WO 99/53050 and in U.S. Pat. No. 6,506,559. The RNA may be introduced in an amount that allows delivery of at least one copy per cell. Higher doses of double-stranded material may yield more effective inhibition.

The isolated expression vector of the invention comprises a polynucleotide encoding a dsRNA molecule as described above, wherein expression of the vector in a host plant cell results in increased resistance to a parasitic nematode as compared to a wild-type variety of the host plant cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host plant cell into which they are introduced. Other vectors are integrated into the genome of a host plant cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., potato virus X, tobacco rattle virus, and Gemini virus), which serve equivalent functions.

The isolated expression vectors of the invention comprise a nucleic acid of the invention in a form suitable for expression of the nucleic acid in a host plant cell, which means that the recombinant expression vector includes one or more regulatory sequences, e.g. promoters, selected on the basis of the host plant cells to be used for expression, which is operatively linked to the nucleic acid sequence to be expressed. As used herein, the terms “operatively linked” and “in operative association” are interchangeable and are intended to mean that the nucleotide sequence of interest is linked to the regulatory sequence(s) in a manner which allows expression of the nucleotide sequence (e.g., in a host plant cell when the vector is introduced into the host plant cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990) and Gruber and Crosby, in: Methods in Plant Molecular Biology and Biotechnology, Eds. Glick and Thompson, Chapter 7, 89-108, CRC Press: Boca Raton, Fla., and the like. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of dsRNA desired, and the like. The expression vectors of the invention can be introduced into plant host cells to thereby produce dsRNA molecules of the invention encoded by nucleic acids as described herein.

In accordance with the invention, the recombinant expression vector comprises a regulatory sequence operatively linked to a nucleotide sequence that is a template for one or both strands of the dsRNA molecules of the invention. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by 3 to 500 base or more pairs, and wherein after transcription, the RNA transcript folds on itself to form a hairpin. In accordance with the invention, the spacer region in the hairpin transcript may be any DNA fragment.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and be transiently expressed or transiently active. Whether present in an extra-chromosomal non-replicating vector or a vector that is integrated into a chromosome, the polynucleotide preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., 1984, EMBO J. 3:835) or functional equivalents thereof, but also all other terminators functionally active in plants are suitable. As plant gene expression is very often not limited on transcriptional levels, a plant expression cassette preferably contains other operatively linked sequences like translational enhancers such as the overdrive-sequence containing the 5′-untranslated leader sequence from tobacco mosaic virus enhancing the polypeptide per RNA ratio (Gallie et al., 1987, Nucl. Acids Research 15:8693-8711). Examples of plant expression vectors include those detailed in: Becker, D. et al., 1992, New plant binary vectors with selectable markers located proximal to the left border, Plant Mol. Biol. 20:1195-1197; Bevan, M. W., 1984, Binary Agrobacterium vectors for plant transformation, Nucl. Acid. Res. 12:8711-8721; and Vectors for Gene Transfer in Higher Plants; in: Transgenic Plants, Vol. 1, Engineering and Utilization, eds.: Kung and R. Wu, Academic Press, 1993, S. 15-38.

Plant gene expression should be operatively linked to an appropriate promoter conferring gene expression in a temporal-preferred, spatial-preferred, cell type-preferred, and/or tissue-preferred manner. Promoters useful in the expression cassettes of the invention include any promoter that is capable of initiating transcription in a plant cell present in the plant's roots. Such promoters include, but are not limited to those that can be obtained from plants, plant viruses and bacteria that contain genes that are expressed in plants, such as Agrobacterium and Rhizobium. Preferably, the expression cassette of the invention comprises a root-specific promoter, a pathogen inducible promoter, or a nematode inducible promoter. More preferably the nematode inducible promoter is or a parasitic nematode feeding site-specific promoter. A parasitic nematode feeding site-specific promoter may be specific for syncytial cells or giant cells or specific for both kinds of cells. A promoter is inducible, if its activity, measured on the amount of RNA produced under control of the promoter, is at least 30%, 40%, 50% preferably at least 60%, 70%, 80%, 90% more preferred at least 100%, 200%, 300% higher in its induced state, than in its un-induced state. A promoter is cell-, tissue- or organ-specific, if its activity, measured on the amount of RNA produced under control of the promoter, is at least 30%, 40%, 50% preferably at least 60%, 70%, 80%, 90% more preferred at least 100%, 200%, 300% higher in a particular cell-type, tissue or organ, then in other cell-types or tissues of the same plant, preferably the other cell-types or tissues are cell types or tissues of the same plant organ, e.g. a root. In the case of organ specific promoters, the promoter activity has to be compared to the promoter activity in other plant organs, e.g. leaves, stems, flowers or seeds.

The promoter may be constitutive, inducible, developmental stage-preferred, cell type-preferred, tissue-preferred or organ-preferred. Constitutive promoters are active under most conditions. Non-limiting examples of constitutive promoters include the CaMV 19S and 35S promoters (Odell et al., 1985, Nature 313:810-812), the sX CaMV 35S promoter (Kay et al., 1987, Science 236:1299-1302), the Sep1 promoter, the rice actin promoter (McElroy et al. 1990, Plant Cell 2:163-171), the Arabidopsis actin promoter, the ubiquitin promoter (Christensen et al., 1989, Plant Molec. Biol. 18:675-689); pEmu (Last et al., 1991, Theor. Appl. Genet. 81:581-588), the figwort mosaic virus 35S promoter, the Smas promoter (Velten et al., 1984, EMBO J. 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like. Promoters that express the dsRNA in a cell that is contacted by parasitic nematodes are preferred. Alternatively, the promoter may drive expression of the dsRNA in a plant tissue remote from the site of contact with the nematode, and the dsRNA may then be transported by the plant to a cell that is contacted by the parasitic nematode, in particular cells of, or close by nematode feeding sites, e.g. syncytial cells or giant cells.

Inducible promoters are active under certain environmental conditions, such as the presence or absence of a nutrient or metabolite, heat or cold, light, pathogen attack, anaerobic conditions, and the like. For example, the promoters TobRB7, AtRPE, AtPyk10, Gemini19, and AtHMG1 have been shown to be induced by nematodes (for a review of nematode-inducible promoters, see Ann. Rev. Phytopathol. (2002) 40:191-219; see also U.S. Pat. No. 6,593,513). Method for isolating additional promoters, which are inducible by nematodes are set forth in U.S. Pat. Nos. 5,589,622 and 5,824,876. Other inducible promoters include the hsp80 promoter from Brassica, being inducible by heat shock; the PPDK promoter is induced by light; the PR-1 promoter from tobacco, Arabidopsis, and maize are inducible by infection with a pathogen; and the Adh1 promoter is induced by hypoxia and cold stress. Plant gene expression can also be facilitated via an inducible promoter (For review, see Gatz, 1997, Annu. Rev. Plant Physiol. Plant Mol. Biol. 48:89-108). Chemically inducible promoters are especially suitable if time-specific gene expression is desired. Non-limiting examples of such promoters are a salicylic acid inducible promoter (PCT Application No. WO 95/19443), a tetracycline inducible promoter (Gatz et al., 1992, Plant J. 2:397-404) and an ethanol inducible promoter (PCT Application No. WO 93/21334).

Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters and the like. Seed preferred promoters are preferentially expressed during seed development and/or germination. For example, seed preferred promoters can be embryo-preferred, endosperm preferred and seed coat-preferred. See Thompson et al., 1989, BioEssays 10:108. Examples of seed preferred promoters include, but are not limited to cellulose synthase (celA), Cim1, gamma-zein, globulin-1, maize 19 kD zein (cZ19B1) and the like.

Other suitable tissue-preferred or organ-preferred promoters include, but are not limited to, the napin-gene promoter from rapeseed (U.S. Pat. No. 5,608,152), the USP-promoter from Vicia faba (Baeumlein et al., 1991, Mol Gen Genet. 225(3):459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., 1992, Plant Journal, 2(2):233-9), as well as promoters conferring seed specific expression in monocot plants like maize, barley, wheat, rye, rice, etc. Suitable promoters to note are the Ipt2 or Ipt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

Of particular utility in the present invention are syncytia site preferred, or nematode feeding site induced, promoters, including, but not limited to promoters from the Mtn3-like promoter disclosed in commonly owned copending WO 2008/095887, the Mtn21-like promoter disclosed in commonly owned copending WO 2007/096275, the peroxidase-like promoter disclosed in commonly owned copending WO 2008/077892, the trehalose-6-phosphate phosphatase-like promoter disclosed in commonly owned copending WO 2008/071726 and the At5g12170-like promoter disclosed in commonly owned copending WO 2008/095888. All of the forgoing applications are incorporated herein by reference.

In accordance with the present invention, the expression vector comprises an expression control sequence operatively linked to a nucleotide sequence that is a template for one or both strands of the dsRNA. The dsRNA template comprises (a) a first stand having a sequence substantially identical to from about 19 to about 400-500, or up to the full length, consecutive nucleotides of SEQ ID NO:1, 3, 4, 6, 8, 10, 12, 13, 15, 17, 19, 20, 22, 24, 26, 27, 29, 31, 32, 34, 36, 37, 39, 41, 43, 45, 46, 47, 49, 51, 53, 55, 57, 59, 61, 63, or 65 and (b) a second strand having a sequence substantially complementary to the first strand. In further embodiments, a promoter flanks either end of the template nucleotide sequence, wherein the promoters drive expression of each individual DNA strand, thereby generating two complementary RNAs that hybridize and form the dsRNA. In alternative embodiments, the nucleotide sequence is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 base pairs, and wherein after transcription, the RNA transcript folds on itself to form a hairpin.

In another embodiment, the vector contains a bidirectional promoter, driving expression of two nucleic acid molecules, whereby one nucleic acid molecule codes for the sequence substantially identical to a portion of a plant CLASP1, Aspartic Proteinase Delta Subunit, Secreted Protein1, Lectin Receptor Kinase-like, Pectin Methylesterase-like, NPY gene and the other nucleic acid molecule codes for a second sequence being substantially complementary to the first strand and capable of forming a dsRNA, when both sequences are transcribed. A bidirectional promoter is a promoter capable of mediating expression in two directions.

In another embodiment, the vector contains two promoters, one mediating transcription of the sequence substantially identical to a portion of a plant CLASP1, Aspartic Proteinase Delta Subunit, Secreted Protein1, Lectin Receptor Kinase-like, Pectin Methylesterase-like, NPY gene and another promoter mediating transcription of a second sequence being substantially complementary to the first strand and capable of forming a dsRNA, when both sequences are transcribed. The second promoter might be a different promoter.

A different promoter means a promoter having a different activity in regard to cell or tissue specificity, or showing expression on different inducers for example, pathogens, abiotic stress or chemicals. For example, one promoter might by constitutive or tissue specific and another might be tissue specific or inducible by pathogens. In one embodiment one promoter mediates the transcription of one nucleic acid molecule suitable for over expression of CLASP1, Aspartic Proteinase Delta Subunit, Secreted Protein1, Lectin Receptor Kinase-like, Pectin Methylesterase-like, NPY gene, while another promoter mediates tissue- or cell-specific transcription or pathogen inducible expression of the complementary nucleic acid.

The invention is also embodied in a transgenic plant capable of expressing the dsRNA of the invention and thereby inhibiting the CLASP1, Aspartic Proteinase Delta Subunit, Secreted Protein1 gene, Lectin Receptor Kinase-like gene, Pectin Methylesterase-like, NPY genes in plants. In accordance with the invention, the plant is a monocotyledonous plant or a dicotyledonous plant. The transgenic plant of the invention may be of any species that can be infected by plant parasitic nematodes, such species including, without limitation, Medicago, Solanum, Brassica, Cucumis, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Onobrychis, trifolium, Trigonella, Vigna, Citrus, Linum, Geranium, Manihot, Daucus, Raphanus, Sinapis, Atropa, Datura, Hyoscyamus, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium. Preferably the plant is a crop plant such as wheat, barley, sorghum, rye, triticale, maize, rice, sugarcane, pea, alfalfa, soybean, carrot, celery, tomato, potato, cotton, tobacco, pepper, canola, oilseed rape, beet, cabbage, cauliflower, broccoli, or lettuce.

Suitable methods for transforming or transfecting host cells including plant cells are well known in the art of plant biotechnology. Any method may be used to transform the recombinant expression vector into plant cells to yield the transgenic plants of the invention. General methods for transforming dicotyledenous plants are disclosed, for example, in U.S. Pat. Nos. 4,940,838; 5,464,763, and the like. Methods for transforming specific dicotyledenous plants, for example, cotton, are set forth in U.S. Pat. Nos. 5,004,863; 5,159,135; and 5,846,797. Soybean transformation methods are set forth in U.S. Pat. Nos. 4,992,375; 5,416,011; 5,569,834; 5,824,877; 6,384,301 and in EP 0301749B1 may be used. Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm M E et al. Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, M13 mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229. The Agrobacterium-mediated transformation is best suited to dicotyledonous plants but has also been adapted to monocotyledonous plants. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225. Transformation may result in transient or stable transformation and expression. Although a nucleotide sequence of the present invention can be inserted into any plant and plant cell falling within these broad classes, it is particularly useful in crop plant cells.

The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. Multiple genes in plants or target pathogen species can be down-regulated by gene silencing mechanisms, specifically RNAi, by using a single transgene targeting multiple linked partial sequences of interest. Stacked, multiple genes under the control of individual promoters can also be over-expressed to attain a desired single or multiple phenotype. Constructs containing gene stacks of both over-expressed genes and silenced targets can also be introduced into plants yielding single or multiple agronomically important phenotypes. In certain embodiments the nucleic acid sequences of the present invention can be stacked with any combination of polynucleotide sequences of interest to create desired phenotypes. The combinations can produce plants with a variety of trait combinations including but not limited to disease resistance, herbicide tolerance, yield enhancement, cold and drought tolerance. These stacked combinations can be created by any method including but not limited to cross breeding plants by conventional methods or by genetic transformation. If the traits are stacked by genetic transformation, the polynucleotide sequences of interest can be combined sequentially or simultaneously in any order. For example if two genes are to be introduced, the two sequences can be contained in separate transformation cassettes or on the same transformation cassette. The expression of the sequences can be driven by the same or different promoters.

In accordance with this embodiment, the transgenic plant of the invention is produced by a method comprising the steps of selecting a plant CLASP1, Aspartic Proteinase Delta Subunit, Secreted Protein1, Lectin Receptor Kinase-like, Pectin Methylesterase-like, or NPY target gene, preparing a dsRNA expression cassette having a first region that is substantially identical to at least 19, 20, or 21 consecutive nucleotides of the selected CLASP1, Aspartic Proteinase Delta Subunit, Secreted Protein1, Lectin Receptor Kinase-like, Pectin Methylesterase-like, or NPY gene and a second region which is complementary to the first region, transforming the expression cassette into a plant, and selecting progeny of the transformed plant which express the dsRNA construct of the invention.

As increased resistance to nematode infection is a general trait wished to be inherited into a wide variety of plants. Increased resistance to nematode infection is a general trait wished to be inherited into a wide variety of plants. The present invention may be used to reduce crop destruction by any plant parasitic nematode. Preferably, the parasitic nematodes belong to nematode families inducing giant or syncytial cells, such as Longidoridae, Trichodoridae, Heterodidae, Meloidogynidae, Pratylenchidae or Tylenchulidae. In particular in the families Heterodidae and Meloidogynidae.

When the parasitic nematodes are of the genus Globodera, exemplary targeted species include, without limitation, G. achilleae, G. artemisiae, G. hypolysi, G. mexicana, G. millefolii, G. mali, G. pallida, G. rostochiensis, G. tabacum, and G. virginiae. When the parasitic nematodes are of the genus Heterodera, exemplary targeted species include, without limitation, H. avenae, H. carotae, H. ciceri, H. cruciferae, H. delvii, H. elachista, H. filipjevi, H. gambiensis, H. glycines, H. goettingiana, H. graduni, H. humuli, H. hordecalis, H. latipons, H. major, H. medicaginis, H. oryzicola, H. pakistanensis, H. rosii, H. sacchari, H. schachtii, H. sorghi, H. trifolii, H. urticae, H. vigni and H. zeae. When the parasitic nematodes are of the genus Meloidogyne, exemplary targeted species include, without limitation, M. acronea, M. arabica, M. arenaria, M. artiellia, M. brevicauda, M. camelliae, M. chitwoodi, M. cofeicola, M. esigua, M. graminicola, M. hapla, M. incognita, M. indica, M. inornata, M. javanica, M. lini, M. mali, M. microcephala, M. microtyla, M. naasi, M. salasi and M. thamesi.

The following examples are not intended to limit the scope of the claims to the invention, but are rather intended to be exemplary of certain embodiments. Any variations in the exemplified methods that occur to the skilled artisan are intended to fall within the scope of the present invention.

### Example 1

### Cloning of Target Genes and Vector Construction

Using available cDNA clone sequence for the soybean target genes, PCR was used to isolate DNA fragments approximately 200-500 bp in length that were used to construct the binary vectors described in Table 1 and discussed in Example 2. The PCR products were cloned into TOPO pCR2.1 vector (Invitrogen, Carlsbad, Calif.) and inserts were confirmed by sequencing. Gene fragments for the target genes GmCLASP1, GmAspartic Proteinase Delta Subunit, GmSecreted Protein1, GmLectin Receptor Kinase-like, GmPectin Methyesterase-like, GmNPY1, and GmNPY-like5 were isolated using this method.

In order to obtain full-length cDNA for soybean target genes GmCLASP1, GmAspartic Proteinase Delta Subunit, GmSecreted Protein1, GmLectin Receptor Kinase-like, GmPectin Methyesterase-like, GmNPY1, and GmNPY-like5, 5′ RACE was performed using total RNA from SCN-infected soybean roots and the GeneRacer Kit (L1502-1) from Invitrogen.

The full length sequences for the soybean target genes GmCLASP1, GmAspartic Proteinase Delta Subunit, GmSecreted Protein1, GmLectin Receptor Kinase-like, GmPectin Methyesterase-like, GmNPY1, and GmNPY-like5 were assembled into cDNAs corresponding to the seven gene targets, designated as SEQ ID NO:1, SEQ ID NO:10, SEQ ID NO:17, SEQ ID NO:24, SEQ ID NO:29, SEQ ID NO:34, and SEQ ID NO:43.

Plant transformation binary vectors to express the dsRNA constructs described by SEQ ID NO:3, 12, 19, 26, 31, 36, 45, and 46 were generated using either a soybean cyst nematode (SCN) inducible promoter or a constitutive promoter. For this, the gene fragments described by SEQ ID NO:3, 12, 19, 26, 31, 36, 45, and 46 were operably linked to the SCN inducible GmMTN3 promoter (WO 2008/095887), the At trehalose-6-phosphate phosphatase-like promoter (WO2008/071726), or the super promoter (U.S. Pat. No. 5,955,646) as designated in Table 1. The resulting plant binary vectors contain a plant transformation selectable marker consisting of a modified Arabidopsis AHAS gene conferring tolerance to the herbicide Arsenal (BASF Corporation, Florham Park, N.J.).

TABLE 1

| | | | dsRNA | | |
|---|---|---|---|---|---|
| | | | stem | | Soybean |
| | | | sense | | Gene |
| | | Promoter | fragment | | Target |
| Construct | | SEQ ID | SEQ | Soybean | SEQ ID |
| tested | Promoter | NO | ID NO | Gene Target | NO: |
| RTP2593-3 | AtTPP | 67 | 3 | GmCLASP1 | 1, 4, 6, 8 |
| RTP3113-1 | AtTPP | 67 | 12 | GmAspartic | 10, 13, |
| | | | | Proteinase | 15 |
| | | | | Delta Subunit | |
| RTP3923-4 | AtTPP | 67 | 19 | GmSecreted | 17, 20, |
| | | | | Protein1 | 22 |
| RTP3924-1 | SUPER | 66 | 19 | GmSecreted | 17, 20, |
| | | | | Protein1 | 22 |
| RTP4280-2 | MtN3- | 68 | 26 | GmLRK-like | 24, 27 |
| | like | | | | |
| RTP4279-1 | SUPER | 66 | 26 | GmLRK-like | 24, 27 |
| RTP3856-4 | MtN3- | 68 | 31 | GmPME-like | 29, 32 |
| | like | | | | |
| RTP2362-1 | AtTPP | 67 | 36 | GmNPY1 | 34, 37, |
| | | | | | 39, 41 |
| RTP2361-4 | SUPER | 66 | 36 | GmNPY1 | 34, 37, |
| | | | | | 39, 41 |
| RTP4082-1 | SUPER | 66 | 45 | GmNPY1- | 43, 47, |
| | | | | like5 | 49 |
| RTP4083-1 | SUPER | 66 | 46 | GmNPY1- | 43, 47, |
| | | | | like5 | 49 |

### Example 2

### Bioassay of dsRNA Targeted to G. max Target Genes

The binary vectors described in Table 1 were used in the rooted plant assay system disclosed in commonly owned copending U.S. Pat. Pub. 2008/0153102. Transgenic roots were generated after transformation with the binary vectors described in Example 1. Multiple transgenic root lines were sub-cultured and inoculated with surface-decontaminated race 3 SCN second stage juveniles (J2) at the level of about 500 J2/well. Four weeks after nematode inoculation, the cyst number in each well was counted. For each transformation construct, the number of cysts per line was calculated to determine the average cyst count and standard error for the construct. The cyst count values for each transformation construct was compared to the cyst count values of an empty vector control tested in parallel to determine if the construct tested results in a reduction in cyst count. Bioassay results of constructs containing the hairpin stem sequences described by SEQ ID NOs 3, 12, 19, 26, 31, 36, 45 and 46 resulted in a general trend of reduced soybean cyst nematode cyst count over many of the lines tested in the designated construct containing a SCN inducible promoter operably linked to each of the genes described.

### Example 3

### Identification of Homologous Potato Target Gene and Vector Construction

As disclosed in Example 2, the construct RTP2593-3 results in the expression of a double stranded RNA molecule that targets SEQ ID NO:1 and results in reduced cyst count when operably linked to a SCN-inducible promoter and expressed in soybean roots. As disclosed in Example 1, the putative full length transcript sequence of the gene described by SEQ ID NO:1 contains an open reading frame with the amino acid sequence disclosed as SEQ ID NO:2. The amino acid sequence described by SEQ ID NO:2 was used to identify homologous genes from other plant species. A sample gene fragment with DNA and amino acid sequences homologous to SEQ ID NO: 1 and SEQ ID NO: 2, respectively, was identified from potato and is described by SEQ ID NO:63 and SEQ ID NO:64.

Gene fragments for the target gene StCLASP BQ506533 was isolated using available cDNA clone sequences to PCR amplify a DNA fragment 267 bp in length. The isolated DNA fragment was used to construct the binary vector described in Table 2 and discussed in Example 4. The PCR product was cloned into TOPO pCR2.1 vector (Invitrogen, Carlsbad, Calif.) and the insert was confirmed by sequencing.

TABLE 2

| | | | dsRNA | | |
|---|---|---|---|---|---|
| | | | stem | | Potato |
| | | Promoter | sense | Soybean | Gene |
| Construct | | SEQ ID | fragment | Gene | Target SEQ |
| tested | Promoter | NO | SEQ ID NO | Target | ID NO: |
| RTP2622 | PcUbi4-2 | 69 | 65 | StCLASP | 63 |
| | | | | BQ506533 | |

### Example 4

### Solanum tuberosum Root-Knot Nematode In Vitro Bioassay of dsRNA Targeted to Potato Target Gene

The binary vector RTP2622 described in Table 2 was used in a potato rooted plant assay system disclosed in commonly owned copending U.S. Pat. Pub. 2008/0153102. Transgenic roots were generated after transformation with the binary vector RTP2622 described in Example 3 and selected on growth media containing the selection agent Arsenal. Multiple transgenic root lines were sub-cultured and inoculated with surface-decontaminated RKN (Medicago incognita) second stage juveniles (J2) at the level of about 200 J2 per sample. Four weeks after nematode inoculation, roots were treated with Erioglaucine Brilliant Blue stain and egg masses were counted for each sample. Egg mass count normalized to fresh root weight was used to calculate the average egg mass count and standard error for the RTP2622 construct. The average egg mass counts for potato roots transformed with the binary construct RTP2622 was compared to the average egg mass counts of an empty vector control tested in parallel to determine if the construct tested results in a reduction in egg mass count. Bioassay data for construct RTP2622 containing the hairpin stem sequence described by SEQ ID NO:65 shows a general trend of reduced root knot nematode egg mass counts over many of the lines tested in the designated construct containing a constitutive promoter operably linked to the gene described.

### Example 5

### Identification of Additional Soybean Sequences Targeted by Binary Constructs

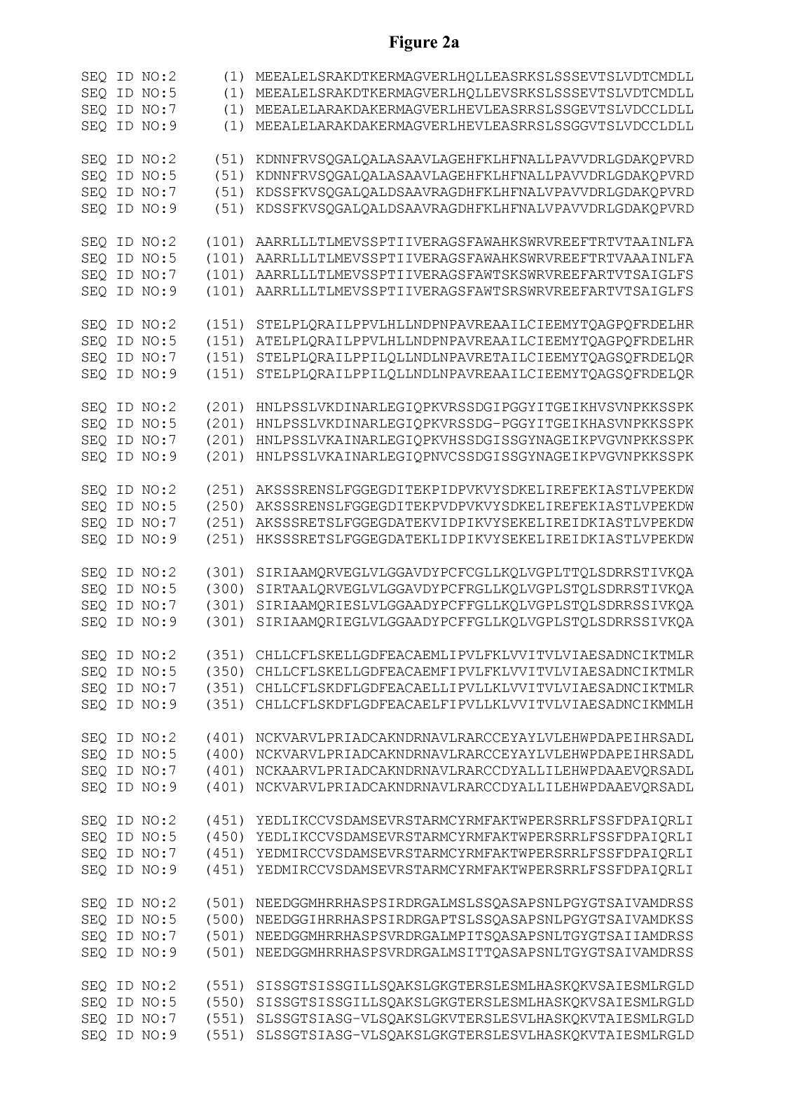

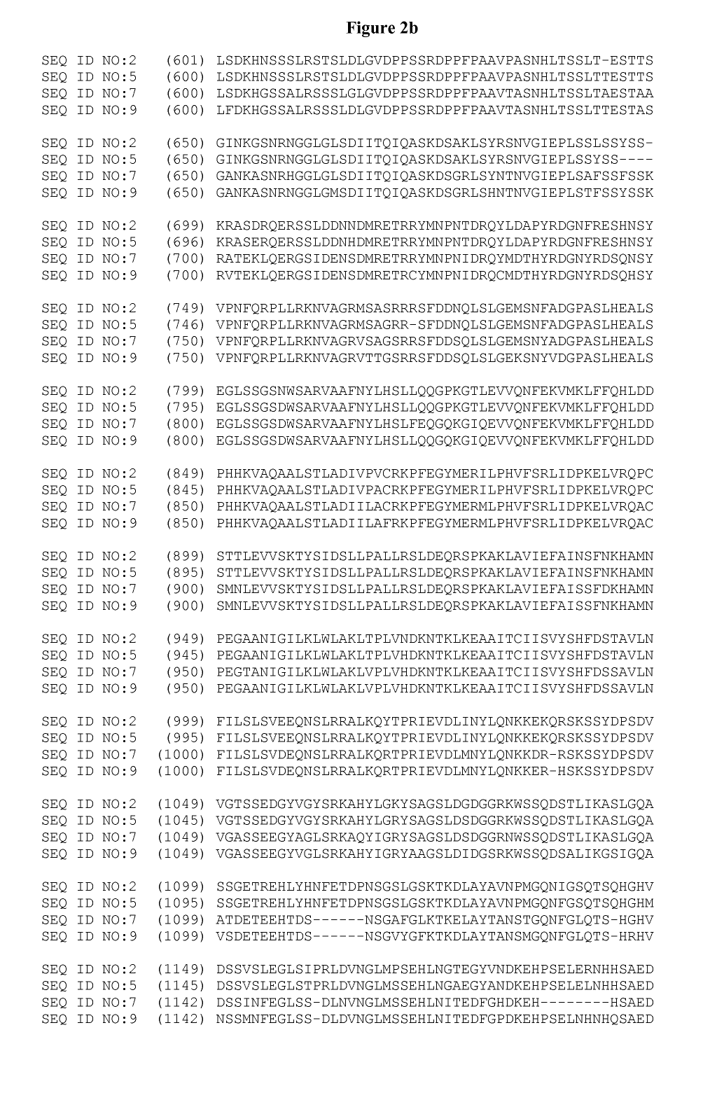

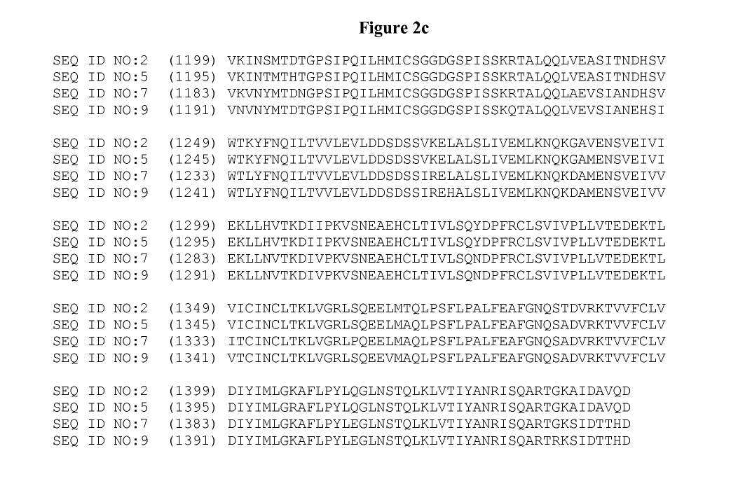

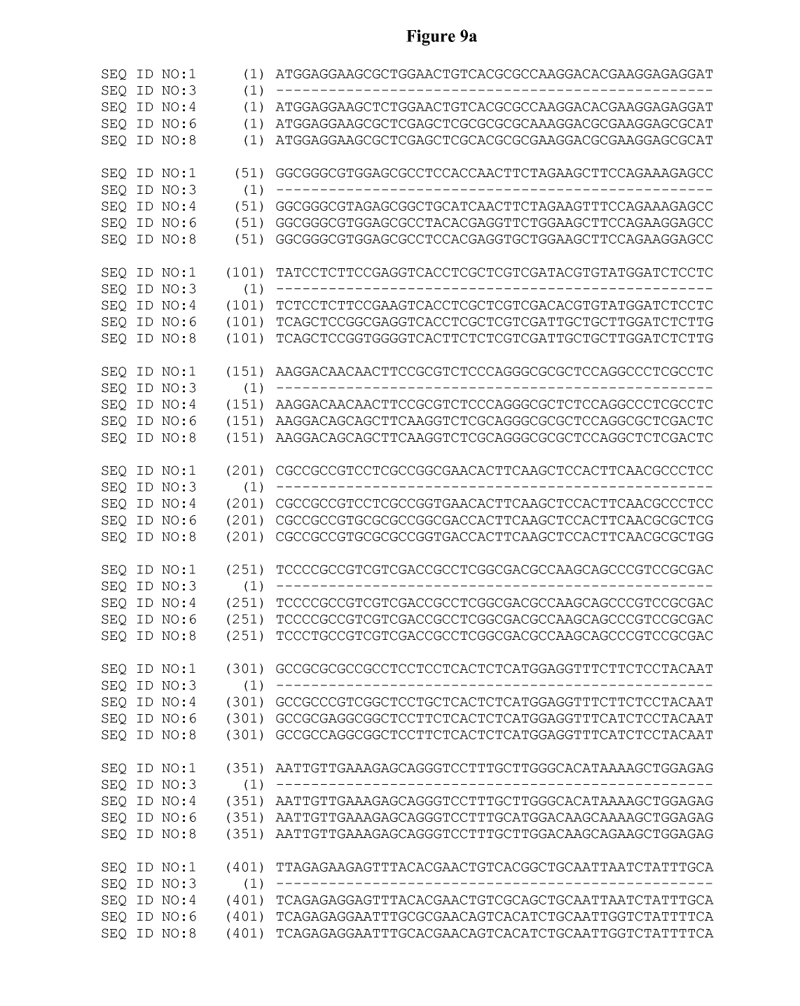

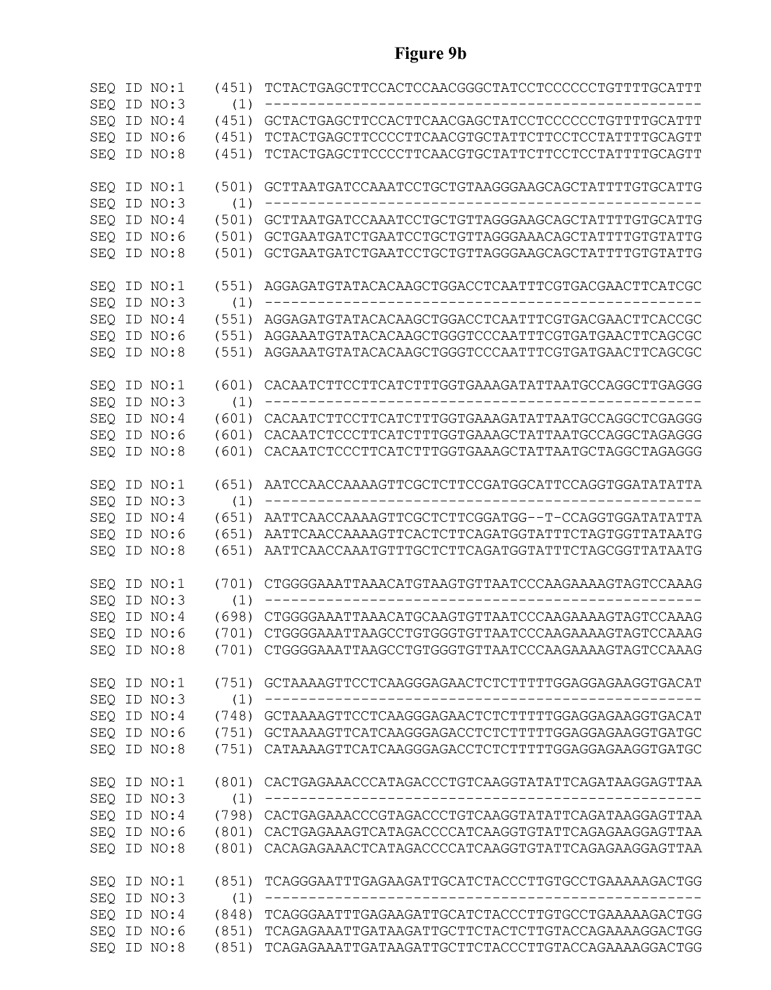

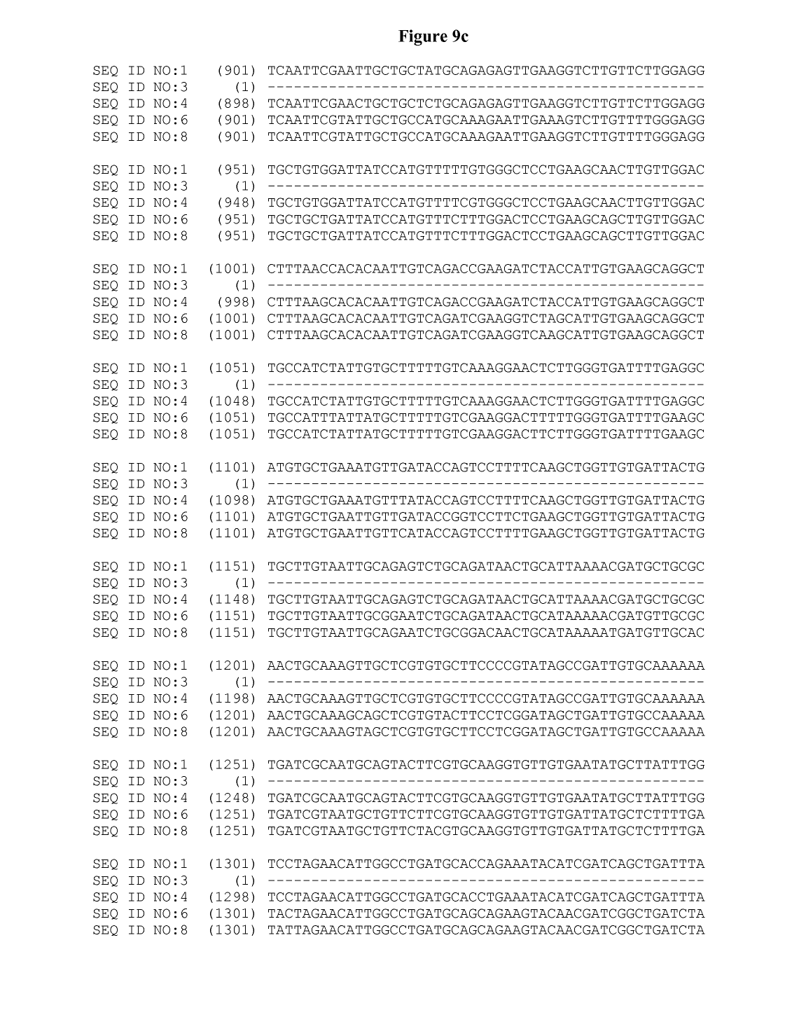

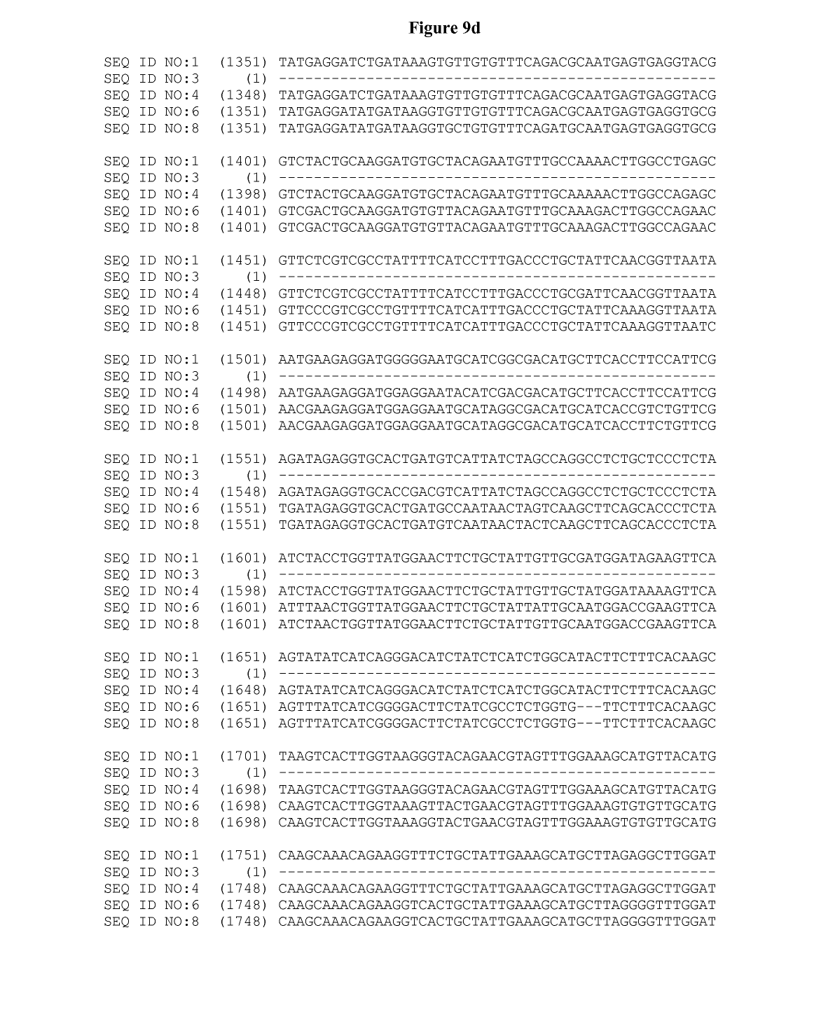

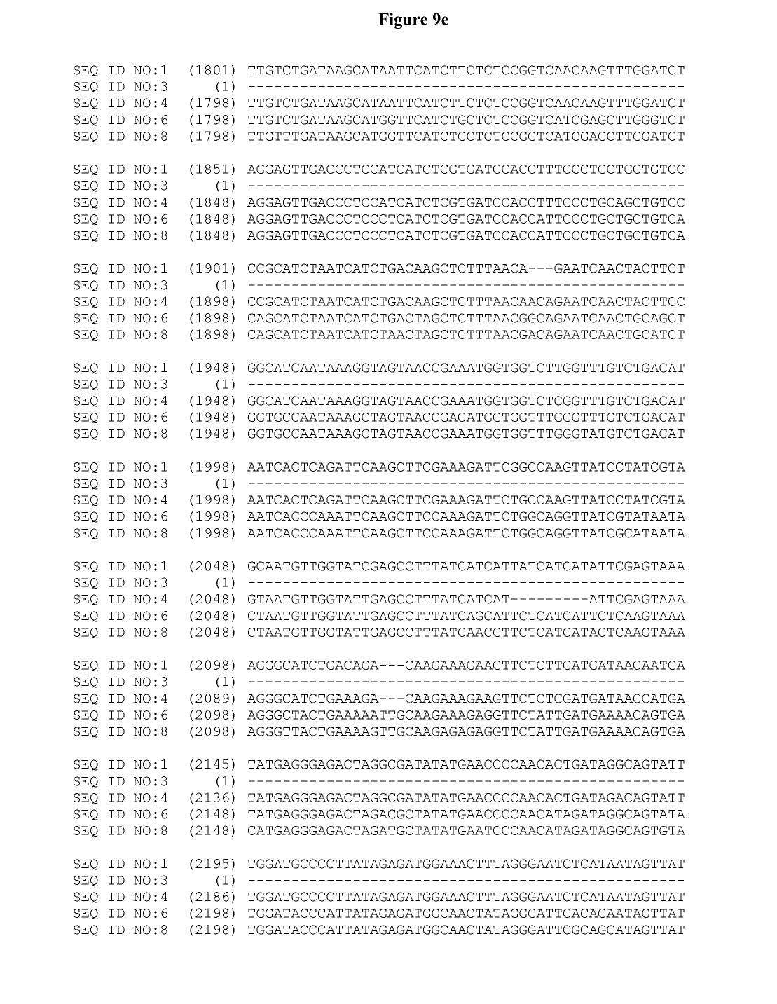

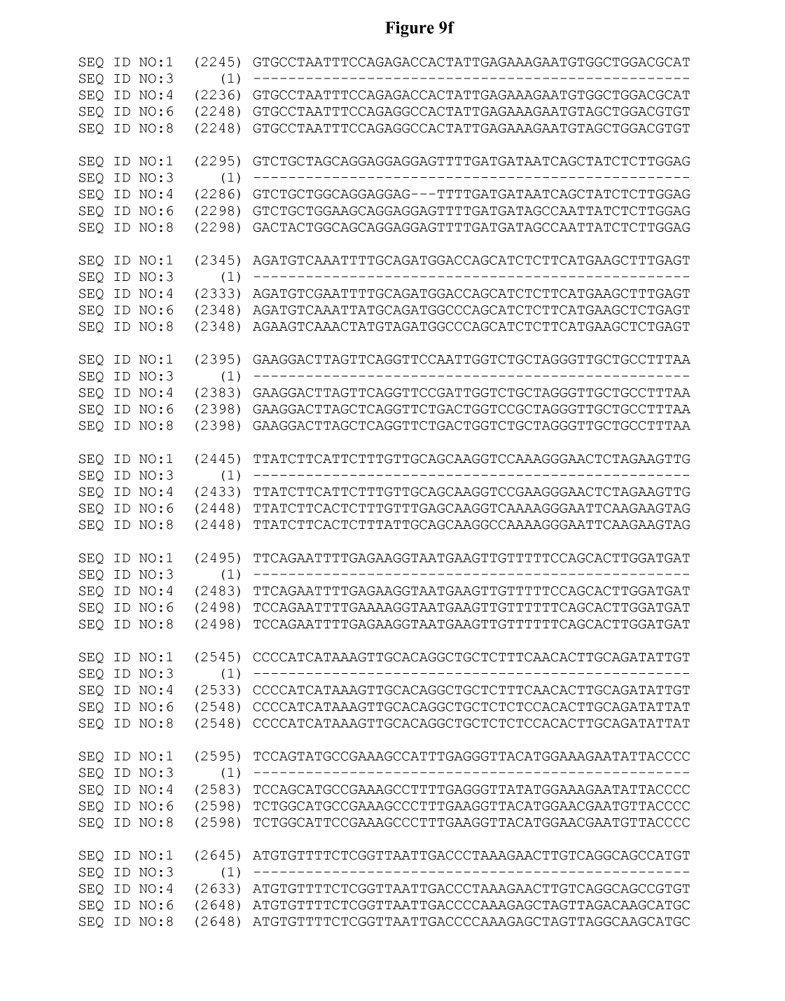

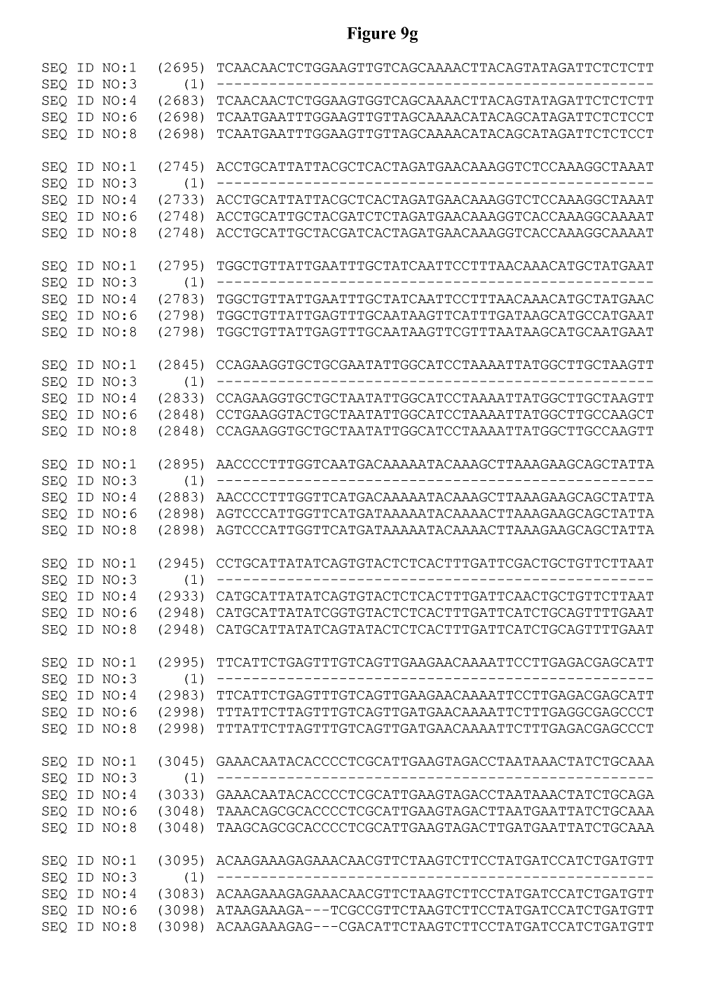

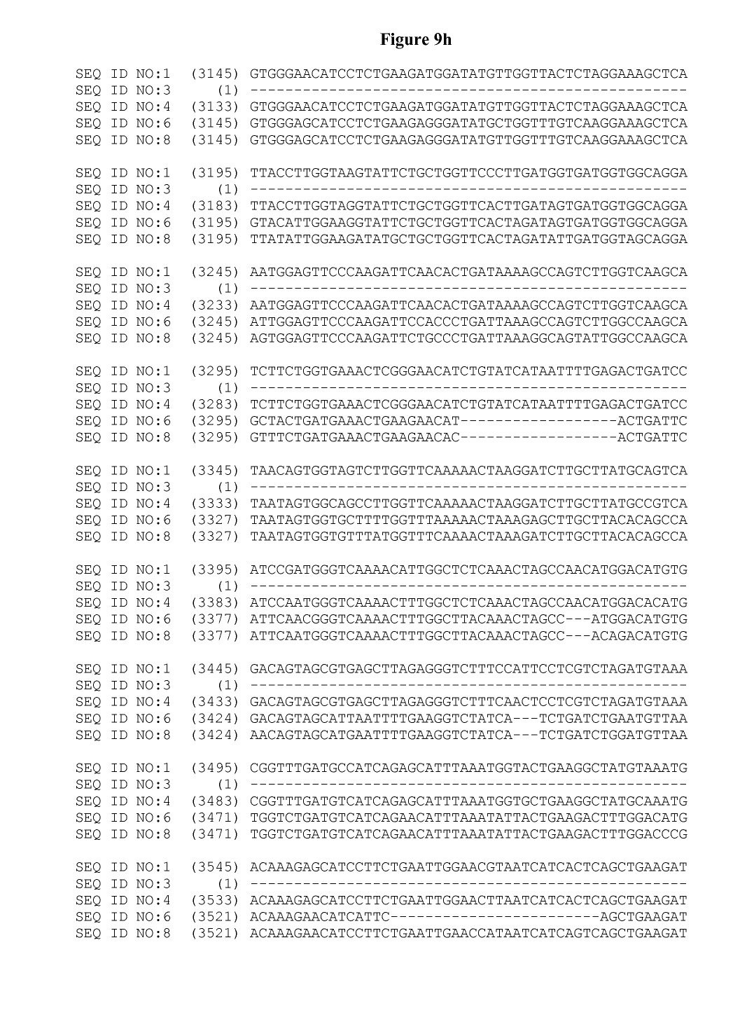

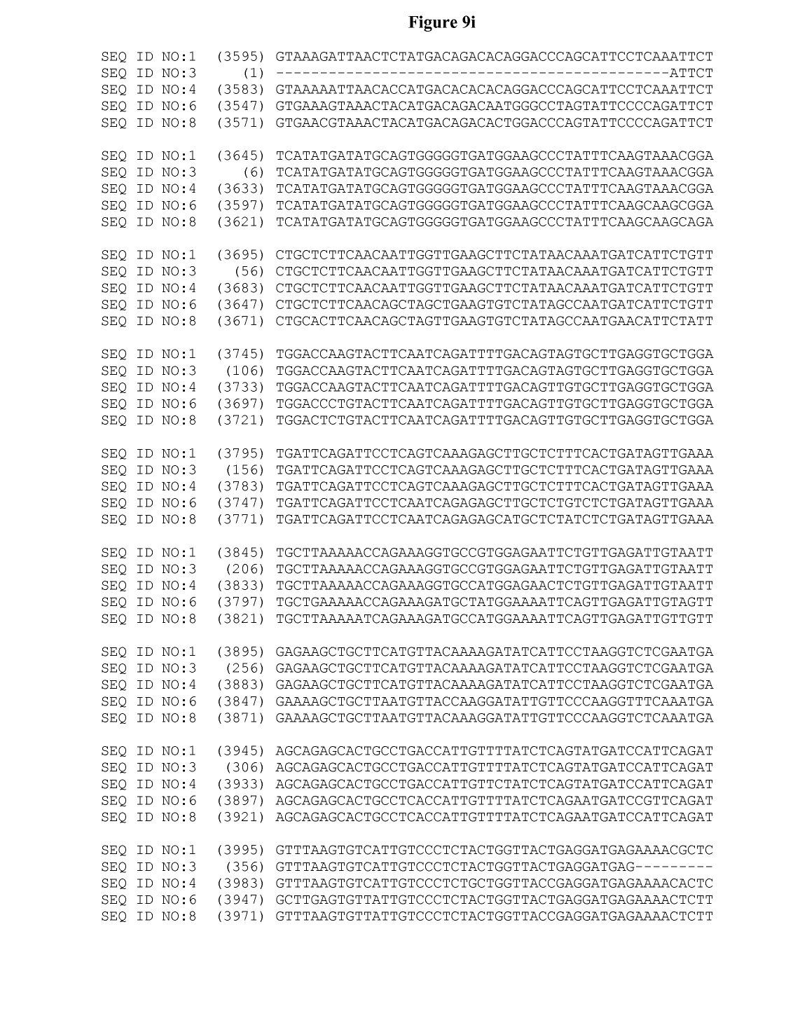

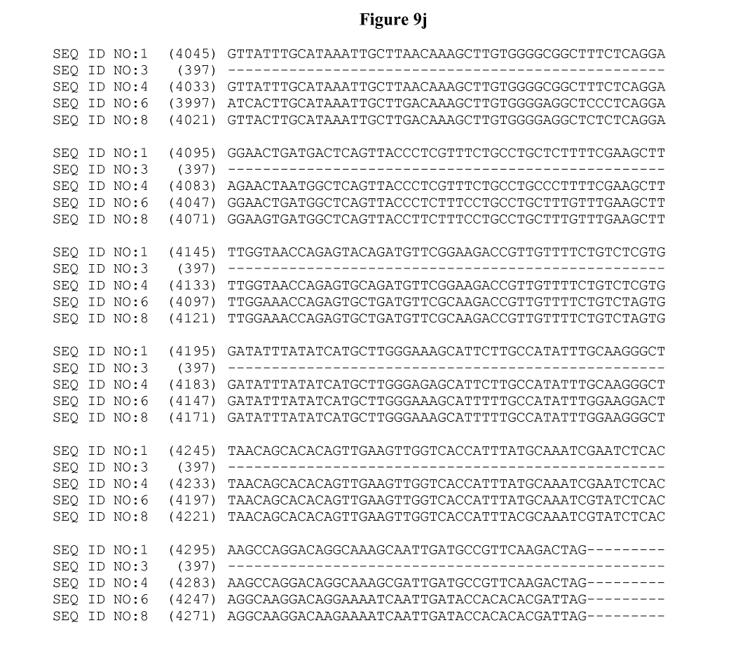

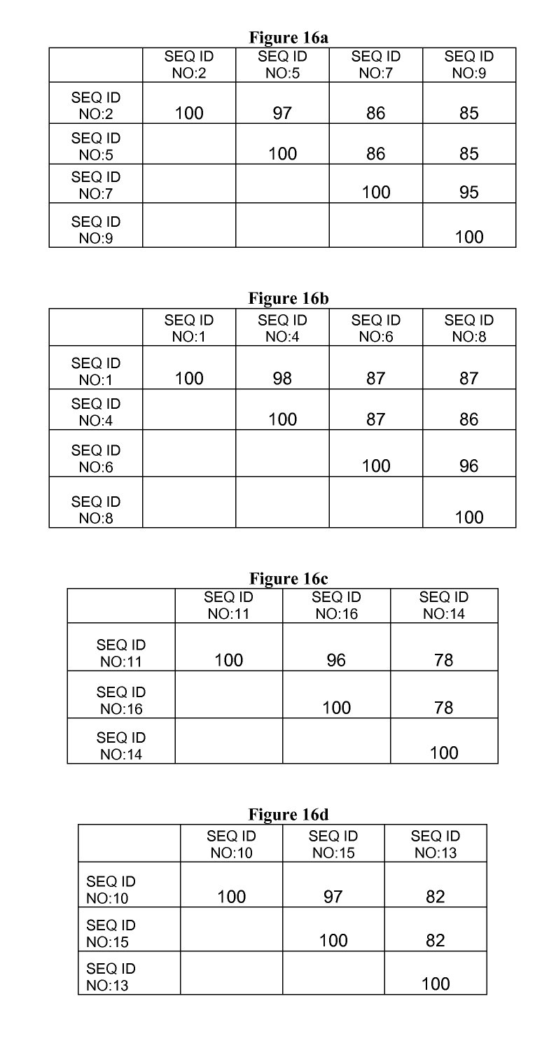

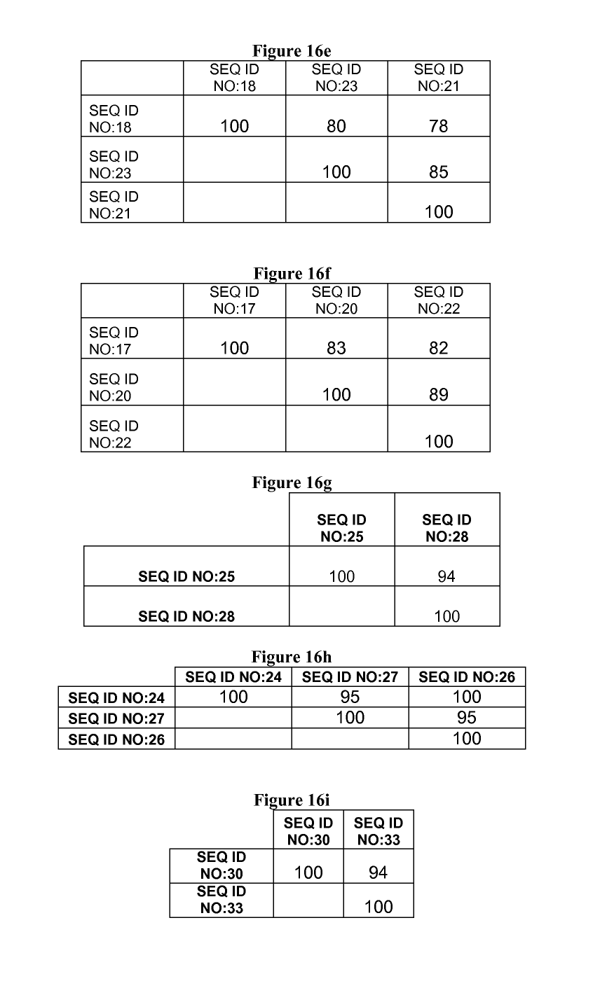

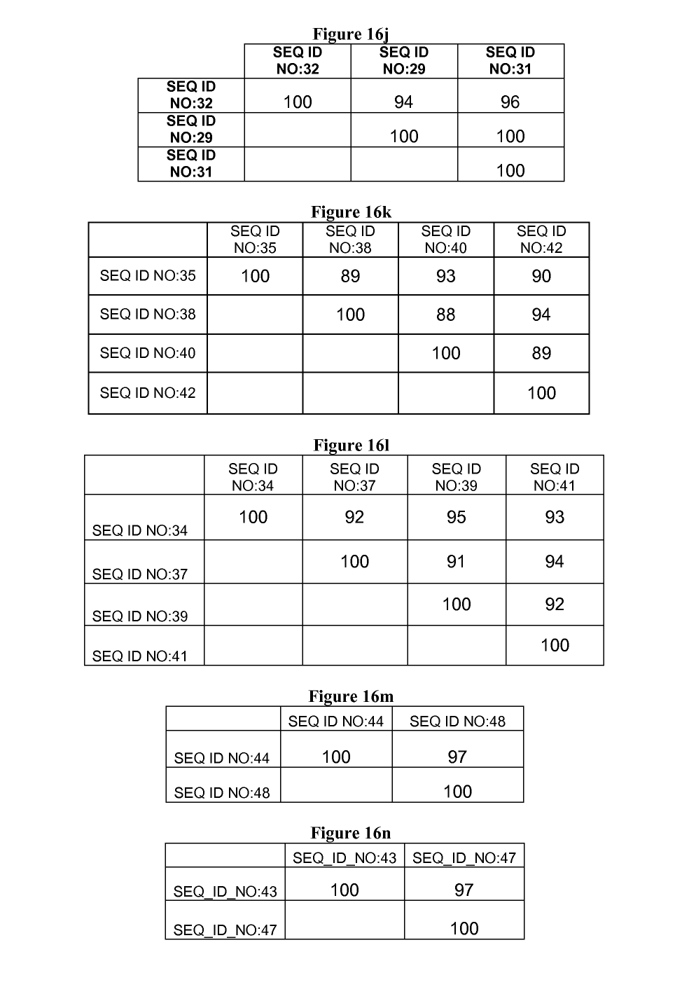

As disclosed in Example 2, the construct RTP2593-3 results in the expression of a double stranded RNA molecule that targets SEQ ID NO:1 and results in reduced cyst counts when operably linked to a SCN-inducible promoter and expressed in soybean roots. The sense fragment of the GmCLASP1 gene contained in RTP2593-3, described by SEQ ID NO:3, corresponds to nucleotides 3661 to 4056 of the full length GmCLASP1 sequence described by SEQ ID NO:1. At least one of the resulting 21mers derived from the processing of the double stranded RNA molecule expressed from RTP2593-3 can target other soybean sequences described by SEQ ID NO:4, 6 and 8. The amino acid alignment of the identified targets of the double stranded RNA molecule expressed from RTP2593-3 described by the GmCLASP1 target gene SEQ ID NO:2, Glyma03g32710.1 described by SEQ ID NO:5, Glyma13g19230.1 described by SEQ ID NO:7 and Glyma10g04850.1 described by SEQ ID NO:9 is shown in FIG. 2. The open reading frame nucleotide alignment of the identified targets of the double stranded RNA molecule expressed from RTP2593-3 described by the GmCLASP1 target gene SEQ ID NO:1, the sense fragment of the GmCLASP1 gene contained in RTP2593-3 described by SEQ ID NO:3, Glyma03g32710.1 described by SEQ ID NO:4, Glyma13g19230.1 described by SEQ ID NO:6 and Glyma10g04850.1 described by SEQ ID NO:8 is shown in FIG. 9. A matrix table showing the amino acid sequence percent identity of the full length amino acid sequence of the GmCLASP1 gene described by SEQ ID NO:2 and additional soybean transcript targets of the double stranded RNA molecule expressed by RTP2593-3 described by SEQ ID NO:5, 7 and 9 to each other is shown in FIG. 16a. A matrix table showing the DNA sequence percent identity of the open reading frame transcript sequence of the GmCLASP1 gene described by SEQ ID NO:1 and additional soybean transcript targets of the double stranded RNA molecule expressed by RTP2593-3 described by SEQ ID NO:4, SEQ ID NO:6 and SEQ ID NO:8 to each other is shown in FIG. 16b.

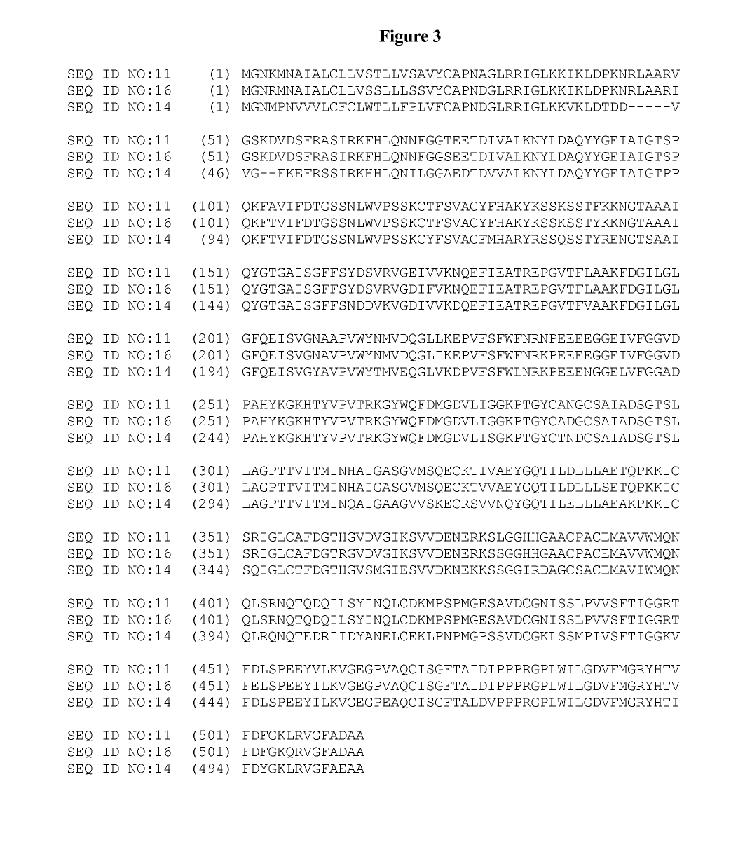

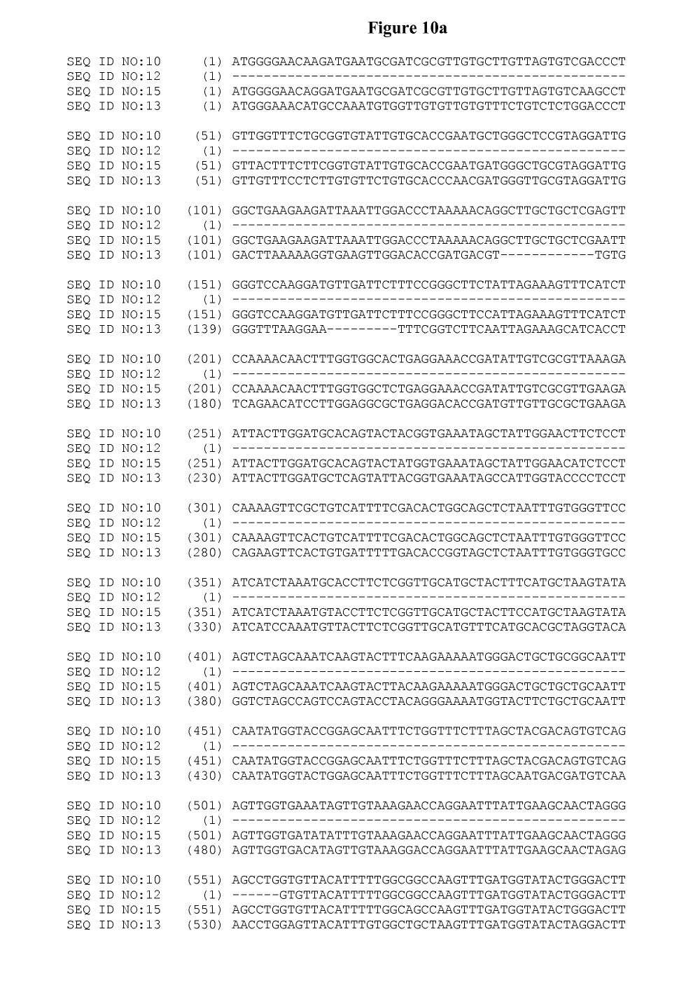

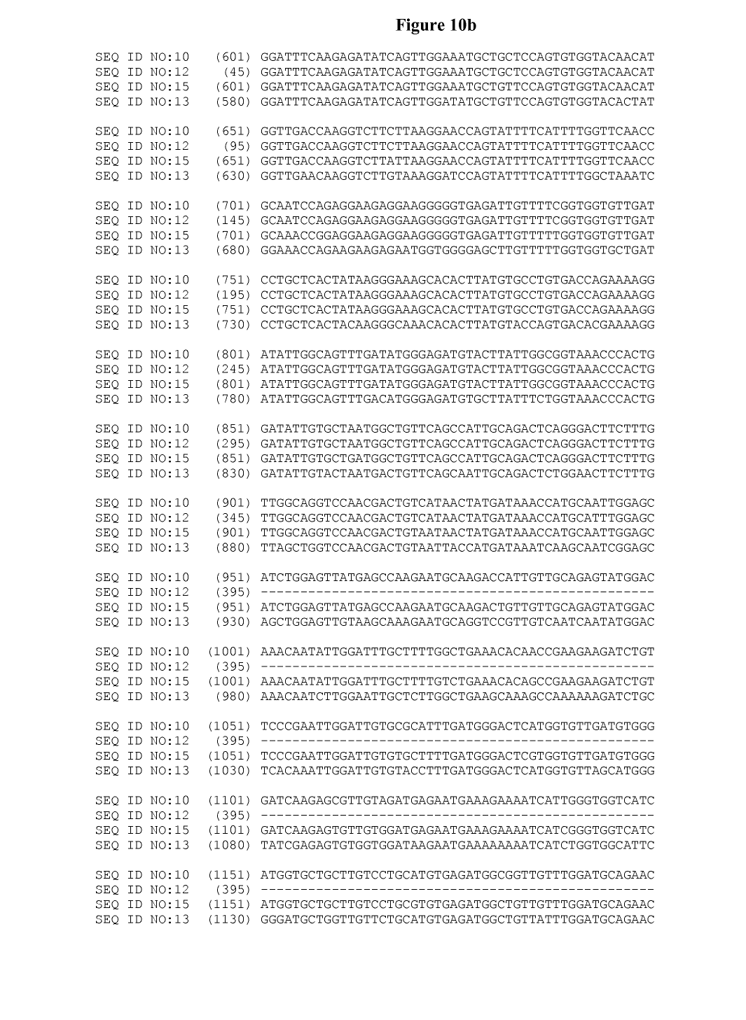

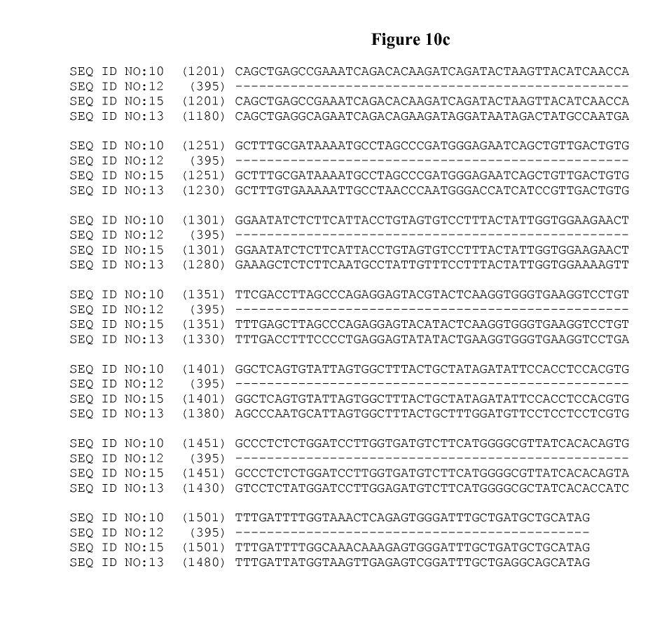

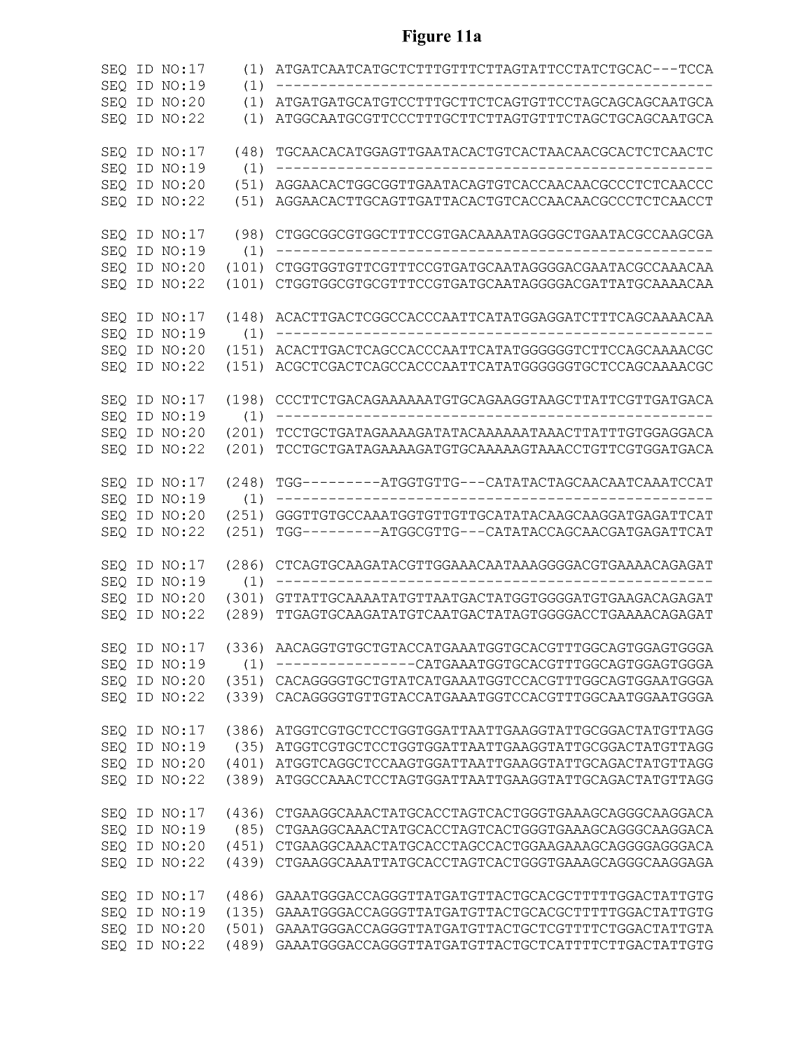

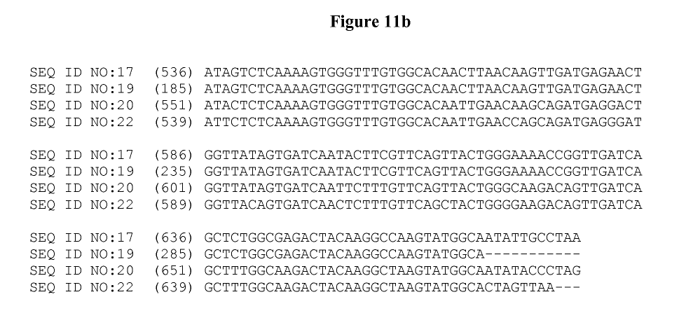

As disclosed in Example 2, the construct RTP3113-1 results in the expression of a double stranded RNA molecule that targets SEQ ID NO:10 and results in reduced cyst counts when operably linked to a SCN-inducible promoter and expressed in soybean roots. The sense fragment of the GmAspartic Proteinase Delta Subunit gene contained in RTP3113-1, described by SEQ ID NO:12 corresponds to nucleotides 557 to 950 of the full length GmAspartic Proteinase Delta Subunit sequence described by SEQ ID NO:10. At least one of the resulting 21mers derived from the processing of the double stranded RNA molecule expressed from RTP3113-1 can target other soybean sequences described by SEQ ID NO:13 and 15. The amino acid alignment of the identified targets of the double stranded RNA molecule expressed from RTP3113-1 described by the GmAspartic Proteinase Delta Subunit target gene SEQ ID NO:11, Glyma15g11670.1 described by SEQ ID NO:14 and Glyma07g39240.1 described by SEQ ID NO:16 is shown in FIG. 3. The open reading frame nucleotide alignment of the identified targets of the double stranded RNA molecule expressed from RTP3113-1 described by the GmAspartic Proteinase Delta Subunit target gene SEQ ID NO:10, the sense fragment of the GmAspartic Proteinase Delta Subunit gene contained in RTP3113-1 described by SEQ ID NO:12, Glyma15g11670.1 described by SEQ ID NO:13 and Glyma07g39240.1 described by SEQ ID NO:15 is shown in FIG. 10. A matrix table showing the amino acid sequence percent identity of the full length amino acid sequence of the GmAspartic Proteinase Delta Subunit gene described by SEQ ID NO:11 and additional soybean transcript targets of the double stranded RNA molecule expressed by RTP3113-1 described by SEQ ID NO:14 and 16 to each other is shown in FIG. 16c. A matrix table showing the DNA sequence percent identity of the open reading frame transcript sequence of the GmAspartic Proteinase Delta Subunit gene described by SEQ ID NO:10 and additional soybean transcript targets of the double stranded RNA molecule expressed by RTP3113-1 described by SEQ ID NO:13 and SEQ ID NO:15 to each other is shown in FIG. 16d.

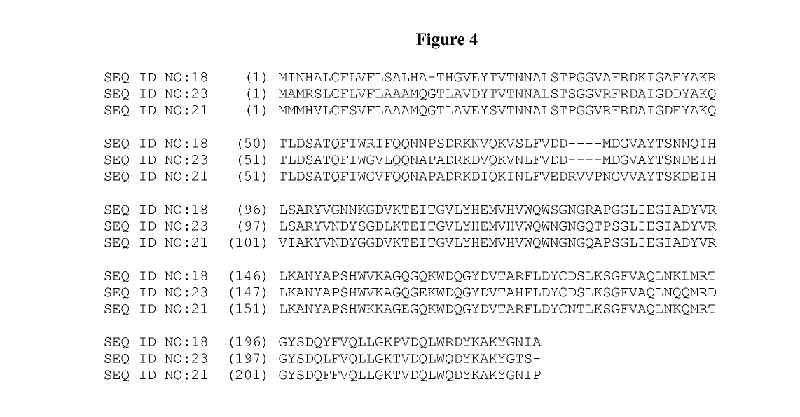

As disclosed in Example 2, the construct RTP3923-4 results in the expression of a double stranded RNA molecule that targets SEQ ID NO:17 and results in reduced cyst counts when operably linked to a SCN-inducible promoter and expressed in soybean roots. As disclosed in Example 2, the construct RTP3924-1 results in the expression of a double stranded RNA molecule that targets SEQ ID NO:17 and results in reduced cyst counts when operably linked to a constitutive promoter and expressed in soybean roots. The sense fragment of the GmSecreted Protein1 gene contained in RTP3923-4 and RTP3924-1, described by SEQ ID NO:19, corresponds to nucleotides 386 to 701 of the full length GmSecreted Protein1 sequence described by SEQ ID NO:17. At least one of the resulting 21mers derived from the processing of the double stranded RNA molecule expressed from RTP3923-4 or RTP3924-1 can target other soybean sequences described by SEQ ID NO:20 and 22. The amino acid alignment of the identified targets of the double stranded RNA molecule expressed from RTP3923-4 and RTP3924-1 described by the GmSecreted Protein1 target gene SEQ ID NO:18, GmSecreted Protein2 gene described by SEQ ID NO:21 and Glyma20g26600.1 described by SEQ ID NO:23 is shown in FIG. 4. The open reading frame nucleotide alignment of the identified targets of the double stranded RNA molecule expressed from RTP3923-4 and RTP3924-1 described by the GmSecreted Protein1 target gene SEQ ID NO:17, the sense fragment of the GmSecreted Protein1 gene contained in RTP3923-4 and RTP3924-1 described by SEQ ID NO:19, the GmSecreted Protein2 gene described by SEQ ID NO:20 and Glyma20g26600.1 described by SEQ ID NO:22 is shown in FIG. 11. A matrix table showing the amino acid sequence percent identity of the full length amino acid sequence of the GmSecreted Protein1 gene described by SEQ ID NO:18 and an additional soybean transcript target of the double stranded RNA molecule expressed by RTP3923-4 and RTP3924-1 described by SEQ ID NO:21 and 23 to each other is shown in FIG. 16e. A matrix table showing the DNA sequence percent identity of the open reading frame transcript sequence of the GmSecreted Protein1 gene described by SEQ ID NO:17, and additional soybean transcript targets of the double stranded RNA molecule expressed by RTP3923-4 and RTP3924-1 described by SEQ ID NO:20 and SEQ ID NO:22 to each other is shown in FIG. 16f.

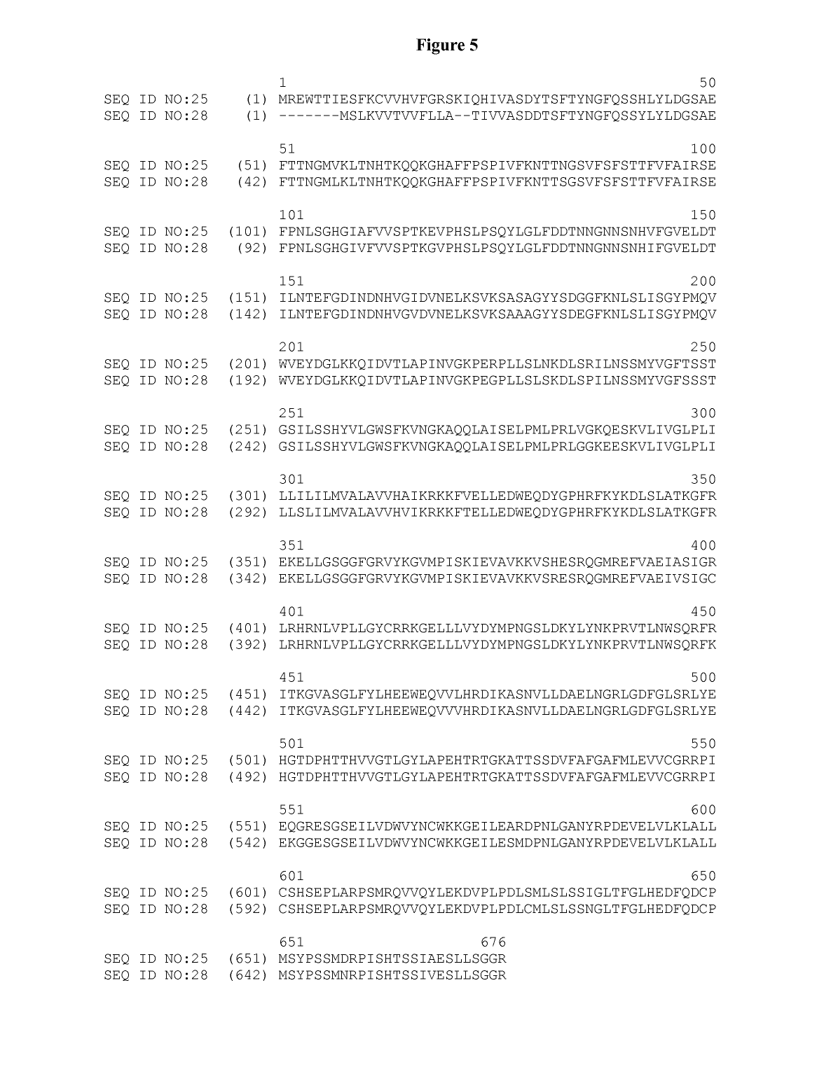

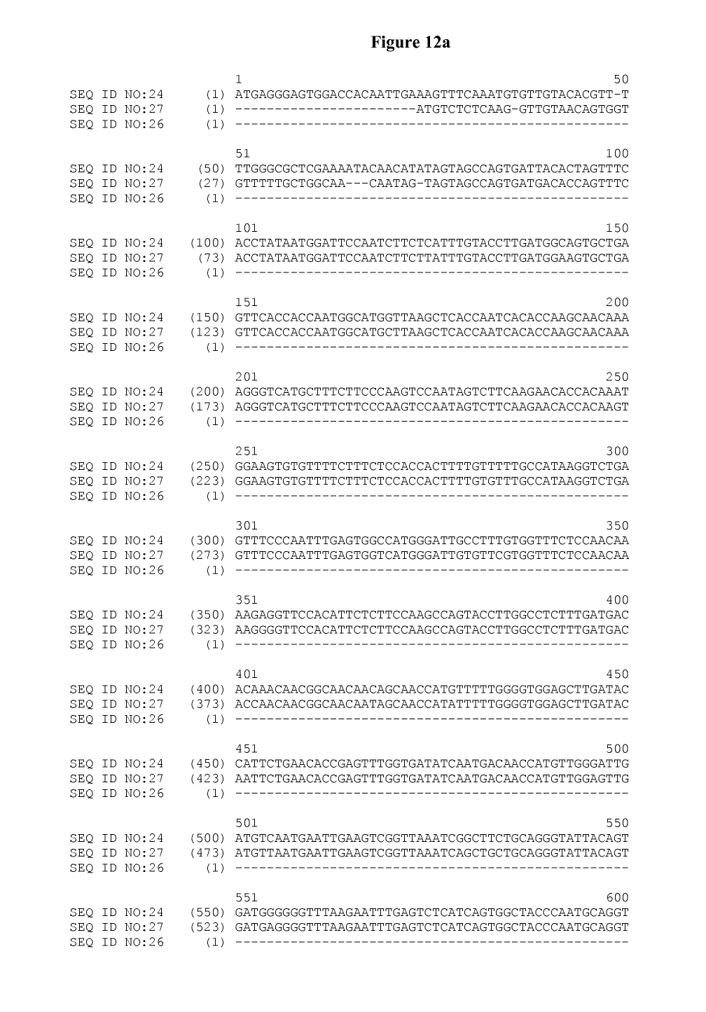

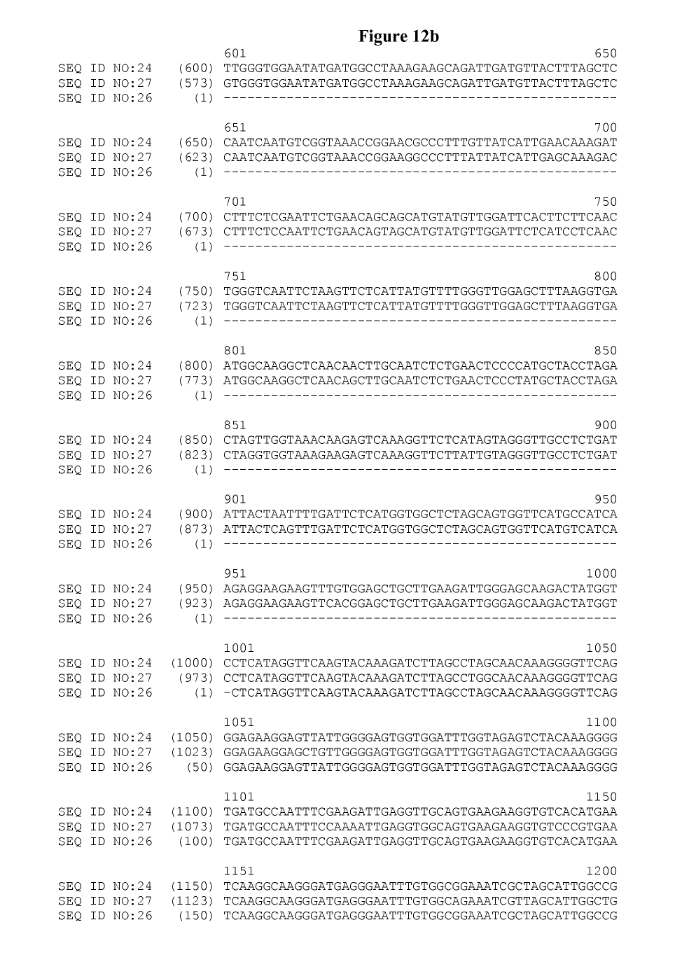

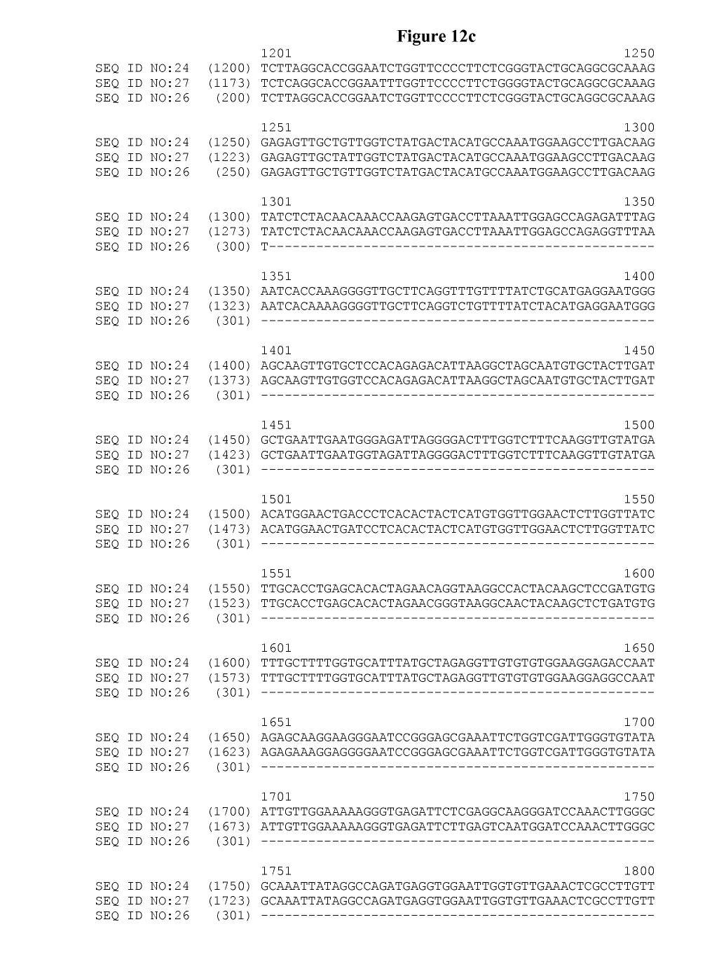

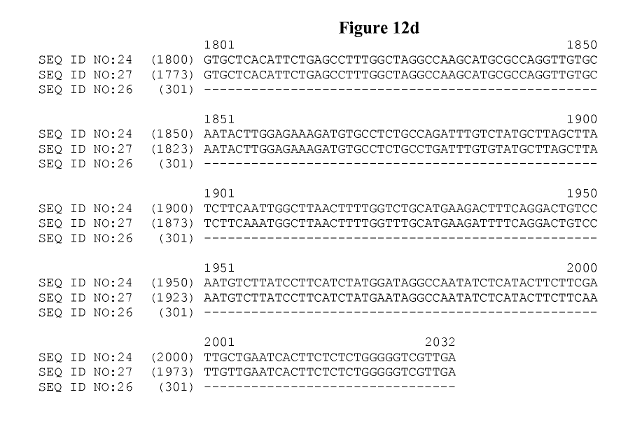

As disclosed in Example 2, the construct RTP4280-2 results in the expression of a double stranded RNA molecule that targets SEQ ID NO:24 and results in reduced cyst counts when operably linked to a SCN-inducible promoter and expressed in soybean roots. As disclosed in Example 2, the construct RTP4279-1 results in the expression of a double stranded RNA molecule that targets SEQ ID NO:24 and results in reduced cyst counts when operably linked to a constitutive promoter and expressed in soybean roots. The sense fragment of the GmLRK-like gene contained in RTP4280-2 and RTP4279-1, described by SEQ ID NO:26, corresponds to nucleotides 1001 to 1300 of the full length GmLRK-like sequence described by SEQ ID NO:24. At least one of the resulting 21 mers derived from the processing of the double stranded RNA molecule expressed from RTP4280-2 or RTP4279-1 can target another soybean sequence described by SEQ ID NO:27. The amino acid alignment of the identified targets of the double stranded RNA molecule expressed from RTP4280-2 and RTP4279-1 described by the GmLRK-like target gene SEQ ID NO:25 and Glyma18g40290.1 described by SEQ ID NO:28 is shown in FIG. 5. The open reading frame nucleotide alignment of the identified targets of the double stranded RNA molecule expressed from RTP4280-2 and RTP4279-1 described by the GmLRK-like target gene SEQ ID NO:24, the sense fragment of the GmLRK-like gene contained in RTP4280-2 and RTP4279-1 described by SEQ ID NO:26, and Glyma18g40290.1 gene described by SEQ ID NO:27 is shown in FIG. 12. A matrix table showing the amino acid sequence percent identity of the full length amino acid sequence of the GmLRK-like gene described by SEQ ID NO:25 and an additional soybean transcript target of the double stranded RNA molecule expressed by RTP4280-2 and RTP4279-1 described by SEQ ID NO:28 to each other is shown in FIG. 16g. A matrix table showing the DNA sequence percent identity of the open reading frame transcript sequence of the GmLRK-like gene described by SEQ ID NO:24, the sense fragment of the GmLRK-like gene contained in RTP4280-2 and RTP4279-1 described by SEQ ID NO:26, and an additional soybean transcript target of the double stranded RNA molecule expressed by RTP4280-2 and RTP4279-1 described by SEQ ID NO:27 to each other is shown in FIG. 16h.

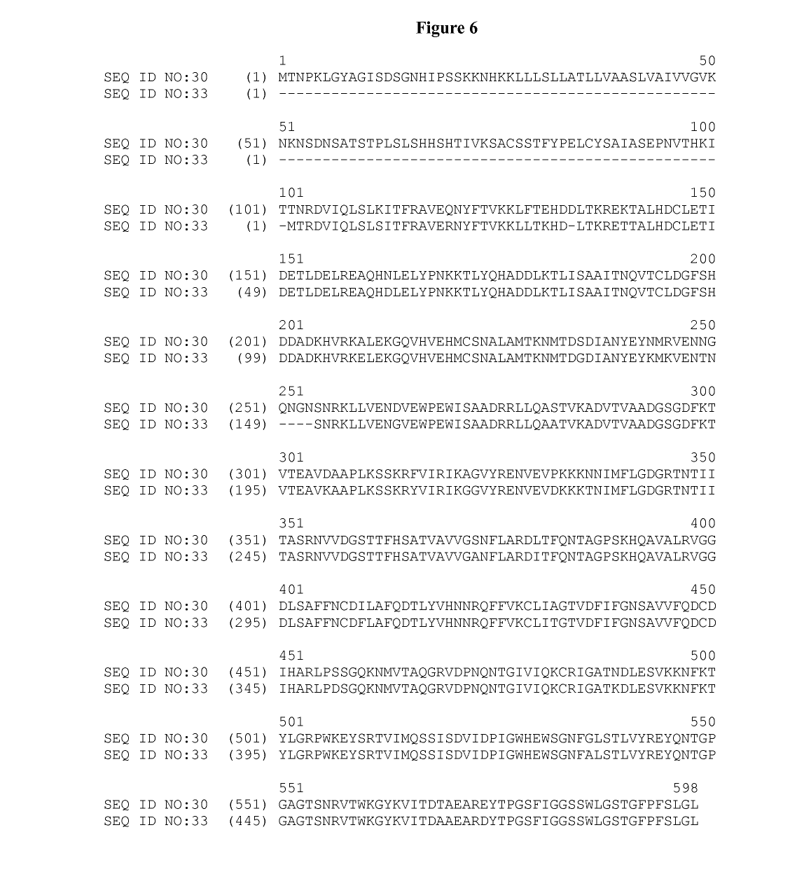

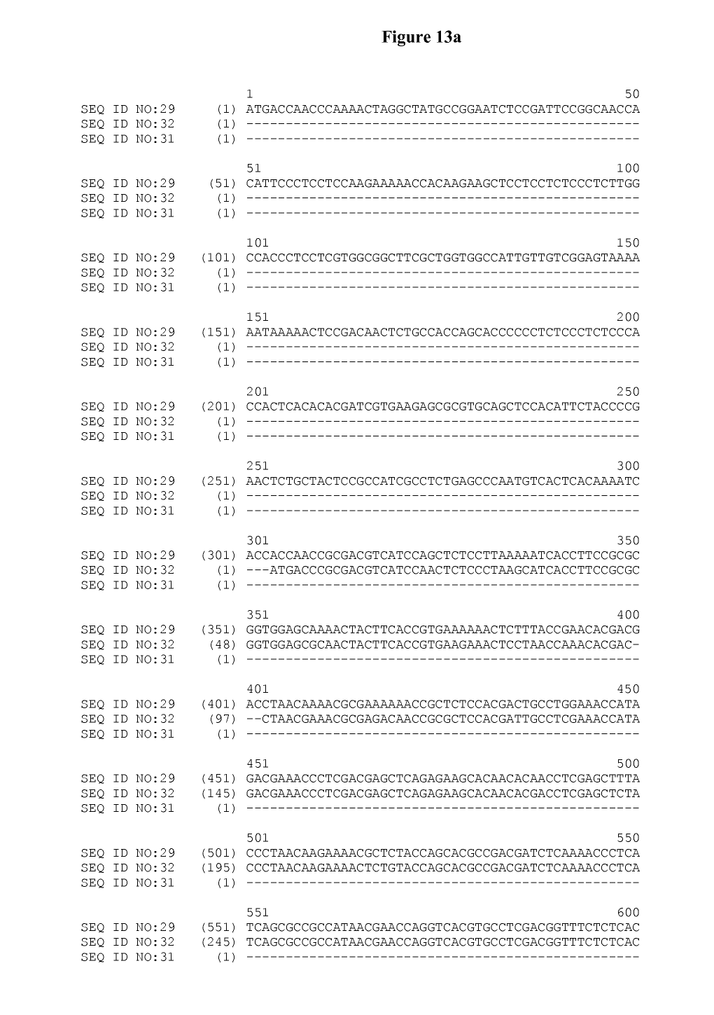

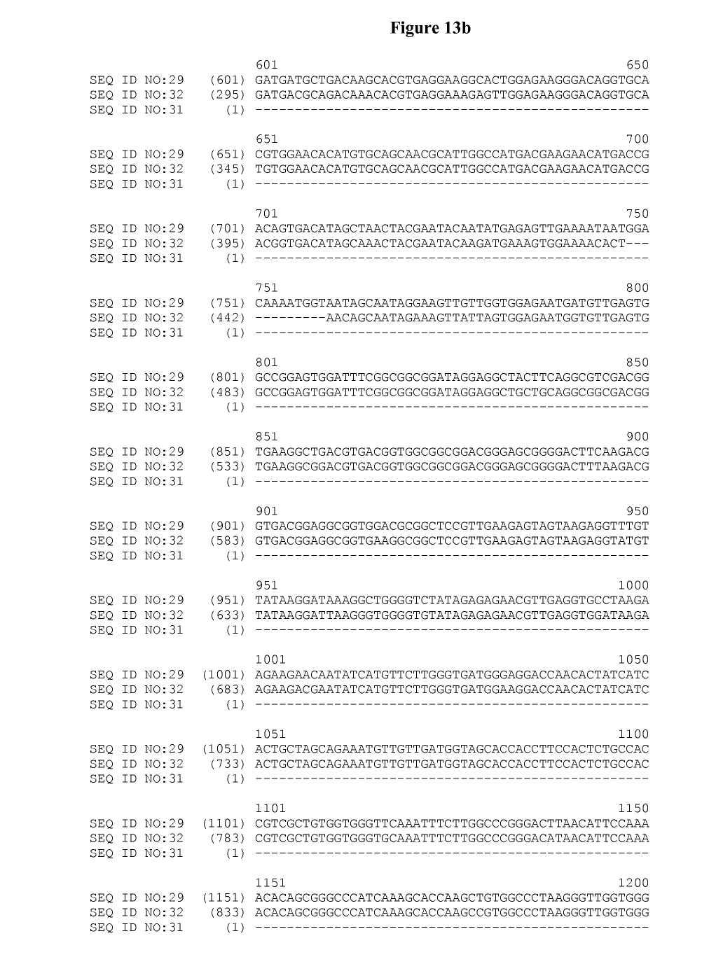

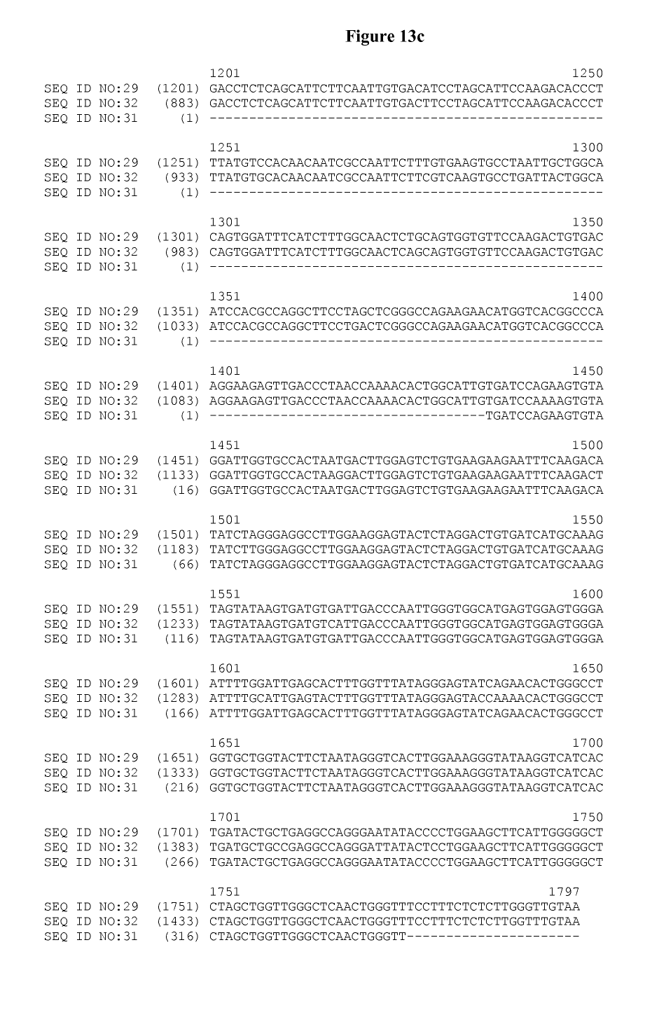

As disclosed in Example 2, the construct RTP3856-4 results in the expression of a double stranded RNA molecule that targets SEQ ID NO:29 and results in reduced cyst count when operably linked to a SCN-inducible promoter and expressed in soybean roots. The sense fragment of the GmPME-like gene contained in RTP3856-4, described by SEQ ID NO:31, corresponds to nucleotides 1474 to 1813 of the full length GmPME-like sequence described by SEQ ID NO:29. At least one of the resulting 21 mers derived from the processing of the double stranded RNA molecule expressed from RTP3856-4 can target another soybean sequence described by SEQ ID NO:32. The amino acid alignment of the identified targets of the double stranded RNA molecule expressed from RTP3856-4 described by the GmPME-like target gene SEQ ID NO:30 and Glyma16g01650.1 described by SEQ ID NO:33 is shown in FIG. 6. The open reading frame nucleotide alignment of the identified targets of the double stranded RNA molecule expressed from RTP3856-4 described by the GmPME-like target gene SEQ ID NO:29, the sense fragment of the GmPME-like gene contained in RTP3856-4 described by SEQ ID NO:31, and Glyma16g01650.1 sequence described by SEQ ID NO:32 is shown in FIG. 13. A matrix table showing the amino acid sequence percent identity of the full length amino acid sequence of the GmPME-like gene described by SEQ ID NO:30 and an additional soybean transcript target of the double stranded RNA molecule expressed by RTP3856-4 described by SEQ ID NO:33 to each other is shown in FIG. 16i. A matrix table showing the DNA sequence percent identity of the open reading frame transcript sequence of the GmPME-like gene described by SEQ ID NO:29, the sense fragment of the GmPME-like gene contained in RTP3856-4 described by SEQ ID NO:31, and an additional soybean transcript target of the double stranded RNA molecule expressed by RTP3856-4 described by SEQ ID NO:32 to each other is shown in FIG. 16j.

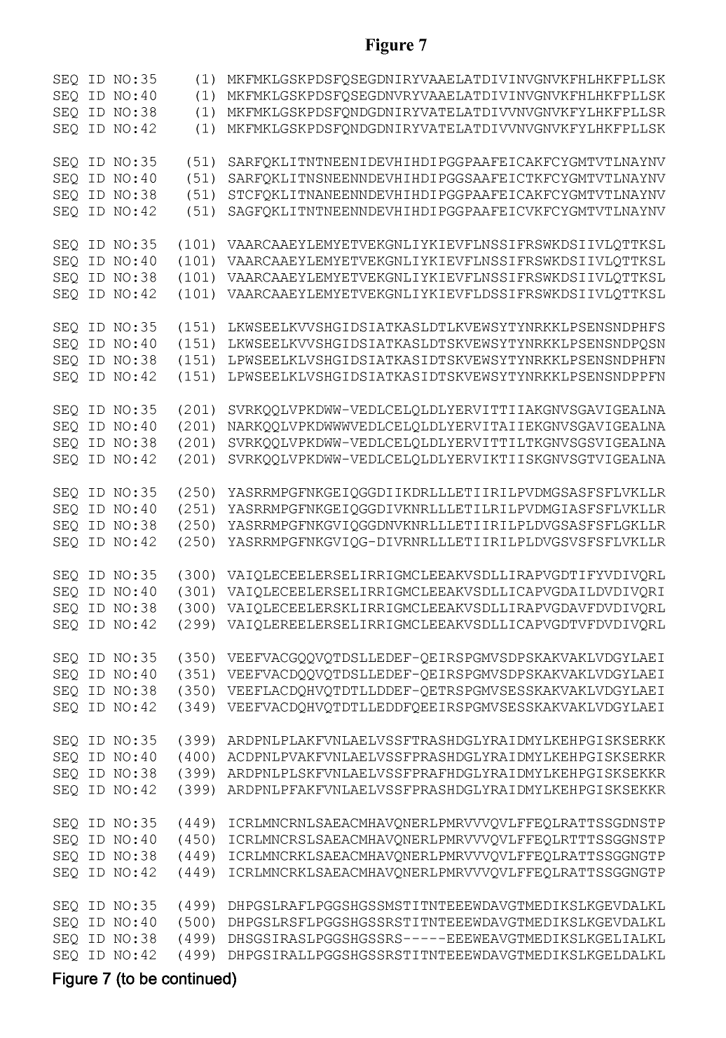

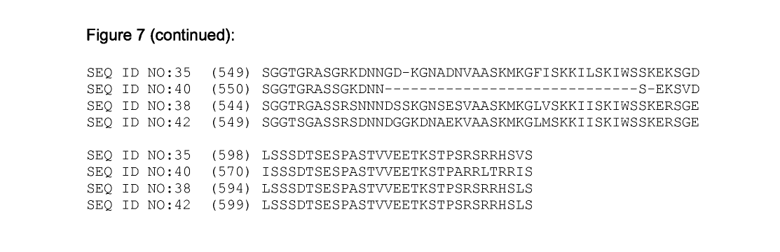

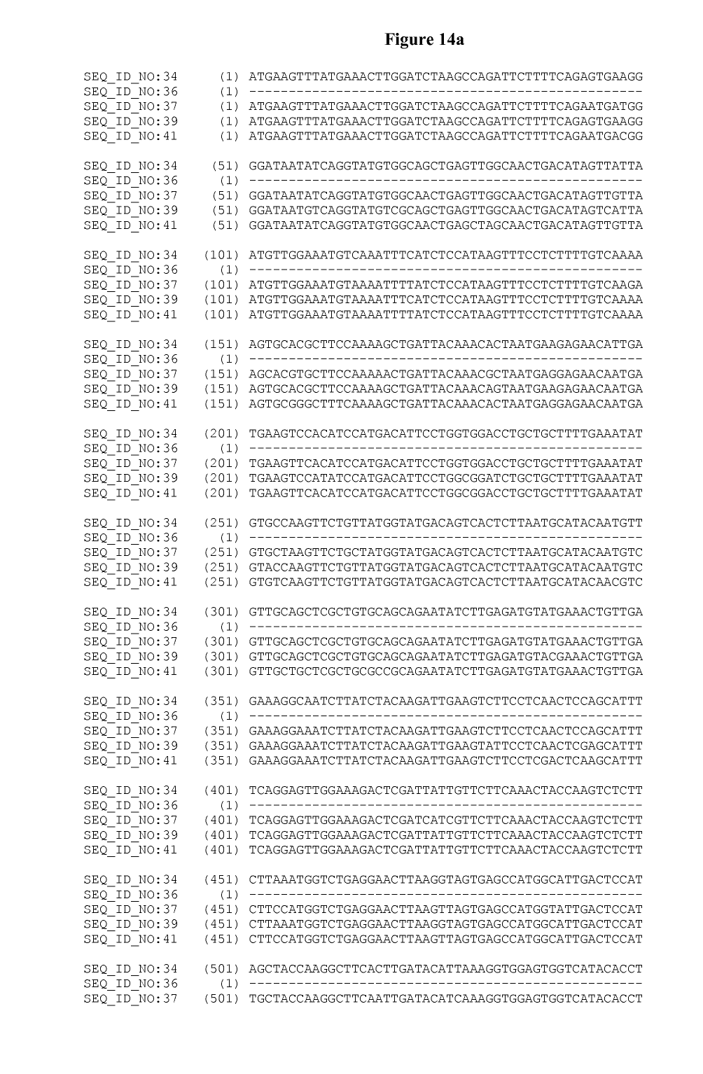

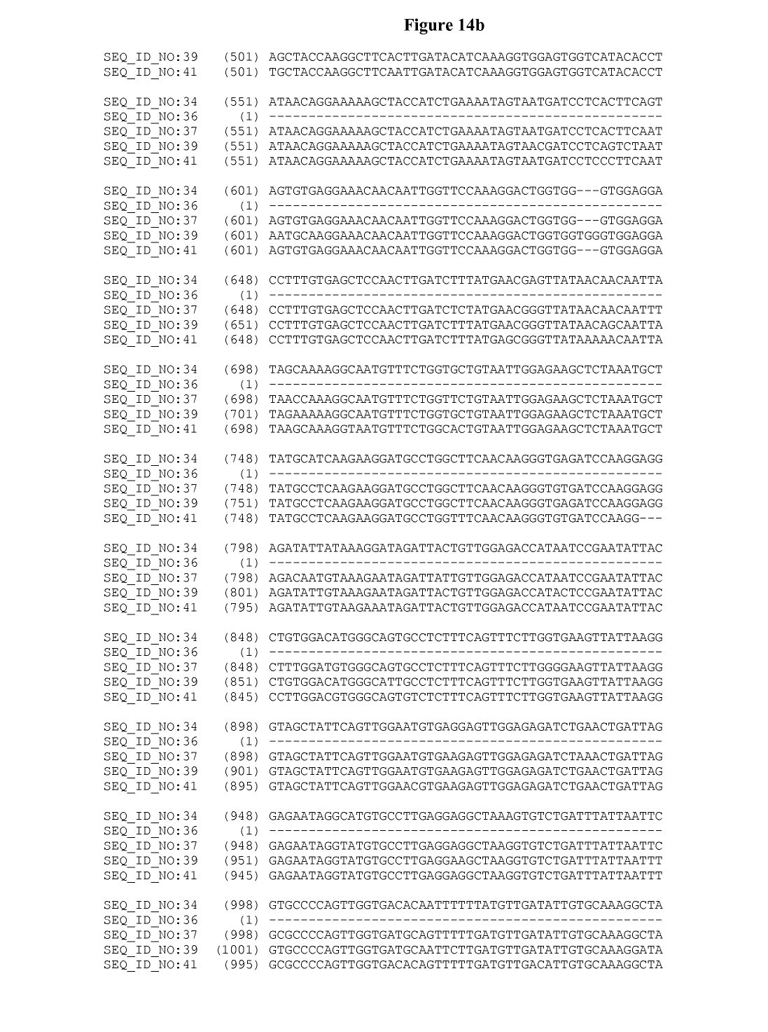

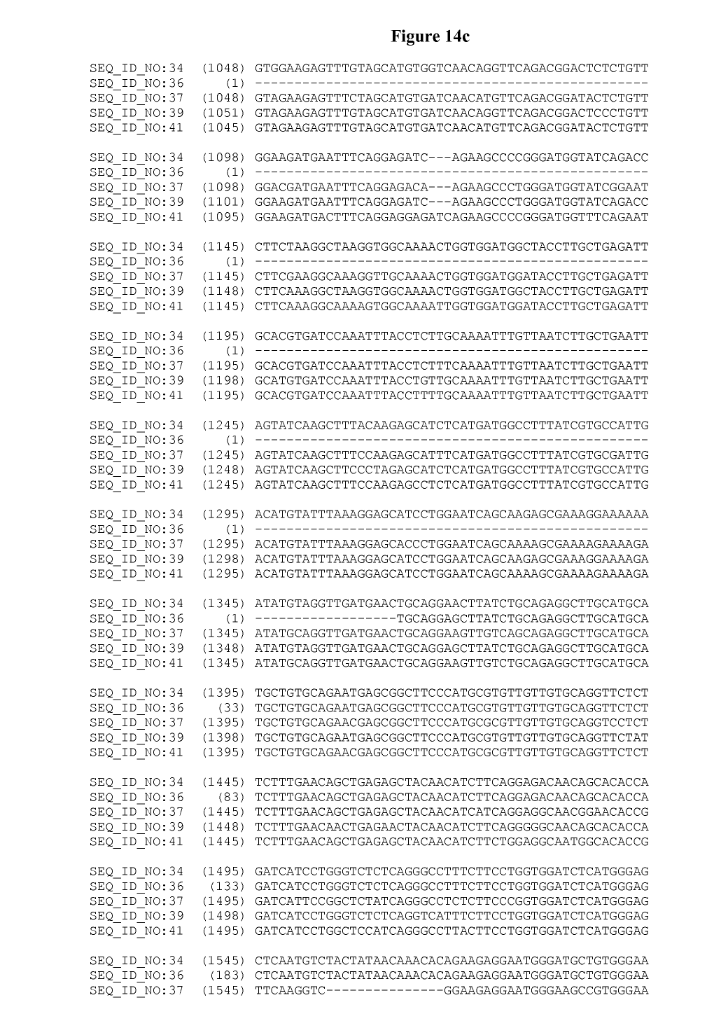

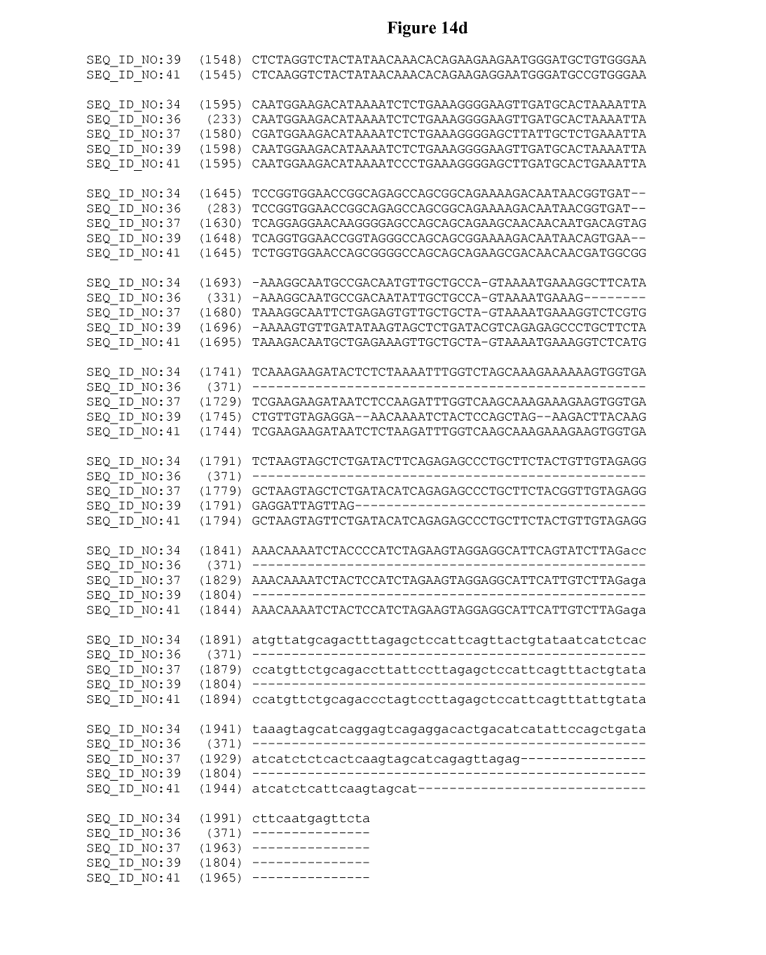

As disclosed in Example 2, the construct RTP2362-1 results in the expression of a double stranded RNA molecule that targets SEQ ID NO:34 and results in reduced cyst count when operably linked to a SCN-inducible promoter and expressed in soybean roots. As disclosed in Example 2, the construct RTP2361-4 results in the expression of a double stranded RNA molecule that targets SEQ ID NO:34 and results in reduced cyst count when operably linked to a constitutive promoter and expressed in soybean roots. The sense fragment of the GmNPY1 gene contained in RTP2362-1 and RTP2361-4, described by SEQ ID NO:36, corresponds to nucleotides 1458 to 1827 of the full length GmNPY1 sequence described by SEQ ID NO:34. At least one of the resulting 21 mers derived from the processing of the double stranded RNA molecule expressed from RTP2362-1 or RTP2361-4 can target other soybean sequences described by SEQ ID NO:37, SEQ ID NO:39 and SEQ ID NO:41. The amino acid alignment of the identified targets of the double stranded RNA molecule expressed from RTP2362-1 and RTP2361-4 described by the GmNPY1 target gene SEQ ID NO:35, GmNPY-like2 described by SEQ ID NO:38, GmNPY-like3 described by SEQ ID NO:40 and GmNPY-like4 described by SEQ ID NO:42 is shown in FIG. 7. The nucleotide alignment of the identified targets of the double stranded RNA molecule expressed from RTP2362-1 and RTP2361-4 described by the GmNPY1 target gene SEQ ID NO:34, the sense fragment of the GmNPY1 gene contained in RTP2362-1 and RTP2361-4 described by SEQ ID NO:36, GmNPY-like2 gene described by SEQ ID NO: 37, the GmNPY-like3 gene described by SEQ ID NO:39 and the GmNPY-like4 gene described by SEQ ID NO:41 is shown in FIG. 14. A matrix table showing the amino acid sequence percent identity of the full length amino acid sequence of the GmNPY1 gene described by SEQ ID NO:35 and additional soybean transcript targets of the double stranded RNA molecule expressed by RTP2362-1 and RTP2361-4 described by SEQ ID NO:38, SEQ ID NO:40 and SEQ ID NO:42 to each other is shown in FIG. 16k. A matrix table showing the DNA sequence percent identity of the open reading frame transcript sequence of the GmNPY1 gene described by SEQ ID NO:34, the sense fragment of the GmNPY1 gene contained in RTP2362-1 and RTP2361-4 described by SEQ ID NO:36, and additional soybean transcript targets of the double stranded RNA molecule expressed by RTP2362-1 and RTP2361-4 described by SEQ ID NO:37, SEQ ID NO:39 and SEQ ID NO:41 to each other is shown in FIG. 16l.

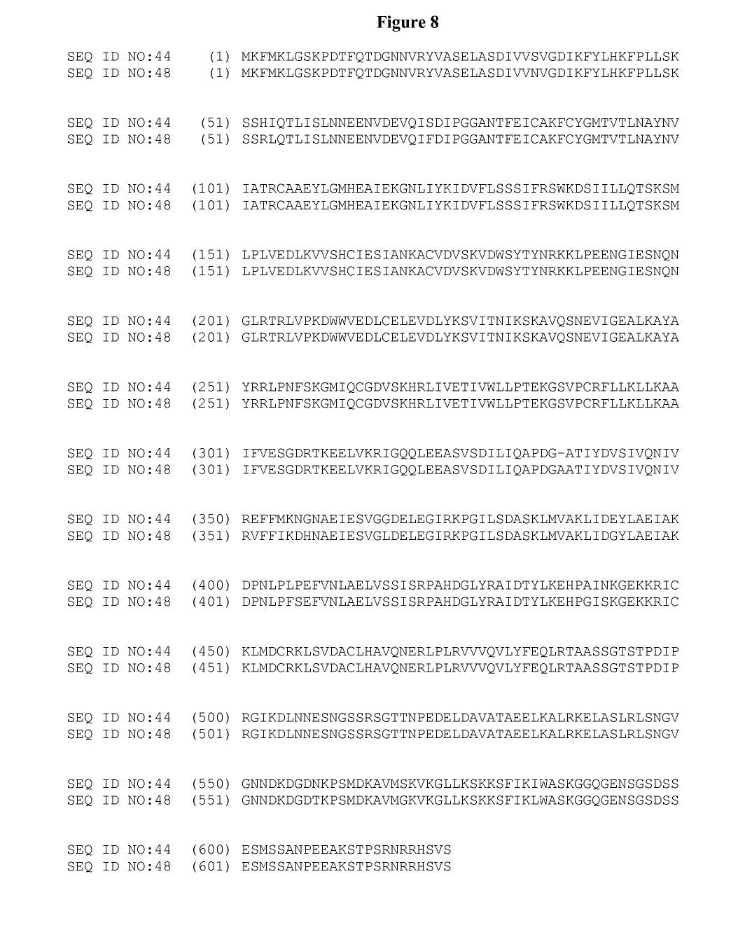

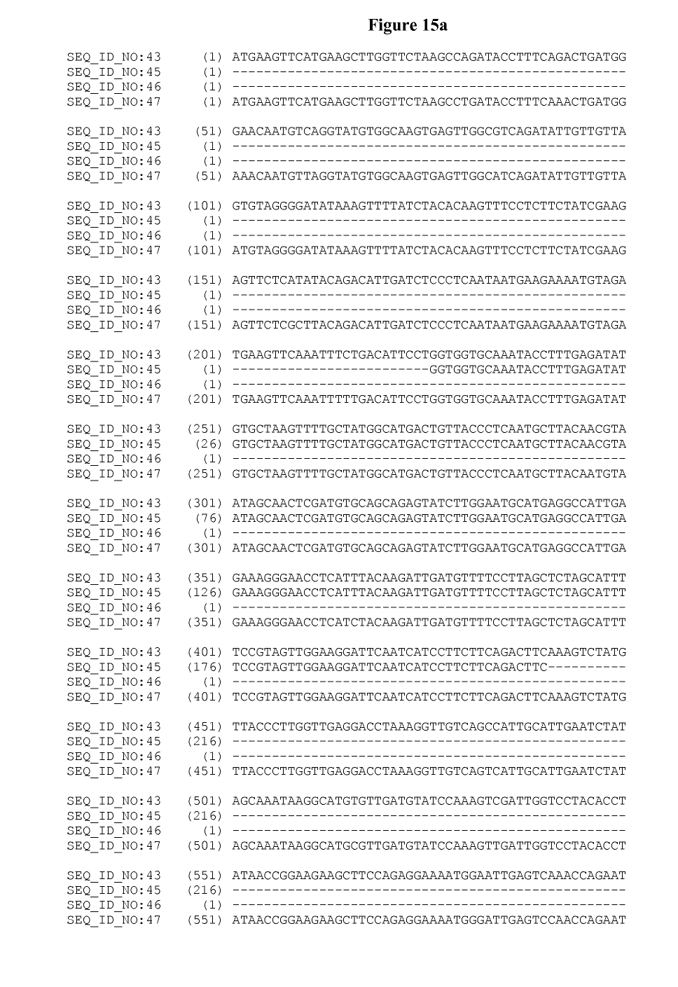

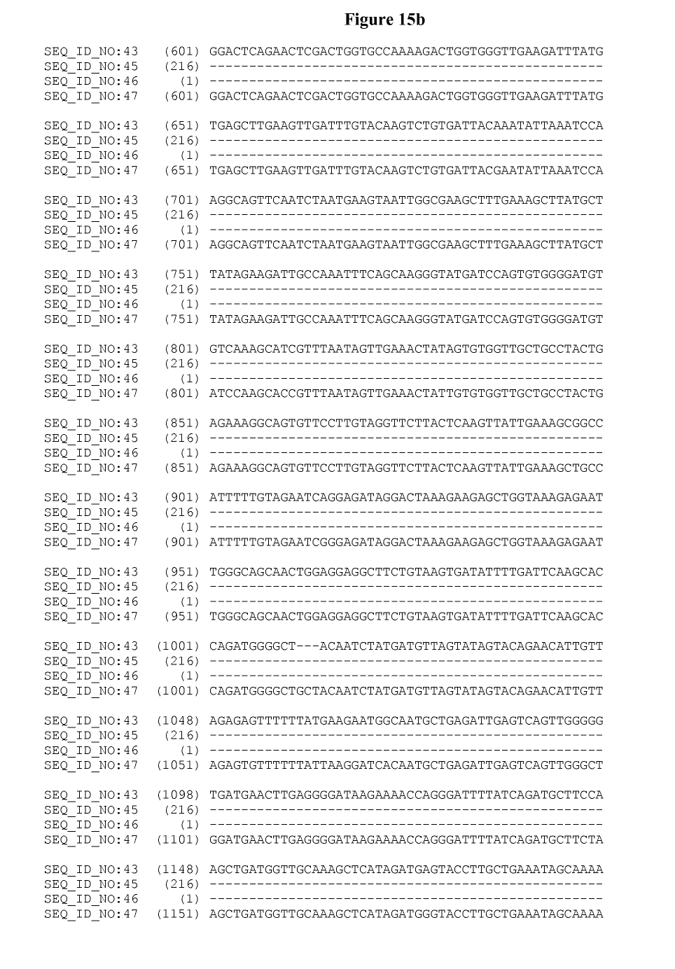

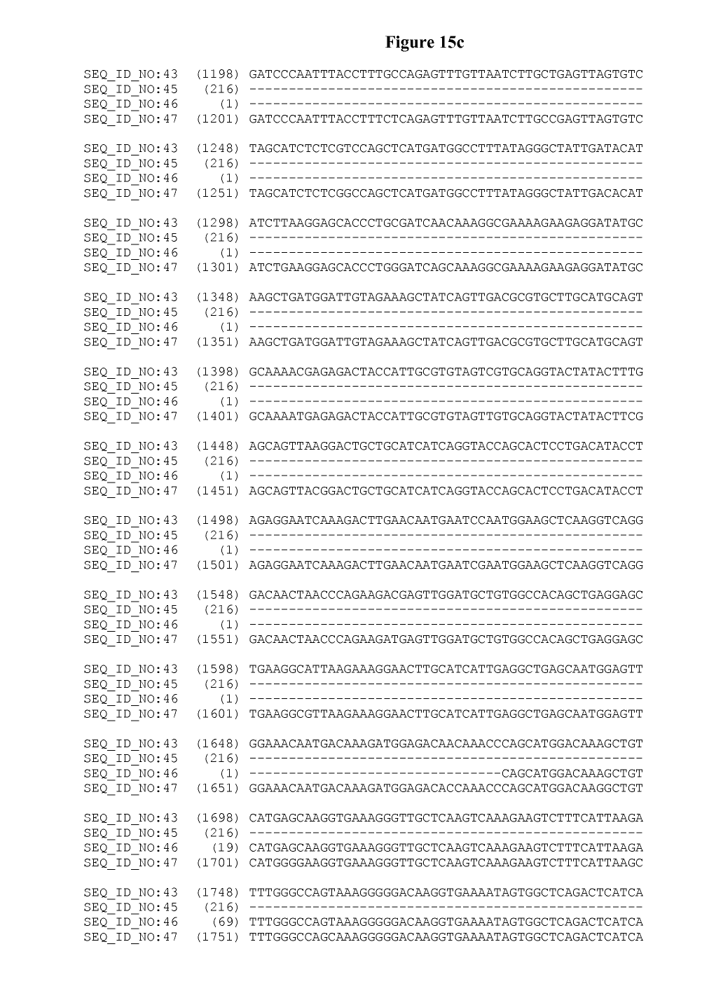

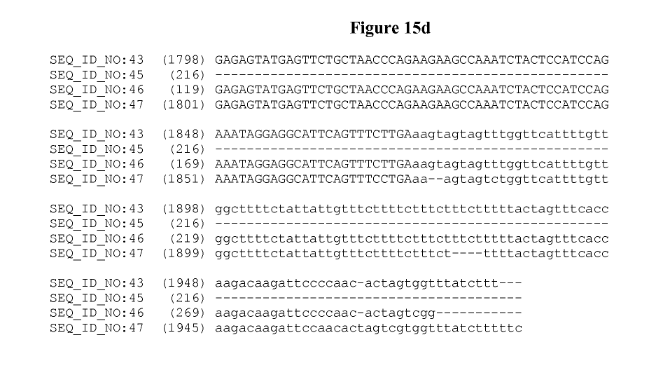

As disclosed in Example 2, the construct RTP4082-1 results in the expression of a double stranded RNA molecule that targets SEQ ID NO:43 and results in reduced cyst count when operably linked to a constitutive and expressed in soybean roots. The sense fragment of the GmNPY-like5 gene contained in RTP4082-1 described by SEQ ID NO:45, corresponds to nucleotides 344 to 558 of the full length GmNPY-like5 sequence described by SEQ ID NO:43. As disclosed in Example 2, the construct RTP4083-1 results in the expression of a double stranded RNA molecule that targets SEQ ID NO:43 and results in reduced cyst count when operably linked to a constitutive promoter and expressed in soybean roots. The sense fragment of the GmNPY-like5 gene contained in RTP4083-1 described by SEQ ID NO:46, corresponds to nucleotides 1798 to 2089 of the full length GmNPY-like5 sequence described by SEQ ID NO:43. The sense fragment of the GmNPY-like5 gene contained in RTP4083-1 includes an exon sequence from nucleotide 1 to 193, corresponding to an exon sequence in GmNPYlike5 described by SEQ ID NO:43 from nucleotide 1798 to 1990. The sense fragment of the GmNPY-like gene contained in RTP4083-1 includes a 3′ UTR sequence from nucleotide 194 to 295, corresponding to a 3′ UTR sequence of the GmNPY-like5 gene described by SEQ ID NO: 43 from nucleotide 1991 to 2091. At least one of the resulting 21 mers derived from the processing of the double stranded RNA molecule expressed from RTP4082-1 or RTP4083-1 can target another soybean sequence described by SEQ ID NO:47. The amino acid alignment of the identified targets of the double stranded RNA molecule expressed from RTP4082-1 or RTP4083-1 described by the GmNPY-like5 target gene SEQ ID NO:44 and GmNPY-like6 described by SEQ ID NO:48 is shown in FIG. 8. The open reading frame nucleotide alignment of the identified targets of the double stranded RNA molecule expressed from RTP4082-1 or RTP4083-1 described by the GmNPY-like5 target gene SEQ ID NO:43, the sense fragment of the GmNPY-like5 gene contained in RTP4082-1 described by SEQ ID NO:45, the sense fragment of the GmNPY-like5 gene contained in RTP4083-1 described by SEQ ID NO:46 and the GmNPY-like6 sequence described by SEQ ID NO:47 is shown in FIG. 15. A matrix table showing the amino acid sequence percent identity of the full length amino acid sequence of the GmNPY-like5 gene described by SEQ ID NO:44 and an additional soybean transcript target of the double stranded RNA molecule expressed by RTP4082-1 and RTP4083-1 described by SEQ ID NO:48 to each other is shown in FIG. 16m. A matrix table showing the DNA sequence percent identity of the open reading frame transcript sequence of the GmNPY-like5 gene described by SEQ ID NO:43 and a additional soybean transcript target of the double stranded RNA molecule expressed by RTP4082-1 and RTP4083-1 described by SEQ ID NO:47 to each other is shown in FIG. 16n.

### Example 6

### Identification of CLASP and NPY1 Homologs

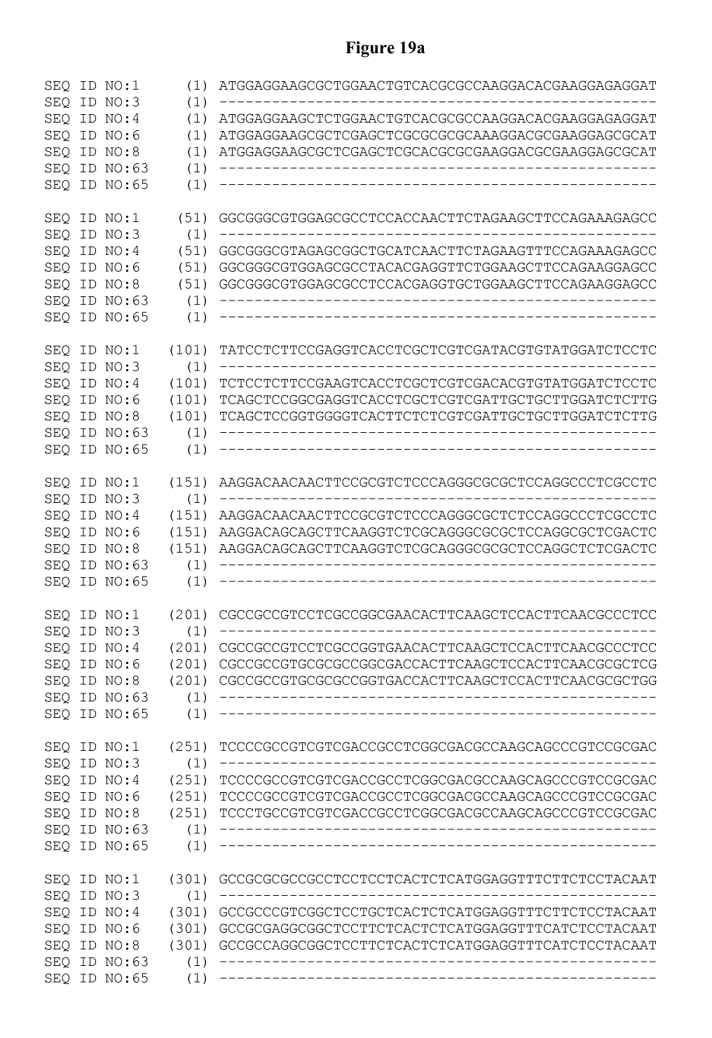

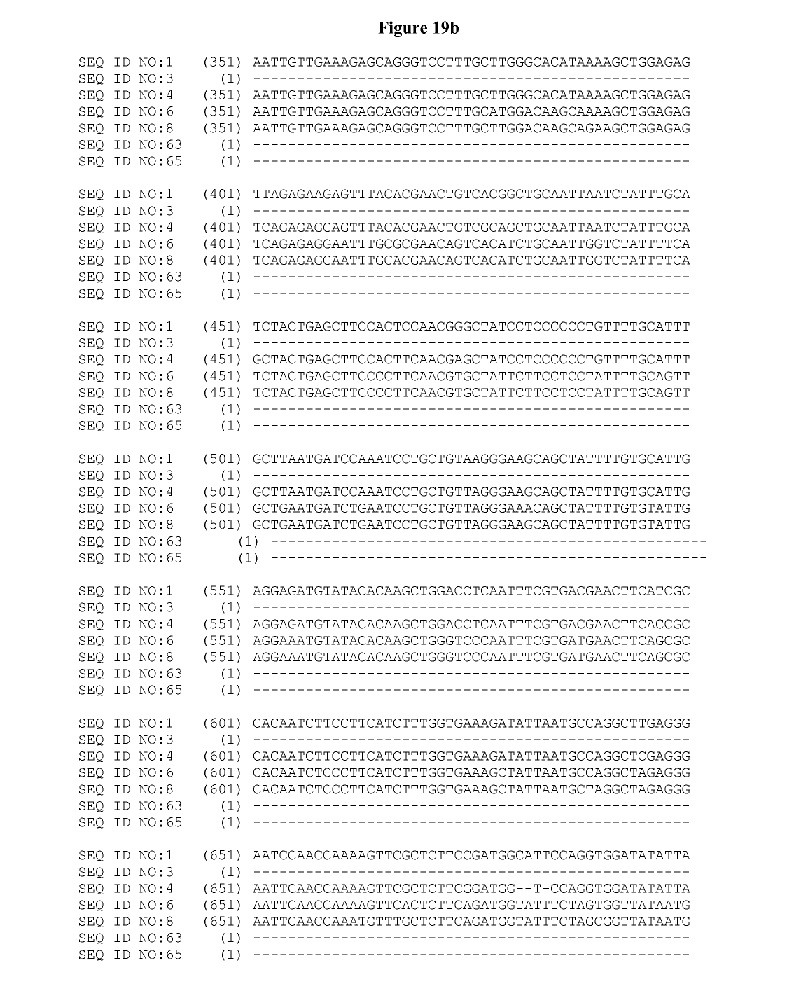

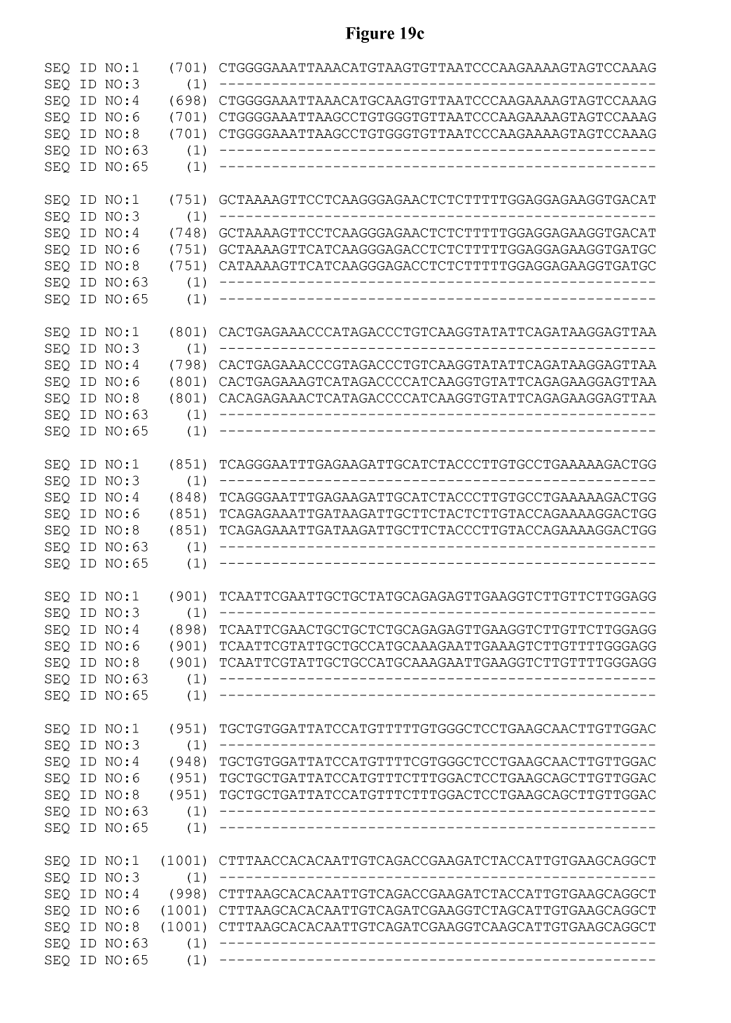

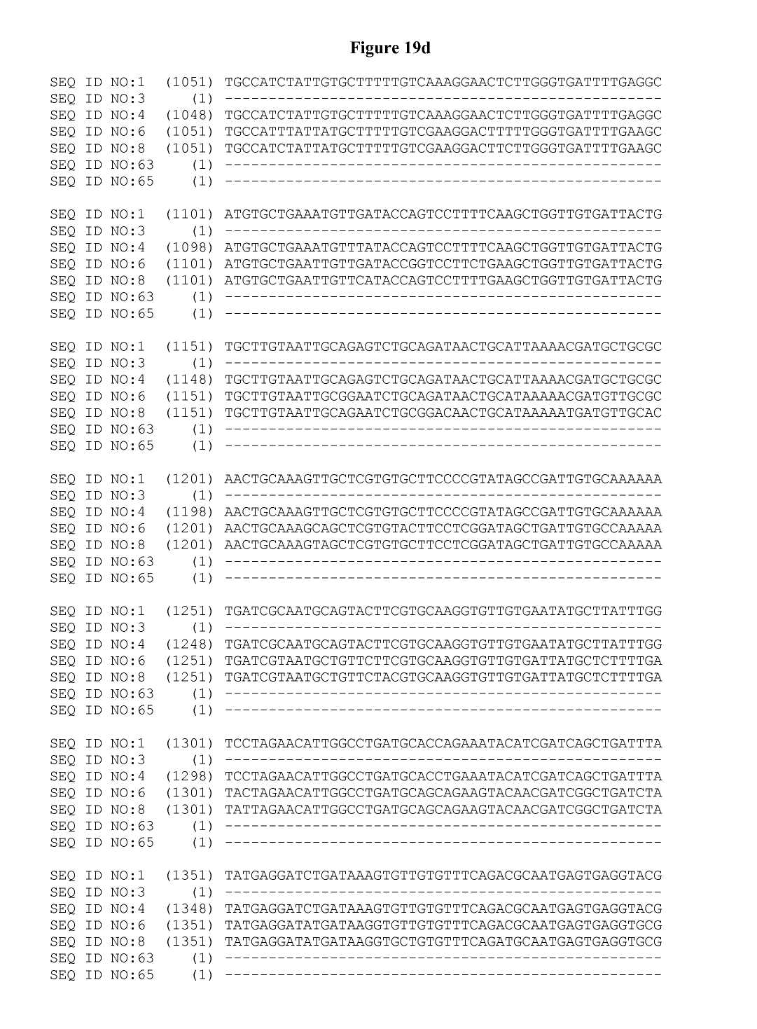

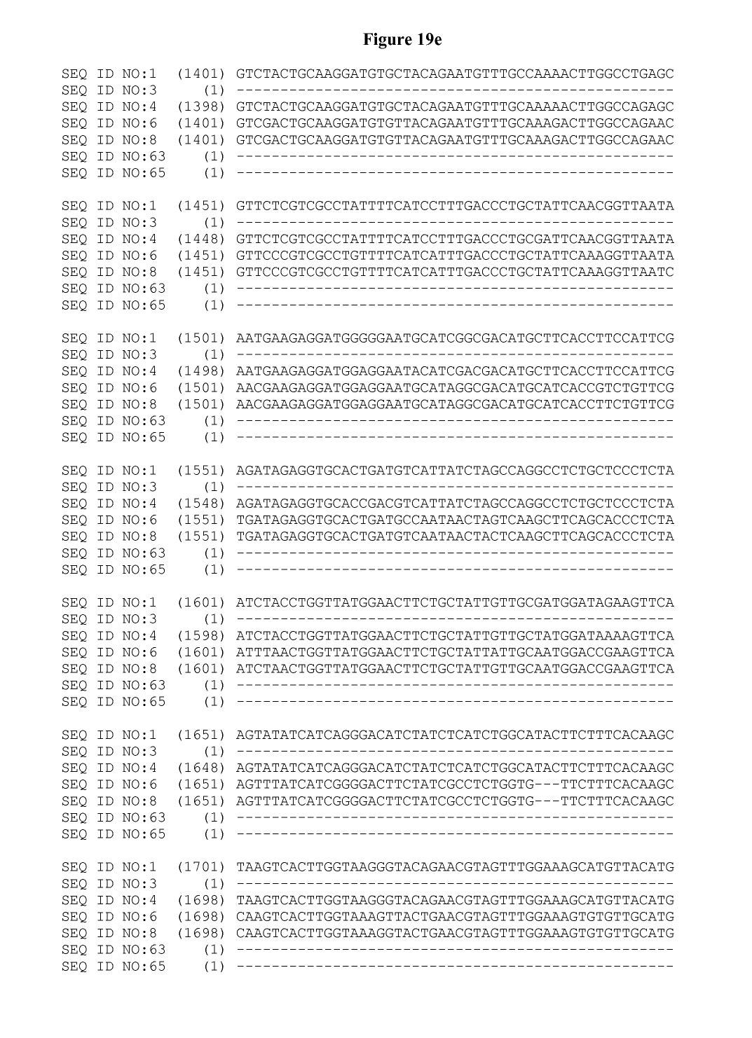

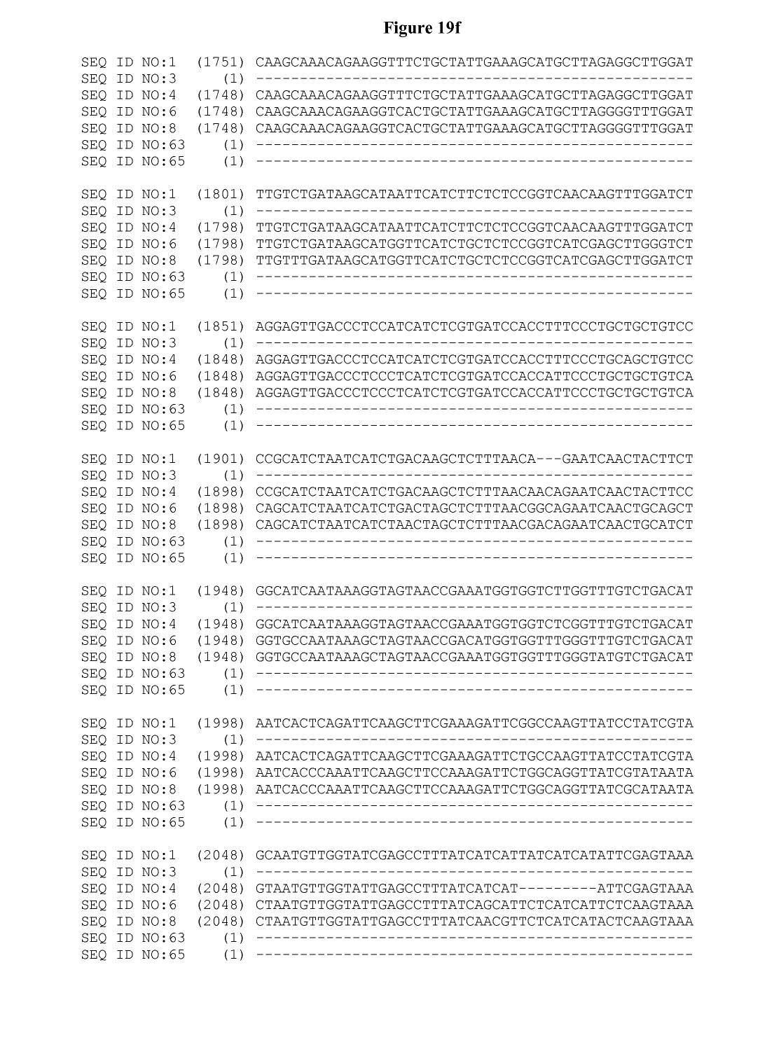

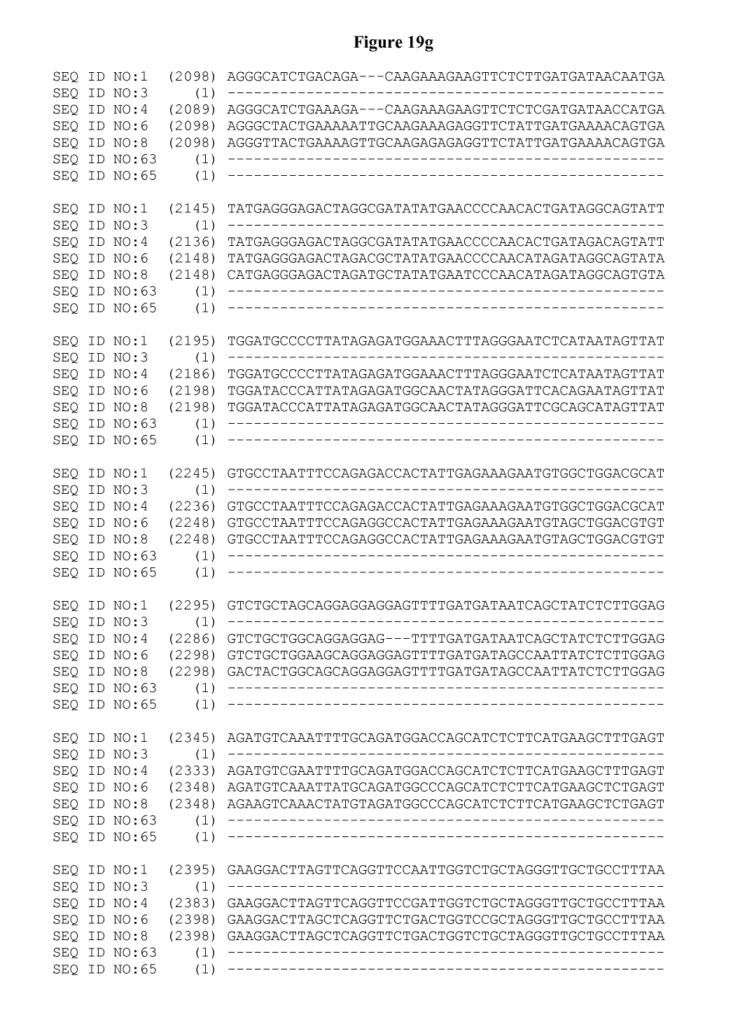

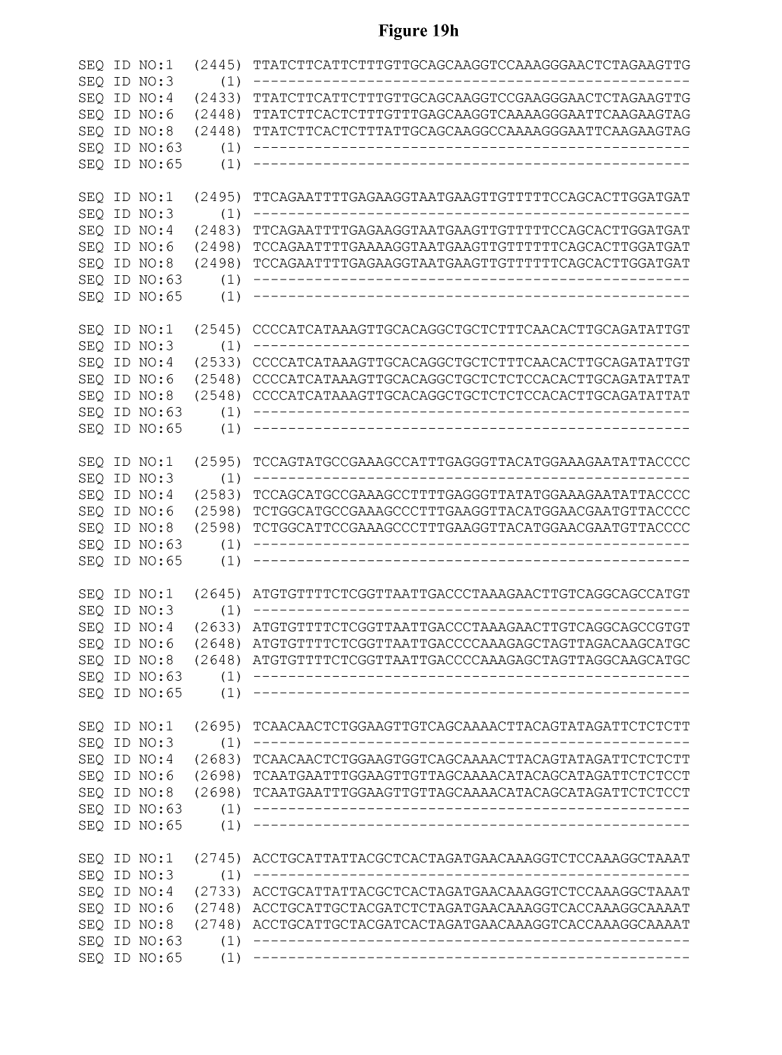

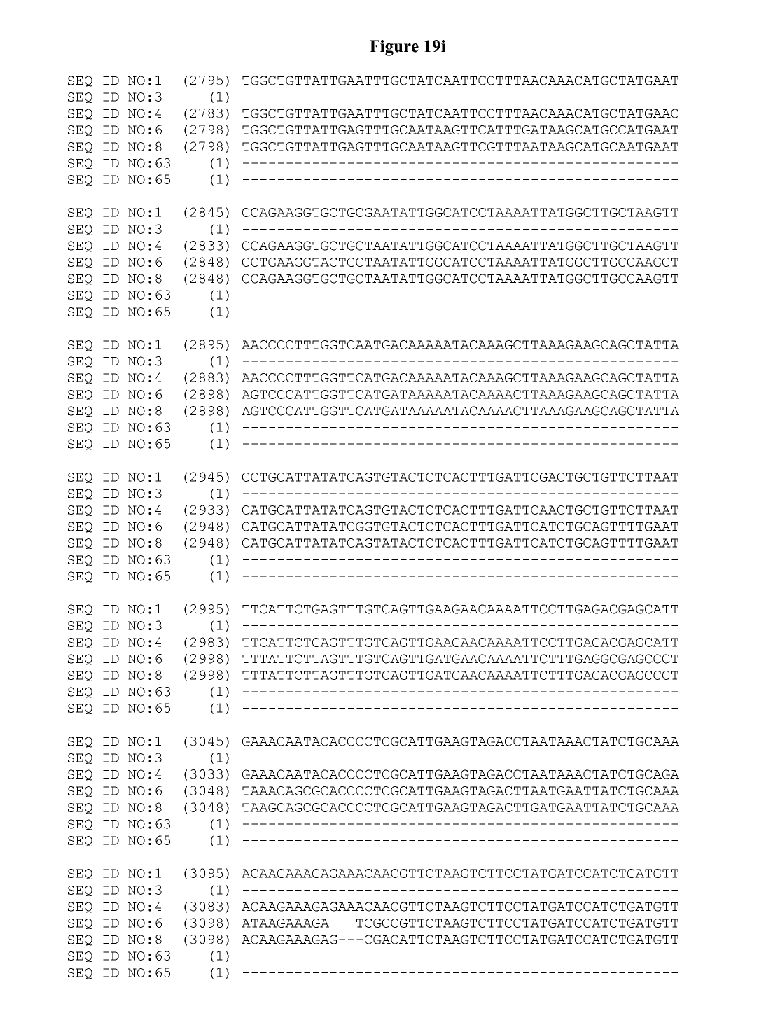

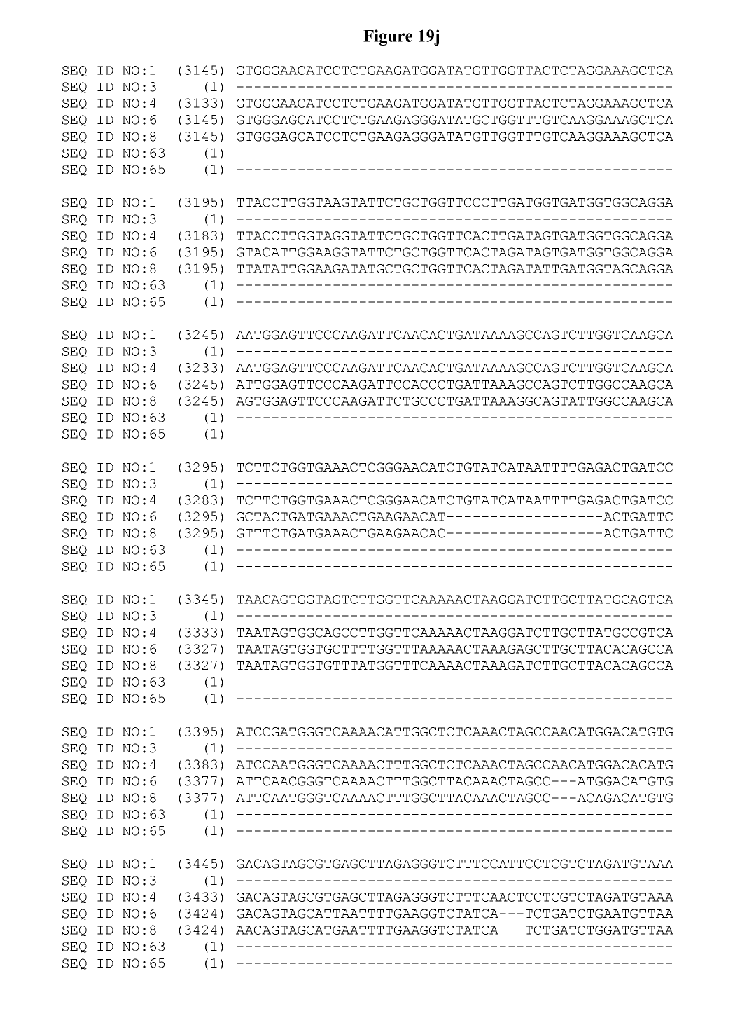

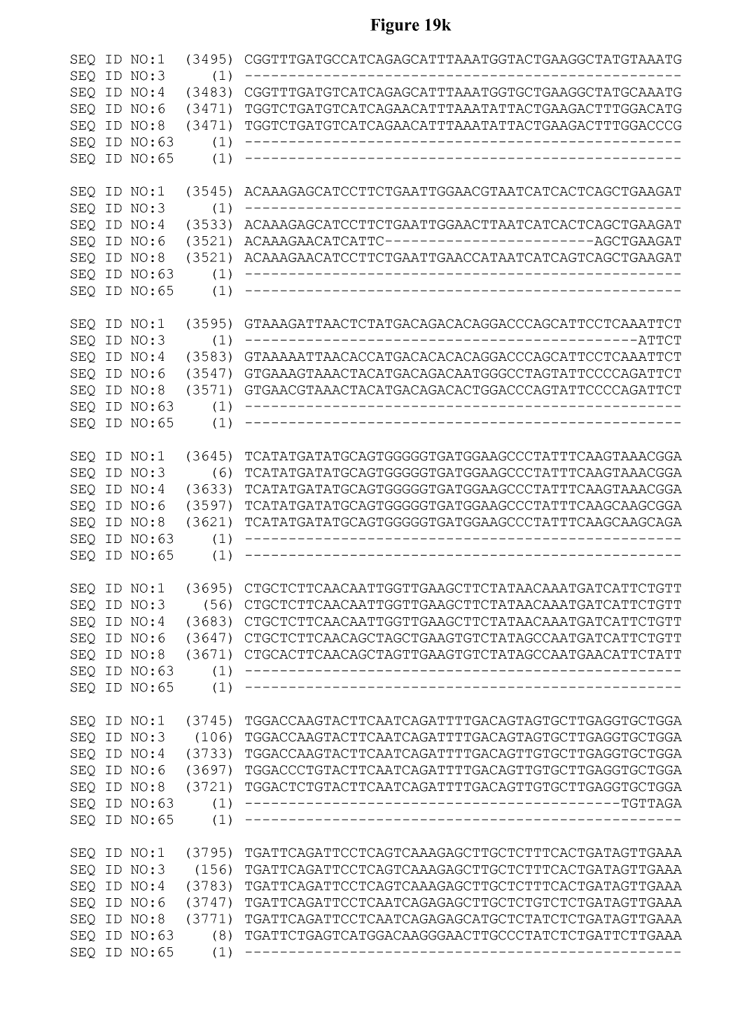

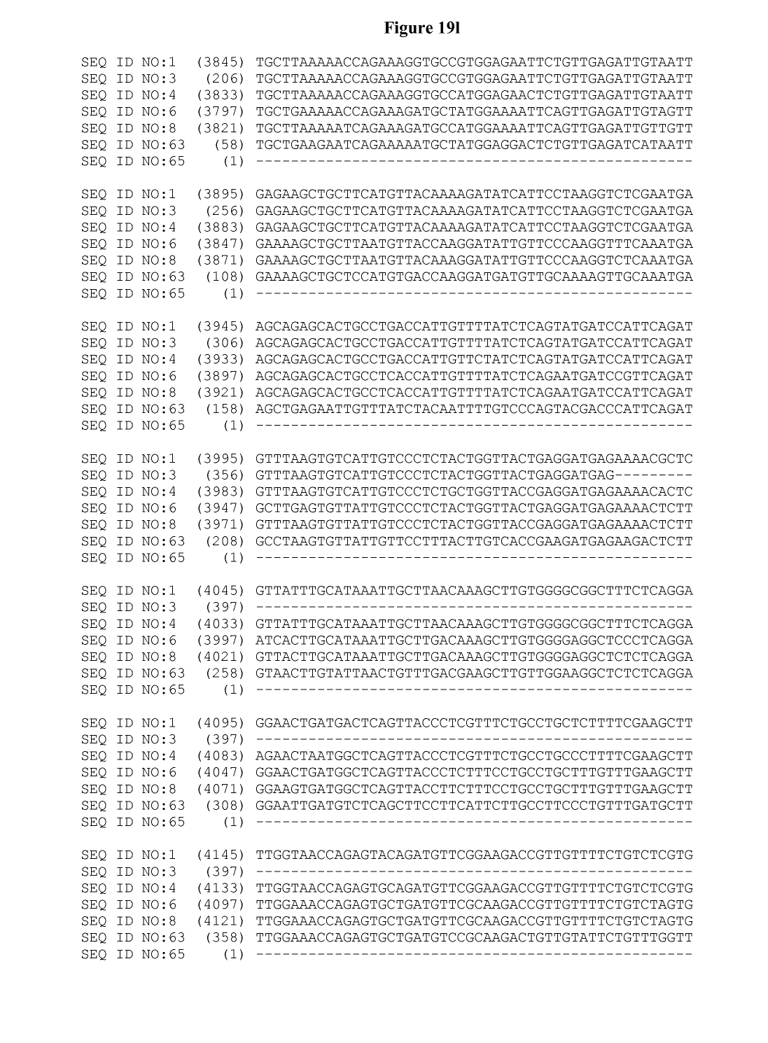

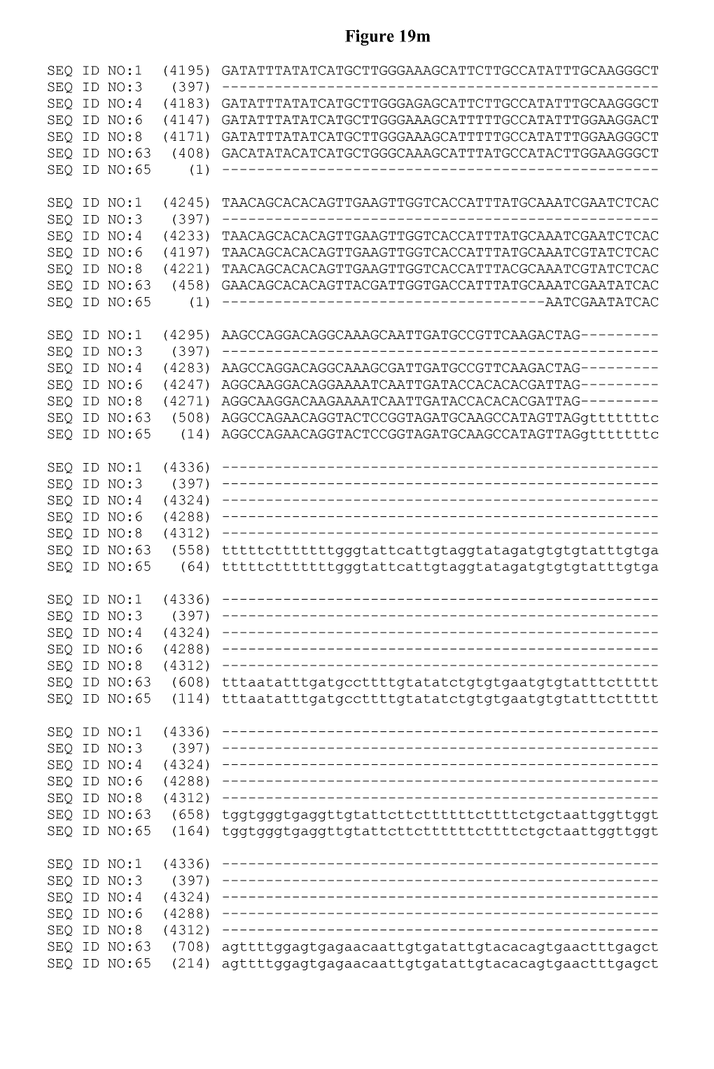

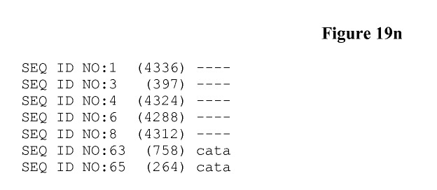

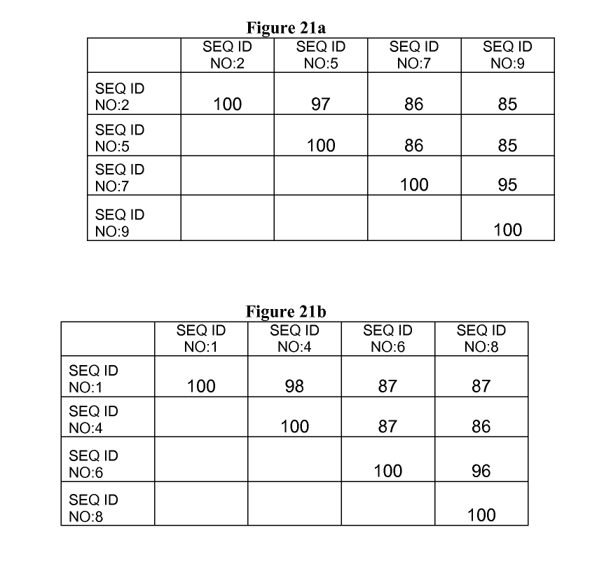

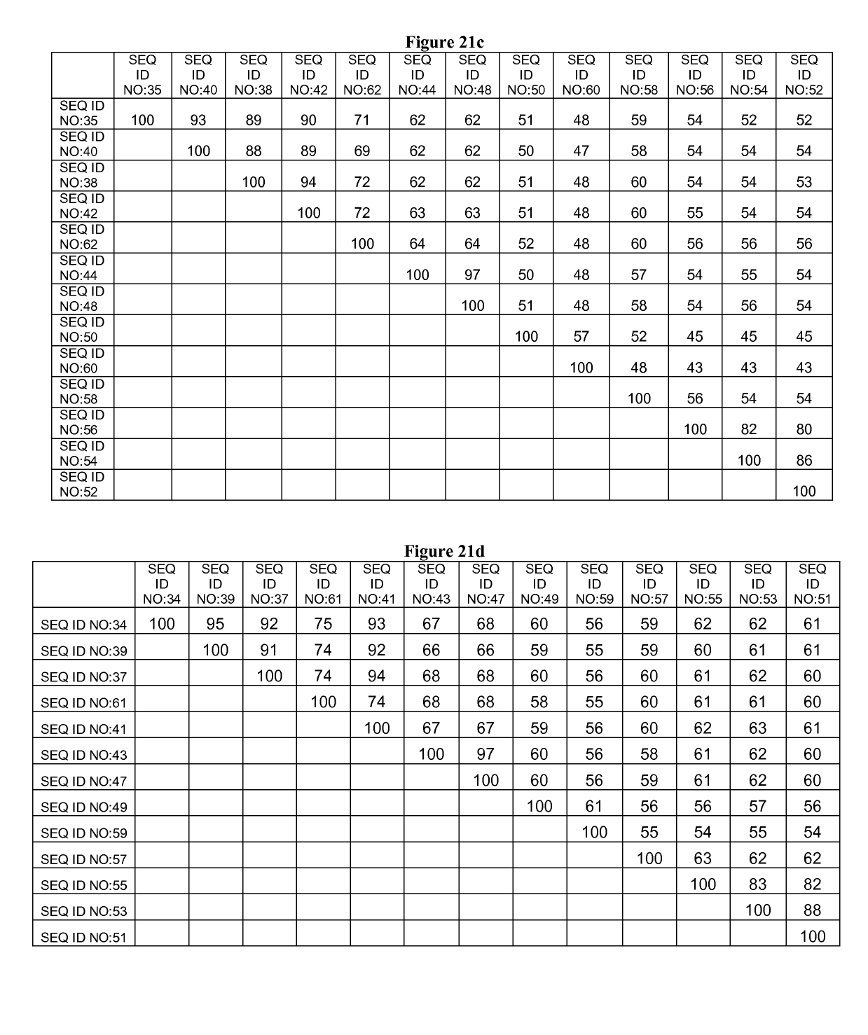

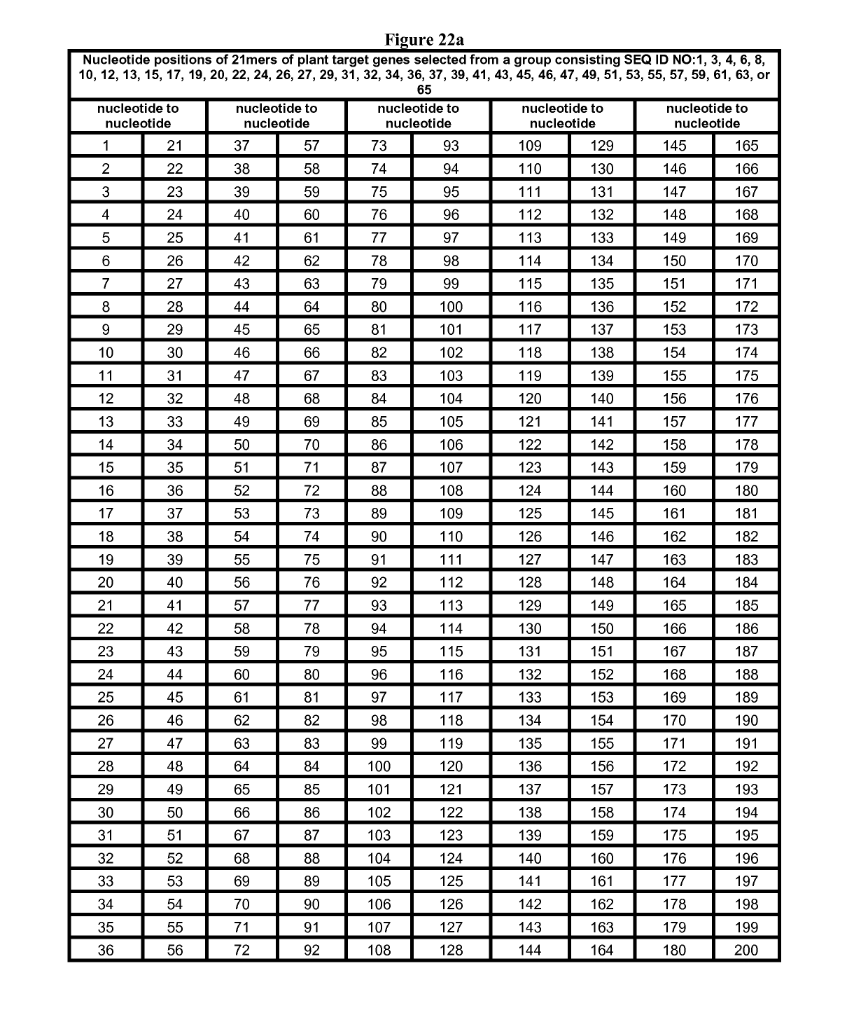

As disclosed in Example 3 the potato CLASP homolog described by SEQ ID NO:64 was identified based on sequence similarity searches to the identified targets, described by soybean sequences SEQ ID NO: 2, 5, 7 and 9, of the double stranded RNA molecule expressed from RTP2593-3 The amino acid alignment of the identified partial potato homolog described by SEQ ID NO:64 to the identified targets of the double stranded RNA molecule expressed from RTP2593-3 described by soybean target sequences SEQ ID NO: 2, 5, 7 and 9 is shown in FIG. 17. A matrix table showing the amino acid percent identity of the identified partial potato homolog described by SEQ ID NO:64 to the identified targets of the double stranded RNA molecule expressed from RTP2593-3 described by soybean target sequences SEQ ID NO: 2, 5, 7 and 9 to each other is shown in FIG. 21a. The DNA sequence alignment of the identified partial potato homolog SEQ ID NO:63 and the sense strand contained in RTP2622 described by SEQ ID NO:65 to the identified targets of the double stranded RNA molecule expressed from RTP2593-3 described by soybean target sequences SEQ ID NO:1, 4, 6 and 8 and to the sense strand contained in RTP2593-3 described by SEQ ID NO:3 is shown in FIG. 19. A matrix table showing the DNA sequence percent identity of the identified targets of the double stranded RNA molecule expressed from RTP2593-3 described by GmCLASP1 target gene SEQ ID NO:1, Glyma03g32710.1 target gene SEQ ID NO:4, Glyma13g19230.1 target gene SEQ ID NO:6, Glyma10g04850.1 target gene SEQ ID NO:8, the sense strand contained in RTP2593-3-1 described by SEQ ID NO:3, the identified partial potato homolog SEQ ID NO:63 and the sense strand contained in RTP2622 described by SEQ ID NO:65 to each other is shown in FIG. 21b.

As disclosed in Example 2, the construct RTP2362-1 results in the expression of a double stranded RNA molecule that targets SEQ ID NO:34 and results in reduced cyst count when operably linked to a SCN-inducible promoter and expressed in soybean roots. As disclosed in Example 2, the construct RTP2361-4 results in the expression of a double stranded RNA molecule that targets SEQ ID NO:34 and results in reduced cyst count when operably linked to a constitutive promoter and expressed in soybean roots. As disclosed in Example 2, the construct RTP4082-1 results in the expression of a double stranded RNA molecule that targets SEQ ID NO:43 and results in reduced cyst count when operably linked to a constitutive promoter and expressed in soybean roots. As disclosed in Example 2, the construct RTP4083-1 results in the expression of a double stranded RNA molecule that targets SEQ ID NO:43 and results in reduced cyst count when operably linked to a constitutive promoter and expressed in soybean roots. As disclosed in Example 1, the putative full length transcript sequence of the gene described by SEQ ID NO:34 contains an open reading frame with the amino acid sequence disclosed as SEQ ID NO:35 and the putative full length transcript sequence of the gene described by SEQ ID NO:43 contains an open reading frame with the amino acid sequence disclosed as SEQ ID NO:44. The amino acid sequences described by SEQ ID NO:35 and SEQ ID NO:44 were used to identify homologous genes from soybean and other plant species. Sample genes with DNA sequences homologous to SEQ ID NO:34 and SEQ IS NO:43 were identified by SEQ ID NO:49, SEQ ID NO:51, SEQ ID NO:53, SEQ ID NO:55, SEQ ID NO:57, SEQ ID NO:59 and SEQ ID NO:61. The putative full length transcript sequences of the genes described by SEQ ID NO:49, SEQ ID NO:51, SEQ ID NO:53, SEQ ID NO:55, SEQ ID NO:57, SEQ ID NO:59 and SEQ ID NO:61 contain open reading frames with the amino acid sequences disclosed as SEQ ID NO:50, SEQ ID NO:52, SEQ ID NO:54, SEQ ID NO:56, SEQ ID NO:58, SEQ ID NO:60 and SEQ ID NO:62, respectively. The amino acid alignment of the identified homologs to the identified targets of the double stranded RNA molecule expressed from RTP2362-1 and from RTP2361-4 are described by soybean target sequences SEQ ID NO:35, SEQ ID NO:38, SEQ ID NO:40 and SEQ ID NO:42, and to the identified targets of the double stranded RNA molecules expressed from RTP4082-1 and RTP4083-1, described by soybean target sequences SEQ ID NO:44, SEQ ID NO:48, is shown in FIG. 18. The nucleotide alignment of the identified homologs to the identified targets of the double stranded RNA molecule expressed from RTP2362-1 and RTP2361-4, described by soybean target sequences SEQ ID NO:34, SEQ ID NO:37, SEQ ID NO:39 and SEQ ID NO:41, to the sense strand contained in RTP2362-1 and RTP2361-4 described by SEQ ID NO:36, to the identified targets of the double stranded RNA molecules expressed from RTP4082-1 and from RTP4083-1, described by soybean target sequences SEQ ID NO:43 and SEQ ID NO:47, to the sense strand contained in RTP4082-1 described by SEQ ID NO:45 and to the sense strand contained in RTP4083-1 described by SEQ ID NO:46 is shown in FIG. 20. A matrix table showing the amino acid percent identity of the identified homologs to the identified targets of the double stranded RNA molecule expressed from RTP2362-1 and from RTP2361-4 described by soybean target sequences SEQ ID NO:35, SEQ ID NO:38, SEQ ID NO:40 and SEQ ID NO:42, and to the identified targets of the double stranded RNA molecules expressed from RTP4082-1 and RTP4083-1, described by soybean target sequences SEQ ID NO:44, SEQ ID NO:48, to each other is shown in FIG. 21c. A matrix table showing the nucleotide percent identity of the identified homologs to the identified targets of the double stranded RNA molecule expressed from RTP2362-1 and from RTP2361-4 described by soybean target sequences SEQ ID NO:34, SEQ ID NO:37, SEQ ID NO:39 and SEQ ID NO:41, and to the identified targets of the double stranded RNA molecules expressed from RTP4082-1 and RTP4083-1, described by soybean target sequences SEQ ID NO:43, SEQ ID NO:47, to each other is shown in FIG. 21d.

Those skilled in the art will recognize, or will be able to ascertain using no more than routine experimentation, many equivalents to the specific embodiments of the invention described herein. Such equivalents are intended to be encompassed by the following claims.

1. An isolated expression vector encoding a double stranded RNA comprising a first strand and a second strand complementary to the first strand, wherein the first strand is substantially identical to at least 19, 20, or 21 consecutive nucleotides of a plant target polynucleotide selected from the group consisting of a plant CLASP1 gene, an Aspartic Proteinase Delta Subunit gene, a Secreted Protein1 gene, a Lectin Receptor Kinase-like gene, a Pectin Methylesterase-like gene, and an NPY gene, wherein the double stranded RNA inhibits expression of the target gene.

2. The isolated expression vector of claim 1, wherein the plant target polynucleotide is selected from the group consisting of
(a) a polynucleotide comprising SEQ ID NO:1, SEQ ID NO:4, SEQ ID NO:6, SEQ ID NO:8, SEQ ID NO: 63, or SEQ ID NO: 65;
(b) a polynucleotide comprising SEQ ID NO:10, SEQ ID NO:13, or SEQ ID NO:15;
(c) a polynucleotide comprising SEQ ID NO:17, SEQ ID NO:20, or SEQ ID NO:22;
(d) a polynucleotide comprising SEQ ID NO:24 or SEQ ID NO:27;
(e) a polynucleotide comprising SEQ ID NO:29 or SEQ ID NO:32; and
(f) a polynucleotide comprising SEQ ID NO:34, SEQ ID NO:37, SEQ ID NO:39, SEQ ID NO:41, SEQ ID NO:43, SEQ ID NO: 45, SEQ ID NO: 46, SEQ ID NO:47; SEQ ID NO: 49; SEQ ID NO:51, SEQ ID NO: 53, SEQ ID NO: 55, SEQ ID NO: 57, SEQ ID NO: 59, or SEQ ID NO: 61.

3. An isolated expression vector comprising a nucleic acid encoding a multiplicity of double stranded RNA molecules each comprising a double stranded region having a length of at least 19, 20, or 21 consecutive nucleotides, wherein one strand of said double stranded region is derived from a plant target polynucleotide selected from the group consisting of a plant CLASP1 gene, an Aspartic Proteinase Delta Subunit gene, a Secreted Protein1 gene, a Lectin Receptor Kinase-like gene, a Pectin Methylesterase-like gene, and an NPY gene, wherein the double stranded RNA inhibits expression of the target gene.

4. The isolated expression vector of claim 3, wherein the plant target polynucleotide is selected from the group consisting of
(a) a polynucleotide comprising SEQ ID NO:1, SEQ ID NO:4, SEQ ID NO:6, SEQ ID NO:8, SEQ ID NO: 63, or SEQ ID NO: 65;
(b) a polynucleotide comprising SEQ ID NO:10, SEQ ID NO:13, or SEQ ID NO:15;
(c) a polynucleotide comprising SEQ ID NO:17, SEQ ID NO:20, or SEQ ID NO:22;
(d) a polynucleotide comprising SEQ ID NO:24 or SEQ ID NO:27;
(e) a polynucleotide comprising SEQ ID NO:29 or SEQ ID NO:32; and
(f) a polynucleotide comprising SEQ ID NO:34, SEQ ID NO:37, SEQ ID NO:39, SEQ ID NO:41, SEQ ID NO:43, SEQ ID NO: 45, SEQ ID NO: 46, SEQ ID NO:47; SEQ ID NO: 49; SEQ ID NO: 51, SEQ ID NO: 53, SEQ ID NO: 55, SEQ ID NO: 57, SEQ ID NO: 59, or SEQ ID NO: 61.

5. A transgenic plant capable of expressing at least one dsRNA that is substantially identical to at least 19, 20, or 21 consecutive nucleotides of a plant target polynucleotide selected from the group consisting of a plant CLASP1 gene, an Aspartic Proteinase Delta Subunit gene, a Secreted Protein1 gene, a Lectin Receptor Kinase-like gene, a Pectin Methylesterase-like gene, and an NPY gene, wherein the dsRNA inhibits expression of the target gene in the plant root.

6. The transgenic plant of claim 5, wherein the plant target polynucleotide is selected from the group consisting of
(a) a polynucleotide comprising SEQ ID NO: 1, SEQ ID NO:4, SEQ ID NO:6, SEQ ID NO:8, SEQ ID NO: 63, or SEQ ID NO: 65;
(b) a polynucleotide comprising SEQ ID NO:10, SEQ ID NO:13, or SEQ ID NO:15;
(c) a polynucleotide comprising SEQ ID NO:17, SEQ ID NO:20, or SEQ ID NO:22;
(d) a polynucleotide comprising SEQ ID NO:24 or SEQ ID NO:27;
(e) a polynucleotide comprising SEQ ID NO:29 or SEQ ID NO:32; and
(f) a polynucleotide comprising SEQ ID NO:34, SEQ ID NO:37, SEQ ID NO:39, SEQ ID NO:41, SEQ ID NO:43, SEQ ID NO: 45, SEQ ID NO: 46, SEQ ID NO:47; SEQ ID NO: 49; SEQ ID NO: 51, SEQ ID NO: 53, SEQ ID NO: 55, SEQ ID NO: 57, SEQ ID NO: 59, or SEQ ID NO: 61.

7. A method of making a transgenic plant capable of expressing a dsRNA comprising a first strand that is substantially identical to portion of a plant target polynucleotide and a second strand complementary to the first strand, wherein the target polynucleotide is selected from the group consisting of a plant CLASP1 gene, an Aspartic Proteinase Delta Subunit gene, a Secreted Protein1 gene, a Lectin Receptor Kinase-like gene, a Pectin Methylesterase-like gene, and an NPY gene said method comprising the steps of:
(i) preparing an expression vector comprising a nucleic acid encoding the dsRNA, wherein the nucleic acid is able to form a double-stranded transcript once expressed in the plant;
(ii) transforming a recipient plant with said expression vector;
(iii) producing one or more transgenic offspring of said recipient plant; and
(iv) selecting the offspring for resistance to nematode infection.

8. The method of claim 7, wherein the plant target polynucleotide is selected from the group consisting of
(a) a polynucleotide comprising SEQ ID NO:1, SEQ ID NO:4, SEQ ID NO:6, SEQ ID NO:8, SEQ ID NO: 63, or SEQ ID NO: 65;
(b) a polynucleotide comprising SEQ ID NO:10, SEQ ID NO:13, or SEQ ID NO:15;
(c) a polynucleotide comprising SEQ ID NO:17, SEQ ID NO:20, or SEQ ID NO:22;
(d) a polynucleotide comprising SEQ ID NO:24 or SEQ ID NO:27;
(e) a polynucleotide comprising a sequence as set forth in SEQ ID NO:29 or SEQ ID NO:32; and
(f) a polynucleotide comprising SEQ ID NO:34, SEQ ID NO:37, SEQ ID NO:39, SEQ ID NO:41, SEQ ID NO:43, SEQ ID NO: 45, SEQ ID NO: 46, SEQ ID NO:47; SEQ ID NO: 49; SEQ ID NO: 51, SEQ ID NO: 53, SEQ ID NO: 55, SEQ ID NO: 57, SEQ ID NO: 59, or SEQ ID NO: 61.

9. A method of conferring nematode resistance to a plant, said method comprising the steps of:
(i) selecting a plant target gene from the group consisting of a plant CLASP1 gene, an Aspartic Proteinase Delta Subunit gene, a Secreted Protein1 gene, a Lectin Receptor Kinase-like gene, a Pectin Methylesterase-like gene, and an NPY gene;
(ii) preparing an expression vector comprising a nucleic acid encoding a dsRNA comprising a first strand that is substantially identical to a portion of the plant target gene and a second strand complementary to the first strand, wherein the nucleic acid is able to form a double-stranded transcript once expressed in the plant;
(iii) transforming a recipient plant with said nucleic acid;
(iv) producing one or more transgenic offspring of said recipient plant; and
(v) selecting the offspring for nematode resistance.

10. The method of claim 9, wherein the plant target polynucleotide is selected from the group consisting of
(a) a polynucleotide comprising SEQ ID NO:1, SEQ ID NO:4, SEQ ID NO:6, SEQ ID NO:8, SEQ ID NO: 63, or SEQ ID NO: 65;
(b) a polynucleotide comprising SEQ ID NO:10, SEQ ID NO:13, or SEQ ID NO:15;
(c) a polynucleotide comprising SEQ ID NO:17, SEQ ID NO:20, or SEQ ID NO:22;
(d) a polynucleotide comprising SEQ ID NO:24 or SEQ ID NO:27;
(e) a polynucleotide comprising SEQ ID NO:29 or SEQ ID NO:32; and
(f) a polynucleotide comprising SEQ ID NO:34, SEQ ID NO:37, SEQ ID NO:39, SEQ ID NO:41, SEQ ID NO:43, SEQ ID NO: 45, SEQ ID NO: 46, SEQ ID NO:47; SEQ ID NO: 49, SEQ ID NO: 51, SEQ ID NO: 53, SEQ ID NO: 55, SEQ ID NO: 57, SEQ ID NO: 59, or SEQ ID NO: 61.


SEQUENCE LISTING (69 sequences)

SEQ ID NO: 1 - DNA, 4530 bp, Glycine max
cagatctgatcgaagaaagaaatggaggaagcgctggaactgtcacgcgccaaggacacgaaggagaggatggcgggcgtggagcgcctccaccaacttctagaagcttccagaaagagcctatcctcttccgaggtcacctcgctcgtcgatacgtgtatggatctcctcaaggacaacaacttccgcgtctcccagggcgcgctccaggccctcgcctccgccgccgtcctcgccggcgaacacttcaagctccacttcaacgccctcctccccgccgtcgtcgaccgcctcggcgacgccaagcagcccgtccgcgacgccgcgcgccgcctcctcctcactctcatggaggtttcttctcctacaataattgttgaaagagcagggtcctttgcttgggcacataaaagctggagagttagagaagagtttacacgaactgtcacggctgcaattaatctatttgcatctactgagcttccactccaacgggctatcctcccccctgttttgcatttgcttaatgatccaaatcctgctgtaagggaagcagctattttgtgcattgaggagatgtatacacaagctggacctcaatttcgtgacgaacttcatcgccacaatcttccttcatctttggtgaaagatattaatgccaggcttgagggaatccaaccaaaagttcgctcttccgatggcattccaggtggatatattactggggaaattaaacatgtaagtgttaatcccaagaaaagtagtccaaaggctaaaagttcctcaagggagaactctctttttggaggagaaggtgacatcactgagaaacccatagaccctgtcaaggtatattcagataaggagttaatcagggaatttgagaagattgcatctacccttgtgcctgaaaaagactggtcaattcgaattgctgctatgcagagagttgaaggtcttgttcttggaggtgctgtggattatccatgtttttgtgggctcctgaagcaacttgttggacctttaaccacacaattgtcagaccgaagatctaccattgtgaagcaggcttgccatctattgtgctttttgtcaaaggaactcttgggtgattttgaggcatgtgctgaaatgttgataccagtccttttcaagctggttgtgattactgtgcttgtaattgcagagtctgcagataactgcattaaaacgatgctgcgcaactgcaaagttgctcgtgtgcttccccgtatagccgattgtgcaaaaaatgatcgcaatgcagtacttcgtgcaaggtgttgtgaatatgcttatttggtcctagaacattggcctgatgcaccagaaatacatcgatcagctgatttatatgaggatctgataaagtgttgtgtttcagacgcaatgagtgaggtacggtctactgcaaggatgtgctacagaatgtttgccaaaacttggcctgagcgttctcgtcgcctattttcatcctttgaccctgctattcaacggttaataaatgaagaggatgggggaatgcatcggcgacatgcttcaccttccattcgagatagaggtgcactgatgtcattatctagccaggcctctgctccctctaatctacctggttatggaacttctgctattgttgcgatggatagaagttcaagtatatcatcagggacatctatctcatctggcatacttctttcacaagctaagtcacttggtaagggtacagaacgtagtttggaaagcatgttacatgcaagcaaacagaaggtttctgctattgaaagcatgcttagaggcttggatttgtctgataagcataattcatcttctctccggtcaacaagtttggatctaggagttgaccctccatcatctcgtgatccacctttccctgctgctgtccccgcatctaatcatctgacaagctctttaacagaatcaactacttctggcatcaataaaggtagtaaccgaaatggtggtcttggtttgtctgacataatcactcagattcaagcttcgaaagattcggccaagttatcctatcgtagcaatgttggtatcgagcctttatcatcattatcatcatattcgagtaaaagggcatctgacagacaagaaagaagttctcttgatgataacaatgatatgagggagactaggcgatatatgaaccccaacactgataggcagtatttggatgccccttatagagatggaaactttagggaatctcataatagttatgtgcctaatttccagagaccactattgagaaagaatgtggctggacgcatgtctgctagcaggaggaggagttttgatgataatcagctatctcttggagagatgtcaaattttgcagatggaccagcatctcttcatgaagctttgagtgaaggacttagttcaggttccaattggtctgctagggttgctgcctttaattatcttcattctttgttgcagcaaggtccaaagggaactctagaagttgttcagaattttgagaaggtaatgaagttgtttttccagcacttggatgatccccatcataaagttgcacaggctgctctttcaacacttgcagatattgttccagtatgccgaaagccatttgagggttacatggaaagaatattaccccatgtgttttctcggttaattgaccctaaagaacttgtcaggcagccatgttcaacaactctggaagttgtcagcaaaacttacagtatagattctctcttacctgcattattacgctcactagatgaacaaaggtctccaaaggctaaattggctgttattgaatttgctatcaattcctttaacaaacatgctatgaatccagaaggtgctgcgaatattggcatcctaaaattatggcttgctaagttaacccctttggtcaatgacaaaaatacaaagcttaaagaagcagctattacctgcattatatcagtgtactctcactttgattcgactgctgttcttaatttcattctgagtttgtcagttgaagaacaaaattccttgagacgagcattgaaacaatacacccctcgcattgaagtagacctaataaactatctgcaaaacaagaaagagaaacaacgttctaagtcttcctatgatccatctgatgttgtgggaacatcctctgaagatggatatgttggttactctaggaaagctcattaccttggtaagtattctgctggttcccttgatggtgatggtggcaggaaatggagttcccaagattcaacactgataaaagccagtcttggtcaagcatcttctggtgaaactcgggaacatctgtatcataattttgagactgatcctaacagtggtagtcttggttcaaaaactaaggatcttgcttatgcagtcaatccgatgggtcaaaacattggctctcaaactagccaacatggacatgtggacagtagcgtgagcttagagggtctttccattcctcgtctagatgtaaacggtttgatgccatcagagcatttaaatggtactgaaggctatgtaaatgacaaagagcatccttctgaattggaacgtaatcatcactcagctgaagatgtaaagattaactctatgacagacacaggacccagcattcctcaaattcttcatatgatatgcagtgggggtgatggaagccctatttcaagtaaacggactgctcttcaacaattggttgaagcttctataacaaatgatcattctgtttggaccaagtacttcaatcagattttgacagtagtgcttgaggtgctggatgattcagattcctcagtcaaagagcttgctctttcactgatagttgaaatgcttaaaaaccagaaaggtgccgtggagaattctgttgagattgtaattgagaagctgcttcatgttacaaaagatatcattcctaaggtctcgaatgaagcagagcactgcctgaccattgttttatctcagtatgatccattcagatgtttaagtgtcattgtccctctactggttactgaggatgagaaaacgctcgttatttgcataaattgcttaacaaagcttgtggggcggctttctcaggaggaactgatgactcagttaccctcgtttctgcctgctcttttcgaagcttttggtaaccagagtacagatgttcggaagaccgttgttttctgtctcgtggatatttatatcatgcttgggaaagcattcttgccatatttgcaagggcttaacagcacacagttgaagttggtcaccatttatgcaaatcgaatctcacaagccaggacaggcaaagcaattgatgccgttcaagactagcaggttcatggggaagaaaaaaaattgtggcttttgggtgttgtctattcattgttgtgtattttgtatatctgcgtgcatgaatttagatgcgggttccattgtctttctccttgggtctctttaaatatatagttttttttttgtctttccttttttcctggcaatgttgga

SEQ ID NO: 2 - protein, 1444 aa, Glycine max
MEEALELSRAKDTKERMAGVERLHQLLEASRKSLSSSEVTSLVDTCMDLLKDNNFRVSQGALQALASAAVLAGEHFKLHFNALLPAVVDRLGDAKQPVRDAARRLLLTLMEVSSPTIIVERAGSFAWAHKSWRVREEFTRTVTAAINLFASTELPLQRAILPPVLHLLNDPNPAVREAAILCIEEMYTQAGPQFRDELHRHNLPSSLVKDINARLEGIQPKVRSSDGIPGGYITGEIKHVSVNPKKSSPKAKSSSRENSLFGGEGDITEKPIDPVKVYSDKELIREFEKIASTLVPEKDWSIRIAAMQRVEGLVLGGAVDYPCFCGLLKQLVGPLTTQLSDRRSTIVKQACHLLCFLSKELLGDFEACAEMLIPVLFKLVVITVLVIAESADNCIKTMLRNCKVARVLPRIADCAKNDRNAVLRARCCEYAYLVLEHWPDAPEIHRSADLYEDLIKCCVSDAMSEVRSTARMCYRMFAKTWPERSRRLFSSFDPAIQRLINEEDGGMHRRHASPSIRDRGALMSLSSQASAPSNLPGYGTSAIVAMDRSSSISSGTSISSGILLSQAKSLGKGTERSLESMLHASKQKVSAIESMLRGLDLSDKHNSSSLRSTSLDLGVDPPSSRDPPFPAAVPASNHLTSSLTESTTSGINKGSNRNGGLGLSDIITQIQASKDSAKLSYRSNVGIEPLSSLSSYSSKRASDRQERSSLDDNNDMRETRRYMNPNTDRQYLDAPYRDGNFRESHNSYVPNFQRPLLRKNVAGRMSASRRRSFDDNQLSLGEMSNFADGPASLHEALSEGLSSGSNWSARVAAFNYLHSLLQQGPKGTLEVVQNFEKVMKLFFQHLDDPHHKVAQAALSTLADIVPVCRKPFEGYMERILPHVFSRLIDPKELVRQPCSTTLEVVSKTYSIDSLLPALLRSLDEQRSPKAKLAVIEFAINSFNKHAMNPEGAANIGILKLWLAKLTPLVNDKNTKLKEAAITCIISVYSHFDSTAVLNFILSLSVEEQNSLRRALKQYTPRIEVDLINYLQNKKEKQRSKSSYDPSDVVGTSSEDGYVGYSRKAHYLGKYSAGSLDGDGGRKWSSQDSTLIKASLGQASSGETREHLYHNFETDPNSGSLGSKTKDLAYAVNPMGQNIGSQTSQHGHVDSSVSLEGLSIPRLDVNGLMPSEHLNGTEGYVNDKEHPSELERNHHSAEDVKINSMTDTGPSIPQILHMICSGGDGSPISSKRTALQQLVEASITNDHSVWTKYFNQILTVVLEVLDDSDSSVKELALSLIVEMLKNQKGAVENSVEIVIEKLLHVTKDIIPKVSNEAEHCLTIVLSQYDPFRCLSVIVPLLVTEDEKTLVICINCLTKLVGRLSQEELMTQLPSFLPALFEAFGNQSTDVRKTVVFCLVDIYIMLGKAFLPYLQGLNSTQLKLVTIYANRISQARTGKAIDAVQD

SEQ ID NO: 3 - DNA, 396 bp, Glycine max
attcttcatatgatatgcagtgggggtgatggaagccctatttcaagtaaacggactgctcttcaacaattggttgaagcttctataacaaatgatcattctgtttggaccaagtacttcaatcagattttgacagtagtgcttgaggtgctggatgattcagattcctcagtcaaagagcttgctctttcactgatagttgaaatgcttaaaaaccagaaaggtgccgtggagaattctgttgagattgtaattgagaagctgcttcatgttacaaaagatatcattcctaaggtctcgaatgaagcagagcactgcctgaccattgttttatctcagtatgatccattcagatgtttaagtgtcattgtccctctactggttactgaggatgag

SEQ ID NO: 4 - DNA, 4539 bp, Glycine max
catcggattcttctctctatcctcatcggtttcaaactcgctcactctctcactcagtcttgttcagatctgatcaaaggaagaagaaatggaggaagctctggaactgtcacgcgccaaggacacgaaggagaggatggcgggcgtagagcggctgcatcaacttctagaagtttccagaaagagcctctcctcttccgaagtcacctcgctcgtcgacacgtgtatggatctcctcaaggacaacaacttccgcgtctcccagggcgctctccaggccctcgcctccgccgccgtcctcgccggtgaacacttcaagctccacttcaacgccctcctccccgccgtcgtcgaccgcctcggcgacgccaagcagcccgtccgcgacgccgcccgtcggctcctgctcactctcatggaggtttcttctcctacaataattgttgaaagagcagggtcctttgcttgggcacataaaagctggagagtcagagaggagtttacacgaactgtcgcagctgcaattaatctatttgcagctactgagcttccacttcaacgagctatcctcccccctgttttgcatttgcttaatgatccaaatcctgctgttagggaagcagctattttgtgcattgaggagatgtatacacaagctggacctcaatttcgtgacgaacttcaccgccacaatcttccttcatctttggtgaaagatattaatgccaggctcgagggaattcaaccaaaagttcgctcttcggatggtccaggtggatatattactggggaaattaaacatgcaagtgttaatcccaagaaaagtagtccaaaggctaaaagttcctcaagggagaactctctttttggaggagaaggtgacatcactgagaaacccgtagaccctgtcaaggtatattcagataaggagttaatcagggaatttgagaagattgcatctacccttgtgcctgaaaaagactggtcaattcgaactgctgctctgcagagagttgaaggtcttgttcttggaggtgctgtggattatccatgttttcgtgggctcctgaagcaacttgttggacctttaagcacacaattgtcagaccgaagatctaccattgtgaagcaggcttgccatctattgtgctttttgtcaaaggaactcttgggtgattttgaggcatgtgctgaaatgtttataccagtccttttcaagctggttgtgattactgtgcttgtaattgcagagtctgcagataactgcattaaaacgatgctgcgcaactgcaaagttgctcgtgtgcttccccgtatagccgattgtgcaaaaaatgatcgcaatgcagtacttcgtgcaaggtgttgtgaatatgcttatttggtcctagaacattggcctgatgcacctgaaatacatcgatcagctgatttatatgaggatctgataaagtgttgtgtttcagacgcaatgagtgaggtacggtctactgcaaggatgtgctacagaatgtttgcaaaaacttggccagagcgttctcgtcgcctattttcatcctttgaccctgcgattcaacggttaataaatgaagaggatggaggaatacatcgacgacatgcttcaccttccattcgagatagaggtgcaccgacgtcattatctagccaggcctctgctccctctaatctacctggttatggaacttctgctattgttgctatggataaaagttcaagtatatcatcagggacatctatctcatctggcatacttctttcacaagctaagtcacttggtaagggtacagaacgtagtttggaaagcatgttacatgcaagcaaacagaaggtttctgctattgaaagcatgcttagaggcttggatttgtctgataagcataattcatcttctctccggtcaacaagtttggatctaggagttgaccctccatcatctcgtgatccacctttccctgcagctgtccccgcatctaatcatctgacaagctctttaacaacagaatcaactacttccggcatcaataaaggtagtaaccgaaatggtggtctcggtttgtctgacataatcactcagattcaagcttcgaaagattctgccaagttatcctatcgtagtaatgttggtattgagcctttatcatcatattcgagtaaaagggcatctgaaagacaagaaagaagttctctcgatgataaccatgatatgagggagactaggcgatatatgaaccccaacactgatagacagtatttggatgccccttatagagatggaaactttagggaatctcataatagttatgtgcctaatttccagagaccactattgagaaagaatgtggctggacgcatgtctgctggcaggaggagttttgatgataatcagctatctcttggagagatgtcgaattttgcagatggaccagcatctcttcatgaagctttgagtgaaggacttagttcaggttccgattggtctgctagggttgctgcctttaattatcttcattctttgttgcagcaaggtccgaagggaactctagaagttgttcagaattttgagaaggtaatgaagttgtttttccagcacttggatgatccccatcataaagttgcacaggctgctctttcaacacttgcagatattgttccagcatgccgaaagccttttgagggttatatggaaagaatattaccccatgtgttttctcggttaattgaccctaaagaacttgtcaggcagccgtgttcaacaactctggaagtggtcagcaaaacttacagtatagattctctcttacctgcattattacgctcactagatgaacaaaggtctccaaaggctaaattggctgttattgaatttgctatcaattcctttaacaaacatgctatgaacccagaaggtgctgctaatattggcatcctaaaattatggcttgctaagttaacccctttggttcatgacaaaaatacaaagcttaaagaagcagctattacatgcattatatcagtgtactctcactttgattcaactgctgttcttaatttcattctgagtttgtcagttgaagaacaaaattccttgagacgagcattgaaacaatacacccctcgcattgaagtagacctaataaactatctgcagaacaagaaagagaaacaacgttctaagtcttcctatgatccatctgatgttgtgggaacatcctctgaagatggatatgttggttactctaggaaagctcattaccttggtaggtattctgctggttcacttgatagtgatggtggcaggaaatggagttcccaagattcaacactgataaaagccagtcttggtcaagcatcttctggtgaaactcgggaacatctgtatcataattttgagactgatcctaatagtggcagccttggttcaaaaactaaggatcttgcttatgccgtcaatccaatgggtcaaaactttggctctcaaactagccaacatggacacatggacagtagcgtgagcttagagggtctttcaactcctcgtctagatgtaaacggtttgatgtcatcagagcatttaaatggtgctgaaggctatgcaaatgacaaagagcatccttctgaattggaacttaatcatcactcagctgaagatgtaaaaattaacaccatgacacacacaggacccagcattcctcaaattcttcatatgatatgcagtgggggtgatggaagccctatttcaagtaaacggactgctcttcaacaattggttgaagcttctataacaaatgatcattctgtttggaccaagtacttcaatcagattttgacagttgtgcttgaggtgctggatgattcagattcctcagtcaaagagcttgctctttcactgatagttgaaatgcttaaaaaccagaaaggtgccatggagaactctgttgagattgtaattgagaagctgcttcatgttacaaaagatatcattcctaaggtctcgaatgaagcagagcactgcctgaccattgttctatctcagtatgatccattcagatgtttaagtgtcattgtccctctgctggttaccgaggatgagaaaacactcgttatttgcataaattgcttaacaaagcttgtggggcggctttctcaggaagaactaatggctcagttaccctcgtttctgcctgcccttttcgaagcttttggtaaccagagtgcagatgttcggaagaccgttgttttctgtctcgtggatatttatatcatgcttgggagagcattcttgccatatttgcaagggcttaacagcacacagttgaagttggtcaccatttatgcaaatcgaatctcacaagccaggacaggcaaagcgattgatgccgttcaagactagaggtttattgggaagaaaaaagttgtggctttttgggtgttgtctattcattgttgtgtatttgtatatctgcgtgcatgaatttagatgtgggttccattgtctttccttctccttgggtctcttta

SEQ ID NO: 5 - protein, 1440 aa, Glycine max
MEEALELSRAKDTKERMAGVERLHQLLEVSRKSLSSSEVTSLVDTCMDLLKDNNFRVSQGALQALASAAVLAGEHFKLHFNALLPAVVDRLGDAKQPVRDAARRLLLTLMEVSSPTIIVERAGSFAWAHKSWRVREEFTRTVAAAINLFAATELPLQRAILPPVLHLLNDPNPAVREAAILCIEEMYTQAGPQFRDELHRHNLPSSLVKDINARLEGIQPKVRSSDGPGGYITGEIKHASVNPKKSSPKAKSSSRENSLFGGEGDITEKPVDPVKVYSDKELIREFEKIASTLVPEKDWSIRTAALQRVEGLVLGGAVDYPCFRGLLKQLVGPLSTQLSDRRSTIVKQACHLLCFLSKELLGDFEACAEMFIPVLFKLVVITVLVIAESADNCIKTMLRNCKVARVLPRIADCAKNDRNAVLRARCCEYAYLVLEHWPDAPEIHRSADLYEDLIKCCVSDAMSEVRSTARMCYRMFAKTWPERSRRLFSSFDPAIQRLINEEDGGIHRRHASPSIRDRGAPTSLSSQASAPSNLPGYGTSAIVAMDKSSSISSGTSISSGILLSQAKSLGKGTERSLESMLHASKQKVSAIESMLRGLDLSDKHNSSSLRSTSLDLGVDPPSSRDPPFPAAVPASNHLTSSLTTESTTSGINKGSNRNGGLGLSDIITQIQASKDSAKLSYRSNVGIEPLSSYSSKRASERQERSSLDDNHDMRETRRYMNPNTDRQYLDAPYRDGNFRESHNSYVPNFQRPLLRKNVAGRMSAGRRSFDDNQLSLGEMSNFADGPASLHEALSEGLSSGSDWSARVAAFNYLHSLLQQGPKGTLEVVQNFEKVMKLFFQHLDDPHHKVAQAALSTLADIVPACRKPFEGYMERILPHVFSRLIDPKELVRQPCSTTLEVVSKTYSIDSLLPALLRSLDEQRSPKAKLAVIEFAINSFNKHAMNPEGAANIGILKLWLAKLTPLVHDKNTKLKEAAITCIISVYSHFDSTAVLNFILSLSVEEQNSLRRALKQYTPRIEVDLINYLQNKKEKQRSKSSYDPSDVVGTSSEDGYVGYSRKAHYLGRYSAGSLDSDGGRKWSSQDSTLIKASLGQASSGETREHLYHNFETDPNSGSLGSKTKDLAYAVNPMGQNFGSQTSQHGHMDSSVSLEGLSTPRLDVNGLMSSEHLNGAEGYANDKEHPSELELNHHSAEDVKINTMTHTGPSIPQILHMICSGGDGSPISSKRTALQQLVEASITNDHSVWTKYFNQILTVVLEVLDDSDSSVKELALSLIVEMLKNQKGAMENSVEIVIEKLLHVTKDIIPKVSNEAEHCLTIVLSQYDPFRCLSVIVPLLVTEDEKTLVICINCLTKLVGRLSQEELMAQLPSFLPALFEAFGNQSADVRKTVVFCLVDIYIMLGRAFLPYLQGLNSTQLKLVTIYANRISQARTGKAIDAVQD

SEQ ID NO: 6 - DNA, 4432 bp, Glycine max
aaccagatctgcacccggccgcgccagatccgatggaggaagcgctcgagctcgcgcgcgcaaaggacgcgaaggagcgcatggcgggcgtggagcgcctacacgaggttctggaagcttccagaaggagcctcagctccggcgaggtcacctcgctcgtcgattgctgcttggatctcttgaaggacagcagcttcaaggtctcgcagggcgcgctccaggcgctcgactccgccgccgtgcgcgccggcgaccacttcaagctccacttcaacgcgctcgtccccgccgtcgtcgaccgcctcggcgacgccaagcagcccgtccgcgacgccgcgaggcggctccttctcactctcatggaggtttcatctcctacaataattgttgaaagagcagggtcctttgcatggacaagcaaaagctggagagtcagagaggaatttgcgcgaacagtcacatctgcaattggtctattttcatctactgagcttccccttcaacgtgctattcttcctcctattttgcagttgctgaatgatctgaatcctgctgttagggaaacagctattttgtgtattgaggaaatgtatacacaagctgggtcccaatttcgtgatgaacttcagcgccacaatctcccttcatctttggtgaaagctattaatgccaggctagagggaattcaaccaaaagttcactcttcagatggtatttctagtggttataatgctggggaaattaagcctgtgggtgttaatcccaagaaaagtagtccaaaggctaaaagttcatcaagggagacctctctttttggaggagaaggtgatgccactgagaaagtcatagaccccatcaaggtgtattcagagaaggagttaatcagagaaattgataagattgcttctactcttgtaccagaaaaggactggtcaattcgtattgctgccatgcaaagaattgaaagtcttgttttgggaggtgctgctgattatccatgtttctttggactcctgaagcagcttgttggacctttaagcacacaattgtcagatcgaaggtctagcattgtgaagcaggcttgccatttattatgctttttgtcgaaggactttttgggtgattttgaagcatgtgctgaattgttgataccggtccttctgaagctggttgtgattactgtgcttgtaattgcggaatctgcagataactgcataaaaacgatgttgcgcaactgcaaagcagctcgtgtacttcctcggatagctgattgtgccaaaaatgatcgtaatgctgttcttcgtgcaaggtgttgtgattatgctcttttgatactagaacattggcctgatgcagcagaagtacaacgatcggctgatctatatgaggatatgataaggtgttgtgtttcagacgcaatgagtgaggtgcggtcgactgcaaggatgtgttacagaatgtttgcaaagacttggccagaacgttcccgtcgcctgttttcatcatttgaccctgctattcaaaggttaataaacgaagaggatggaggaatgcataggcgacatgcatcaccgtctgttcgtgatagaggtgcactgatgccaataactagtcaagcttcagcaccctctaatttaactggttatggaacttctgctattattgcaatggaccgaagttcaagtttatcatcggggacttctatcgcctctggtgttctttcacaagccaagtcacttggtaaagttactgaacgtagtttggaaagtgtgttgcatgcaagcaaacagaaggtcactgctattgaaagcatgcttaggggtttggatttgtctgataagcatggttcatctgctctccggtcatcgagcttgggtctaggagttgaccctccctcatctcgtgatccaccattccctgctgctgtcacagcatctaatcatctgactagctctttaacggcagaatcaactgcagctggtgccaataaagctagtaaccgacatggtggtttgggtttgtctgacataatcacccaaattcaagcttccaaagattctggcaggttatcgtataatactaatgttggtattgagcctttatcagcattctcatcattctcaagtaaaagggctactgaaaaattgcaagaaagaggttctattgatgaaaacagtgatatgagggagactagacgctatatgaaccccaacatagataggcagtatatggatacccattatagagatggcaactatagggattcacagaatagttatgtgcctaatttccagaggccactattgagaaagaatgtagctggacgtgtgtctgctggaagcaggaggagttttgatgatagccaattatctcttggagagatgtcaaattatgcagatggcccagcatctcttcatgaagctctgagtgaaggacttagctcaggttctgactggtccgctagggttgctgcctttaattatcttcactctttgtttgagcaaggtcaaaagggaattcaagaagtagtccagaattttgaaaaggtaatgaagttgttttttcagcacttggatgatccccatcataaagttgcacaggctgctctctccacacttgcagatattattctggcatgccgaaagccctttgaaggttacatggaacgaatgttaccccatgtgttttctcggttaattgaccccaaagagctagttagacaagcatgctcaatgaatttggaagttgttagcaaaacatacagcatagattctctcctacctgcattgctacgatctctagatgaacaaaggtcaccaaaggcaaaattggctgttattgagtttgcaataagttcatttgataagcatgccatgaatcctgaaggtactgctaatattggcatcctaaaattatggcttgccaagctagtcccattggttcatgataaaaatacaaaacttaaagaagcagctattacatgcattatatcggtgtactctcactttgattcatctgcagttttgaattttattcttagtttgtcagttgatgaacaaaattctttgaggcgagcccttaaacagcgcacccctcgcattgaagtagacttaatgaattatctgcaaaataagaaagatcgccgttctaagtcttcctatgatccatctgatgttgtgggagcatcctctgaagagggatatgctggtttgtcaaggaaagctcagtacattggaaggtattctgctggttcactagatagtgatggtggcaggaattggagttcccaagattccaccctgattaaagccagtcttggccaagcagctactgatgaaactgaagaacatactgattctaatagtggtgcttttggtttaaaaactaaagagcttgcttacacagccaattcaacgggtcaaaactttggcttacaaactagccatggacatgtggacagtagcattaattttgaaggtctatcatctgatctgaatgttaatggtctgatgtcatcagaacatttaaatattactgaagactttggacatgacaaagaacatcattcagctgaagatgtgaaagtaaactacatgacagacaatgggcctagtattccccagattcttcatatgatatgcagtgggggtgatggaagccctatttcaagcaagcggactgctcttcaacagctagctgaagtgtctatagccaatgatcattctgtttggaccctgtacttcaatcagattttgacagttgtgcttgaggtgctggatgattcagattcctcaatcagagagcttgctctgtctctgatagttgaaatgctgaaaaaccagaaagatgctatggaaaattcagttgagattgtagttgaaaagctgcttaatgttaccaaggatattgttcccaaggtttcaaatgaagcagagcactgcctcaccattgttttatctcagaatgatccgttcagatgcttgagtgttattgtccctctactggttactgaggatgagaaaactcttatcacttgcataaattgcttgacaaagcttgtggggaggctccctcaggaggaactgatggctcagttaccctctttcctgcctgctttgtttgaagcttttggaaaccagagtgctgatgttcgcaagaccgttgttttctgtctagtggatatttatatcatgcttgggaaagcatttttgccatatttggaaggacttaacagcacacagttgaagttggtcaccatttatgcaaatcgtatctcacaggcaaggacaggaaaatcaattgataccacacacgattagaggttcattcagatgaaaattcgtcggttttgaatgctttcttattcattgctgtgtattttgtagattgtgtgcatgaaattttttaggtgggttttccttgtgtttctttc

SEQ ID NO: 7 - protein, 1428 aa, Glycine max
MEEALELARAKDAKERMAGVERLHEVLEASRRSLSSGEVTSLVDCCLDLLKDSSFKVSQGALQALDSAAVRAGDHFKLHFNALVPAVVDRLGDAKQPVRDAARRLLLTLMEVSSPTIIVERAGSFAWTSKSWRVREEFARTVTSAIGLFSSTELPLQRAILPPILQLLNDLNPAVRETAILCIEEMYTQAGSQFRDELQRHNLPSSLVKAINARLEGIQPKVHSSDGISSGYNAGEIKPVGVNPKKSSPKAKSSSRETSLFGGEGDATEKVIDPIKVYSEKELIREIDKIASTLVPEKDWSIRIAAMQRIESLVLGGAADYPCFFGLLKQLVGPLSTQLSDRRSSIVKQACHLLCFLSKDFLGDFEACAELLIPVLLKLVVITVLVIAESADNCIKTMLRNCKAARVLPRIADCAKNDRNAVLRARCCDYALLILEHWPDAAEVQRSADLYEDMIRCCVSDAMSEVRSTARMCYRMFAKTWPERSRRLFSSFDPAIQRLINEEDGGMHRRHASPSVRDRGALMPITSQASAPSNLTGYGTSAIIAMDRSSSLSSGTSIASGVLSQAKSLGKVTERSLESVLHASKQKVTAIESMLRGLDLSDKHGSSALRSSSLGLGVDPPSSRDPPFPAAVTASNHLTSSLTAESTAAGANKASNRHGGLGLSDIITQIQASKDSGRLSYNTNVGIEPLSAFSSFSSKRATEKLQERGSIDENSDMRETRRYMNPNIDRQYMDTHYRDGNYRDSQNSYVPNFQRPLLRKNVAGRVSAGSRRSFDDSQLSLGEMSNYADGPASLHEALSEGLSSGSDWSARVAAFNYLHSLFEQGQKGIQEVVQNFEKVMKLFFQHLDDPHHKVAQAALSTLADIILACRKPFEGYMERMLPHVFSRLIDPKELVRQACSMNLEVVSKTYSIDSLLPALLRSLDEQRSPKAKLAVIEFAISSFDKHAMNPEGTANIGILKLWLAKLVPLVHDKNTKLKEAAITCIISVYSHFDSSAVLNFILSLSVDEQNSLRRALKQRTPRIEVDLMNYLQNKKDRRSKSSYDPSDVVGASSEEGYAGLSRKAQYIGRYSAGSLDSDGGRNWSSQDSTLIKASLGQAATDETEEHTDSNSGAFGLKTKELAYTANSTGQNFGLQTSHGHVDSSINFEGLSSDLNVNGLMSSEHLNITEDFGHDKEHHSAEDVKVNYMTDNGPSIPQILHMICSGGDGSPISSKRTALQQLAEVSIANDHSVWTLYFNQILTVVLEVLDDSDSSIRELALSLIVEMLKNQKDAMENSVEIVVEKLLNVTKDIVPKVSNEAEHCLTIVLSQNDPFRCLSVIVPLLVTEDEKTLITCINCLTKLVGRLPQEELMAQLPSFLPALFEAFGNQSADVRKTVVFCLVDIYIMLGKAFLPYLEGLNSTQLKLVTIYANRISQARTGKSIDTTHD

SEQ ID NO: 8 - DNA, 4502 bp, Glycine max
ttcgcatttcccaacttcccggcgccgttatgtggtggtgaatccgctgacatttcaaaccagatctgcacccggccgtgccagatccgatggaggaagcgctcgagctcgcacgcgcgaaggacgcgaaggagcgcatggcgggcgtggagcgcctccacgaggtgctggaagcttccagaaggagcctcagctccggtggggtcacttctctcgtcgattgctgcttggatctcttgaaggacagcagcttcaaggtctcgcagggcgcgctccaggctctcgactccgccgccgtgcgcgccggtgaccacttcaagctccacttcaacgcgctggtccctgccgtcgtcgaccgcctcggcgacgccaagcagcccgtccgcgacgccgccaggcggctccttctcactctcatggaggtttcatctcctacaataattgttgaaagagcagggtcctttgcttggacaagcagaagctggagagtcagagaggaatttgcacgaacagtcacatctgcaattggtctattttcatctactgagcttccccttcaacgtgctattcttcctcctattttgcagttgctgaatgatctgaatcctgctgttagggaagcagctattttgtgtattgaggaaatgtatacacaagctgggtcccaatttcgtgatgaacttcagcgccacaatctcccttcatctttggtgaaagctattaatgctaggctagagggaattcaaccaaatgtttgctcttcagatggtatttctagcggttataatgctggggaaattaagcctgtgggtgttaatcccaagaaaagtagtccaaagcataaaagttcatcaagggagacctctctttttggaggagaaggtgatgccacagagaaactcatagaccccatcaaggtgtattcagagaaggagttaatcagagaaattgataagattgcttctacccttgtaccagaaaaggactggtcaattcgtattgctgccatgcaaagaattgaaggtcttgttttgggaggtgctgctgattatccatgtttctttggactcctgaagcagcttgttggacctttaagcacacaattgtcagatcgaaggtcaagcattgtgaagcaggcttgccatctattatgctttttgtcgaaggacttcttgggtgattttgaagcatgtgctgaattgttcataccagtccttttgaagctggttgtgattactgtgcttgtaattgcagaatctgcggacaactgcataaaaatgatgttgcacaactgcaaagtagctcgtgtgcttcctcggatagctgattgtgccaaaaatgatcgtaatgctgttctacgtgcaaggtgttgtgattatgctcttttgatattagaacattggcctgatgcagcagaagtacaacgatcggctgatctatatgaggatatgataaggtgctgtgtttcagatgcaatgagtgaggtgcggtcgactgcaaggatgtgttacagaatgtttgcaaagacttggccagaacgttcccgtcgcctgttttcatcatttgaccctgctattcaaaggttaatcaacgaagaggatggaggaatgcataggcgacatgcatcaccttctgttcgtgatagaggtgcactgatgtcaataactactcaagcttcagcaccctctaatctaactggttatggaacttctgctattgttgcaatggaccgaagttcaagtttatcatcggggacttctatcgcctctggtgttctttcacaagccaagtcacttggtaaaggtactgaacgtagtttggaaagtgtgttgcatgcaagcaaacagaaggtcactgctattgaaagcatgcttaggggtttggatttgtttgataagcatggttcatctgctctccggtcatcgagcttggatctaggagttgaccctccctcatctcgtgatccaccattccctgctgctgtcacagcatctaatcatctaactagctctttaacgacagaatcaactgcatctggtgccaataaagctagtaaccgaaatggtggtttgggtatgtctgacataatcacccaaattcaagcttccaaagattctggcaggttatcgcataatactaatgttggtattgagcctttatcaacgttctcatcatactcaagtaaaagggttactgaaaagttgcaagagagaggttctattgatgaaaacagtgacatgagggagactagatgctatatgaatcccaacatagataggcagtgtatggatacccattatagagatggcaactatagggattcgcagcatagttatgtgcctaatttccagaggccactattgagaaagaatgtagctggacgtgtgactactggcagcaggaggagttttgatgatagccaattatctcttggagagaagtcaaactatgtagatggcccagcatctcttcatgaagctctgagtgaaggacttagctcaggttctgactggtctgctagggttgctgcctttaattatcttcactctttattgcagcaaggccaaaagggaattcaagaagtagtccagaattttgagaaggtaatgaagttgttttttcagcacttggatgatccccatcataaagttgcacaggctgctctctccacacttgcagatattattctggcattccgaaagccctttgaaggttacatggaacgaatgttaccccatgtgttttctcggttaattgaccccaaagagctagttaggcaagcatgctcaatgaatttggaagttgttagcaaaacatacagcatagattctctcctacctgcattgctacgatcactagatgaacaaaggtcaccaaaggcaaaattggctgttattgagtttgcaataagttcgtttaataagcatgcaatgaatccagaaggtgctgctaatattggcatcctaaaattatggcttgccaagttagtcccattggttcatgataaaaatacaaaacttaaagaagcagctattacatgcattatatcagtatactctcactttgattcatctgcagttttgaattttattcttagtttgtcagttgatgaacaaaattctttgagacgagcccttaagcagcgcacccctcgcattgaagtagacttgatgaattatctgcaaaacaagaaagagcgacattctaagtcttcctatgatccatctgatgttgtgggagcatcctctgaagagggatatgttggtttgtcaaggaaagctcattatattggaagatatgctgctggttcactagatattgatggtagcaggaagtggagttcccaagattctgccctgattaaaggcagtattggccaagcagtttctgatgaaactgaagaacacactgattctaatagtggtgtttatggtttcaaaactaaagatcttgcttacacagccaattcaatgggtcaaaactttggcttacaaactagccacagacatgtgaacagtagcatgaattttgaaggtctatcatctgatctggatgttaatggtctgatgtcatcagaacatttaaatattactgaagactttggacccgacaaagaacatccttctgaattgaaccataatcatcagtcagctgaagatgtgaacgtaaactacatgacagacactggacccagtattccccagattcttcatatgatatgcagtgggggtgatggaagccctatttcaagcaagcagactgcacttcaacagctagttgaagtgtctatagccaatgaacattctatttggactctgtacttcaatcagattttgacagttgtgcttgaggtgctggatgattcagattcctcaatcagagagcatgctctatctctgatagttgaaatgcttaaaaatcagaaagatgccatggaaaattcagttgagattgttgttgaaaagctgcttaatgttacaaaggatattgttcccaaggtctcaaatgaagcagagcactgcctcaccattgttttatctcagaatgatccattcagatgtttaagtgttattgtccctctactggttaccgaggatgagaaaactcttgttacttgcataaattgcttgacaaagcttgtggggaggctctctcaggaggaagtgatggctcagttaccttctttcctgcctgctttgtttgaagcttttggaaaccagagtgctgatgttcgcaagaccgttgttttctgtctagtggatatttatatcatgcttgggaaagcatttttgccatatttggaagggcttaacagcacacagttgaagttggtcaccatttacgcaaatcgtatctcacaggcaaggacaagaaaatcaattgataccacacacgattagcggttcattcagatgaaaattcgtcggttttgaatgctttcttattcattgttgtgtattttgtatattgtgtgcatggaattttttaggtgggttttcctt

SEQ ID NO: 9 - protein, 1436 aa, Glycine max
MEEALELARAKDAKERMAGVERLHEVLEASRRSLSSGGVTSLVDCCLDLLKDSSFKVSQGALQALDSAAVRAGDHFKLHFNALVPAVVDRLGDAKQPVRDAARRLLLTLMEVSSPTIIVERAGSFAWTSRSWRVREEFARTVTSAIGLFSSTELPLQRAILPPILQLLNDLNPAVREAAILCIEEMYTQAGSQFRDELQRHNLPSSLVKAINARLEGIQPNVCSSDGISSGYNAGEIKPVGVNPKKSSPKHKSSSRETSLFGGEGDATEKLIDPIKVYSEKELIREIDKIASTLVPEKDWSIRIAAMQRIEGLVLGGAADYPCFFGLLKQLVGPLSTQLSDRRSSIVKQACHLLCFLSKDFLGDFEACAELFIPVLLKLVVITVLVIAESADNCIKMMLHNCKVARVLPRIADCAKNDRNAVLRARCCDYALLILEHWPDAAEVQRSADLYEDMIRCCVSDAMSEVRSTARMCYRMFAKTWPERSRRLFSSFDPAIQRLINEEDGGMHRRHASPSVRDRGALMSITTQASAPSNLTGYGTSAIVAMDRSSSLSSGTSIASGVLSQAKSLGKGTERSLESVLHASKQKVTAIESMLRGLDLFDKHGSSALRSSSLDLGVDPPSSRDPPFPAAVTASNHLTSSLTTESTASGANKASNRNGGLGMSDIITQIQASKDSGRLSHNTNVGIEPLSTFSSYSSKRVTEKLQERGSIDENSDMRETRCYMNPNIDRQCMDTHYRDGNYRDSQHSYVPNFQRPLLRKNVAGRVTTGSRRSFDDSQLSLGEKSNYVDGPASLHEALSEGLSSGSDWSARVAAFNYLHSLLQQGQKGIQEVVQNFEKVMKLFFQHLDDPHHKVAQAALSTLADIILAFRKPFEGYMERMLPHVFSRLIDPKELVRQACSMNLEVVSKTYSIDSLLPALLRSLDEQRSPKAKLAVIEFAISSFNKHAMNPEGAANIGILKLWLAKLVPLVHDKNTKLKEAAITCIISVYSHFDSSAVLNFILSLSVDEQNSLRRALKQRTPRIEVDLMNYLQNKKERHSKSSYDPSDVVGASSEEGYVGLSRKAHYIGRYAAGSLDIDGSRKWSSQDSALIKGSIGQAVSDETEEHTDSNSGVYGFKTKDLAYTANSMGQNFGLQTSHRHVNSSMNFEGLSSDLDVNGLMSSEHLNITEDFGPDKEHPSELNHNHQSAEDVNVNYMTDTGPSIPQILHMICSGGDGSPISSKQTALQQLVEVSIANEHSIWTLYFNQILTVVLEVLDDSDSSIREHALSLIVEMLKNQKDAMENSVEIVVEKLLNVTKDIVPKVSNEAEHCLTIVLSQNDPFRCLSVIVPLLVTEDEKTLVTCINCLTKLVGRLSQEEVMAQLPSFLPALFEAFGNQSADVRKTVVFCLVDIYIMLGKAFLPYLEGLNSTQLKLVTIYANRISQARTRKSIDTTHD

SEQ ID NO: 10 - DNA, 1545 bp, Glycine max
atggggaacaagatgaatgcgatcgcgttgtgcttgttagtgtcgaccctgttggtttctgcggtgtattgtgcaccgaatgctgggctccgtaggattgggctgaagaagattaaattggaccctaaaaacaggcttgctgctcgagttgggtccaaggatgttgattctttccgggcttctattagaaagtttcatctccaaaacaactttggtggcactgaggaaaccgatattgtcgcgttaaagaattacttggatgcacagtactacggtgaaatagctattggaacttctcctcaaaagttcgctgtcattttcgacactggcagctctaatttgtgggttccatcatctaaatgcaccttctcggttgcatgctactttcatgctaagtataagtctagcaaatcaagtactttcaagaaaaatgggactgctgcggcaattcaatatggtaccggagcaatttctggtttctttagctacgacagtgtcagagttggtgaaatagttgtaaagaaccaggaatttattgaagcaactagggagcctggtgttacatttttggcggccaagtttgatggtatactgggacttggatttcaagagatatcagttggaaatgctgctccagtgtggtacaacatggttgaccaaggtcttcttaaggaaccagtattttcattttggttcaaccgcaatccagaggaagaggaagggggtgagattgttttcggtggtgttgatcctgctcactataagggaaagcacacttatgtgcctgtgaccagaaaaggatattggcagtttgatatgggagatgtacttattggcggtaaacccactggatattgtgctaatggctgttcagccattgcagactcagggacttctttgttggcaggtccaacgactgtcataactatgataaaccatgcaattggagcatctggagttatgagccaagaatgcaagaccattgttgcagagtatggacaaacaatattggatttgcttttggctgaaacacaaccgaagaagatctgttcccgaattggattgtgcgcatttgatgggactcatggtgttgatgtggggatcaagagcgttgtagatgagaatgaaagaaaatcattgggtggtcatcatggtgctgcttgtcctgcatgtgagatggcggttgtttggatgcagaaccagctgagccgaaatcagacacaagatcagatactaagttacatcaaccagctttgcgataaaatgcctagcccgatgggagaatcagctgttgactgtgggaatatctcttcattacctgtagtgtcctttactattggtggaagaactttcgaccttagcccagaggagtacgtactcaaggtgggtgaaggtcctgtggctcagtgtattagtggctttactgctatagatattccacctccacgtggccctctctggatccttggtgatgtcttcatggggcgttatcacacagtgtttgattttggtaaactcagagtgggatttgctgatgctgcatag

SEQ ID NO: 11 - protein, 514 aa, Glycine max
MGNKMNAIALCLLVSTLLVSAVYCAPNAGLRRIGLKKIKLDPKNRLAARVGSKDVDSFRASIRKFHLQNNFGGTEETDIVALKNYLDAQYYGEIAIGTSPQKFAVIFDTGSSNLWVPSSKCTFSVACYFHAKYKSSKSSTFKKNGTAAAIQYGTGAISGFFSYDSVRVGEIVVKNQEFIEATREPGVTFLAAKFDGILGLGFQEISVGNAAPVWYNMVDQGLLKEPVFSFWFNRNPEEEEGGEIVFGGVDPAHYKGKHTYVPVTRKGYWQFDMGDVLIGGKPTGYCANGCSAIADSGTSLLAGPTTVITMINHAIGASGVMSQECKTIVAEYGQTILDLLLAETQPKKICSRIGLCAFDGTHGVDVGIKSVVDENERKSLGGHHGAACPACEMAVVWMQNQLSRNQTQDQILSYINQLCDKMPSPMGESAVDCGNISSLPVVSFTIGGRTFDLSPEEYVLKVGEGPVAQCISGFTAIDIPPPRGPLWILGDVFMGRYHTVFDFGKLRVGFADAA

SEQ ID NO: 12 - DNA, 394 bp, Glycine max
gtgttacatttttggcggccaagtttgatggtatactgggacttggatttcaagagatatcagttggaaatgctgctccagtgtggtacaacatggttgaccaaggtcttcttaaggaaccagtattttcattttggttcaaccgcaatccagaggaagaggaagggggtgagattgttttcggtggtgttgatcctgctcactataagggaaagcacacttatgtgcctgtgaccagaaaaggatattggcagtttgatatgggagatgtacttattggcggtaaacccactggatattgtgctaatggctgttcagccattgcagactcagggacttctttgttggcaggtccaacgactgtcataactatgataaaccatgcatttggagc

SEQ ID NO: 13 - DNA, 1677 bp, Glycine max
atgggaaacatgccaaatgtggttgtgttgtgtttctgtctctggaccctgttgtttcctcttgtgttctgtgcacccaacgatgggttgcgtaggattggacttaaaaaggtgaagttggacaccgatgacgttgtggggtttaaggaatttcggtcttcaattagaaagcatcaccttcagaacatccttggaggcgctgaggacaccgatgttgttgcgctgaagaattacttggatgctcagtattacggtgaaatagccattggtacccctcctcagaagttcactgtgatttttgacaccggtagctctaatttgtgggtgccatcatccaaatgttacttctcggttgcatgtttcatgcacgctaggtacaggtctagccagtccagtacctacagggaaaatggtacttctgctgcaattcaatatggtactggagcaatttctggtttctttagcaatgacgatgtcaaagttggtgacatagttgtaaaggaccaggaatttattgaagcaactagagaacctggagttacatttgtggctgctaagtttgatggtatactaggacttggatttcaagagatatcagttggatatgctgttccagtgtggtacactatggttgaacaaggtcttgtaaaggatccagtattttcattttggctaaatcggaaaccagaagaagagaatggtggggagcttgtttttggtggtgctgatcctgctcactacaagggcaaacacacttatgtaccagtgacacgaaaaggatattggcagtttgacatgggagatgtgcttatttctggtaaacccactggatattgtactaatgactgttcagcaattgcagactctggaacttctttgttagctggtccaacgactgtaattaccatgataaatcaagcaatcggagcagctggagttgtaagcaaagaatgcaggtccgttgtcaatcaatatggacaaacaatcttggaattgctcttggctgaagcaaagccaaaaaagatctgctcacaaattggattgtgtacctttgatgggactcatggtgttagcatgggtatcgagagtgtggtggataagaatgaaaaaaaatcatctggtggcattcgggatgctggttgttctgcatgtgagatggctgttatttggatgcagaaccagctgaggcagaatcagacagaagataggataatagactatgccaatgagctttgtgaaaaattgcctaacccaatgggaccatcatccgttgactgtggaaagctctcttcaatgcctattgtttcctttactattggtggaaaagtttttgacctttcccctgaggagtatatactgaaggtgggtgaaggtcctgaagcccaatgcattagtggctttactgctttggatgttcctcctcctcgtggtcctctatggatccttggagatgtcttcatggggcgctatcacaccatctttgattatggtaagttgagagtcggatttgctgaggcagcatagagaatgttctcatgtctctcagggcagcaaccgcacttttctagtagtttattttcggttcagaaaattggttataccaaacaatatattacatcatggaagcacagttgtgcttccatactgaatattttcaatcgttatctgcttatagtt

SEQ ID NO: 14 - protein, 507 aa, Glycine max
MGNMPNVVVLCFCLWTLLFPLVFCAPNDGLRRIGLKKVKLDTDDVVGFKEFRSSIRKHHLQNILGGAEDTDVVALKNYLDAQYYGEIAIGTPPQKFTVIFDTGSSNLWVPSSKCYFSVACFMHARYRSSQSSTYRENGTSAAIQYGTGAISGFFSNDDVKVGDIVVKDQEFIEATREPGVTFVAAKFDGILGLGFQEISVGYAVPVWYTMVEQGLVKDPVFSFWLNRKPEEENGGELVFGGADPAHYKGKHTYVPVTRKGYWQFDMGDVLISGKPTGYCTNDCSAIADSGTSLLAGPTTVITMINQAIGAAGVVSKECRSVVNQYGQTILELLLAEAKPKKICSQIGLCTFDGTHGVSMGIESVVDKNEKKSSGGIRDAGCSACEMAVIWMQNQLRQNQTEDRIIDYANELCEKLPNPMGPSSVDCGKLSSMPIVSFTIGGKVFDLSPEEYILKVGEGPEAQCISGFTALDVPPPRGPLWILGDVFMGRYHTIFDYGKLRVGFAEAA

SEQ ID NO: 15 - DNA, 1698 bp, Glycine max
atggggaacaggatgaatgcgatcgcgttgtgcttgttagtgtcaagcctgttactttcttcggtgtattgtgcaccgaatgatgggctgcgtaggattgggctgaagaagattaaattggaccctaaaaacaggcttgctgctcgaattgggtccaaggatgttgattctttccgggcttccattagaaagtttcatctccaaaacaactttggtggctctgaggaaaccgatattgtcgcgttgaagaattacttggatgcacagtactatggtgaaatagctattggaacatctcctcaaaagttcactgtcattttcgacactggcagctctaatttgtgggttccatcatctaaatgtaccttctcggttgcatgctacttccatgctaagtataagtctagcaaatcaagtacttacaagaaaaatgggactgctgctgcaattcaatatggtaccggagcaatttctggtttctttagctacgacagtgtcagagttggtgatatatttgtaaagaaccaggaatttattgaagcaactagggagcctggtgttacatttttggcagccaagtttgatggtatactgggacttggatttcaagagatatcagttggaaatgctgttccagtgtggtacaacatggttgaccaaggtcttattaaggaaccagtattttcattttggttcaaccgcaaaccggaggaagaggaagggggtgagattgtttttggtggtgttgatcctgctcactataagggaaagcacacttatgtgcctgtgaccagaaaaggatattggcagtttgatatgggagatgtacttattggcggtaaacccactggatattgtgctgatggctgttcagccattgcagactcagggacttctttgttggcaggtccaacgactgtaataactatgataaaccatgcaattggagcatctggagttatgagccaagaatgcaagactgttgttgcagagtatggacaaacaatattggatttgcttttgtctgaaacacagccgaagaagatctgttcccgaattggattgtgtgcttttgatgggactcgtggtgttgatgtggggatcaagagtgttgtggatgagaatgaaagaaaatcatcgggtggtcatcatggtgctgcttgtcctgcgtgtgagatggctgttgtttggatgcagaaccagctgagccgaaatcagacacaagatcagatactaagttacatcaaccagctttgcgataaaatgcctagcccgatgggagaatcagctgttgactgtgggaatatctcttcattacctgtagtgtcctttactattggtggaagaacttttgagcttagcccagaggagtacatactcaaggtgggtgaaggtcctgtggctcagtgtattagtggctttactgctatagatattccacctccacgtggccctctctggatccttggtgatgtcttcatggggcgttatcacacagtatttgattttggcaaacaaagagtgggatttgctgatgctgcatagagtgtacgcgtgtctcagaatatggcgatccatgctattggtttatatactcttgcagtcgaaatggacctaaaaagttctggcttgtacagctttatattattaatgttaatactttgggtgttccataatagcttaacagatgtatctgtg

SEQ ID NO: 16 - protein, 514 aa, Glycine max
MGNRMNAIALCLLVSSLLLSSVYCAPNDGLRRIGLKKIKLDPKNRLAARIGSKDVDSFRASIRKFHLQNNFGGSEETDIVALKNYLDAQYYGEIAIGTSPQKFTVIFDTGSSNLWVPSSKCTFSVACYFHAKYKSSKSSTYKKNGTAAAIQYGTGAISGFFSYDSVRVGDIFVKNQEFIEATREPGVTFLAAKFDGILGLGFQEISVGNAVPVWYNMVDQGLIKEPVFSFWFNRKPEEEEGGEIVFGGVDPAHYKGKHTYVPVTRKGYWQFDMGDVLIGGKPTGYCADGCSAIADSGTSLLAGPTTVITMINHAIGASGVMSQECKTVVAEYGQTILDLLLSETQPKKICSRIGLCAFDGTRGVDVGIKSVVDENERKSSGGHHGAACPACEMAVVWMQNQLSRNQTQDQILSYINQLCDKMPSPMGESAVDCGNISSLPVVSFTIGGRTFELSPEEYILKVGEGPVAQCISGFTAIDIPPPRGPLWILGDVFMGRYHTVFDFGKQRVGFADAA

SEQ ID NO: 17 - DNA, 882 bp, Glycine max
accaaagagaaaccaaaaggctcaaaacacaaccatgatcaatcatgctctttgtttcttagtattcctatctgcactccatgcaacacatggagttgaatacactgtcactaacaacgcactctcaactcctggcggcgtggctttccgtgacaaaataggggctgaatacgccaagcgaacacttgactcggccacccaattcatatggaggatctttcagcaaaacaacccttctgacagaaaaaatgtgcagaaggtaagcttattcgttgatgacatggatggtgttgcatatactagcaacaatcaaatccatctcagtgcaagatacgttggaaacaataaaggggacgtgaaaacagagataacaggtgtgctgtaccatgaaatggtgcacgtttggcagtggagtgggaatggtcgtgctcctggtggattaattgaaggtattgcggactatgttaggctgaaggcaaactatgcacctagtcactgggtgaaagcagggcaaggacagaaatgggaccagggttatgatgttactgcacgctttttggactattgtgatagtctcaaaagtgggtttgtggcacaacttaacaagttgatgagaactggttatagtgatcaatacttcgttcagttactgggaaaaccggttgatcagctctggcgagactacaaggccaagtatggcaatattgcctaataacctaagagctttttcatttaaagaataagcgagtatatcgcaatgtttttcaataagattgtatcagctctaagattctgctattacaagttttggtgttttatttaaaataaaaatattctgagtttttaaccataagcatgacaaaattagaaaaacaaacggat

SEQ ID NO: 18 - protein, 225 aa, Glycine max
MINHALCFLVFLSALHATHGVEYTVTNNALSTPGGVAFRDKIGAEYAKRTLDSATQFIWRIFQQNNPSDRKNVQKVSLFVDDMDGVAYTSNNQIHLSARYVGNNKGDVKTEITGVLYHEMVHVWQWSGNGRAPGGLIEGIADYVRLKANYAPSHWVKAGQGQKWDQGYDVTARFLDYCDSLKSGFVAQLNKLMRTGYSDQYFVQLLGKPVDQLWRDYKAKYGNIA

SEQ ID NO: 19 - DNA, 316 bp, Glycine max
catgaaatggtgcacgtttggcagtggagtgggaatggtcgtgctcctggtggattaattgaaggtattgcggactatgttaggctgaaggcaaactatgcacctagtcactgggtgaaagcagggcaaggacagaaatgggaccagggttatgatgttactgcacgctttttggactattgtgatagtctcaaaagtgggtttgtggcacaacttaacaagttgatgagaactggttatagtgatcaatacttcgttcagttactgggaaaaccggttgatcagctctggcgagactacaaggccaagtatggca

SEQ ID NO: 20 - DNA, 905 bp, Glycine max
atgatgatgcatgtcctttgcttctcagtgttcctagcagcagcaatgcaaggaacactggcggttgaatacagtgtcaccaacaacgccctctcaacccctggtggtgttcgtttccgtgatgcaataggggacgaatacgccaaacaaacacttgactcagccacccaattcatatggggggtcttccagcaaaacgctcctgctgatagaaaagatatacaaaaaataaacttatttgtggaggacagggttgtgccaaatggtgttgttgcatatacaagcaaggatgagattcatgttattgcaaaatatgttaatgactatggtggggatgtgaagacagagatcacaggggtgctgtatcatgaaatggtccacgtttggcagtggaatgggaatggtcaggctccaagtggattaattgaaggtattgcagactatgttaggctgaaggcaaactatgcacctagccactggaagaaagcaggggagggacagaaatgggaccagggttatgatgttactgctcgttttctggactattgtaatactctcaaaagtgggtttgtggcacaattgaacaagcagatgaggactggttatagtgatcaattctttgttcagttactgggcaagacagttgatcagctttggcaagactacaaggctaagtatggcaatataccctagtaaatggctaagagcttgctaatttaaaaataagcgactagtatgtcgcaatctctattgtaggcttgtatcaactctaaatatttaaagttgcaacttatagtaagttatagtggaatggatgttctgattctgatttgtaagtcatgtgtacgccaaaaatttacaagttcaagaaaaaactatttctatatttttattggtgatgtgat

SEQ ID NO: 21 - protein, 230 aa, Glycine max
MMMHVLCFSVFLAAAMQGTLAVEYSVTNNALSTPGGVRFRDAIGDEYAKQTLDSATQFIWGVFQQNAPADRKDIQKINLFVEDRVVPNGVVAYTSKDEIHVIAKYVNDYGGDVKTEITGVLYHEMVHVWQWNGNGQAPSGLIEGIADYVRLKANYAPSHWKKAGEGQKWDQGYDVTARFLDYCNTLKSGFVAQLNKQMRTGYSDQFFVQLLGKTVDQLWQDYKAKYGNIP

SEQ ID NO: 22 - DNA, 865 bp, Glycine max
aagcaaccaaacacttgctctataagagaaaccaaaagcaaccatggcaatgcgttccctttgcttcttagtgtttctagctgcagcaatgcaaggaacacttgcagttgattacactgtcaccaacaacgccctctcaacctctggtggcgtgcgtttccgtgatgcaataggggacgattatgcaaaacaaacgctcgactcagccacccaattcatatggggggtgctccagcaaaacgctcctgctgatagaaaagatgtgcaaaaagtaaacctgttcgtggatgacatggatggcgttgcatataccagcaacgatgagattcatttgagtgcaagatatgtcaatgactatagtggggacctgaaaacagagatcacaggggtgttgtaccatgaaatggtccacgtttggcaatggaatgggaatggccaaactcctagtggattaattgaaggtattgcagactatgttaggctgaaggcaaattatgcacctagtcactgggtgaaagcagggcaaggagagaaatgggaccagggttatgatgttactgctcattttcttgactattgtgattctctcaaaagtgggtttgtggcacaattgaaccagcagatgagggatggttacagtgatcaactctttgttcagctactggggaagacagttgatcagctttggcaagactacaaggctaagtatggcactagttaagaggctatgagagctttgtacgatatttttaaaaataagcgagaatgtcgcaatctatttgtagattaatttgtattaagtctatgtgtattcagcagaatggatgttgtacctaactctgatctgtaagccgtgtgtgtgtgc

SEQ ID NO: 23 - protein, 225 aa, Glycine max
MAMRSLCFLVFLAAAMQGTLAVDYTVTNNALSTSGGVRFRDAIGDDYAKQTLDSATQFIWGVLQQNAPADRKDVQKVNLFVDDMDGVAYTSNDEIHLSARYVNDYSGDLKTEITGVLYHEMVHVWQWNGNGQTPSGLIEGIADYVRLKANYAPSHWVKAGQGEKWDQGYDVTAHFLDYCDSLKSGFVAQLNQQMRDGYSDQLFVQLLGKTVDQLWQDYKAKYGTS

SEQ ID NO: 24 - DNA, 2168 bp, Glycine max
atgagggagtggaccacaattgaaagtttcaaatgtgttgtacacgtttttgggcgctcgaaaatacaacatatagtagccagtgattacactagtttcacctataatggattccaatcttctcatttgtaccttgatggcagtgctgagttcaccaccaatggcatggttaagctcaccaatcacaccaagcaacaaaagggtcatgctttcttcccaagtccaatagtcttcaagaacaccacaaatggaagtgtgttttctttctccaccacttttgtttttgccataaggtctgagtttcccaatttgagtggccatgggattgcctttgtggtttctccaacaaaagaggttccacattctcttccaagccagtaccttggcctctttgatgacacaaacaacggcaacaacagcaaccatgtttttggggtggagcttgataccattctgaacaccgagtttggtgatatcaatgacaaccatgttgggattgatgtcaatgaattgaagtcggttaaatcggcttctgcagggtattacagtgatggggggtttaagaatttgagtctcatcagtggctacccaatgcaggtttgggtggaatatgatggcctaaagaagcagattgatgttactttagctccaatcaatgtcggtaaaccggaacgccctttgttatcattgaacaaagatctttctcgaattctgaacagcagcatgtatgttggattcacttcttcaactgggtcaattctaagttctcattatgttttgggttggagctttaaggtgaatggcaaggctcaacaacttgcaatctctgaactccccatgctacctagactagttggtaaacaagagtcaaaggttctcatagtagggttgcctctgatattactaattttgattctcatggtggctctagcagtggttcatgccatcaagaggaagaagtttgtggagctgcttgaagattgggagcaagactatggtcctcataggttcaagtacaaagatcttagcctagcaacaaaggggttcagggagaaggagttattggggagtggtggatttggtagagtctacaaaggggtgatgccaatttcgaagattgaggttgcagtgaagaaggtgtcacatgaatcaaggcaagggatgagggaatttgtggcggaaatcgctagcattggccgtcttaggcaccggaatctggttccccttctcgggtactgcaggcgcaaaggagagttgctgttggtctatgactacatgccaaatggaagccttgacaagtatctctacaacaaaccaagagtgaccttaaattggagccagagatttagaatcaccaaaggggttgcttcaggtttgttttatctgcatgaggaatgggagcaagttgtgctccacagagacattaaggctagcaatgtgctacttgatgctgaattgaatgggagattaggggactttggtctttcaaggttgtatgaacatggaactgaccctcacactactcatgtggttggaactcttggttatcttgcacctgagcacactagaacaggtaaggccactacaagctccgatgtgtttgcttttggtgcatttatgctagaggttgtgtgtggaaggagaccaatagagcaaggaagggaatccgggagcgaaattctggtcgattgggtgtataattgttggaaaaagggtgagattctcgaggcaagggatccaaacttgggcgcaaattataggccagatgaggtggaattggtgttgaaactcgccttgttgtgctcacattctgagcctttggctaggccaagcatgcgccaggttgtgcaatacttggagaaagatgtgcctctgccagatttgtctatgcttagcttatcttcaattggcttaacttttggtctgcatgaagactttcaggactgtccaatgtcttatccttcatctatggataggccaatatctcatacttcttcgattgctgaatcacttctctctgggggtcgttgatttttagaagacacatgaaatctgcatgcatgcatgagcttgtttcattgtatttgatttcatcttattcttaattggtctatttgttatttgattatgtattatattctaaaatgaaatggattatcgaaaataga

SEQ ID NO: 25 - protein, 676 aa, Glycine max
MREWTTIESFKCVVHVFGRSKIQHIVASDYTSFTYNGFQSSHLYLDGSAEFTTNGMVKLTNHTKQQKGHAFFPSPIVFKNTTNGSVFSFSTTFVFAIRSEFPNLSGHGIAFVVSPTKEVPHSLPSQYLGLFDDTNNGNNSNHVFGVELDTILNTEFGDINDNHVGIDVNELKSVKSASAGYYSDGGFKNLSLISGYPMQVWVEYDGLKKQIDVTLAPINVGKPERPLLSLNKDLSRILNSSMYVGFTSSTGSILSSHYVLGWSFKVNGKAQQLAISELPMLPRLVGKQESKVLIVGLPLILLILILMVALAVVHAIKRKKFVELLEDWEQDYGPHRFKYKDLSLATKGFREKELLGSGGFGRVYKGVMPISKIEVAVKKVSHESRQGMREFVAEIASIGRLRHRNLVPLLGYCRRKGELLLVYDYMPNGSLDKYLYNKPRVTLNWSQRFRITKGVASGLFYLHEEWEQVVLHRDIKASNVLLDAELNGRLGDFGLSRLYEHGTDPHTTHVVGTLGYLAPEHTRTGKATTSSDVFAFGAFMLEVVCGRRPIEQGRESGSEILVDWVYNCWKKGEILEARDPNLGANYRPDEVELVLKLALLCSHSEPLARPSMRQVVQYLEKDVPLPDLSMLSLSSIGLTFGLHEDFQDCPMSYPSSMDRPISHTSSIAESLLSGGR

SEQ ID NO: 26 - DNA, 300 bp, Glycine max
ctcataggttcaagtacaaagatcttagcctagcaacaaaggggttcagggagaaggagttattggggagtggtggatttggtagagtctacaaaggggtgatgccaatttcgaagattgaggttgcagtgaagaaggtgtcacatgaatcaaggcaagggatgagggaatttgtggcggaaatcgctagcattggccgtcttaggcaccggaatctggttccccttctcgggtactgcaggcgcaaaggagagttgctgttggtctatgactacatgccaaatggaagccttgacaagt

SEQ ID NO: 27 - DNA, 2145 bp, Glycine max
atgtctctcaaggttgtaacagtggtgtttttgctggcaacaatagtagtagccagtgatgacaccagtttcacctataatggattccaatcttcttatttgtaccttgatggaagtgctgagttcaccaccaatggcatgcttaagctcaccaatcacaccaagcaacaaaagggtcatgctttcttcccaagtccaatagtcttcaagaacaccacaagtggaagtgtgttttctttctccaccacttttgtgtttgccataaggtctgagtttcccaatttgagtggtcatgggattgtgttcgtggtttctccaacaaaaggggttccacattctcttccaagccagtaccttggcctctttgatgacaccaacaacggcaacaatagcaaccatatttttggggtggagcttgatacaattctgaacaccgagtttggtgatatcaatgacaaccatgttggagttgatgttaatgaattgaagtcggttaaatcagctgctgcagggtattacagtgatgaggggtttaagaatttgagtctcatcagtggctacccaatgcaggtgtgggtggaatatgatggcctaaagaagcagattgatgttactttagctccaatcaatgtcggtaaaccggaaggccctttattatcattgagcaaagacctttctccaattctgaacagtagcatgtatgttggattctcatcctcaactgggtcaattctaagttctcattatgttttgggttggagctttaaggtgaatggcaaggctcaacagcttgcaatctctgaactccctatgctacctagactaggtggtaaagaagagtcaaaggttcttattgtagggttgcctctgatattactcagtttgattctcatggtggctctagcagtggttcatgtcatcaagaggaagaagttcacggagctgcttgaagattgggagcaagactatggtcctcataggttcaagtacaaagatcttagcctggcaacaaaggggttcagggagaaggagctgttggggagtggtggatttggtagagtctacaaaggggtgatgccaatttccaaaattgaggtggcagtgaagaaggtgtcccgtgaatcaaggcaagggatgagggaatttgtggcagaaatcgttagcattggctgtctcaggcaccggaatttggttccccttctggggtactgcaggcgcaaaggagagttgctattggtctatgactacatgccaaatggaagccttgacaagtatctctacaacaaaccaagagtgaccttaaattggagccagaggtttaaaatcacaaaaggggttgcttcaggtctgttttatctacatgaggaatgggagcaagttgtggtccacagagacattaaggctagcaatgtgctacttgatgctgaattgaatggtagattaggggactttggtctttcaaggttgtatgaacatggaactgatcctcacactactcatgtggttggaactcttggttatcttgcacctgagcacactagaacgggtaaggcaactacaagctctgatgtgtttgcttttggtgcatttatgctagaggttgtgtgtggaaggaggccaatagagaaaggaggggaatccgggagcgaaattctggtcgattgggtgtataattgttggaaaaagggtgagattcttgagtcaatggatccaaacttgggcgcaaattataggccagatgaggtggaattggtgttgaaactcgccttgttgtgctcacattctgagcctttggctaggccaagcatgcgccaggttgtgcaatacttggagaaagatgtgcctctgcctgatttgtgtatgcttagcttatcttcaaatggcttaacttttggtttgcatgaagattttcaggactgtccaatgtcttatccttcatctatgaataggccaatatctcatacttcttcaattgttgaatcacttctctctgggggtcgttgatttttagaagacacatgaaatgtgcatgcatgcatgagcagtttcattgtgtttggtttcatcttattcttaattggtctatttgttatttgtttatgtattatattctaaaatgaaatagattatcgaaaatattaaaga

SEQ ID NO: 28 - protein, 667 aa, Glycine max
MSLKVVTVVFLLATIVVASDDTSFTYNGFQSSYLYLDGSAEFTTNGMLKLTNHTKQQKGHAFFPSPIVFKNTTSGSVFSFSTTFVFAIRSEFPNLSGHGIVFVVSPTKGVPHSLPSQYLGLFDDTNNGNNSNHIFGVELDTILNTEFGDINDNHVGVDVNELKSVKSAAAGYYSDEGFKNLSLISGYPMQVWVEYDGLKKQIDVTLAPINVGKPEGPLLSLSKDLSPILNSSMYVGFSSSTGSILSSHYVLGWSFKVNGKAQQLAISELPMLPRLGGKEESKVLIVGLPLILLSLILMVALAVVHVIKRKKFTELLEDWEQDYGPHRFKYKDLSLATKGFREKELLGSGGFGRVYKGVMPISKIEVAVKKVSRESRQGMREFVAEIVSIGCLRHRNLVPLLGYCRRKGELLLVYDYMPNGSLDKYLYNKPRVTLNWSQRFKITKGVASGLFYLHEEWEQVVVHRDIKASNVLLDAELNGRLGDFGLSRLYEHGTDPHTTHVVGTLGYLAPEHTRTGKATTSSDVFAFGAFMLEVVCGRRPIEKGGESGSEILVDWVYNCWKKGEILESMDPNLGANYRPDEVELVLKLALLCSHSEPLARPSMRQVVQYLEKDVPLPDLCMLSLSSNGLTFGLHEDFQDCPMSYPSSMNRPISHTSSIVESLLSGGR

SEQ ID NO: 29 - DNA, 2260 bp, Glycine max
acacccatttctttttcttcctcctcttgatcttaaccatgaccaacccaaaactaggctatgccggaatctccgattccggcaaccacattccctcctccaagaaaaaccacaagaagctcctcctctccctcttggccaccctcctcgtggcggcttcgctggtggccattgttgtcggagtaaaaaataaaaactccgacaactctgccaccagcacccccctctccctctcccaccactcacacacgatcgtgaagagcgcgtgcagctccacattctaccccgaactctgctactccgccatcgcctctgagcccaatgtcactcacaaaatcaccaccaaccgcgacgtcatccagctctccttaaaaatcaccttccgcgcggtggagcaaaactacttcaccgtgaaaaaactctttaccgaacacgacgacctaacaaaacgcgaaaaaaccgctctccacgactgcctggaaaccatagacgaaaccctcgacgagctcagagaagcacaacacaacctcgagctttaccctaacaagaaaacgctctaccagcacgccgacgatctcaaaaccctcatcagcgccgccataacgaaccaggtcacgtgcctcgacggtttctctcacgatgatgctgacaagcacgtgaggaaggcactggagaagggacaggtgcacgtggaacacatgtgcagcaacgcattggccatgacgaagaacatgaccgacagtgacatagctaactacgaatacaatatgagagttgaaaataatggacaaaatggtaatagcaataggaagttgttggtggagaatgatgttgagtggccggagtggatttcggcggcggataggaggctacttcaggcgtcgacggtgaaggctgacgtgacggtggcggcggacgggagcggggacttcaagacggtgacggaggcggtggacgcggctccgttgaagagtagtaagaggtttgttataaggataaaggctggggtctatagagagaacgttgaggtgcctaagaagaagaacaatatcatgttcttgggtgatgggaggaccaacactatcatcactgctagcagaaatgttgttgatggtagcaccaccttccactctgccaccgtcgctgtggtgggttcaaatttcttggcccgggacttaacattccaaaacacagcgggcccatcaaagcaccaagctgtggccctaagggttggtggggacctctcagcattcttcaattgtgacatcctagcattccaagacaccctttatgtccacaacaatcgccaattctttgtgaagtgcctaattgctggcacagtggatttcatctttggcaactctgcagtggtgttccaagactgtgacatccacgccaggcttcctagctcgggccagaagaacatggtcacggcccaaggaagagttgaccctaaccaaaacactggcattgtgatccagaagtgtaggattggtgccactaatgacttggagtctgtgaagaagaatttcaagacatatctagggaggccttggaaggagtactctaggactgtgatcatgcaaagtagtataagtgatgtgattgacccaattgggtggcatgagtggagtgggaattttggattgagcactttggtttatagggagtatcagaacactgggcctggtgctggtacttctaatagggtcacttggaaagggtataaggtcatcactgatactgctgaggccagggaatatacccctggaagcttcattgggggctctagctggttgggctcaactgggtttcctttctctcttgggttgtaaattatgttcaacttttgagcattataattgttacctattattgttatgttttttattagcaaaagggctgaagcccaagctagaaccctctacagaaaggcactccctagagaaacaaagttgaccaaaaaatttaaaagaaaacaaaaaaggaaagagggagagagagattattgctggagaggagggggttttagtttgtctttggttgggttttagaacacaccgtgttcttctaccttaggctatttattctgattttgccactggctaaaataatcatgataataagtgtatcagtgtaagccaaagttttggctcgttcaaaaattggattttgttttcattttatggataaattattctatggtccggaccaccactgaccaattaaagagcgacacatggacaacttgttttatcat

SEQ ID NO: 30 - protein, 598 aa, Glycine max
MTNPKLGYAGISDSGNHIPSSKKNHKKLLLSLLATLLVAASLVAIVVGVKNKNSDNSATSTPLSLSHHSHTIVKSACSSTFYPELCYSAIASEPNVTHKITTNRDVIQLSLKITFRAVEQNYFTVKKLFTEHDDLTKREKTALHDCLETIDETLDELREAQHNLELYPNKKTLYQHADDLKTLISAAITNQVTCLDGFSHDDADKHVRKALEKGQVHVEHMCSNALAMTKNMTDSDIANYEYNMRVENNGQNGNSNRKLLVENDVEWPEWISAADRRLLQASTVKADVTVAADGSGDFKTVTEAVDAAPLKSSKRFVIRIKAGVYRENVEVPKKKNNIMFLGDGRTNTIITASRNVVDGSTTFHSATVAVVGSNFLARDLTFQNTAGPSKHQAVALRVGGDLSAFFNCDILAFQDTLYVHNNRQFFVKCLIAGTVDFIFGNSAVVFQDCDIHARLPSSGQKNMVTAQGRVDPNQNTGIVIQKCRIGATNDLESVKKNFKTYLGRPWKEYSRTVIMQSSISDVIDPIGWHEWSGNFGLSTLVYREYQNTGPGAGTSNRVTWKGYKVITDTAEAREYTPGSFIGGSSWLGSTGFPFSLGL

SEQ ID NO: 31 - DNA, 340 bp, Glycine max
tgatccagaagtgtaggattggtgccactaatgacttggagtctgtgaagaagaatttcaagacatatctagggaggccttggaaggagtactctaggactgtgatcatgcaaagtagtataagtgatgtgattgacccaattgggtggcatgagtggagtgggaattttggattgagcactttggtttatagggagtatcagaacactgggcctggtgctggtacttctaatagggtcacttggaaagggtataaggtcatcactgatactgctgaggccagggaatatacccctggaagcttcattgggggctctagctggttgggctcaactgggtt

SEQ ID NO: 32 - DNA, 1896 bp, Glycine max
ctgacgatgacccgcgacgtcatccaactctccctaagcatcaccttccgcgcggtggagcgcaactacttcaccgtgaagaaactcctaaccaaacacgacctaacgaaacgcgagacaaccgcgctccacgattgcctcgaaaccatagacgaaaccctcgacgagctcagagaagcacaacacgacctcgagctctaccctaacaagaaaactctgtaccagcacgccgacgatctcaaaaccctcatcagcgccgccataacgaaccaggtcacgtgcctcgacggtttctctcacgatgacgcagacaaacacgtgaggaaagagttggagaagggacaggtgcatgtggaacacatgtgcagcaacgcattggccatgacgaagaacatgaccgacggtgacatagcaaactacgaatacaagatgaaagtggaaaacactaacagcaatagaaagttattagtggagaatggtgttgagtggccggagtggatttcggcggcggataggaggctgctgcaggcggcgacggtgaaggcggacgtgacggtggcggcggacgggagcggggactttaagacggtgacggaggcggtgaaggcggctccgttgaagagtagtaagaggtatgttataaggattaagggtggggtgtatagagagaacgttgaggtggataagaagaagacgaatatcatgttcttgggtgatggaaggaccaacactatcatcactgctagcagaaatgttgttgatggtagcaccaccttccactctgccaccgtcgctgtggtgggtgcaaatttcttggcccgggacataacattccaaaacacagcgggcccatcaaagcaccaagccgtggccctaagggttggtggggacctctcagcattcttcaattgtgacttcctagcattccaagacaccctttatgtgcacaacaatcgccaattcttcgtcaagtgcctgattactggcacagtggatttcatctttggcaactcagcagtggtgttccaagactgtgacatccacgccaggcttcctgactcgggccagaagaacatggtcacggcccaaggaagagttgaccctaaccaaaacactggcattgtgatccaaaagtgtaggattggtgccactaaggacttggagtctgtgaagaagaatttcaagacttatcttgggaggccttggaaggagtactctaggactgtgatcatgcaaagtagtataagtgatgtcattgacccaattgggtggcatgagtggagtgggaattttgcattgagtactttggtttatagggagtaccaaaacactgggcctggtgctggtacttctaatagggtcacttggaaagggtataaggtcatcactgatgctgccgaggccagggattatactcctggaagcttcattgggggctctagctggttgggctcaactgggtttcctttctctcttggtttgtaaattatgtgaaacttttgagtcttgtaattgttacctattgttattgttatgtggcagagagaaacaaattaagttggcccaaatgtttagaagaaaacaaaaaaaggaaagagggagagagagagagattattgctggagaggtgggtggggggttttagtttatctttcacttgtattggttgggttttggaacacaccatgttcttctaccttaggctatttattgtgatagtgccattggctaaaataatcatgataataagtgtatcaatgtaagccaaagttttggctcgttcaaaaattggattaaaataatcatgataataagtgtatcaatgtaagccaaagttttggctcgttcaaaaattggatttttgtgttatttaataattgtatgtcatgccgtaca

SEQ ID NO: 33 - protein, 492 aa, Glycine max
MTRDVIQLSLSITFRAVERNYFTVKKLLTKHDLTKRETTALHDCLETIDETLDELREAQHDLELYPNKKTLYQHADDLKTLISAAITNQVTCLDGFSHDDADKHVRKELEKGQVHVEHMCSNALAMTKNMTDGDIANYEYKMKVENTNSNRKLLVENGVEWPEWISAADRRLLQAATVKADVTVAADGSGDFKTVTEAVKAAPLKSSKRYVIRIKGGVYRENVEVDKKKTNIMFLGDGRTNTIITASRNVVDGSTTFHSATVAVVGANFLARDITFQNTAGPSKHQAVALRVGGDLSAFFNCDFLAFQDTLYVHNNRQFFVKCLITGTVDFIFGNSAVVFQDCDIHARLPDSGQKNMVTAQGRVDPNQNTGIVIQKCRIGATKDLESVKKNFKTYLGRPWKEYSRTVIMQSSISDVIDPIGWHEWSGNFALSTLVYREYQNTGPGAGTSNRVTWKGYKVITDAAEARDYTPGSFIGGSSWLGSTGFPFSLGL

SEQ ID NO: 34 - DNA, 2100 bp, Glycine max
aggtggtgacataggtgtttgcagttgcagtagaacctaagatttgtgcccagatagaaaaaaaaagagcaacatagtcatttcctttgaccaagatgaagtttatgaaacttggatctaagccagattcttttcagagtgaaggggataatatcaggtatgtggcagctgagttggcaactgacatagttattaatgttggaaatgtcaaatttcatctccataagtttcctcttttgtcaaaaagtgcacgcttccaaaagctgattacaaacactaatgaagagaacattgatgaagtccacatccatgacattcctggtggacctgctgcttttgaaatatgtgccaagttctgttatggtatgacagtcactcttaatgcatacaatgttgttgcagctcgctgtgcagcagaatatcttgagatgtatgaaactgttgagaaaggcaatcttatctacaagattgaagtcttcctcaactccagcattttcaggagttggaaagactcgattattgttcttcaaactaccaagtctcttcttaaatggtctgaggaacttaaggtagtgagccatggcattgactccatagctaccaaggcttcacttgatacattaaaggtggagtggtcatacacctataacaggaaaaagctaccatctgaaaatagtaatgatcctcacttcagtagtgtgaggaaacaacaattggttccaaaggactggtgggtggaggacctttgtgagctccaacttgatctttatgaacgagttataacaacaattatagcaaaaggcaatgtttctggtgctgtaattggagaagctctaaatgcttatgcatcaagaaggatgcctggcttcaacaagggtgagatccaaggaggagatattataaaggatagattactgttggagaccataatccgaatattacctgtggacatgggcagtgcctctttcagtttcttggtgaagttattaagggtagctattcagttggaatgtgaggagttggagagatctgaactgattaggagaataggcatgtgccttgaggaggctaaagtgtctgatttattaattcgtgccccagttggtgacacaattttttatgttgatattgtgcaaaggctagtggaagagtttgtagcatgtggtcaacaggttcagacggactctctgttggaagatgaatttcaggagatcagaagccccgggatggtatcagacccttctaaggctaaggtggcaaaactggtggatggctaccttgctgagattgcacgtgatccaaatttacctcttgcaaaatttgttaatcttgctgaattagtatcaagctttacaagagcatctcatgatggcctttatcgtgccattgacatgtatttaaaggagcatcctggaatcagcaagagcgaaaggaaaaaaatatgtaggttgatgaactgcaggaacttatctgcagaggcttgcatgcatgctgtgcagaatgagcggcttcccatgcgtgttgttgtgcaggttctcttctttgaacagctgagagctacaacatcttcaggagacaacagcacaccagatcatcctgggtctctcagggcctttcttcctggtggatctcatgggagctcaatgtctactataacaaacacagaagaggaatgggatgctgtgggaacaatggaagacataaaatctctgaaaggggaagttgatgcactaaaattatccggtggaaccggcagagccagcggcagaaaagacaataacggtgataaaggcaatgccgacaatgttgctgccagtaaaatgaaaggcttcatatcaaagaagatactctctaaaatttggtctagcaaagaaaaaagtggtgatctaagtagctctgatacttcagagagccctgcttctactgttgtagaggaaacaaaatctaccccatctagaagtaggaggcattcagtatcttagaccatgttatgcagactttagagctccattcagttactgtataatcatctcactaaagtagcatcaggagtcagaggacactgacatcatattccagctgatacttcaatgagttcta

SEQ ID NO: 35 - protein, 628 aa, Glycine max
MKFMKLGSKPDSFQSEGDNIRYVAAELATDIVINVGNVKFHLHKFPLLSKSARFQKLITNTNEENIDEVHIHDIPGGPAAFEICAKFCYGMTVTLNAYNVVAARCAAEYLEMYETVEKGNLIYKIEVFLNSSIFRSWKDSIIVLQTTKSLLKWSEELKVVSHGIDSIATKASLDTLKVEWSYTYNRKKLPSENSNDPHFSSVRKQQLVPKDWWVEDLCELQLDLYERVITTIIAKGNVSGAVIGEALNAYASRRMPGFNKGEIQGGDIIKDRLLLETIIRILPVDMGSASFSFLVKLLRVAIQLECEELERSELIRRIGMCLEEAKVSDLLIRAPVGDTIFYVDIVQRLVEEFVACGQQVQTDSLLEDEFQEIRSPGMVSDPSKAKVAKLVDGYLAEIARDPNLPLAKFVNLAELVSSFTRASHDGLYRAIDMYLKEHPGISKSERKKICRLMNCRNLSAEACMHAVQNERLPMRVVVQVLFFEQLRATTSSGDNSTPDHPGSLRAFLPGGSHGSSMSTITNTEEEWDAVGTMEDIKSLKGEVDALKLSGGTGRASGRKDNNGDKGNADNVAASKMKGFISKKILSKIWSSKEKSGDLSSSDTSESPASTVVEETKSTPSRSRRHSVS

SEQ ID NO: 36 - DNA, 370 bp, Glycine max
tgcaggagcttatctgcagaggcttgcatgcatgctgtgcagaatgagcggcttcccatgcgtgttgttgtgcaggttctcttctttgaacagctgagagctacaacatcttcaggagacaacagcacaccagatcatcctgggtctctcagggcctttcttcctggtggatctcatgggagctcaatgtctactataacaaacacagaagaggaatgggatgctgtgggaacaatggaagacataaaatctctgaaaggggaagttgatgcactaaaattatccggtggaaccggcagagccagcggcagaaaagacaataacggtgataaaggcaatgccgacaatattgctgccagtaaaatgaaag

SEQ ID NO: 37 - DNA, 2040 bp, Glycine max
tttgggtcggtggtttgtctttcagaggtagaacaacatagggaaaaaggagaaacgtagtcatttcctttggccaagatgaagtttatgaaacttggatctaagccagattcttttcagaatgatggggataatatcaggtatgtggcaactgagttggcaactgacatagttgttaatgttggaaatgtaaaattttatctccataagtttcctcttttgtcaagaagcacgtgcttccaaaaactgattacaaacgctaatgaggagaacaatgatgaagttcacatccatgacattcctggtggacctgctgcttttgaaatatgtgctaagttctgctatggtatgacagtcactcttaatgcatacaatgtcgttgcagctcgctgtgcagcagaatatcttgagatgtatgaaactgttgagaaaggaaatcttatctacaagattgaagtcttcctcaactccagcattttcaggagttggaaagactcgatcatcgttcttcaaactaccaagtctcttcttccatggtctgaggaacttaagttagtgagccatggtattgactccattgctaccaaggcttcaattgatacatcaaaggtggagtggtcatacacctataacaggaaaaagctaccatctgaaaatagtaatgatcctcacttcaatagtgtgaggaaacaacaattggttccaaaggactggtgggtggaggacctttgtgagctccaacttgatctctatgaacgggttataacaacaattttaaccaaaggcaatgtttctggttctgtaattggagaagctctaaatgcttatgcctcaagaaggatgcctggcttcaacaagggtgtgatccaaggaggagacaatgtaaagaatagattattgttggagaccataatccgaatattacctttggatgtgggcagtgcctctttcagtttcttggggaagttattaagggtagctattcagttggaatgtgaagagttggagagatctaaactgattaggagaataggtatgtgccttgaggaggctaaggtgtctgatttattaattcgcgccccagttggtgatgcagtttttgatgttgatattgtgcaaaggctagtagaagagtttctagcatgtgatcaacatgttcagacggatactctgttggacgatgaatttcaggagacaagaagccctgggatggtatcggaatcttcgaaggcaaaggttgcaaaactggtggatggataccttgctgagattgcacgtgatccaaatttacctctttcaaaatttgttaatcttgctgaattagtatcaagctttccaagagcatttcatgatggcctttatcgtgcgattgacatgtatttaaaggagcaccctggaatcagcaaaagcgaaaagaaaagaatatgcaggttgatgaactgcaggaagttgtcagcagaggcttgcatgcatgctgtgcagaacgagcggcttcccatgcgcgttgttgtgcaggtcctcttctttgaacagctgagagctacaacatcatcaggaggcaacggaacaccggatcattccggctctatcagggcctctcttcccggtggatctcatgggagttcaaggtcggaagaggaatgggaagccgtgggaacgatggaagacataaaatctctgaaaggggagcttattgctctgaaattatcaggaggaacaaggggagccagcagcagaagcaacaacaatgacagtagtaaaggcaattctgagagtgttgctgctagtaaaatgaaaggtctcgtgtcgaagaagataatctccaagatttggtcaagcaaagaaagaagtggtgagctaagtagctctgatacatcagagagccctgcttctacggttgtagaggaaacaaaatctactccatctagaagtaggaggcattcattgtcttagagaccatgttctgcagaccttattccttagagctccattcagtttactgtataatcatctctcactcaagtagcatcagagttagag

SEQ ID NO: 38 - protein, 624 aa, Glycine max
MKFMKLGSKPDSFQNDGDNIRYVATELATDIVVNVGNVKFYLHKFPLLSRSTCFQKLITNANEENNDEVHIHDIPGGPAAFEICAKFCYGMTVTLNAYNVVAARCAAEYLEMYETVEKGNLIYKIEVFLNSSIFRSWKDSIIVLQTTKSLLPWSEELKLVSHGIDSIATKASIDTSKVEWSYTYNRKKLPSENSNDPHFNSVRKQQLVPKDWWVEDLCELQLDLYERVITTILTKGNVSGSVIGEALNAYASRRMPGFNKGVIQGGDNVKNRLLLETIIRILPLDVGSASFSFLGKLLRVAIQLECEELERSKLIRRIGMCLEEAKVSDLLIRAPVGDAVFDVDIVQRLVEEFLACDQHVQTDTLLDDEFQETRSPGMVSESSKAKVAKLVDGYLAEIARDPNLPLSKFVNLAELVSSFPRAFHDGLYRAIDMYLKEHPGISKSEKKRICRLMNCRKLSAEACMHAVQNERLPMRVVVQVLFFEQLRATTSSGGNGTPDHSGSIRASLPGGSHGSSRSEEEWEAVGTMEDIKSLKGELIALKLSGGTRGASSRSNNNDSSKGNSESVAASKMKGLVSKKIISKIWSSKERSGELSSSDTSESPASTVVEETKSTPSRSRRHSLS

SEQ ID NO: 39 - DNA, 1878 bp, Glycine max
aacctaagatttgtgttcagatataaaaaagaaaagaaaaaaaaaggagcaacatagtcatttcctttgaccaagatgaagtttatgaaacttggatctaagccagattcttttcagagtgaaggggataatgtcaggtatgtcgcagctgagttggcaactgacatagtcattaatgttggaaatgtaaaatttcatctccataagtttcctcttttgtcaaaaagtgcacgcttccaaaagctgattacaaacagtaatgaagagaacaatgatgaagtccatatccatgacattcctggcggatctgctgcttttgaaatatgtaccaagttctgttatggtatgacagtcactcttaatgcatacaatgtcgttgcagctcgctgtgcagcagaatatcttgagatgtacgaaactgttgagaaaggaaatcttatctacaagattgaagtattcctcaactcgagcattttcaggagttggaaagactcgattattgttcttcaaactaccaagtctcttcttaaatggtctgaggaacttaaggtagtgagccatggcattgactccatagctaccaaggcttcacttgatacatcaaaggtggagtggtcatacacctataacaggaaaaagctaccatctgaaaatagtaacgatcctcagtctaataatgcaaggaaacaacaattggttccaaaggactggtggtgggtggaggacctttgtgagctccaacttgatctttatgaacgggttataacagcaattatagaaaaaggcaatgtttctggtgctgtaattggagaagctctaaatgcttatgcctcaagaaggatgcctggcttcaacaagggtgagatccaaggaggagatattgtaaagaatagattactgttggagaccatactccgaatattacctgtggacatgggcattgcctctttcagtttcttggtgaagttattaagggtagctattcagttggaatgtgaagagttggagagatctgaactgattaggagaataggtatgtgccttgaggaagctaaggtgtctgatttattaatttgtgccccagttggtgatgcaattcttgatgttgatattgtgcaaaggatagtagaagagtttgtagcatgtgatcaacaggttcagacggactccctgttggaagatgaatttcaggagatcagaagccctgggatggtatcagacccttcaaaggctaaggtggcaaaactggtggatggctaccttgctgagattgcatgtgatccaaatttacctgttgcaaaatttgttaatcttgctgaattagtatcaagcttccctagagcatctcatgatggcctttatcgtgccattgacatgtatttaaaggagcatcctggaatcagcaagagcgaaaggaaaagaatatgtaggttgatgaactgcaggagcttatctgcagaggcttgcatgcatgctgtgcagaatgagcggcttcccatgcgtgttgttgtgcaggttctattctttgaacaactgagaactacaacatcttcagggggcaacagcacaccagatcatcctgggtctctcaggtcatttcttcctggtggatctcatgggagctctaggtctactataacaaacacagaagaagaatgggatgctgtgggaacaatggaagacataaaatctctgaaaggggaagttgatgcactaaaattatcaggtggaaccggtagggccagcagcggaaaagacaataacagtgaaaaaagtgttgatataagtagctctgatacgtcagagagccctgcttctactgttgtagaggaaacaaaatctactccagctagaagacttacaaggaggattagttag

SEQ ID NO: 40 - protein, 600 aa, Glycine max
MKFMKLGSKPDSFQSEGDNVRYVAAELATDIVINVGNVKFHLHKFPLLSKSARFQKLITNSNEENNDEVHIHDIPGGSAAFEICTKFCYGMTVTLNAYNVVAARCAAEYLEMYETVEKGNLIYKIEVFLNSSIFRSWKDSIIVLQTTKSLLKWSEELKVVSHGIDSIATKASLDTSKVEWSYTYNRKKLPSENSNDPQSNNARKQQLVPKDWWWVEDLCELQLDLYERVITAIIEKGNVSGAVIGEALNAYASRRMPGFNKGEIQGGDIVKNRLLLETILRILPVDMGIASFSFLVKLLRVAIQLECEELERSELIRRIGMCLEEAKVSDLLICAPVGDAILDVDIVQRIVEEFVACDQQVQTDSLLEDEFQEIRSPGMVSDPSKAKVAKLVDGYLAEIACDPNLPVAKFVNLAELVSSFPRASHDGLYRAIDMYLKEHPGISKSERKRICRLMNCRSLSAEACMHAVQNERLPMRVVVQVLFFEQLRTTTSSGGNSTPDHPGSLRSFLPGGSHGSSRSTITNTEEEWDAVGTMEDIKSLKGEVDALKLSGGTGRASSGKDNNSEKSVDISSSDTSESPASTVVEETKSTPARRLTRRIS

SEQ ID NO: 41 - DNA, 2040 bp, Glycine max
gctcggtggtttgtctttcagagaggtagaacaacatagggaaaaaggagaaacatagtcatttcctttggccaagatgaagtttatgaaacttggatctaagccagattcttttcagaatgacggggataatatcaggtatgtggcaactgagctagcaactgacatagttgttaatgttggaaatgtaaaattttatctccataagtttcctcttttgtcaaaaagtgcgggctttcaaaagctgattacaaacactaatgaggagaacaatgatgaagttcacatccatgacattcctggcggacctgctgcttttgaaatatgtgtcaagttctgttatggtatgacagtcactcttaatgcatacaacgtcgttgctgctcgctgcgccgcagaatatcttgagatgtatgaaactgttgagaaaggaaatcttatctacaagattgaagtcttcctcgactcaagcattttcaggagttggaaagactcgattattgttcttcaaactaccaagtctcttcttccatggtctgaggaacttaagttagtgagccatggcattgactccattgctaccaaggcttcaattgatacatcaaaggtggagtggtcatacacctataacaggaaaaagctaccatctgaaaatagtaatgatcctcccttcaatagtgtgaggaaacaacaattggttccaaaggactggtgggtggaggacctttgtgagctccaacttgatctttatgagcgggttataaaaacaattataagcaaaggtaatgtttctggcactgtaattggagaagctctaaatgcttatgcctcaagaaggatgcctggtttcaacaagggtgtgatccaaggagatattgtaagaaatagattactgttggagaccataatccgaatattacccttggacgtgggcagtgtctctttcagtttcttggtgaagttattaagggtagctattcagttggaacgtgaagagttggagagatctgaactgattaggagaataggtatgtgccttgaggaggctaaggtgtctgatttattaatttgcgccccagttggtgacacagtttttgatgttgacattgtgcaaaggctagtagaagagtttgtagcatgtgatcaacatgttcagacggatactctgttggaagatgactttcaggaggagatcagaagccccgggatggtttcagaatcttcaaaggcaaaagtggcaaaattggtggatggataccttgctgagattgcacgtgatccaaatttaccttttgcaaaatttgttaatcttgctgaattagtatcaagctttccaagagcctctcatgatggcctttatcgtgccattgacatgtatttaaaggagcatcctggaatcagcaaaagcgaaaagaaaagaatatgcaggttgatgaactgcaggaagttgtctgcagaggcttgcatgcatgctgtgcagaacgagcggcttcccatgcgcgttgttgtgcaggttctcttctttgaacagctgagagctacaacatcttctggaggcaatggcacaccggatcatcctggctccatcagggccttacttcctggtggatctcatgggagctcaaggtctactataacaaacacagaagaggaatgggatgccgtgggaacaatggaagacataaaatccctgaaaggggagcttgatgcactgaaattatctggtggaaccagcggggccagcagcagaagcgacaacaacgatggcggtaaagacaatgctgagaaagttgctgctagtaaaatgaaaggtctcatgtcgaagaagataatctctaagatttggtcaagcaaagaaagaagtggtgagctaagtagttctgatacatcagagagccctgcttctactgttgtagaggaaacaaaatctactccatctagaagtaggaggcattcattgtcttagagaccatgttctgcagaccctagtccttagagctccattcagtttattgtataatcatctcattcaagtagcat

SEQ ID NO: 42 - protein, 629 aa, Glycine max
MKFMKLGSKPDSFQNDGDNIRYVATELATDIVVNVGNVKFYLHKFPLLSKSAGFQKLITNTNEENNDEVHIHDIPGGPAAFEICVKFCYGMTVTLNAYNVVAARCAAEYLEMYETVEKGNLIYKIEVFLDSSIFRSWKDSIIVLQTTKSLLPWSEELKLVSHGIDSIATKASIDTSKVEWSYTYNRKKLPSENSNDPPFNSVRKQQLVPKDWWVEDLCELQLDLYERVIKTIISKGNVSGTVIGEALNAYASRRMPGFNKGVIQGDIVRNRLLLETIIRILPLDVGSVSFSFLVKLLRVAIQLEREELERSELIRRIGMCLEEAKVSDLLICAPVGDTVFDVDIVQRLVEEFVACDQHVQTDTLLEDDFQEEIRSPGMVSESSKAKVAKLVDGYLAEIARDPNLPFAKFVNLAELVSSFPRASHDGLYRAIDMYLKEHPGISKSEKKRICRLMNCRKLSAEACMHAVQNERLPMRVVVQVLFFEQLRATTSSGGNGTPDHPGSIRALLPGGSHGSSRSTITNTEEEWDAVGTMEDIKSLKGELDALKLSGGTSGASSRSDNNDGGKDNAEKVAASKMKGLMSKKIISKIWSSKERSGELSSSDTSESPASTVVEETKSTPSRSRRHSLS

SEQ ID NO: 43 - DNA, 2100 bp, Glycine max
ggggctagctgcgacttattggatttagaaatcggctattgagtagtggtgattttttttttagttgcttcagttgagtcggcatcatctgagaagaagcagatcttatattgtcaagatgaagttcatgaagcttggttctaagccagatacctttcagactgatgggaacaatgtcaggtatgtggcaagtgagttggcgtcagatattgttgttagtgtaggggatataaagttttatctacacaagtttcctcttctatcgaagagttctcatatacagacattgatctccctcaataatgaagaaaatgtagatgaagttcaaatttctgacattcctggtggtgcaaatacctttgagatatgtgctaagttttgctatggcatgactgttaccctcaatgcttacaacgtaatagcaactcgatgtgcagcagagtatcttggaatgcatgaggccattgagaaagggaacctcatttacaagattgatgttttccttagctctagcattttccgtagttggaaggattcaatcatccttcttcagacttcaaagtctatgttacccttggttgaggacctaaaggttgtcagccattgcattgaatctatagcaaataaggcatgtgttgatgtatccaaagtcgattggtcctacacctataaccggaagaagcttccagaggaaaatggaattgagtcaaaccagaatggactcagaactcgactggtgccaaaagactggtgggttgaagatttatgtgagcttgaagttgatttgtacaagtctgtgattacaaatattaaatccaaggcagttcaatctaatgaagtaattggcgaagctttgaaagcttatgcttatagaagattgccaaatttcagcaagggtatgatccagtgtggggatgtgtcaaagcatcgtttaatagttgaaactatagtgtggttgctgcctactgagaaaggcagtgttccttgtaggttcttactcaagttattgaaagcggccatttttgtagaatcaggagataggactaaagaagagctggtaaagagaattgggcagcaactggaggaggcttctgtaagtgatattttgattcaagcaccagatggggctacaatctatgatgttagtatagtacagaacattgttagagagttttttatgaagaatggcaatgctgagattgagtcagttgggggtgatgaacttgaggggataagaaaaccagggattttatcagatgcttccaagctgatggttgcaaagctcatagatgagtaccttgctgaaatagcaaaagatcccaatttacctttgccagagtttgttaatcttgctgagttagtgtctagcatctctcgtccagctcatgatggcctttatagggctattgatacatatcttaaggagcaccctgcgatcaacaaaggcgaaaagaagaggatatgcaagctgatggattgtagaaagctatcagttgacgcgtgcttgcatgcagtgcaaaacgagagactaccattgcgtgtagtcgtgcaggtactatactttgagcagttaaggactgctgcatcatcaggtaccagcactcctgacatacctagaggaatcaaagacttgaacaatgaatccaatggaagctcaaggtcagggacaactaacccagaagacgagttggatgctgtggccacagctgaggagctgaaggcattaagaaaggaacttgcatcattgaggctgagcaatggagttggaaacaatgacaaagatggagacaacaaacccagcatggacaaagctgtcatgagcaaggtgaaagggttgctcaagtcaaagaagtctttcattaagatttgggccagtaaagggggacaaggtgaaaatagtggctcagactcatcagagagtatgagttctgctaacccagaagaagccaaatctactccatccagaaataggaggcattcagtttcttgaaagtagtagtttggttcattttgttggcttttctattattgtttcttttctttctttctttttactagtttcaccaagacaagattccccaacactagtggtttatcttt

SEQ ID NO: 44 - protein, 623 aa, Glycine max
MKFMKLGSKPDTFQTDGNNVRYVASELASDIVVSVGDIKFYLHKFPLLSKSSHIQTLISLNNEENVDEVQISDIPGGANTFEICAKFCYGMTVTLNAYNVIATRCAAEYLGMHEAIEKGNLIYKIDVFLSSSIFRSWKDSIILLQTSKSMLPLVEDLKVVSHCIESIANKACVDVSKVDWSYTYNRKKLPEENGIESNQNGLRTRLVPKDWWVEDLCELEVDLYKSVITNIKSKAVQSNEVIGEALKAYAYRRLPNFSKGMIQCGDVSKHRLIVETIVWLLPTEKGSVPCRFLLKLLKAAIFVESGDRTKEELVKRIGQQLEEASVSDILIQAPDGATIYDVSIVQNIVREFFMKNGNAEIESVGGDELEGIRKPGILSDASKLMVAKLIDEYLAEIAKDPNLPLPEFVNLAELVSSISRPAHDGLYRAIDTYLKEHPAINKGEKKRICKLMDCRKLSVDACLHAVQNERLPLRVVVQVLYFEQLRTAASSGTSTPDIPRGIKDLNNESNGSSRSGTTNPEDELDAVATAEELKALRKELASLRLSNGVGNNDKDGDNKPSMDKAVMSKVKGLLKSKKSFIKIWASKGGQGENSGSDSSESMSSANPEEAKSTPSRNRRHSVS

SEQ ID NO: 45 - DNA, 215 bp, Glycine max
ggtggtgcaaatacctttgagatatgtgctaagttttgctatggcatgactgttaccctcaatgcttacaacgtaatagcaactcgatgtgcagcagagtatcttggaatgcatgaggccattgagaaagggaacctcatttacaagattgatgttttccttagctctagcattttccgtagttggaaggattcaatcatccttcttcagacttc

SEQ ID NO: 46 - DNA, 295 bp, Glycine max
cagcatggacaaagctgtcatgagcaaggtgaaagggttgctcaagtcaaagaagtctttcattaagatttgggccagtaaagggggacaaggtgaaaatagtggctcagactcatcagagagtatgagttctgctaacccagaagaagccaaatctactccatccagaaataggaggcattcagtttcttgaaagtagtagtttggttcattttgttggcttttctattattgtttcttttctttctttctttttactagtttcaccaagacaagattccccaacactagtcgg

SEQ ID NO: 47 - DNA, 2040 bp, Glycine max
agttttttcggttgagtcggcatcaactgagaggaagcagatcttattattgtcaagatgaagttcatgaagcttggttctaagcctgatacctttcaaactgatggaaacaatgttaggtatgtggcaagtgagttggcatcagatattgttgttaatgtaggggatataaagttttatctacacaagtttcctcttctatcgaagagttctcgcttacagacattgatctccctcaataatgaagaaaatgtagatgaagttcaaatttttgacattcctggtggtgcaaatacctttgagatatgtgctaagttttgctatggcatgactgttaccctcaatgcttacaatgtaatagcaactcgatgtgcagcagagtatcttggaatgcatgaggccattgagaaagggaacctcatctacaagattgatgttttccttagctctagcattttccgtagttggaaggattcaatcatccttcttcagacttcaaagtctatgttacccttggttgaggacctaaaggttgtcagtcattgcattgaatctatagcaaataaggcatgcgttgatgtatccaaagttgattggtcctacacctataaccggaagaagcttccagaggaaaatgggattgagtccaaccagaatggactcagaactcgactggtgccaaaagactggtgggttgaagatttatgtgagcttgaagttgatttgtacaagtctgtgattacgaatattaaatccaaggcagttcaatctaatgaagtaattggcgaagctttgaaagcttatgcttatagaagattgccaaatttcagcaagggtatgatccagtgtggggatgtatccaagcaccgtttaatagttgaaactattgtgtggttgctgcctactgagaaaggcagtgttccttgtaggttcttactcaagttattgaaagctgccatttttgtagaatcgggagataggactaaagaagagctggtaaagagaattgggcagcaactggaggaggcttctgtaagtgatattttgattcaagcaccagatggggctgctacaatctatgatgttagtatagtacagaacattgttagagtgttttttattaaggatcacaatgctgagattgagtcagttgggctggatgaacttgaggggataagaaaaccagggattttatcagatgcttctaagctgatggttgcaaagctcatagatgggtaccttgctgaaatagcaaaagatcccaatttacctttctcagagtttgttaatcttgccgagttagtgtctagcatctctcggccagctcatgatggcctttatagggctattgacacatatctgaaggagcaccctgggatcagcaaaggcgaaaagaagaggatatgcaagctgatggattgtagaaagctatcagttgacgcgtgcttgcatgcagtgcaaaatgagagactaccattgcgtgtagttgtgcaggtactatacttcgagcagttacggactgctgcatcatcaggtaccagcactcctgacatacctagaggaatcaaagacttgaacaatgaatcgaatggaagctcaaggtcagggacaactaacccagaagatgagttggatgctgtggccacagctgaggagctgaaggcgttaagaaaggaacttgcatcattgaggctgagcaatggagttggaaacaatgacaaagatggagacaccaaacccagcatggacaaggctgtcatggggaaggtgaaagggttgctcaagtcaaagaagtctttcattaagctttgggccagcaaagggggacaaggtgaaaatagtggctcagactcatcagagagtatgagttctgctaacccagaagaagccaaatctactccatccagaaataggaggcattcagtttcctgaaaagtagtctggttcattttgttggcttttctattattgtttcttttctttctttttactagtttcaccaagacaagattccaacactagtcgtggtttatctttttc

SEQ ID NO: 48 - protein, 624 aa, Glycine max
MKFMKLGSKPDTFQTDGNNVRYVASELASDIVVNVGDIKFYLHKFPLLSKSSRLQTLISLNNEENVDEVQIFDIPGGANTFEICAKFCYGMTVTLNAYNVIATRCAAEYLGMHEAIEKGNLIYKIDVFLSSSIFRSWKDSIILLQTSKSMLPLVEDLKVVSHCIESIANKACVDVSKVDWSYTYNRKKLPEENGIESNQNGLRTRLVPKDWWVEDLCELEVDLYKSVITNIKSKAVQSNEVIGEALKAYAYRRLPNFSKGMIQCGDVSKHRLIVETIVWLLPTEKGSVPCRFLLKLLKAAIFVESGDRTKEELVKRIGQQLEEASVSDILIQAPDGAATIYDVSIVQNIVRVFFIKDHNAEIESVGLDELEGIRKPGILSDASKLMVAKLIDGYLAEIAKDPNLPFSEFVNLAELVSSISRPAHDGLYRAIDTYLKEHPGISKGEKKRICKLMDCRKLSVDACLHAVQNERLPLRVVVQVLYFEQLRTAASSGTSTPDIPRGIKDLNNESNGSSRSGTTNPEDELDAVATAEELKALRKELASLRLSNGVGNNDKDGDTKPSMDKAVMGKVKGLLKSKKSFIKLWASKGGQGENSGSDSSESMSSANPEEAKSTPSRNRRHSVS

SEQ ID NO: 49 - DNA, 1980 bp, Glycine max
gagagaaaaattagaaagagaaaaaaatgaagtttatgaagttgggttcgaagcctgatgctcttcaaagcgatggaaaatctatcaggtatatatcatctgaattggctacagatattatcataactgttggtgaagttaagtttcacctgcacaagttccctctactgtccaagagtaatagtttacaaaagttgctgtcaaaggctaatgaagagaatgctgatgaaattcaattggatgattttcctggtggtcccaaggcctttgaaatatgtgcaaagttctgctatggaatgactgttactcttaatgcttacaatgttgtggctgctcgttgcgccgctgagtaccttgagatgactgaggatattgatagaggaaacttgattttcaaaattgaggtgtttcttacctcgagtatctttcgcagctggaaggattctataattgttctccaaactactaaatctcttctaccgtgggctgaggacttaaagattgttgggagatgtatagattccatagcttctaaaacttctgtggatcctgcaaacatcacttggtcttacacttataaccgtaaattatcggagcttgataaaatcgttgaggacaagataacaccacaagagaaaattgagcctgttcctaaggattggtgggttgaagatatatgtgagctggacattgatctttataaacgtgtaatgattactgtcaagtcaaagggaagaatggacggagttgtgattggcgaggcactaaaaatctatgctgtaagatggttgcctgattctgttgatgcattggtttctgatgctcatgcctggaggaacaaatctcttgtagaaacaattgtctgtttgttgccgtgtgataatggcatgggttgttcgtgtagtttcttgctgaagctactgaaagtggccatattagttgaagctgatgagtcttctaggggacaattgatgaagagcattggtctaaaatttcatgaagcttctgtcaaagatttactgattccagcaaggttcccccagaataccaaatatgatgttgatttggtgcaagatcttttgaatctatacatgactaacataaagggaagccgtgatgtggtggttgaggagaagaaagatagagcaaatgatgagtcaattttaggacagaggtctttgttgaatgttggcaagttggttgatggttatcttggagaaattgcacatgatccaaatctcagcctctctagctttgttgctctatcacagtcaattccagagtttgctagaccaaatcatgatggtctgtacagagctatagacgtatacttgaaggagcacccaagtttgacaaaatctgaaaggaagaacatatgtggactaatggatgtcaagaaattgacagtagaagcatcgatgcatgctgcacagaatgaacgccttcctcttcgagttgtggtgcaggttctctattttgagcaggtcagagctgcttccaatgctcgtgcactcggcaacagtccacacaattctacaaacactcatgtcaacggagacgaggagtgtgcgaaatcggggggagaaagctgccaatccctcgacaaccagatgtgtcacatgaagattagagatgaacagttgcaaaagaatgggaagttgaacaagaagagtagcaagaacagcagaagtggcatgcagttgctgccatctaggtcaaggagaatctttgacaagctatggattgttggtaagggacaaggagaaaatagaagctcagagacctcagggagttccaatagccctacttctgtagtccctggagataccaaatcatctggctcatcattgagacacaggaggcattctatctcttagggaattttcataatttaaacttgtgcaaggagagttaagttgctgtgaagtgtaaaaagtgttaggatgttaaatcttgtgttatttaatatgcatatgcaca

SEQ ID NO: 50 - protein, 616 aa, Glycine max
MKFMKLGSKPDALQSDGKSIRYISSELATDIIITVGEVKFHLHKFPLLSKSNSLQKLLSKANEENADEIQLDDFPGGPKAFEICAKFCYGMTVTLNAYNVVAARCAAEYLEMTEDIDRGNLIFKIEVFLTSSIFRSWKDSIIVLQTTKSLLPWAEDLKIVGRCIDSIASKTSVDPANITWSYTYNRKLSELDKIVEDKITPQEKIEPVPKDWWVEDICELDIDLYKRVMITVKSKGRMDGVVIGEALKIYAVRWLPDSVDALVSDAHAWRNKSLVETIVCLLPCDNGMGCSCSFLLKLLKVAILVEADESSRGQLMKSIGLKFHEASVKDLLIPARFPQNTKYDVDLVQDLLNLYMTNIKGSRDVVVEEKKDRANDESILGQRSLLNVGKLVDGYLGEIAHDPNLSLSSFVALSQSIPEFARPNHDGLYRAIDVYLKEHPSLTKSERKNICGLMDVKKLTVEASMHAAQNERLPLRVVVQVLYFEQVRAASNARALGNSPHNSTNTHVNGDEECAKSGGESCQSLDNQMCHMKIRDEQLQKNGKLNKKSSKNSRSGMQLLPSRSRRIFDKLWIVGKGQGENRSSETSGSSNSPTSVVPGDTKSSGSSLRHRRHSIS

SEQ ID NO: 51 - DNA, 1875 bp, Zea mays
atgaagttcatgaagcttggatcaaatccggatacatttcagggagatgggaatgaagtcagtattgcggcatctgaattggtgagcgacatcactgttcgtatagggactacaaagttttaccttcacaagtttccgctcctatccaagtgtgctcgcttccagaagttgattccaactactggcgatgaaaatattgagatccacatccatgatattcctggtggtgccaaggcttttgagatctgtgccaagttttgctatggcatgattgtcacactcaatgcctacaatgtgattgccgcccgctgtgccgcagagtacctggagatgaacgagactgttgacaagggcaatctgatctacaagatcgatatcttcctcagctcaagcatacaccggagctggaaagactccataattgttcttggtacaacaaaggctcatttaccttgggcagaggatcttaagctggtcagccactgcattgactctgttgcttcaaaagcctctattgacgtttcaaaagttgaatggtcatacacatacaaccgtaagaagctcacagttgagaatggtcatgattcgccatggaatggagtcaagcagcagcagtttgttccaaaggattggtgggtcgaagatctgacagatctcgacattgacgcctacaagcaagtcataacagcagtcaaaaccaagggtacggtgcccaaggatgtgatcggtgcggcgattaaagcctacacttacaagaagctcccgtcactaagcaaggtctcgatgatccatggcgacgcaaaagttcgggctatgctagttaccatcacatgcctgttgccatccgagaaaggttcggtttcgtgcagcttcctcttgaagttactgaaggcaacaaacttgctcaagtgtggagagatgtgcaggaaagagcttatgaagcgcatcgcacggcaactggaagaagcgtcagtgtcagatcttctgatccctacggtggatggggataccaccgtttacgacatcgacctgattcttagcattgttgaagaatttgtcagacaagatagcaagaatgcccagagacataacggtggtgaggtgaacgaccacgtatctgctcctagtgcgtcaatggttgcagtggccaaaactgtcgatggatatctcgcagaggttgcaagggacccaaatataccggtctacaagtttttcagcctagctgagacggtttcgggaggttcaaggccggttcatgatggactgtatcgtgccattgatatgtatctgaaggagcacccaagcttgggcaagagcgacaagaagaggctatgtgccctgatggactgcaagaagttgtcgcccgacgcgtgcgctcatgccgtgcagaacgagcgcctgcctctgaggatcgtggtgcaggtgctgtaccacgagcagacaagggcctgtgccgcagccaccgccatccgagccgacagcatcggcatcggctcctacgagagctcaaggtcaggcgccacaacgaacacagaggacgagtgggacggcgtcatggccgtggaggacctcagcctgtccaagacgacgaagctggtggacaaatgcgacacggcggccagtacggtcgtcgtggggaaggacaaggaccacggtggcaacaagggcgccaccggcagggtggccaaaggtggcgctggcgcggcgacaccaaagaaggcgctcgggaaaacggtgtccggcaaagggcagaccggggagcgcagcagctcggactcatccgacagcgccgtcgccgtcgcagtcttgaccagccaggagcatcccaagaggacgacccgcgcaaggaacgccgccaagtcggctgctgcgtag

SEQ ID NO: 52 - protein, 624 aa, Zea mays
MKFMKLGSNPDTFQGDGNEVSIAASELVSDITVRIGTTKFYLHKFPLLSKCARFQKLIPTTGDENIEIHIHDIPGGAKAFEICAKFCYGMIVTLNAYNVIAARCAAEYLEMNETVDKGNLIYKIDIFLSSSIHRSWKDSIIVLGTTKAHLPWAEDLKLVSHCIDSVASKASIDVSKVEWSYTYNRKKLTVENGHDSPWNGVKQQQFVPKDWWVEDLTDLDIDAYKQVITAVKTKGTVPKDVIGAAIKAYTYKKLPSLSKVSMIHGDAKVRAMLVTITCLLPSEKGSVSCSFLLKLLKATNLLKCGEMCRKELMKRIARQLEEASVSDLLIPTVDGDTTVYDIDLILSIVEEFVRQDSKNAQRHNGGEVNDHVSAPSASMVAVAKTVDGYLAEVARDPNIPVYKFFSLAETVSGGSRPVHDGLYRAIDMYLKEHPSLGKSDKKRLCALMDCKKLSPDACAHAVQNERLPLRIVVQVLYHEQTRACAAATAIRADSIGIGSYESSRSGATTNTEDEWDGVMAVEDLSLSKTTKLVDKCDTAASTVVVGKDKDHGGNKGATGRVAKGGAGAATPKKALGKTVSGKGQTGERSSSDSSDSAVAVAVLTSQEHPKRTTRARNAAKSAAA

SEQ ID NO: 53 - DNA, 1980 bp, Zea mays
ctcttctttcttgttcaagatgaagttcatgaagcttggatcgaatccggatacctttcaggacgatgggaatgaagtcagtattgcggcatctgaattggtgagcgacatcactgttcgtatagggactacaaagttttaccttcacaagtttccgctcctctccaagtgtgctcgctttcagaagttgattccaaccactggtgatgaaaatattgagatccacatccatgatattcctggtggcgcaaaggcttttggggtctgtgccaagttttgctacggcatgattgtcacactcaatgcctacaatgtgattgcagcccgctgtgctgcagagtacttggagatgaatgagactgttgacaagggcaatctgatctacaagattgatgtcttcctcagctcaagcatattccggagctggaaagactctattattgttcttggtacaacaaaggctcatctaccttgggcagaggatcttaagctggttagccaatgcattgactctgttgcttcaaaagcctctatcgacgtttcaaaagttgaatggtcatacacatacaatcggaagaagctcccaactgagaatggtcatgattcgccgtggaatggagtcaaccagcggcaggttgttccgaaggattggtgggtagaagatctgacggatctcgacattgatgcctacaagcaagtcataacagcagtcaaaaccaagggtatggtggccaaggacgtgatcggtgaggcaattaaagcctacacttacaagaagctgccgtcactaagcaaggtctcaatgatccatggcgacgcaaaagttcgggcgatgctagttacaatcacatgcctgttgccatctgagaaaggctcagtttcatgcagcttcctcttgaagctactgaaggcaaccaatttgctgaagtgcggcgatatgtgcaggaaagagcttatgaagcgcattgcacggcaattggaagaagcatcagtgtcagatcttctgatccctgcagtggatgaggacaccagtgtttacgacattgacctgattcttagcattgttgaagaatttatcagacaggatagtaagaatgcccagagacataatggtggcgaagtgaatgaccatgtatcagctcctagtgcatcaatgattacagtggcaaagattgttgatgggtatctggcagaggttgcgaaggacccaaatataccggtctacaagtttttcagtctcgctgagacagtctcggccaattcaaggccagtccatgacgggctctatcgcgccattgatatgtatctgaaggagcacccaagcttgggcaagagcgacaagaagaggctctgtgccctcatggactgcaggaagttgtcgcccgatgcatgcgctcacgctgtgcagaacgagcgcctgcctctgagggttgtggtgcaggtgctgtaccacgagcagacgagggcttctgcagcagccaccgtccgagctgacagcatcggcatcggctcctacgagagctcaaggtcaggggccacaacaaacacagaagacgagtgggacggcgtcatggccgtggaagacctcagtctgtcgtccaagacgacgaagctagagaaatgtgacgcagctagcaccgtctcggagaagaaccacggcggcaacaagggtgccaacggcagggcgaaaggtggcgtgatgccgaagaaggcggtcgggaggacgacgatgtcaggcaacaaagggcaggctggggagcgcagcagctcggactcatccgacagcgccattttgccttgccagctggagcatcccaagagggccgcagcaaggagcaccaccaagtcagccgctgtgtagtagtagctgcccggccctttgctcagtgagatgctcaaggagcactaggttttagcagtcgtcgtccgttcttttctttgcgttatgaatgttgcatagaagcaaggaccatgttctta

SEQ ID NO: 54 - protein, 613 aa, Zea mays
MKFMKLGSNPDTFQDDGNEVSIAASELVSDITVRIGTTKFYLHKFPLLSKCARFQKLIPTTGDENIEIHIHDIPGGAKAFGVCAKFCYGMIVTLNAYNVIAARCAAEYLEMNETVDKGNLIYKIDVFLSSSIFRSWKDSIIVLGTTKAHLPWAEDLKLVSQCIDSVASKASIDVSKVEWSYTYNRKKLPTENGHDSPWNGVNQRQVVPKDWWVEDLTDLDIDAYKQVITAVKTKGMVAKDVIGEAIKAYTYKKLPSLSKVSMIHGDAKVRAMLVTITCLLPSEKGSVSCSFLLKLLKATNLLKCGDMCRKELMKRIARQLEEASVSDLLIPAVDEDTSVYDIDLILSIVEEFIRQDSKNAQRHNGGEVNDHVSAPSASMITVAKIVDGYLAEVAKDPNIPVYKFFSLAETVSANSRPVHDGLYRAIDMYLKEHPSLGKSDKKRLCALMDCRKLSPDACAHAVQNERLPLRVVVQVLYHEQTRASAAATVRADSIGIGSYESSRSGATTNTEDEWDGVMAVEDLSLSSKTTKLEKCDAASTVSEKNHGGNKGANGRAKGGVMPKKAVGRTTMSGNKGQAGERSSSDSSDSAILPCQLEHPKRAAARSTTKSAAV

SEQ ID NO: 55 - DNA, 1980 bp, Oryza sativa
agagataagatttaagatcagcgcgtgtgcgtctgttctttgcctgtttcaagatgaaattcatgaagcttggatcgaacccggatacttttcaggatgatggcaatgaagtcagcattgtggcaactgaattagtgagcgatgtcactgttcgcatagggactacaaagttttaccttcacaagttcccacttctgtccaagtgcgctcgctttcagaagatgatcccaactactggtgatgaaaacattgagattcacatccatgatattcctggtggcgccaaggcttttgagatctgtgccaagttttgttatggtatgattgtcacgctcaatgcctacaatgtcattgcagcccgctgcgccgcagagtacttggagatgcatgaaactgtcgacaagggtaatctcatctacaagattgaggtcttcctcagctcaagcattttccggagttggaaggactctatcattgttcttggtactacaaaggctcacttgccttggtcagaggattttaagctggttagccactgcatcgactccattgcttcgaaagcctccactgacacctccaaggttgagtggtcatattcatacaatcgcaagaagctcccaactgagaatggccttgatttggaatggaatggagtcaagaagcagcagtttgtgccgcatgattggtgggtcgaggatctcgcagatctcgacattgattcatacaagcaagtcataacagcaatcaagaccaagggtatggtgcccaaggatgtgattggggaggcaatcaaggcttacacatacaagaagctcccttccttaagtaaggtttcaatggtccatggcgacgcaaaagttcgggccatgctagttaccatcacatgcttgttgccatcagagaaaggatcagtttcatgcagcttcctcttaaagctactgaaagcaacaaatttgctaaaatgtggagagctctgcaggaaagagcttatgaagcgtattggacggcaactagatgaagcgtccgtgtcggatcttctgattcctacagtggatggggagaccacagtttatgacattgacatgatactcagtatagtggaagaatttgtcaggcaagacagtaagaatgctcagaaacataatggtggtgaagtggacagtcatgtccaagcacctagtgcatccatgatcaaagtggctaaagtggttgatgggtatcttgctgaggttgcaaaagatccaaatacacccattttgaagttcattcatctagccgagacaatgtcgataaattcaaggccagttcatgatgggctataccgcgccatcgacatgtatctgaaggagcacccaagcttgggaaagaatgagaagaagaagctgtgcagcctcatggactgcaagaaactctcaccggacgcgtgtgctcacgcggtgcagaacgagcggctgccactgaggaccgtggtgcaggtgctgtaccacgagcagacgagggcttctgcggcggtcaccatccgagccgacagcatctgcgtcggctcgtacgagagctcgaggtcaggcgccaccaccaacacggaggacgagtgggacggcgtcatggtcgtggaagacctcagcctgtccaccaagaccaccaccaagctagacggcgcagccaatagccattgcagcaacggcaaggcgaagaaaggcggcgcgtcgacgccgaagaaggcggctcaccggaggacgacgacggtgccaaccggcaaagggcagtccggtgagcgcagcagctcggactcgtcggacagtgcgatcttgcagaagctggagcttcccaagaggaccccctccaggagcacgaagccggctgctgtgtagcgttagcttcctgcatgaattctcagtggattcgtaaggatcgttaggttcttagctgctgcttcttttctttctttcttcatgaatgttgcatagaagcaagaactaa

SEQ ID NO: 56 - protein, 605 aa, Oryza sativa
MKFMKLGSNPDTFQDDGNEVSIVATELVSDVTVRIGTTKFYLHKFPLLSKCARFQKMIPTTGDENIEIHIHDIPGGAKAFEICAKFCYGMIVTLNAYNVIAARCAAEYLEMHETVDKGNLIYKIEVFLSSSIFRSWKDSIIVLGTTKAHLPWSEDFKLVSHCIDSIASKASTDTSKVEWSYSYNRKKLPTENGLDLEWNGVKKQQFVPHDWWVEDLADLDIDSYKQVITAIKTKGMVPKDVIGEAIKAYTYKKLPSLSKVSMVHGDAKVRAMLVTITCLLPSEKGSVSCSFLLKLLKATNLLKCGELCRKELMKRIGRQLDEASVSDLLIPTVDGETTVYDIDMILSIVEEFVRQDSKNAQKHNGGEVDSHVQAPSASMIKVAKVVDGYLAEVAKDPNTPILKFIHLAETMSINSRPVHDGLYRAIDMYLKEHPSLGKNEKKKLCSLMDCKKLSPDACAHAVQNERLPLRTVVQVLYHEQTRASAAVTIRADSICVGSYESSRSGATTNTEDEWDGVMVVEDLSLSTKTTTKLDGAANSHCSNGKAKKGGASTPKKAAHRRTTTVPTGKGQSGERSSSDSSDSAILQKLELPKRTPSRSTKPAAV

SEQ ID NO: 57 - DNA, 1917 bp, Oryza sativa
atgaagtatatgaagcttggatcaaagccagatgtgtttcagacagagggaaacaatatcaggtttgtcgcaactgagttggcaacggacatcgtaatcattgtaggggaagtgaagttttatcttcacaagttccctcttttgtcgaagagttcgcgattgcaaacattggtggcatcgacaaacgaagagagcaacgacgaaatagacatctccgacatcccggggggacctgcagcattcgagatctgtgccaagttctgctatggcatgatcgtcaccctcaacgcgtacaacgtcctcgcagcccgctgcgcagccgagtatcttgagatgttcgagacgatcgacaaggggaacctcatctacaagatcgatgtgttcctgtcctcaagcatattcagaacctggaaggactccatcatcgttcttcagacaaccaagtcactgctgccatggtcggagaacctcaaggtgatcaaccactgcgtcgattcgatcgcgacgaaggcctccatcgatccgtcagaggttgagtggtcatacacctacaacaggaggaagctaccgtcggagaacggcctcgactcgcactggaacggcgtaaggaagcagcagatggtgcccagagactggtgggtagaggatctctgtgaccttgagatgtgcctgtacaagaaggtcatcatggccatcaaggccaaggggagaatcagcagtgaggtgattggtgaggcactgagggcttatgctcacaggaggctgttcagctcactggaaagtgctgtcagcaatgggcttgactgcacaaggcactctgcagctcttgagaccatcatctccctgcttccatctgaggaaggttcagtcccatgctctttcctccttaagcttctcagagcctcatgcttgttgggatcagatgaggcgtgccgcgacaatcttacgaagaggatcggcgcgaagctggatgaggcttcggtctcggacctccttatccccgctaactccgacgaggccgccatgtacaatgtggacatgatctcggcgatgctggaggagttcatggcgcagcaccgcgaggacgatgatggcgccaagctacaagaggatgatgatcaggaagccatggatggtgatgatgacaacctgaatggtgtctcccgcagctcgaagctcgcgatcgcgaagctggttgatgggtatctggctgagattgcgaaggaccccaacctcccactctcgaagttcatcgccctcactgagatggttcccctcgcaactcggcccgtgcacgacgggctataccgcgccatcgacatgtatctcaaggagcatccgggtttaacgaagggggagaagaagaggctgtgcgggctgatggactgcaagaagctgtcgccggaggcgagcatgcacgccgtgcagaacgagcgcctcccgctccgcgtcgtcgtgcaggtcctcttcttcgagcaggtccgggcggcgtcgtcggcgtcggcggcggcggccgccgacatgccgccggcagcgcgctccctcctccccagggagcaggacggcaactcgtacggcagctcgaggtcgacggcgaccgaggacgaccagtgggcgccgccgccgacgacgacgtccgtcgacgtcacctccttccggtcgatgagcctcgccaacaacaagaacggcggcgtctccggcggcggcggcgatcaggccgcctgcaagaagccgccgccgtcgtcggcgtcagcgaaggggagcggcggcggcgggctgatgccgaagaagatactgagcaagctgtggtctggcaaggcgagcagcggcgagaacagcagctccgacacgtcggagagccccggcgaggagacgaggtcgacgccgtcgcggaacacgaggcattcggtgtcctag

SEQ ID NO: 58 - protein, 638 aa, Oryza sativa
MKYMKLGSKPDVFQTEGNNIRFVATELATDIVIIVGEVKFYLHKFPLLSKSSRLQTLVASTNEESNDEIDISDIPGGPAAFEICAKFCYGMIVTLNAYNVLAARCAAEYLEMFETIDKGNLIYKIDVFLSSSIFRTWKDSIIVLQTTKSLLPWSENLKVINHCVDSIATKASIDPSEVEWSYTYNRRKLPSENGLDSHWNGVRKQQMVPRDWWVEDLCDLEMCLYKKVIMAIKAKGRISSEVIGEALRAYAHRRLFSSLESAVSNGLDCTRHSAALETIISLLPSEEGSVPCSFLLKLLRASCLLGSDEACRDNLTKRIGAKLDEASVSDLLIPANSDEAAMYNVDMISAMLEEFMAQHREDDDGAKLQEDDDQEAMDGDDDNLNGVSRSSKLAIAKLVDGYLAEIAKDPNLPLSKFIALTEMVPLATRPVHDGLYRAIDMYLKEHPGLTKGEKKRLCGLMDCKKLSPEASMHAVQNERLPLRVVVQVLFFEQVRAASSASAAAAADMPPAARSLLPREQDGNSYGSSRSTATEDDQWAPPPTTTSVDVTSFRSMSLANNKNGGVSGGGGDQAACKKPPPSSASAKGSGGGGLMPKKILSKLWSGKASSGENSSSDTSESPGEETRSTPSRNTRHSVS

SEQ ID NO: 59 - DNA, 1980 bp, Oryza sativa
ctctgagagggagggaggaggaggaagaagaagaagaagatgaagttcatgaagctgggctccaagcccgacgccttccagtccgacggcgccgacgtcagatatgtgatctcagacctcgcaacagatgtcattgtgcatgtcagcgaagtcaaattttacttacataagtttccccttctgtccaagagcagcaagcttcagaggttagttattaaggccaccgaggaggggactgatgaggtccacatagatggctttcctggaggagtgacagcgttcgaaatatgcgctaagttctgctatgggatggttgttaccctcagtcctcacaatgttgttgcagcaagatgtgcagcagagtaccttgaaatgactgaagatgtggacaagggaaacttgatattcaaaatcgatgtcttcataaattctagcatcctccgtagctggaaagactctatcattgttctccagagcacaaaagcactattaccttggtccgaagagctgaaggtcatcggcagatgtatagatgctattgcttcaaagacatctgtggatccagctaatgtaacctggtcatacagtcacagcaggaaaggaatgagctgtactgaaatagttgaatcaacaggaagaacatcaatagctcctaaagactggtgggttgaggacttatgcgagctcgatgttgacctctacaagagagtgatggttgctgtcaaatccaaggggagaatgtcacctgaattgataggggaagcccttaaagcatatgctgtcagatggctgccagattcttatgatgctttggtcgcggaggattacatgagaaggaaccaatgtttggtcgagactataatatggttattgccgtcagataagacatcaggttgttcttgcaggtttctcttgaaactgttgaaagttgctatattggttggagctgggcaacatgtgaaggaagaacttatgagaaggattagtttccagttgcacaaagcttcggttaaggatctcttactacctgctgcttcacccagtgatggcgcacacgatgttaagttagtccataatcttgtgcagagatttgttgcaagaacagcgatgtcccacaatggtggttttgttgagaaatctgatgacaaaatgattgagcttaactttgaacaagaatccactttggctttaggagagctggttgatggctatctatctgaagtagcttcagatccagacctttcattgtctacatttgttgaactagcaacagcggttccagaagcagctaggcctgttcatgacagcttgtactcagctgttgatgcatatctcaaggagcatccaaacataagtaaagccgacaagaagaagatctgcggtctgatagatgtcaagaagctttcaacagatgcaagcatgcacgccacgcaaaacgaccgcctaccgctccggttggtggtgcaggtcctcttcttccaacagctgagggcgggttccagcaatgcgcttgctctaacggatggtggaggacacacgtgcgcaaagcctattatgaaggatcagagtgacatttgcgaaagacggataccaaggcatccaaactcgctgaacaagcaagcgaccagcctaagcgcgagagaggtcgagcaccggaagagcgagcacaggggaggaggcaggaacagcttcaaggaccagcttggcggcttccttctgcagtcaaggtccaggaggatctttgacaagatatggagcagcaaggggcagggagagaatggcaaaggttcagagacgtcaggtagctcacagagcccgccattgtcggctaagcctgcagatgtgaagccctctcccctcccacctcttaggaacaggagatattctgtttcatagggttcttgtttacttcttttaatttagctctgggttaggttgtacagaaaagataagttgatcagctggctgcacatcattttt

SEQ ID NO: 60 - protein, 618 aa, Oryza sativa
MKFMKLGSKPDAFQSDGADVRYVISDLATDVIVHVSEVKFYLHKFPLLSKSSKLQRLVIKATEEGTDEVHIDGFPGGVTAFEICAKFCYGMVVTLSPHNVVAARCAAEYLEMTEDVDKGNLIFKIDVFINSSILRSWKDSIIVLQSTKALLPWSEELKVIGRCIDAIASKTSVDPANVTWSYSHSRKGMSCTEIVESTGRTSIAPKDWWVEDLCELDVDLYKRVMVAVKSKGRMSPELIGEALKAYAVRWLPDSYDALVAEDYMRRNQCLVETIIWLLPSDKTSGCSCRFLLKLLKVAILVGAGQHVKEELMRRISFQLHKASVKDLLLPAASPSDGAHDVKLVHNLVQRFVARTAMSHNGGFVEKSDDKMIELNFEQESTLALGELVDGYLSEVASDPDLSLSTFVELATAVPEAARPVHDSLYSAVDAYLKEHPNISKADKKKICGLIDVKKLSTDASMHATQNDRLPLRLVVQVLFFQQLRAGSSNALALTDGGGHTCAKPIMKDQSDICERRIPRHPNSLNKQATSLSAREVEHRKSEHRGGGRNSFKDQLGGFLLQSRSRRIFDKIWSSKGQGENGKGSETSGSSQSPPLSAKPADVKPSPLPPLRNRRYSVS

SEQ ID NO: 61 - DNA, 1980 bp, Gossypium hirsutum
tgccactgcaatatttgtagatataagaggaagtgaaattgtgtgagtgttggaaaatacaatttgtttagccaagatgaagtttatgaaactgggatccaagcctgattcatttcagactgatggaggttatatcaggttcgttgcaagtgagttagcaactgacatggtcgttaacgttggggatgtgaaattctatctgcataagtttccgttgttatccaaaagtgcacgcttgcagaagctggttgcagcaacaaatgatgcgaatggtgatgaaatccacattcaagatatacctggtggaccagcagcatttgaagtatgtgctaagttttgttatggaatgactgtcactctcaatgtgtacaatgttgtcgcagctcgatgtgctgctgaatacctcgagatgtatgaaactattgagaaaggaaatctcatctataagattgatgtcttcctgaattcgagcatattcaggagctggaaagattccatcattgttcttcaaacaacaaaatctctgcttccatggtcggaggaactaaaggttgtcagccactgccttgattcgatagcttccaaggcttcaattgatacttccaaggtggaatggtcatatacctataattggaaaaagatagcatcagagaatggaaatagtctacagtggaatgatgcaagaaaaccacaaatggttccgaaggactggtgggtagaagatctttgtgagcttcctattgatctatacaagcgcgtgattgtgacgattaaaacaaaaggcagagtttctggagatgtaattggagaagccctaaatgcatacaccctaagaagattgcctggttttagcaagggtgtgaaccagaatattgatttcgtaaagtatcggtcattggtggagaccattgtgtggctgctaccaactgagaaaggagttgtttcttgtagcttcttgcttagattattaagagcagcaatttttttggattgtggagaaacagagagaaatgagttgatgaggagaattgctcagcagcttccggaggcaaagatgaatgatctattaattcgagctccacctggtgaagcaacagtatatgatgttgacatagttcaaaacttggttgaggagtttgtgactcacagttctcaaaccgaacccgtagataatgaattcctcgggagtagaagccccaagtttggacctgatgcttctaaagtgttggtagcaaagctaattgactgttacctagctgaaattgcacaagatccgaatttacacctttcaaaatttgtcaatcttggtgaaactatagctagcttctcgagaccttctcatgatggactttaccgtgccatagacatgtacctcaaggaacatcctggaatcagcaagagtgagagaaagagaatatgcaagttaatggactgcaggaagctatcggctgaggcctgcatgcatgctgtgcaaaatgagagactcccgttgcgagtcgtcgtgcaggtcctcttctttgagcaagtcagggctacagcatcagctgaaaacagcacgtccgatctcccagggtccatcagggcccttctccctggtgggtctcatggcagctcaaggtccactacaacgaacaccgaagaggattgggattcggtgccaacagctgatgatatcaaagctttgaaaggggagcttgctactctaagattaggcagtaatgatagtggaaatgaagcagctaaatccgaagtcgaaaaggttgctgctagtcgaatgaaaggtttggtgatgtcaaaaatattctcgaaactttggtcgagtaaggaaagacatggtgaaattagcagctcggatacatcagacagccatggatctgtgaatgcggaagaaacaaagtccaccccttctagaagtaggagacattcactatcttagttcaggatgatgcccttagcagagcattaaaacttgccttttgt

SEQ ID NO: 62 - protein, 619 aa, Gossypium hirsutum
MKFMKLGSKPDSFQTDGGYIRFVASELATDMVVNVGDVKFYLHKFPLLSKSARLQKLVAATNDANGDEIHIQDIPGGPAAFEVCAKFCYGMTVTLNVYNVVAARCAAEYLEMYETIEKGNLIYKIDVFLNSSIFRSWKDSIIVLQTTKSLLPWSEELKVVSHCLDSIASKASIDTSKVEWSYTYNWKKIASENGNSLQWNDARKPQMVPKDWWVEDLCELPIDLYKRVIVTIKTKGRVSGDVIGEALNAYTLRRLPGFSKGVNQNIDFVKYRSLVETIVWLLPTEKGVVSCSFLLRLLRAAIFLDCGETERNELMRRIAQQLPEAKMNDLLIRAPPGEATVYDVDIVQNLVEEFVTHSSQTEPVDNEFLGSRSPKFGPDASKVLVAKLIDCYLAEIAQDPNLHLSKFVNLGETIASFSRPSHDGLYRAIDMYLKEHPGISKSERKRICKLMDCRKLSAEACMHAVQNERLPLRVVVQVLFFEQVRATASAENSTSDLPGSIRALLPGGSHGSSRSTTTNTEEDWDSVPTADDIKALKGELATLRLGSNDSGNEAAKSEVEKVAASRMKGLVMSKIFSKLWSSKERHGEISSSDTSDSHGSVNAEETKSTPSRSRRHSLS

SEQ ID NO: 63 - DNA, 761 bp, Solanum tuberosum
tgttagatgattctgagtcatggacaagggaacttgccctatctctgattcttgaaatgctgaagaatcagaaaaatgctatggaggactctgttgagatcataattgaaaagctgctccatgtgaccaaggatgatgttgcaaaagttgcaaatgaagctgagaattgtttatctacaattttgtcccagtacgacccattcagatgcctaagtgttattgttcctttacttgtcaccgaagatgagaagactcttgtaacttgtattaactgtttgacgaagcttgttggaaggctctctcaggaggaattgatgtctcagcttccttcattcttgccttccctgtttgatgcttttggaaaccagagtgctgatgtccgcaagactgttgtattctgtttggttgacatatacatcatgctgggcaaagcatttatgccatacttggaagggctgaacagcacacagttacgattggtgaccatttatgcaaatcgaatatcacaggccagaacaggtactccggtagatgcaagccatagttaggtttttttctttttctttttttgggtattcattgtaggtatagatgtgtgtatttgtgatttaatatttgatgccttttgtatatctgtgtgaatgtgtatttcttttttggtgggtgaggttgtattcttcttttttcttttctgctaattggttggtagttttggagtgagaacaattgtgatattgtacacagtgaactttgagctcata

SEQ ID NO: 64 - protein, 181 aa, Solanum tuberosum
LDDSESWTRELALSLILEMLKNQKNAMEDSVEIIIEKLLHVTKDDVAKVANEAENCLSTILSQYDPFRCLSVIVPLLVTEDEKTLVTCINCLTKLVGRLSQEELMSQLPSFLPSLFDAFGNQSADVRKTVVFCLVDIYIMLGKAFMPYLEGLNSTQLRLVTIYANRISQARTGTPVDASHS

SEQ ID NO: 65 - DNA, 267 bp, Solanum tuberosum
aatcgaatatcacaggccagaacaggtactccggtagatgcaagccatagttaggtttttttctttttctttttttgggtattcattgtaggtatagatgtgtgtatttgtgatttaatatttgatgccttttgtatatctgtgtgaatgtgtatttcttttttggtgggtgaggttgtattcttcttttttcttttctgctaattggttggtagttttggagtgagaacaattgtgatattgtacacagtgaactttgagctcata

SEQ ID NO: 66 - DNA, 1112 bp, Artificial sequence
  synthetic
ggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcga

SEQ ID NO: 67 - DNA, 1999 bp, Arabidopsis thaliana
gtagtgcccttcatggataccaaaagagaaaatttgatttagtgcatacatataacaatataacgccgcataataatactgtataaaacagtcatgtaacgatatgacagcagtaatacagttccaagagacgttataatcgtatgcaatcatatgcttgcgtagattttccaacagttttgtttcgttgataggaggaactcaacactctagggtagtgattggtagacactattagcacaaaaaatattaattttactctgatgtttaccaaaaaagttaccaatcaaatatttaagagatcgtactcttccacggcgactctaaaaaccaaagatataggttagactcataactactttataaagaaaatgtttaacgataactaccgagatctaataaataaaccttcattttcaagtatattatatttgcttcttttgtttatatatcaaaccaagttctggtttataaaaatattagataaaactcgtctaaataggtaggtgtaaaataaaattttaaatttttatcgataatatttaaaatttgaaaagttaataatgatccacacattttttctaatatttaatttagtaatttttgtattaaataaaatttcaatcatatacattcgatttttctatacattttaactatctatttctgcataataaactgtattttcattttatacgcttcatcttatggatgatatttaaattttaaatagtaattcatacactttttaatatttaatttagtattttcttaaatccaaattttaatcttacaatttaaatatctactttaacataatacaaatacaatttaatttcattgtattaaattcaaatataatttgattataataaaatacaatttaattctaaaaagtccatcttagattttaattttcctttttagttttgaaaattaaaaatttaaatttattagatatatatgttactttttcagttttcctatttatttaagaaaaaaatattttttaacacatgtcaacttgtaaacaatagactgaacacgtcattttatattatgtttagttttgaaaattaaagttaattaaatatttatatttcttttttttagcttttctaattatttttaaaatagtaaatatttttaatacaaatcaatatctgaacaatagatttgatacataacataatcctataaattattaacttggaaaacgatagtttatataataaaattattttcttaagttctctaaccataacaattaaactatattttagcgaagaaaagaagagaataccgagagaacgcaacttgcactaaaagctaccactttggcaaatcactcatttatattattatatactatcacctcaattcaatcgaaacctcaaaataacactaatatatacacaaagaaacaacagaataacaccgaagaatataggtttaggaaaatccagaatttgttgagactaaagagatcaaattttcgatacaaggttttgctcaatttgtattttcataataaaattctttatttcaccatagacttacatgattagtttttcttttaataaaaaaaaacacgcgacatgaaaattatattatctcagtgttgtcgaatttgaatttgaattttgagttaaatactacacatttgttgacaacttattaaactttacaagtctgctacaaatattgtcaaatatttactaattaatggaccaaaatcctctaacttgcaaatttgtatctacatcaacttaaaaattaggaatatgcgacccaaaaaaaaaaaaactaggaataataataaaaaaatggaatgatgtggaggaagctctttactctttgagaggaagtttataaattgaccacacatttagtctattatcatcacatgtattaagacttgacaacttgtctttctcacaccaaacccctctcctctgtttcataacatctgctctttcttttttttcctaagccccta

SEQ ID NO: 68 - DNA, 609 bp, Glycine max
gaagccacgtcatgaagagtatatcatttcagtaatgttttgagacgcctctataatgctttaccaacaaaacaaaacaaaaaaaagaacatttgaaaccatttgtattaaaaaaaaaaaggtatattaggccataatattataggtaacatgaaatatcaaatgacacgcaagagttttgtcaaaaatgaaaccatcacacatcagagattatggcaaataatgttttgtgtgtctcttgcttcacccataacataagcctctataactggagagaagaaaaaaaaaagtggaggggctagggtgggaatttggaagaatacagttatattgagcattgagcaagttgatagaaagcttctcaatttgtacaaaatttgcatccacatgattattaaagacgtagacagcacttcttccttctttttttctataagtttcttatatattgttcttcatgttttaatattattactttatgtacgcgtctaacagtagtcctcccaaactgctataaatagagcctcttcaacgcacctcttggcagtacaaaaattattcatctcttctaagttctaattttctaagcattcagtaaaagaactaacc

SEQ ID NO: 69 - DNA, 325 bp, Petroselinum crispum
aattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctc